(19) United States
(12) Patent Application Publication
(10) Pub. No.: US 2025/0306018 A1
(43) Pub. Date: Oct. 2, 2025

(54) METHODS FOR CHARACTERIZING INTERACTIONS BETWEEN ANALYTES

(71) Applicant: Nautilus Subsidiary, Inc., Seattle, WA (US)

(72) Inventors: Gregory KAPP, San Carlos, CA (US); James Henry JOLY, San Mateo, CA (US); James SHERMAN, Kingston, WA (US); Torri Elise RINKER, San Francisco, CA (US); Raymond MAK, San Francisco, CA (US)

(21) Appl. No.: 19/235,056

(22) Filed: Jun. 11, 2025

Related U.S. Application Data
(63) Continuation of application No. 17/933,424, filed on Sep. 19, 2022.
(60) Provisional application No. 63/247,160, filed on Sep. 22, 2021.

(51) Int. Cl. G01N 33/543; B01J 19/00; C40B 60/06; G01N 21/64; G01N 21/77; G16B 20/30

(52) U.S. Cl. CPC G01N 33/54366; B01J 19/0046; C40B 60/06; G01N 21/6428; G01N 21/77; G16B 20/30; B01J 2219/00623; B01J 2219/00659

(57) ABSTRACT

Methods and systems for identifying and/or quantifying polypeptide binding interactions of ligand-binding polypeptides are disclosed. Detailed methods include methods for identifying binding ligands of ligand-binding polypeptides and methods for assessing changes in binding behavior due to alterations of ligand-binding polypeptides. Detailed systems include array-based systems that permit detection of ligand binding interactions at single-analyte resolution.

### CROSS REFERENCE TO RELATED U.S. APPLICATIONS

This is a continuation application of the U.S. application Ser. No. 17/933,424, filed on Sep. 19, 2022, which claims priority to U.S. Provisional Application No. 63/247,160, filed on Sep. 22, 2021, each of which are incorporated herein by reference in their entirety BACKGROUND

Ligand-binding polypeptides include numerous species of polypeptides that are capable of forming binding interactions with other molecules, including other polypeptides and various small molecule compounds. Ligand-binding polypeptides can form biologically- or clinically-relevant binding interactions with particular binding ligands, such as hormones, inflammatory polypeptides, and pharmaceutical compounds. Pre- or post-translational alterations to a ligand-binding polypeptide can affect the binding behavior of the ligand-binding polypeptide.

### SUMMARY

In an aspect, provided herein is a method, comprising: a) providing a single-analyte array comprising a plurality of analyte binding sites, wherein each analyte binding site of the plurality of analyte binding sites is optically resolvable at single-analyte resolution, and wherein each analyte binding site comprises one and only one binding ligand of a plurality of binding ligands, b) contacting the single-analyte array with a plurality of polypeptides, wherein the plurality of polypeptides comprises a proteomic sample, c) binding a polypeptide of the plurality of polypeptides to the one and only one binding ligand at an analyte binding site of the plurality of analyte binding sites, d) detecting a presence of the polypeptide of the plurality of polypeptides at the analyte binding site of the plurality of analyte binding sites, and e) determining an identity of the polypeptide of the plurality of polypeptides bound to the one and only one binding ligand at the analyte binding site of the plurality of analyte binding sites.

In another aspect, provided herein is a method, comprising: a) cross-linking a first polypeptide to a second polypeptide to form a polypeptide complex, b) coupling the first polypeptide to a solid support, and c) after coupling the first polypeptide to the solid support, determining an identity of the first polypeptide and an identity of the second polypeptide.

In another aspect, provided herein is a composition, comprising: a) a solid support comprising an analyte binding site, b) a linking moiety coupled to the analyte binding site of the solid support, wherein the linking moiety comprises a first linker and a second linker, wherein the first linker comprises a cleavable moiety, and wherein the second linker comprises an unbound coupling group, c) a first polypeptide coupled to the first linker, and d) a second polypeptide, wherein the second polypeptide is bound to the first polypeptide, and wherein the second polypeptide comprises an unbound complementary coupling group, wherein the complementary coupling group is configured to bind the coupling group, wherein the first polypeptide and the second polypeptide do not contact the solid support.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which as as follows.

FIGS. 1A, 1B, and 1C illustrate steps of a method of identifying a polypeptide binding interaction at single-analyte resolution utilizing detectable probes comprising ligand-binding polypeptides, in accordance with some embodiments.

FIGS. 2A, 2B, and 2C depict detectable probes comprising ligand-binding polypeptide and detectable labels, in accordance with some embodiments.

FIGS. 3A, 3B, 3C, 3D, 3E, 3F, 3G, and 3H show steps of a method for identifying polypeptide binding interactions utilizing an array comprising ligand-binding polypeptides, in accordance with some embodiments.

FIGS. 4A, 4B, 4C, 4D, 4E, and 4F display configurations of arrays of ligand-binding polypeptides with and without polypeptide binding interactions with various binding entities, in accordance with some embodiments.

FIGS. 5A, 5B, and 5C illustrate arrays of binding targets in contact with free binding entities, in accordance with some embodiments.

FIGS. 6A, 6B, 6C, and 6D depict steps of a method of identifying polypeptide binding interactions on an array comprising a heterogeneous mixture of binding targets, in accordance with some embodiments.

FIGS. 7A and 7B show polypeptide complex configurations and arrays thereof, in accordance with some embodiments.

FIGS. 8A and 8B display configurations of polypeptide complexes containing one or more than one bound binding ligand, in accordance with some embodiments.

FIGS. 9A and 9B illustrate configurations of polypeptide complexes containing binding ligands or competitive binding ligands, in accordance with some embodiments.

FIG. 10 depicts a system for identifying polypeptide binding interactions at single-analyte resolution, in accordance with some embodiments.

FIGS. 11A, 11B, 11C, 11D, 11E, 11F, 11G, 11H, and 11I shows steps of a method of identifying dissociation of a polypeptide complex, in accordance with some embodiments.

FIGS. 12A, 12B, and 12C display a method of identifying a binding ligand of a polypeptide complex, in accordance with some embodiments.

FIGS. 13A, 13B, and 13C illustrate steps of a method of identifying a binding specificity of a binding site of a ligand-binding polypeptide, in accordance with some embodiments.

FIG. 14 depicts steps of a method of forming an array of binding targets from a sample extracted from a human subject, in accordance with some embodiments.

FIGS. 15A, 15B, 15C, 15D, 15E, 15F, 15G, 15H, 15I, and 15J show steps of a method identifying a polypeptide binding interaction on an array comprising a mixture of binding targets and standard or control binding targets, in accordance with some embodiments.

FIGS. 16A, 16B, 16C, 16D, 16E, 16F, 16G, and 16H display methods of performing competitive binding assays under differing conditions, in accordance with some embodiments.

FIGS. 17A, 17B, and 17C illustrate a method of separating out a fraction of polypeptides from a polypeptide sample using a single-analyte array of ligand-binding polypeptides, in accordance with some embodiments.

FIGS. 18A, 18B, and 18C illustrate a method of separating out a fraction of ligand-binding polypeptides from a polypeptide sample using a single-analyte array of binding ligands, in accordance with some embodiments.

FIGS. 19A and 19B depict a method of identifying a subfraction of a plurality of analytes that are capable of forming a binding interaction with a binding ligand, in accordance with some embodiments.

FIGS. 20A, 20B, 20C, 20D, 20E, and 20F show a method of forming and analyzing analyte binding interactions between a first proteomic sample and a second proteomic sample, in accordance with some embodiments.

FIG. 21 displays a method for depositing analytes of an analyte complex at adjacent but separate analyte binding sites of a single-analyte array, in accordance with some embodiments.

FIG. 22 illustrates a view of an array with detected sites containing cross-linked analytes, in accordance with some embodiments.

FIGS. 23A, 23B, 23C, 23D, 23E, 23F, 23G, and 23H illustrate steps of a method for analyzing two or more analytes that are co-localized at a single array address by an analyte binding interaction, in accordance with some embodiments.

FIGS. 24A, 24B, 24C, and 24D depict a method of identifying different proteoforms of a polypeptide, in accordance with some embodiments.

FIGS. 25A, 25B, 25C, and 25D illustrate a method of identifying different folding conformations of a polypeptide, in accordance with some embodiments.

### DETAILED DESCRIPTION

In many biological systems, certain proteins or other polypeptides form binding interactions with a binding ligand. A complete network of binding interactions within a biological system, or a subset thereof, may be referred to as an interactome. In some cases, a protein or polypeptide may form a binding interaction with a second polypeptide (i.e., a protein-protein interaction). In other cases, a protein or polypeptide may form a binding interaction with a biomolecule other than a polypeptide (e.g., a nucleic acid, a saccharide or polysaccharide, a lipid, a metabolite, a cofactor or covitamin, etc.). In yet other cases, a protein or polypeptide may form a binding interaction with a molecule or moiety that comprises a biological activity, such as a pharmaceutical compound or a toxin. Protein binding interactions may include short-term or transient interactions (e.g., enzymatic catalysis of a chemical reaction), medium-term interactions (e.g., binding of signaling molecules such as hormones), or long-term interactions such as binding of polypeptides into structural complexes (e.g., collagen, elastin, keratin, microtubules, etc.).

Differences in specificity of polypeptide binding interactions can arise due to particular structures of both polypeptides and associated binding ligands. For example, protein polymorphisms arising due to genetic differences between two individuals can affect the efficacy of a pharmaceutical compound administered to the first individual relative to the second individual. In another example, structural differences between protein isoforms within a single individual can affect the relative strength of a binding interaction between a binding ligand and each specific protein isoform. The function and/or activity of many biological systems can be facilitated by polypeptide binding interactions and, conversely, disfunction of biological systems can be associated with disfunction in polypeptide binding interactions.

A common example of an interactome is a transportome, a network of proteins or other polypeptides that transport other species within a biological system. Such transport proteins and polypeptides can include intracellular transport proteins, transmembrane transport proteins, and extracellular transport proteins. Transport proteins and/or polypeptides, depending upon the species and/or physical conditions, can bind numerous chemical species within a biological system. Transport proteins and/or polypeptides may bind and transport a variety of chemical species within a biological system, including native chemical species and externally-introduced chemical species. For example, transport proteins may bind native chemical species such as other polypeptides and constituents thereof (e.g., amino acids), nucleic acids and constituents thereof (e.g., nucleotides), lipids (e.g., fatty acids, phospholipids, steroids), saccharides and polysaccharides, monatomic or polyatomic ionic species, vitamins, cofactors, hormones, and numerous metabolic compounds. Likewise, transport proteins may bind externally-introduced chemical species such as dietary components (e.g., plant-derived compounds such as alkaloids, glycosides, polyphenols, and terpenes), pharmaceutical compounds, externally-introduced polypeptides (e.g., venoms), and toxic compounds.

The binding specificity of proteins and/or polypeptides (e.g., transport proteins, receptor proteins, immune proteins, regulatory proteins, etc.) can vary widely. For example, serum albumins are known to be capable of binding numerous chemical species, including ions, lipids, pharmaceutical compounds, and polypeptides, while immunoglobulins are typically characterized by a binding specificity for a limited number of chemical species, such as a single protein epitope. Likewise, proteins and/or polypeptides can possess one or multiple binding sites that are each capable of binding a chemical species. Each binding site of a protein and/or polypeptide with multiple binding sites may have a binding specificity for a particular chemical species or subset of chemical species that is unique from the other binding sites within the same protein or polypeptide. For example, serum albumins may possess separate and unique binding sites for lipids and polypeptides. In some cases, proteins or polypeptides may utilize allosteric or sequenced binding to enhance or inhibit the binding of chemical species to the protein or polypeptide.

Accordingly, proteins or polypeptides and the chemical species that form binding interactions with them can form “interactomes”—collections of proteins or polypeptides and associated chemical species that provide information regarding the status and function of a biological system from which the interactome is isolated. Exemplary interactomes may include the “albuminome”—the collection of albumins and interacting chemical species—and the “globulinome”—the collection of globulins and interacting chemical species. Interactomes such as transportomes may be prominent in circulating fluids such as blood, but may also be found within other fluids (e.g., cerebrospinal fluid) and interstitial regions of organisms, including humans.

In a specific example, the albuminome may be a target for further study due to its complex biology and its potential for diagnostic applications. Albumins comprise a family of globular, water-soluble proteins that typically possess ligand-binding sites. Of particular interest are serum albumins, a common protein found in animal blood. The primary purpose of serum albumins within a blood vessel is to generate an oncotic flow of water into the blood vessel, thereby counteracting the hydrostatic flow of water out of the blood vessel. Additionally, serum albumins play a role as transport proteins for numerous ligands, including monovalent and divalent cations, steroids, fatty acids, hormones (e.g., insulin), and other circulating polypeptides. Serum albumins can also serve as a transport protein for other species, such as pharmaceutical compounds and inflammatory metabolites. Due in part to their wide-ranging ligand-binding properties, serum albumins have an important role in regulating the chemistry of blood and other biological fluids. Serum albumins are the most common protein in blood, often comprising at least 50% by weight of the free protein fraction of blood serum. Besides blood, serum albumins can commonly be found in other bodily fluids, such as cerebrospinal fluid, as well as within interstitial spaces in tissues. Albumins, such as serum albumins, can contain multiple ligand-binding sites, for example at least 3, 4, 5, 6, 7, 8, 9, 10, or more ligand-binding sites. Albumins may be capable of transporting multiple bound ligands simultaneously. Although albumins may be capable of simultaneously binding to multiple species of ligand, each of multiple ligand-binding sites may possess a binding specificity for a specific chemical species or a family of chemical species. For example, a serum albumin may possess a first ligand-binding site that is configured to favor the binding of lipids (e.g., steroids, fatty acids) and a second ligand-binding site that is configured to favor the binding of polypeptides (e.g., hormones). In another example, Vitamin D-binding protein, an albumin, is capable of binding vitamin D and a range of vitamin D metabolites, as well as some fatty acids.

Albumins, such as serum albumins, may have an important mechanistic role in certain biological processes (e.g., anti-inflammatory processes, immune processes, etc.) due to their role in regulating the chemistry of bodily fluids. Moreover, albumins may bind clinically-relevant biomarkers, including low copy-number biomarkers. For example, human serum albumin may bind known cancer biomarkers such as CDH5, CVAM1, and IGFBP3. Changes in the characteristics of albumins may play a significant role in altering the composition of the albuminome. Serum albumins, such as human serum albumin—a typically non-glycoslyated protein—may be exported into extracellular spaces in glycosylated isoforms. Likewise, post-secretory environments may induce additional post-translational modifications of albumins, such as S-cysteinylation, S-nitrosylation, S-guanylation, or dehydralanine conversion. Atypical post-translational modifications of albumins (i.e., post-translational modifications that are not characteristic of wild-type albumins) may alter the ligand-binding characteristics of albumins, depending upon type and location. For example, post-translational modification of serum albumins may impact the binding of certain pharmaceutical compounds to the modified serum albumins, thereby potentially altering the bioavailability, half-life, and/or metabolism of the pharmaceutical compounds.

Like albumins, globulins comprise a family of free, globular transport proteins that are common in circulatory fluids like blood, as well as in other bodily fluids and interstitial spaces. Depending upon structure, globulins most commonly are classified as alpha-globulins, beta-globulins, or gamma-globulins. Gamma-globulins include the multi-chain immunoglobulins, which have a well-known role in antigen targeting for animal immune responses. However, numerous other globulins have transport protein characteristics. In contrast to serum albumins, the binding specificity of globulins may be narrower than albumins, such as being limited to a specific chemical species or family of chemical species. For example, sex-hormone binding proteins, typically beta-globulins, specifically bind androgens and estrogens, while transcortins, typically alpha-globulins, bind other steroid hormones such as cortisol, progesterone, and other corticosteroids.

Interactomes, such as the albuminome or the globulinome, may contain substantial information that may be identified and/or quantified by a proteomic assay performed at single-analyte resolution. For example, a single-analyte proteomic assay may be useful for determining the identity of low copy-number polypeptides that form binding interactions with transport proteins. Such interactions may fall below the detection limit of more common ensemble-based or bulk proteomic assays. In another example, a single-analyte proteomic assay may be useful for identifying transient or weak interactions between transport proteins and other polypeptides. Again, such interactions may not be identifiable within the time-scale of ensemble-based or bulk proteomic assays.

Provided herein are proteomic systems and methods that may be useful for identifying and/or quantifying interactomes, such as the albuminome and/or the globulinome. Some methods and compositions set forth herein utilize single-analyte arrays of binding targets such as ligand-binding polypeptides (e.g., albumins or globulins) or binding ligands thereof to identify polypeptide binding interactions with single-analyte resolution. Some methods set forth herein involve the contacting of arrays of single analytes with pluralities of binding entities that may be capable of forming polypeptide binding interactions with a single-analyte binding target on the array. A polypeptide binding interaction may subsequently be detected at single-analyte resolution. Such methods may be useful for determining the binding specificity of a ligand-binding polypeptide for various binding ligands, including low copy number polypeptides (e.g., cancer biomarkers, etc.), as well as determining the relative binding specificities of a ligand-binding polypeptide for non-polypeptide molecules, such as pharmaceutical compounds.

Additional methods and compositions provided herein may be useful for identifying dissociative polypeptide binding interactions. Some methods as set forth herein may utilize single-analyte arrays of polypeptide complexes to study dissociation of the polypeptide complexes in the presence of a binding competitor. Methods as set forth herein may involve static (e.g., single-time measurements) or dynamic (e.g., time-series) measurements of polypeptide binding interactions. The information on polypeptide binding interactions may be utilized to understand the dynamics of polypeptide binding interactions in heterogeneous systems. For example, the methods may be utilized to identify the possible impact of a pharmaceutical compound on the albumin-associated binding of a second pharmaceutical compound.

Additionally, methods and composition for individualized (e.g. personalized) analysis of ligand-binding polypeptides are provided. Methods of forming and utilizing arrays or detectable probes comprising ligand-binding polypeptides extracted from an individual subject are described. The methods may be useful for obtaining personalized information on polypeptide binding interactions based upon an individual's underlying proteome. For example, albumin may be extracted from an individual to form an albumin array that is used to estimate the binding affinity of various pharmaceutical formulations with the personalized albumin array.

### Definitions

Terms used herein will be understood to take on their ordinary meaning in the relevant art unless specified otherwise. Several terms used herein and their meanings are set forth below.

As used herein, the term “polypeptide” refers to a molecule comprising two or more amino acids joined by a peptide bond. A polypeptide may also be referred to as a protein, oligopeptide or peptide. Although the terms “protein,” “polypeptide,” “oligopeptide” and “peptide” may optionally be used to refer to molecules having different characteristics, such as amino acid sequence composition or length, molecular weight, origin of the molecule or the like, the terms are not intended to inherently include such distinctions in all contexts. A polypeptide can be a naturally occurring molecule, or synthetic molecule. A polypeptide may include one or more non-natural amino acids, modified amino acids, or non-amino acid linkers. A polypeptide may contain D-amino acid enantiomers, L-amino acid enantiomers or both. Amino acids of a polypeptide may be modified naturally or synthetically, such as by post-translational modifications.

As used herein, the term “albumin” refers to a polypeptide whose structure, sequence, and/or function is classified as belonging to the albumin family. An albumin may be a globular protein that is derived from a bodily fluid or an interstitial space of an organism. An albumin may have a known or unknown binding specificity for one or more binding ligands. An albumin may possess one or more binding sites with a known or unknown binding specificity. In particular cases, an albumin may be a serum albumin, such as human serum albumin or bovine serum albumin. As used herein, the term “globulin” refers to a polypeptide whose structure, sequence, or function is classified as belonging to the globulin family. A globulin may be a globular protein that is derived from a bodily fluid or an interstitial space of an organism. A globulin may be characterized by an insolubility in water and/or a solubility in dilute salt solution. An albumin may have a known or unknown binding specificity for one or more binding ligands. An albumin may possess one or more binding sites with a known or unknown binding specificity. In some cases, a globulin may include an immunoglobulin (e.g., IgA, IgD, IgE, IgG, IgM, etc.). In other cases, a globulin may not include an immunoglobulin.

As used herein, the term “ligand-binding polypeptide” refers to a polypeptide that is configured to bind a ligand at a specific binding site. A ligand can be bound transiently or permanently. A ligand-binding polypeptide may bind two or more species of ligands. A ligand-binding polypeptide may alter a bound ligand (e.g., enzymatically, refolding a molecular structure, etc.). A ligand-binding polypeptide need not alter a bound ligand. A ligand-binding polypeptide may comprise a transport polypeptide that is configured to deliver a ligand from a first location to a second location. A ligand-binding polypeptide may comprise one or more binding sites that are configured to bind a ligand. A ligand-binding polypeptide may comprise an affinity agent. Exemplary ligand-binding polypeptides include albumin and globulin. As used herein, the term “binding site,” when used in reference to a ligand-binding polypeptide, refers to a region of the structure of the ligand-binding polypeptide that forms a binding interaction with a ligand. The binding site may comprise one or more epitopes that form a binding interaction with a ligand. A binding site may form a transient binding interaction with a ligand, such as an electrostatic interaction or a temporary covalent, ionic, hydrogen, or coordination bond with a ligand.

As used herein, the term “binding ligand” refers to an entity that forms a binding interaction with a binding site of a receptor such as a ligand-binding polypeptide. A ligand may include any of a variety of chemical species, including for example, polypeptides and non-polypeptide chemical species. Exemplary polypeptide binding ligands may include signaling polypeptides (e.g., hormones), receptor polypeptides, and receptor polypeptide fragments. Non-polypeptide chemical species can include any of a variety of chemical species, including for example, non-polypeptide biomolecules (e.g., polynucleotides, nucleotides, polysaccharides, saccharides, lipids, vitamins, cofactors, metabolites, etc.), external intakes and metabolites thereof (e.g., flavonoids, retinoids, polyphenols, alkaloids, cannabinoids, etc.), toxins (e.g., neurotoxins, endocrine disruptors, etc.), pharmaceutical compounds, candidate pharmaceutical compounds (e.g., enantiomers, R-group substitutions of studied compounds, etc.), ionic compounds (e.g., monatomic ions, polyatomic ions, organic ions, inorganic ions, metal ions, etc.), and nanoparticles (e.g., organic nanoparticles, inorganic nanoparticles, semiconductor nanoparticles, metal nanoparticles, carbon nanoparticles, polymer nanoparticles, microplastics, etc.) and functionalized version thereof. A binding ligand may comprise one or more moieties that form a binding interaction at a binding site of a ligand-binding polypeptide. Exemplary binding ligands include ligands that bind to albumin or globulin. As used herein, the term “candidate binding ligand” refers to an entity whose binding interactions with a ligand-binding polypeptide is suspected or unknown. A candidate binding ligand may be an unknown analyte (e.g., a random polypeptide extracted from a subject) whose binding interactions with one or more ligand-binding polypeptides is to be characterized. For example, a plurality of polypeptides may be derived from a blood sample and deposited on a single-analyte array to individually test the binding characteristics of each polypeptide of the plurality of polypeptides against a ligand-binding polypeptide (e.g., an albumin, a globulin, etc.). A candidate binding ligand may be a molecule or other entity (e.g., a new pharmaceutical compound) whose binding interactions with one or more ligand-binding polypeptides are to be characterized. For example, a new pharmaceutical compound may be characterized against a series of native polypeptide mutants to determine any binding interactions of the new pharmaceutical compound with each native polypeptide mutant of the series of native polypeptide mutants.

As used herein, the term “polypeptide binding interaction” refers to a detectable association between a ligand-binding polypeptide and one or more binding ligands of the ligand-binding polypeptide. A polypeptide binding interaction may include specific and non-specific bindings of ligand-binding polypeptides with binding ligands. Polypeptide binding interactions can include covalent interactions or non-covalent interactions between a ligand-binding polypeptide and a binding ligand. In some cases, a polypeptide binding interaction may occur between an immobilized first binding entity (e.g., ligand-binding polypeptide, binding ligand) and a solution-phase second binding entity. In other cases, a polypeptide binding interaction may occur between a solution-phase first binding entity and a solution-phase second binding entity. A polypeptide binding interaction may be characterized as an association between a ligand-binding polypeptide and one or more binding ligands that is detectable for an amount of time that, for example, exceeds a diffusional time scale of a free binding entity involved in forming the polypeptide binding interaction, or exceeds a time scale for a detection system. In some cases, a polypeptide binding interaction may be characterized as an association between a ligand-binding polypeptide and one or more binding ligands that is detectable for an amount of time that permits multiple detections of the interaction.

As used herein, the term “binding entity” refers to a molecule, moiety particle, or other chemical species involved in forming a polypeptide binding interaction. A binding entity may include a component of a polypeptide complex. A binding entity may include a polypeptide complex. A binding entity may have at least one complementary binding entity with which the binding entity forms a polypeptide binding interaction. A binding entity may comprise a ligand-binding polypeptide or a binding ligand. In some cases, a binding entity may not comprise an affinity agent (e.g., an antibody or fragment thereof, an aptamer, a peptamer, etc.). As used herein, the term “binding target,” when used in reference to a single-analyte array, refers to a binding entity that is coupled to an address on the single-analyte array. A binding target may have at least one complementary binding entity with which the binding target forms a polypeptide binding interaction. A binding target may comprise a ligand-binding polypeptide or a binding ligand.

As used herein, the term “free” refers to a binding entity that is not immobilized. For example, a free entity is not coupled to an array (e.g., a solid support) or coupled to a binding entity that is coupled to an array. A free binding entity may be in fluid phase, for example, being freely diffusible in the fluid phase. A free binding entity may comprise a ligand-binding polypeptide, a binding ligand, or a polypeptide complex. As used herein, the term “coupled,” when used in reference to a binding entity, refers to attachment of the binding entity to an object (e.g., a solid support or array address), to a binding entity that is coupled to an array, or to a second binding entity. A coupled binding entity may be contained within a fluidic medium. A coupled binding entity may be bound by a covalent or non-covalent binding interaction. Exemplary types of coupling may include, but are not limited to, covalent conjugation, coordination bonding, ionic bonding, hydrogen bonding, electrostatic interactions, magnetic interactions, and nucleic acid hybridization.

As used herein, the term “retaining component” refers to a moiety of an affinity agent or other substance that links two other components to each other. A retaining component can maintain the two other moieties within a particular distance of each other. For example, the two other moieties can be maintained at a distance of at most 1000 nm, 500 nm, 100 nm, 50 nm, 10 nm, 5 nm, 1 nm or less. Alternatively or additionally, a retaining component can separate the two other moieties at a particular distance from each other. For example, the two other moieties can be maintained at a distance of at least 1 nm, 5 nm, 10 nm, 50 nm, 100 nm, 500 nm, 1000 nm or more. A retaining component can include, for example, a nucleic acid, structured nucleic acid particle (SNAP), nucleic acid nanoball, nucleic acid origami, protein nucleic acid, polypeptide, synthetic polymer, polysaccharide, organic particle, inorganic particle, gel, hydrogel, coated particle, or the like. A retaining component can optionally have a polymeric structure. Alternatively, a retaining component need not have a polymeric structure. In some embodiments, a retaining component has a composition that is similar to other components to which it is attached. For example, a plurality of binding components that are composed of polypeptide material can be attached to a polypeptide retaining component. Alternatively, a retaining component can have a composition that differs substantially from the composition of other components to which it is attached. For example, a plurality of binding components that are composed of polypeptide material can be attached to a retaining component that is composed partially or entirely of a material other than polypeptide, such as nucleic acid material, or an organic or inorganic nanoparticle (e.g., carbon nanosphere, silicon dioxide nanosphere, etc.).

As used herein, the term “structured nucleic acid particle” (or “SNAP”) refers to any single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same sequence length as the SNAP. The compacted three-dimensional structure can optionally be characterized with regard to tertiary structure. For example, a SNAP can be configured to have an increased number of interactions between regions of a polynucleotide strand, less distance between the regions, increased number of bends in the strand, and/or more acute bends in the strand, as compared to the same nucleic acid molecule in a random coil or other non-structured state. Alternatively or additionally, the compacted three-dimensional structure can optionally be characterized with regard to quaternary structure. For example, a SNAP can be configured to have an increased number of interactions between polynucleotide strands or less distance between the strands, as compared to the same nucleic acid molecule in a random coil or other non-structured state. In some configurations, the secondary structure (i.e. the helical twist or direction of the polynucleotide strand) of a SNAP can be configured to be more dense than the same nucleic acid molecule in a random coil or other non-structured state. A SNAP can optionally be modified to permit attachment of additional molecules to the SNAP. A SNAP may comprise DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are conjugated to other molecules (e.g., affinity agents, detectable labels) or are configured to be conjugated to other molecules (e.g., by reactive handles). A SNAP may include engineered or rationally-designed structures, such as nucleic acid origami and nucleic acid nanoballs.

As used herein, the term “nucleic acid origami” refers to a nucleic acid construct comprising an engineered tertiary or quaternary structures in addition to the naturally-occurring helical structure of nucleic acid(s). A nucleic acid origami may include DNA, RNA, PNA, modified or non-natural nucleic acids, or combinations thereof. A nucleic acid origami may comprise a plurality of oligonucleotides that hybridize via sequence complementarity to produce the engineered structuring of the origami particle. A nucleic acid origami may comprise sections of single-stranded or double-stranded nucleic acid, or combinations thereof. Exemplary nucleic acid origami structures may include nanotubes, nanowires, cages, tiles, nanospheres, blocks, and combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

As used herein, the term “nucleic acid nanoball” refers to a globular or spherical nucleic acid structure. A nucleic acid nanoball may comprise a concatemer of sequence regions that arranges in a globular structure. A nucleic acid nanoball may include a rolling circle amplification product. A nucleic acid nanoball may include DNA, RNA, PNA, modified or non-natural nucleic acids, or combinations thereof. A nucleic acid nanoball can have a compact structure, thereby forming a structured nucleic acid particle (SNAP) or portion thereof.

As used herein, the term “detectable probe” refers to an affinity agent that has an observable characteristic or produces an observable signal. A detectable probe can be detectable at single-analyte resolution. A detectable probe may comprise an affinity agent coupled to a detectable moiety (e.g., a conjugated nucleic acid barcode, fluorescent dyes, etc.). A detectable probe may comprise an affinity agent that is coupled to a detectable label by a retaining component. A detectable probe may comprise a plurality of affinity reagents and/or a plurality of detectable labels. As used herein, the term “detectable label” refers to a moiety that is has an observable characteristic or produces an observable signal. The observable characteristic or signal can be, for example, an optical signal such as absorbance of radiation, luminescence or fluorescence emission, luminescence or fluorescence lifetime, luminescence or fluorescence polarization, or the like; Rayleigh and/or Mie scattering; binding affinity for a ligand or receptor; magnetic properties; electrical properties; charge; mass; radioactivity or the like. A label component can be a detectable chemical entity that is conjugated to or capable of being conjugated to another molecule or substance. Exemplary molecules that can be conjugated to a label component include an affinity agent or a binding partner. A label component may produce a signal that is detected in real-time (e.g., fluorescence, luminescence, radioactivity). A label component may produce a signal that is detected off-line (e.g., a nucleic acid barcode) or in a time-resolved manner (e.g., time-resolved fluorescence). A label component may produce a signal with a characteristic frequency, intensity, polarity, duration, wavelength, sequence, or fingerprint. Exemplary labels include, without limitation, a fluorophore, luminophore, chromophore, nanoparticle (e.g., gold, silver, carbon nanotubes), heavy atom, radioactive isotope, mass label, charge label, spin label, receptor, ligand, nucleic acid barcode, polypeptide barcode, polysaccharide barcode, or the like.

As used herein, the term “subject” refers to a living or decedent source from which a polypeptide or a sample of polypeptides is extracted or derived. A subject may be a human. A subject may be a non-human organism, such as a domesticated animal, a non-domesticated animal, a plant, a fungi, a bacterium, a protozoan, an archaea, or a virus. A subject may be an organism with an unmodified genome. A subject may be an organism with a modified genome. A subject may produce one or more native polypeptides. A subject may produce one or more polypeptides comprising a mutation. A subject may produce one or more engineered polypeptides or engineered mutations within a polypeptide.

As used herein, the term “native,” when used in reference to an organism or polypeptide, refers to an organism having a non-engineered or unmodified genome, or a polypeptide that arises from a non-engineered or unmodified genome. A native polypeptide may include a polypeptide with one or more amino acid residues whose identity may vary within a cohort of organisms belonging to the same species as a subject from which the polypeptide is extracted. A “wild-type polypeptide” may refer to a polypeptide with an amino acid sequence that arises due to a gene with a major allele frequency within a cohort of organisms belonging to the same species as a subject from which the polypeptide is extracted. A “mutation” may refer to a deviance in the identity of an amino acid residue within a polypeptide relative to a wild-type polypeptide, for example as determined by a minor allele frequency or a similar measure. Similarly, a “mutant” may refer to a polypeptide comprising a mutation relative to a wild-type polypeptide. As used herein, the term “engineered” may refer to an organism or a polypeptide produced by the intentional (e.g. random or rational) or designed modification of a genome of an organism. An engineered polypeptide may be produced by modification of the genome of the organism from which the polypeptide is naturally produced. An engineered polypeptide may be produced by transgenic polypeptide production. An “engineered mutation” may refer to a mutation of a polypeptide that occurs due to the engineering of an organism or the genome of an organism.

As used herein, the term “proteomic,” when used in reference to a plurality of polypeptides, refers to the plurality of polypeptides possessing a diversity of polypeptide species that is representative of a subject (e.g., an organism such as an animal, a plant, a fungi, a bacterium, a virus, etc.) or a component thereof (e.g., a tissue, an organelle, a fluid, an extracellular material, an excreta, etc.) from which the plurality of polypeptides is derived. A proteomic sample can contain a diversity of polypeptide species of a subject or a component thereof representing at least about 5%, 10%, 15%, 20%, 25%, 30%, 35%, 40%, 45%, 50%, 55%, 60%, 65%, 70%, 75%, 80%, 85%, 90%, 95%, 99%, 99.9%, or more than 99.9% of all polypeptide species of the subject or the component thereof. Alternatively or additionally, a proteomic sample can contain a diversity of polypeptide species of a subject or a component thereof representing no more than about 99.9%, 99%, 95%, 90%, 85%, 80%, 75%, 70%, 65%, 60%, 55%, 50%, 45%, 40%, 35%, 30%, 25%, 20%, 15%, 10%, 5%, or less than 5% of all polypeptide species of the subject or the component thereof.

As used herein, the term “species,” when used in reference to a polypeptide, refers to the primary structure of the polypeptide. Two polypeptides with identical primary structure would be members of the same polypeptide species. Two polypeptides with differing primary structures would be members of separate species. Multiple species may arise from multiple copies of a single initial polypeptide structure due to the formation of isoforms (e.g., splice isoforms, post-translationally modified polypeptides, polypeptide truncation, etc.) during post-translational processing.

As used herein, the term “polypeptide complex” refers to a structure formed by the binding of a ligand-binding polypeptide with a binding ligand. A polypeptide complex may comprise a ligand-binding polypeptide and a polypeptide ligand. A polypeptide complex may comprise a ligand-binding polypeptide and a non-polypeptide ligand (e.g., a nucleic acid, a lipid, a plant-derived compound, a pharmaceutical compound, etc.). A polypeptide complex may induce a conformational change within a ligand-binding polypeptide or a binding ligand. A polypeptide complex may be conformationally distinct and/or identifiable due to the occlusion of one or more epitopes or moieties of the ligand-binding polypeptide and/or the binding ligand. A polypeptide complex may be conformationally distinct and/or identifiable due to the exposing of one or more epitopes or moieties of the ligand-binding polypeptide and/or the binding ligand.

As used herein, the term “interstitial region,” when used in reference to an array, refers to a location in an array where a particular molecule is not present or that is configured to not attract or not bind the particular molecule. An interstitial region can be adjacent to two or more addresses of an array. An interstitial region may be optically resolvable at single-analyte resolution by absence of a detectable signal within the region, or presence of a differing signal within the region. An interstitial region can contain a surface chemistry that limits or prevents the deposition or coupling of analytes within the interstitial region. An interstitial region can contain a passivating surface chemistry that limits or prevents non-specific binding of molecules, including analytes, detectable probes, and binding ligands, within the interstitial region.

As used herein, the term “linker” refers to a distinct moiety, molecule or particle that couples a first entity to a second entity or that is configured to do so. As used in reference to an array, a linker can refer to a separate moiety, molecule or particle that couples an analyte to a site or address of a solid support. As used in reference to a polypeptide complex, a linker can refer to a separate moiety, molecule or particle that couples a ligand-binding polypeptide to a binding ligand. A linker may comprise a bifunctional, trifunctional, or polyfunctional linker. A linker may form a covalent or non-covalent coupling between a first entity and a second entity. Covalent linkers may comprise one or more reactive functional groups that are configured to chemically react with a complementary reactive functional group on an entity (e.g., a click reaction group). Non-covalent linkers may comprise one or more non-reactive functional groups that are configured to form a non-covalent interaction with a complementary non-reactive functional group on an entity (e.g., a streptavidin-biotin coupling). A linker may form covalent and non-covalent interactions between a first entity and a second entity. For example, a nucleic acid linker may comprise two complementary oligonucleotides that covalently attach to their respective entities, and are configured to form a non-covalent or reversible coupling by complementary base-pair coupling of the oligonucleotides. In some cases, a linker may comprise a structured nucleic acid particle (SNAP) or a nanoparticle. A nanoparticle linker may comprise a polymeric nanoparticle, a nucleic acid nanoparticle, an organic nanoparticle, a metallic nanoparticle, or a semiconductor nanoparticle. A linker may comprise one or more linking moieties and one or more non-linking moieties. A linker may comprise one or more detectable labels that are configured to provide a detectable signal. As used herein, the term “linking moiety” refers to a portion of a linker that forms a coupling interaction. A linking moiety may comprise a reactive group, such as a reactive functional group (e.g., an epoxide, an azide, a carboxyl, an amine, etc.). A linking moiety may comprise half of a complementary binding pair (e.g., streptavidin-biotin, SpyCatcher-SpyTag, SnoopCatcher-SnoopTag, SdyCatcher-SdyTag, etc.). A linking moiety may include a particle or group that is configured to form a non-covalent interaction (e.g., a magnetic nanoparticle, an electrically-charged particle, etc.).

As used herein, the term “binding specificity” refers to the tendency of an affinity reagent to preferentially interact with a binding partner, affinity target, or target moiety. An affinity reagent or ligand-binding polypeptide may have a calculated, observed, known, or predicted binding specificity for any possible binding partner, affinity target, or target moiety. Binding specificity may refer to selectivity for a single binding partner, affinity target, or target moiety in a sample over at least one other analyte in the sample. Moreover, binding specificity may refer to selectivity for a subset of binding partners, affinity targets, or target moieties in a sample over at least one other analyte in the sample. Binding specificity may be characterized as an affinity agent or ligand-binding polypeptide possessing a threshold binding affinity for a binding partner, affinity target, or target moiety, for example a dissociation constant of no more than about 1 micromolar (μM), 500 nanomolar (nM), 250 nM, 100 nM, 50 nM, 25 nM, 10 nM, 5 nM, 2.5 nM, 1 nM, 500 picomolar (pM), 250 pM, 100 pM, 50 pM, 25 pM, 10 pM, 5 pM, 2.5 pM, 1 pM, or less. Binding specificity may refer to an affinity agent or ligand-binding polypeptide possessing a stronger binding affinity for a binding partner, affinity target, or target moiety compared to another molecule. For example, a ligand-binding polypeptide may have a binding specificity for a second binding ligand if it has a 100 nM dissocation constant with a first binding ligand and a 10 nM dissociation constant with the second binding ligand.

As used herein, the term “binding affinity” or “affinity” refers to the strength or extent of binding between an affinity reagent and a binding partner, affinity target or target moiety. In some cases, the binding affinity of an affinity reagent for a binding partner, affinity target, or target moiety may be vanishingly small or effectively zero. A binding affinity of an affinity reagent—of an affinity reagent for a binding partner, affinity target, or target moiety may be qualified as being a “high affinity,” “medium affinity,” or “low affinity.” A binding affinity—of an affinity reagent for a binding partner, affinity target, or target moiety may be quantified as being “high affinity” if the interaction has a dissociation constant of less than about 100 nM, “medium affinity” if the interaction has a dissociation constant between about 100 nM and 1 mM, and “low affinity” if the interaction has a dissociation constant of greater than about 1 mM. Binding affinity—can be described in terms known in the art of biochemistry such as equilibrium dissociation constant (K_{D}), equilibrium association constant (K_{A}), association rate constant (kₒₙ), dissociation rate constant (k_{off}) and the like. See, for example, Segel, Enzyme Kinetics John Wiley and Sons, New York (1975), which is incorporated herein by reference in its entirety.

As used herein, the term “substrate” refers to a medium or material within which, or upon which, an array or a solid support is disposed. An array or solid support may be joined to or embedded within a substrate. An array or solid support may be fabricated from the substrate material or medium, for example by a process such as lithography. A substrate may be any of a variety of materials, including for example, glass, polymer, metal, metal oxide, semiconductor, mineral, resin, or a composite thereof. A substrate can posses one or more physical properties that enable or enhance a method set forth herein. A substrate can possess one or more physical properties that enable or enhance single-analyte detection. Exemplary physical properties of a substrate may include optical properties (e.g., opacity, reflectivity, index of refraction, autofluorescence, etc.), electrical properties (e.g., resistance, capacitance, band gap, etc.), magnetic properties (e.g., ferromagnetism, diamagnetism, paramagnetism, etc.), thermal properties (e.g., heat capacity, thermal conductivity, emissivity, etc.), fluidic properties (e.g., hydrophobicity, hydrophilicity, coefficient of friction, etc.), and mechanical properties (e.g., tensile strength, hardness, Young's modulus, etc.).

As set forth herein, the term “occupancy rate,” as used in reference to an array, refers to the fraction of addresses on an array that contain an analyte. For example, an array with 1000 total addresses of which 500 contain an analyte would have an occupancy rate of 0.5. As used herein, the term “polypeptide occupancy rate” refers to the fraction of addresses on an array that contain a polypeptide. As used herein, the term “ligand-binding polypeptide occupancy rate” refers to the fraction of addresses on an array that contain a ligand-binding polypeptide. As used herein, the term “binding ligand occupancy rate” refers to fraction of addresses on an array that contain a binding ligand. As used herein, the term “polypeptide complex occupancy rate” refers to the fraction of addresses on an array that contain a polypeptide complex.

As used herein, the term “single-analyte” refers to a chemical entity that is individually manipulated or distinguished from other chemical entities. The analyte can be, for example, a polypeptide, ligand-binding polypeptide, binding ligand, probe or other analyte set forth herein. A single analyte may possess a distinguishing property such as volume, surface area, diameter, electrical charge, electrical field, magnetic field, electronic structure, electromagnetic absorbance, electromagnetic transmittance, electromagnetic emission, radioactivity, atomic structure, molecular structure, crystalline structure, or a combination thereof. The distinguishing property of a single analyte may be a property of the single analyte that is detectable by a detection method that possesses sufficient spatial resolution to detect the individual single analyte from any adjacent single analytes. An analyte may comprise a single molecule, a single complex of molecules, a single particle, or a single chemical entity comprising multiple conjugated molecules or particles. A single analyte may be distinguished based on spatial or temporal separation from other analytes, for example, in a system or method set forth herein. Moreover, reference herein to a ‘single analyte’ in the context of a composition, system or method does not necessarily exclude application of the composition, system or method to multiple single analytes that are manipulated or distinguished individually, unless indicated contextually or explicitly to the contrary.

As used herein, the term “comprising” is intended herein to be open-ended, including not only the recited elements, but further encompassing any additional elements.

As used herein, the term “each,” when used in reference to a collection of items, is intended to identify an individual item in the collection but does not necessarily refer to every item in the collection. Exceptions can occur if explicit disclosure or context clearly dictates otherwise.

### Methods of Assaying Polypeptide Binding Systems

Provided herein are methods for identifying and/or quantifying interactions between ligand-binding polypeptides and binding ligands. The methods apply array-based techniques with single-analyte resolution to characterize presence or absence of a polypeptide binding interaction at each optically resolvable address of an array. The methods set forth herein are applicable to any polypeptide or polypeptide system where polypeptide binding interactions may be present. Methods set forth herein may be of particular interest for identifying polypeptide binding interactions of free or globular proteins, such as albumins and/or globulins, but may be readily extended to other ligand-binding polypeptides such as receptor proteins and chaperonins. The methods set forth herein may have several advantageous applications including: 1) determining specific and non-specific binding interactions between a ligand-binding polypeptide and one or more binding ligands; 2) determining one or more characteristics (e.g., dissociation constant, dissocation rate constant) of binding interactions between a ligand-binding polypeptide and one or more binding ligand; 3) determining the effect of altering a ligand-binding polypeptide on one or more of the binding ligands with which the ligand-binding polypeptide may form binding interactions (or vice versa); 4) determining changes in a characteristic of a binding interaction between an altered ligand-binding polypeptide and one or more binding ligands (or vice versa); and 5) determining one or more binding interactions and/or characteristics thereof of a ligand-binding polypeptide or a plurality of ligand-binding polypeptides that are extracted from a subject, such as a medical patient.

In some cases, a method may comprise forming a polypeptide binding interaction in a biological system, then identifying the interaction via a single-analyte assay. Such a method may comprise the steps of: a) forming a polypeptide binding interaction between a ligand-binding polypeptide and a binding ligand in a biological system (e.g., an in vivo system, an in vitro system, etc.), b) capturing information related to an occurrence of the polypeptide binding interaction (e.g., cross-linking the ligand-binding polypeptide to the binding ligand; attaching decodable tags to the ligand-binding polypeptide and the binding ligand, etc.), and c) identifying the information related to the occurrence of the polypeptide binding interaction by a single-analyte method, as set forth herein, thereby detecting the polypeptide binding interaction.

In other cases, a method may comprise forming a polypeptide binding interaction during a single-analyte assay. Such a method may comprise the steps of: a) capturing a plurality of moieties (e.g., a plurality of ligand-binding polypeptide, a plurality of binding ligands) on a single-analyte array, as set forth herein; b) contacting the single-analyte array comprising the plurality of moieties with a plurality of candidate binding partners (e.g., a plurality of candidate binding ligands, a plurality of candidate ligand-binding polypeptides); and c) identifying a presence or absence of a polypeptide binding interaction for each moiety of the plurality of moieties by a single-analyte method, as set forth herein.

Several methods set forth herein are exemplified by a polypeptide assay method that utilizes affinity agent binding profiles to identify single polypeptides. Such a method may be advantageous due to its non-destructive nature (i.e., polypeptides are left intact after affinity agent binding profiles are obtained). Accordingly, polypeptide identification information may be obtained before and/or after obtaining polypeptide binding interaction information in a non-destructive proteomic assay. However, other proteomic methods (e.g., barcode-based affinity agent binding methods, fluorosequencing methods, and Edman-type degradation-based methods), as set forth herein, may be applied to the polypeptide interaction methods set forth herein. The skilled person will readily recognize that destructive proteomic methods (e.g., Edman-type degradation-based methods) will necessarily limit the ordering of method steps due to the loss of mass (and associated information) from assayed analytes. For example, polypeptide binding interactions will need to be identified before a polypeptide is fluorosequenced due to the step-wise removal of amino acids from the sequenced polypeptide, thereby altering the polypeptide binding interactions of the sequenced polypeptide.

In an aspect, provided herein is a method of detecting a polypeptide binding interaction comprising contacting a ligand-binding polypeptide and a binding ligand of the polypeptide in the presence of a solid support, and detecting presence or absence of the polypeptide binding interaction on the solid support at single-analyte resolution, in which the polypeptide binding interaction comprises the binding of the ligand-binding polypeptide to the binding ligand. In a particular embodiment, provided herein is a method of detecting a polypeptide binding interaction comprising contacting an albumin and a binding ligand of the albumin in the presence of a solid support, and detecting presence or absence of the polypeptide binding interaction on the solid support at single-analyte resolution, in which the polypeptide binding interaction comprises the binding of the albumin to the binding ligand of the albumin. In another particular embodiment, provided herein is a method of detecting a polypeptide binding interaction comprising contacting a globulin and a binding ligand of the globulin in the presence of a solid support, and detecting presence or absence of the polypeptide binding interaction on the solid support at single-analyte resolution, in which the polypeptide binding interaction comprises the binding of the globulin to the candidate binding ligand of globulin. The skilled person will readily recognize that the methods set forth herein may readily be extended to ligand-binding polypeptides other than albumins and globulins.

A method of identifying, quantifying, and/or characterizing a binding interaction between a ligand-binding polypeptide and a binding ligand may comprise contacting detectable probes comprising ligand-binding polypeptides with an array of binding ligands and determining presence or absence of a binding interaction between a detectable probe and a binding ligand at one or more addresses on the array. In some configurations, the method may comprise contacting detectable probes comprising binding ligands with an array of candidate binding ligands. Particular configurations of these methods may additionally comprise identifying one or more single-analytes on an array by contacting the array of single-analytes with one or more detectable probes and determining an identify of one or more single analytes on the array based upon the measurement outcomes of the one or more detectable probes interacting with one or more single analytes. Other particular configurations of these methods may further comprise characterizing one or more characteristics of a binding interaction (e.g., a dissociation equilibrium constant, a dissociation rate constant, an association rate constant, etc.) by contacting an array with one or more detectable probes and determining a change in a binding interaction at one or more addresses on the array.

A single-analyte, array-based method comprising a ligand-binding polypeptide may be useful for characterizing binding specificities of ligand-binding polypeptides against large pools of candidate binding ligands. A ligand-binding polypeptide, such as an albumin or a globulin, may be capable of forming polypeptide binding interactions with conceivably thousands of polypeptide and non-polypeptide species. Single-analyte arrays provide a platform to conceivably provide billions of unique candidate binding ligands and identify presence or absence of a polypeptide binding interaction for each unique candidate. Such methods may be especially useful for identifying interactions between low copy number polypeptides. In some cases, a single-analyte array-based method may be utilized to identify previously-unknown polypeptide binding interactions between low copy number biomarkers (e.g., cancer biomarkers, diabetes biomarkers, etc.). Moreover, a single-analyte array-based method may be utilized to identify differences in polypeptide binding interactions between a ligand-binding polypeptide and variants of binding ligands (e.g., mutant versions of polypeptides, pharmaceutical variants and derivatives, etc.). Additionally, a single-analyte array-based method may be utilized to identify weak or non-specific interactions that occur between ligand-binding polypeptides and binding ligands. For example, although globulins typically have a binding specificity for a limited number of binding ligands, variants of globulins (e.g., mutants, atypical post-translational modifications, etc.) may have altered binding specificity that affects the behavior of systems in which the globulin is available.

In an aspect, provided herein is a method of detecting a polypeptide binding interaction, comprising: a) providing an array comprising a plurality of polypeptides, in which each polypeptide of the plurality of polypeptides is located at an address on the array, and in which each address of the array is optically resolvable from the other addresses on the array, b) contacting the array with a plurality of detectable probes, in which each detectable probe comprises a ligand-binding polypeptide and a detectable label, and optionally in which the ligand-binding polypeptide comprises an albumin or a globulin, and c) detecting presence or absence of the polypeptide binding interaction at each address at single-analyte resolution, in which the polypeptide binding interaction comprises binding of a detectable probe of the plurality of detectable probes to a polypeptide of the array of polypeptides.

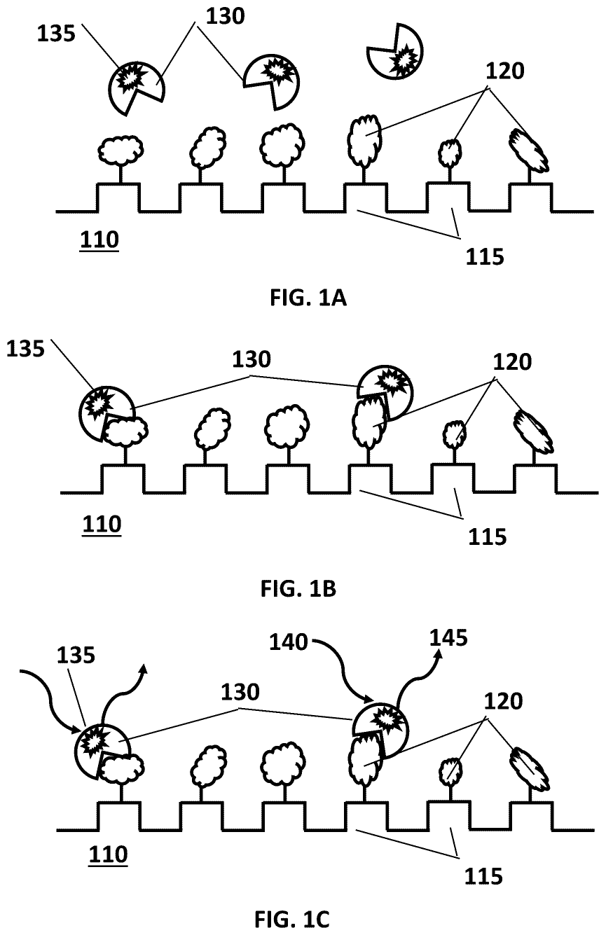

FIG. 1A-1C illustrate a method of detecting a polypeptide-binding interaction. As shown in FIG. 1A, an array is provided, in which the array comprises a solid support 110 and a plurality of addresses 115 that are optically resolvable at single-analyte resolution. Each address of the plurality of addresses 115 is coupled to a polypeptide 120 of a plurality of polypeptides. The solid support 110 is contacted with a plurality of detectable probes, optionally within a fluidic medium. Each detectable probe 130 of the plurality of detectable probes may comprise a ligand-binding polypeptide and a detectable label 135 (e.g., a fluorophore) that can produce a detectable signal. As shown in FIG. 1B, one or more detectable probes 130 of the plurality of detectable probes may bind to one or more polypeptides 120 of the plurality of polypeptides. As shown in FIG. 1C, a signal 145 may be produced by a detectable label 135, optionally by an excitation source 140 (e.g., incident light). Presence or absence of signal 145 may be detected at each address of the plurality of addresses 115 on the solid support 110, thereby indicating the presence or absence at each address of the plurality of addresses 115 of a polypeptide binding interaction between a ligand-binding polypeptide of a detectable probe 130 and a polypeptide 120 of a plurality of polypeptides. Optionally, a method as set forth herein may further include identifying each polypeptide 120 at each address of the plurality of addresses 115.

In another aspect, provided herein is a method comprising: a) providing an array comprising a plurality of addresses, in which each address of the plurality of addresses comprises a ligand-binding polypeptide, in which each address on the array is resolvable from each other address on the array, and optionally in which the ligand-binding polypeptide comprises an albumin or a globulin; b) contacting the array with a plurality of binding entities (e.g., candidate binding ligands, a plurality of polypeptides, a plurality of small molecules, a plurality of non-polypeptide biomolecules, and/or combinations thereof); and c) detecting presence or absence of a polypeptide binding interaction at each address at single-analyte resolution, in which the polypeptide binding interaction comprises binding of a binding entity of the plurality of binding entities with the ligand-binding polypeptide.

In a particular embodiment of the above method, an array may be provided with a plurality of ligand-binding polypeptides, in which the plurality of ligand-binding polypeptides comprises a proteomic sample. In another particular embodiment of the above method, an array may be contacted with a plurality of binding entities, in which the plurality of binding entities comprises a proteomic sample. In some cases, an array comprising a plurality of ligand-binding polypeptides may be contacted with a plurality of binding entities, in which the plurality of ligand-binding polypeptides comprises a first proteomic sample, and the plurality of binding entities comprises a second proteomic sample, thereby forming a binding interaction between a ligand-binding polypeptide of the first proteomic sample and a binding entity of the second proteomic sample. In a particular case, a first proteomic sample and a second proteomic sample may each be derived from the same subject or individual. In another particular case, a first proteomic sample may be derived from a first subject or individual and a second proteomic sample may be derived from a second subject or individual. For example, an array may comprise a plurality of polypeptides derived from a human subject, and the array may be contacted with a plurality of polypeptides from a pathogen of the human subject (e.g., a bacterium, virus, protist, fungus, etc.).

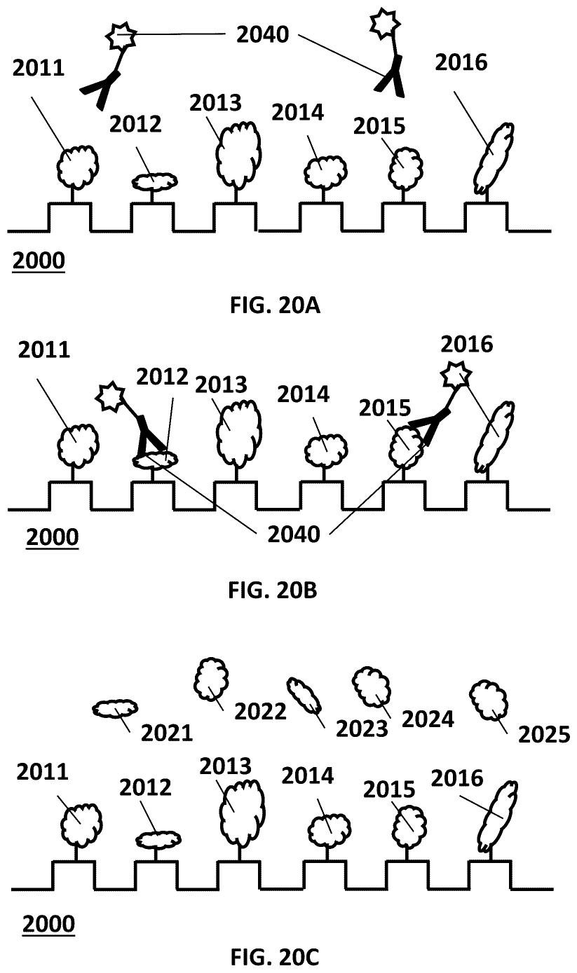

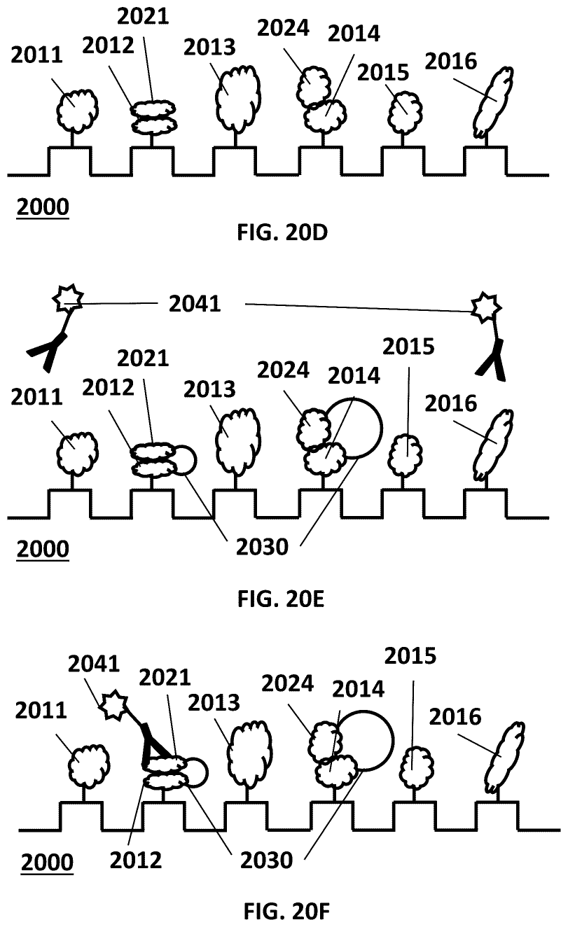

FIGS. 20A-20F depict a method of identifying binding interactions between a first proteomic sample and a second proteomic sample. FIGS. 20A-20B depict an optional method of identifying each polypeptide of a polypeptide array. FIG. 20A depicts an array comprising a solid support 2000 and a plurality of analyte binding sites, with each analyte binding site comprising a single polypeptide analyte derived from a first proteomic sample (2011, 2012, 2013, 2014, 2015, 2016, respectively). The array is contacted with a plurality of detectable probes 2040 that are configured to bind to one or more polypeptides of the first proteomic sample. FIG. 20B depicts a configuration of the array in which detectable probes 2040 have bound to analytes 2012 and 2015, thereby permitting detection of a detectable signal from array addresses at which those two analytes are coupled. Optionally, the method of FIGS. 20A-20B may be repeated with additional cycles of differing detectable probes 2040 until each analyte can be identified. FIG. 20C depicts contacting the array containing the first proteomic sample with a second proteomic sample comprising a plurality of polypeptides (2021, 2022, 2023, 2024, 2025, respectively). FIG. 20D depicts an array configuration in which polypeptide 2021 has formed a binding interaction with polypeptide 2012, and polypeptide 2024 has formed a binding interaction with polypeptide 2014. Optionally, the polypeptides may be contacted with a cross-linking reagent to covalently couple a polypeptide complexes. FIG. 20E depicts an array configuration in which cross-links 2030 have been formed between polypeptides 2012 and 2021, and 2014 and 2024, respectively. The array is contacted with a plurality of detectable probes 2041 that are configured to bind to one or more polypeptides of the first or second proteomic sample. FIG. 20F depicts an array configuration in which a detectable probe 2041 has bound to polypeptide 2021, thereby permitting detection of the co-localized analyte at the same array address as polypeptide 2012. Optionally, the method of FIGS. 20E-20F may be repeated with additional cycles of differing detectable probes 2041 until each bound analyte of the second proteomic sample can be identified.

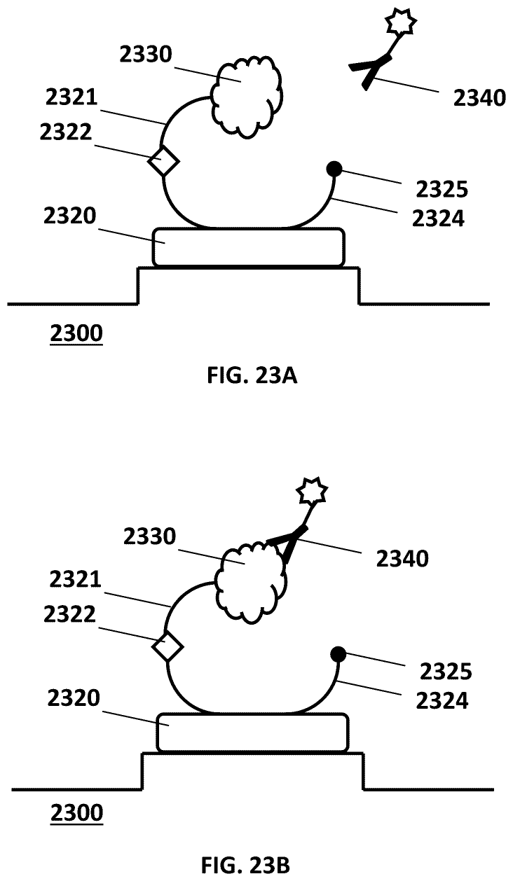

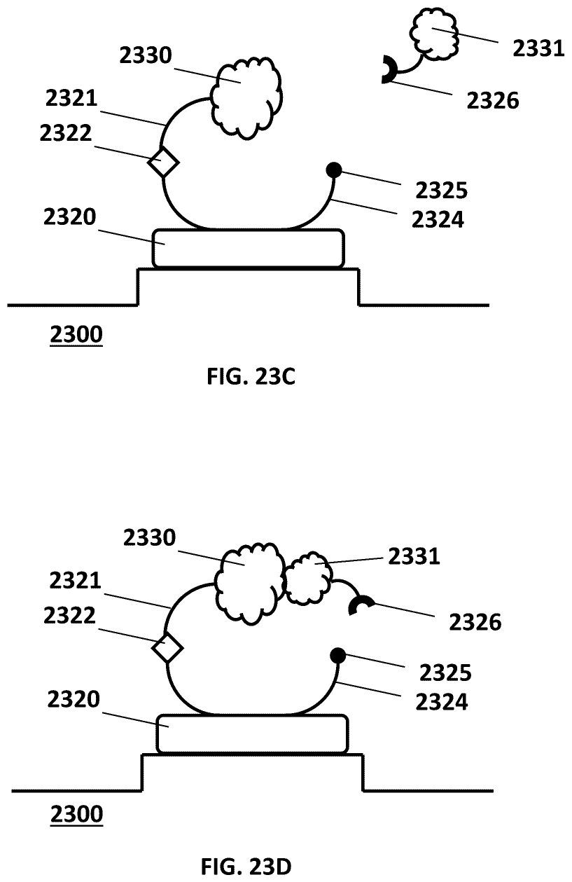

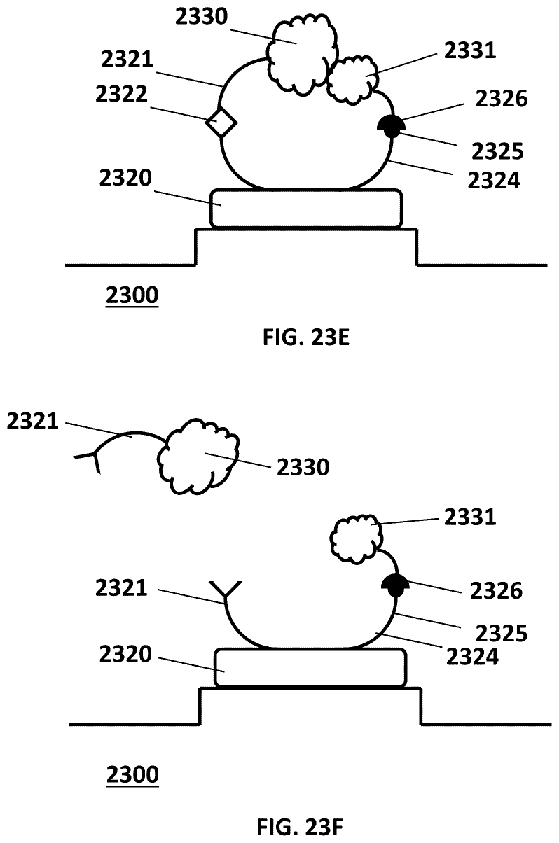

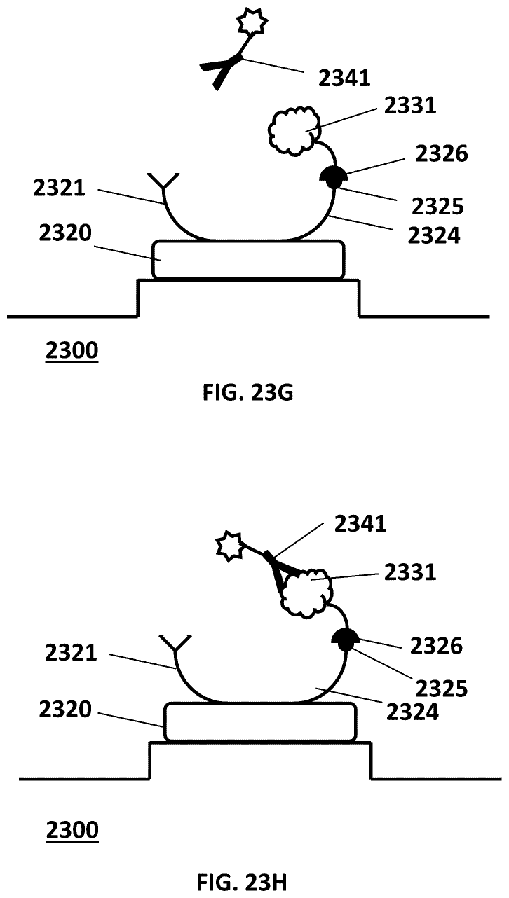

A method of detecting a co-localized binding interaction, such as the method depicted in FIGS. 20A-20F, may be facilitated by the use of a cleavable linker between an array-bound analyte and the array or a linking moiety that coupled the analyte to the array. FIGS. 23A-23H depicts a method of utilizing a cleavable linker to identifying two analytes that become co-localized by a binding interaction between the two analytes. FIG. 23A depicts a solid support 2300 comprising a bound polypeptide analyte 2330 at an array address. The polypeptide analyte 2330 is coupled to the solid support by a linking moiety 2320 comprising a linker 2321 with a cleavable group 2322. The linking moiety 2320 also comprises a second unoccupied linker 2324 comprising a coupling group 2325 (e.g., a covalent coupling group, a member of a receptor-ligand binding pair, etc.). Optionally, the array is contacted with a detectable probe 2340 that is configured to facilitate identification of the polypeptide analyte 2330. FIG. 23B depicts the detectable probe 2340 in a bound configuration with the polypeptide analyte 2330, thereby permitting detection of the probe 2340 co-localized with polypeptide analyte 2330 at the array address. Optionally, the method of FIGS. 23A-23B may be repeated until an identity is determined for the polypeptide analyte 2330. FIG. 23C depicts contacting the array with a second polypeptide analyte 2331 that is functionalized with a complementary coupling group 2326 that is configured to bind to coupling group 2325. FIG. 20D depicts an array configuration in which polypeptide analyte 2330 has formed a binding interaction with polypeptide analyte 2331, thereby co-localizing both analytes at the same array address. FIG. 23E depicts another array configuration in which a coupling interaction has occurred between the coupling group 2325 and the complementary coupling group 2326, thereby binding polypeptide analyte 2331 to the linking moiety via linker 2324. FIG. 23F depicts a decoupling of polypeptide analyte 2330 from the array address by disrupting the cleavable group 2322 (e.g., enzymatically, photolytically, chemically, etc.). FIGS. 23G and 23H depict a method for identifying the second polypeptide analyte after the release of the first polypeptide analyte 2330. As shown in FIG. 23G, the array is contacted with a detectable probe 2341 that is configured to facilitate identification of the polypeptide analyte 2331. FIG. 23H depicts the detectable probe 2341 in a bound configuration with the polypeptide analyte 2331, thereby permitting detection of the probe 2341 co-localized with the polypeptide analyte 2331 at the array address. Optionally, the method of FIGS. 23G-23H may be repeated until an identity is determined for the polypeptide analyte 2331.

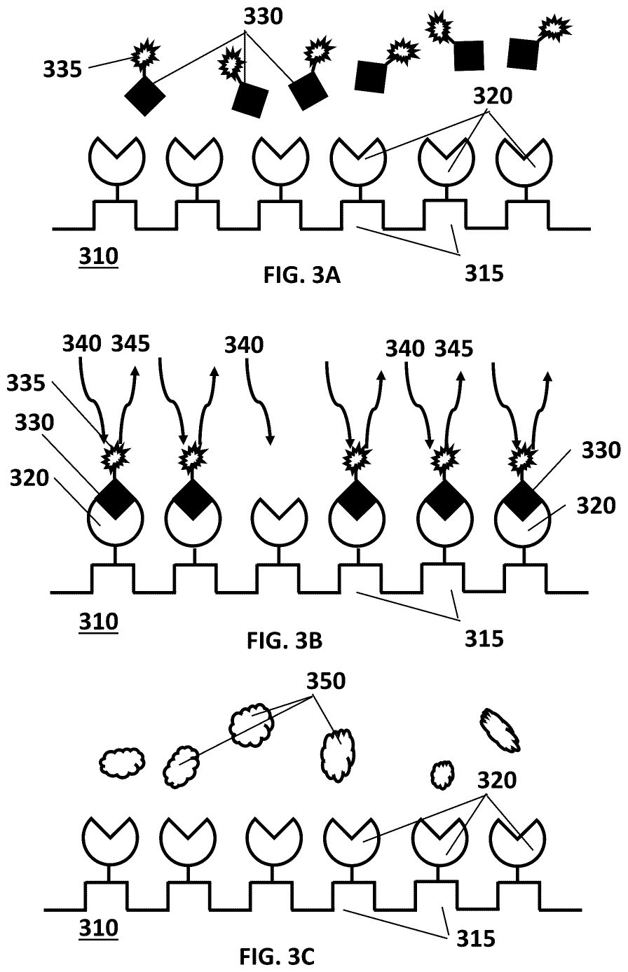

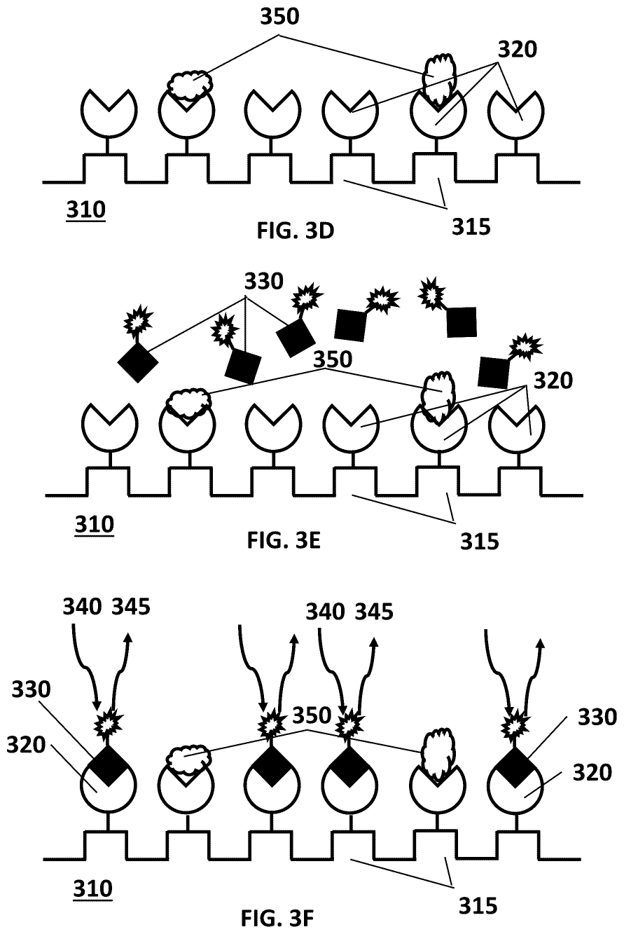

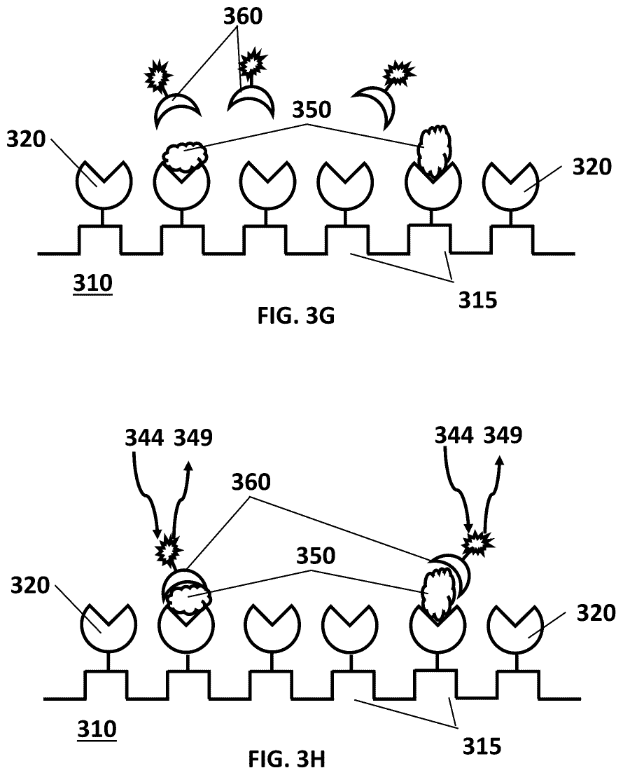

FIGS. 3A-3H illustrate a method of identifying a polypeptide binding interaction. As shown in FIG. 3A, an array is provided, in which the array comprises a solid support 310 and a plurality of addresses 315 that are resolvable at single-analyte resolution. Each address of the plurality of addresses 315 comprises a single ligand-binding polypeptide 320 of a plurality of ligand-binding polypeptides. The solid support 310 of the array is contacted with a plurality of detectable probes, optionally within a fluidic medium, in which each detectable probe 330 comprises a detectable label 335. Each detectable probe 330 of the plurality of detectable probes may comprise a binding specificity for a ligand-binding polypeptide 320 of the plurality of ligand-binding polypeptides. As shown in FIG. 3B, one or more detectable probes 330 may be bound by ligand-binding polypeptides 320. Presence or absence of a detectable probe at each address of the plurality of addresses 315 on the solid support 310 may be detected by a signal 345 generated by a detectable label 335 on a detectable probe at a particular address. Optionally, a signal may be generated in response to an excitation source 340 (e.g., a light source). Optionally, the detection of presence or absence of binding of a detectable probe 330 with a binding specificity for the ligand-binding polypeptide 320 may be omitted, or repeated to increase confidence in a physical measurement. Optionally, one or more detectable probes 330 may be unbound from a polypeptide binding interaction with a ligand-binding polypeptide 320. As shown in FIG. 3C, a plurality of binding entities (e.g., polypeptides derived from a subject, binding ligands, candidate binding ligands, etc.) may be contacted with the array, optionally within a fluidic medium. As shown in FIG. 3D, one or more binding entities 350 may form polypeptide binding interactions with ligand-binding polypeptides 320 at particular addresses of the plurality of addresses 315 on the solid support 310. FIGS. 3E-3F depict optional steps for determining addresses that have not formed a polypeptide binding interaction with a binding entity. As shown in FIG. 3E, a second plurality of detectable probes may be contacted with the array, optionally within a fluidic medium. As shown in FIG. 3F, the presence of a signal 345, optionally induced by an excitation source 340, may be detected from each address that has a ligand-binding polypeptide 320 forming a polypeptide binding interaction with a detectable probe 330. Likewise, absence of a signal 345 may be detected from each address that has a ligand-binding polypeptide 320 forming a polypeptide binding interaction with a binding entity 350. FIGS. 3G-3H illustrate optional steps of identifying a binding entity 350 forming a polypeptide binding interaction with a ligand-binding polypeptide 320 at a particular address of the plurality of addresses 315. FIG. 3G shows contacting a plurality of detectable affinity agents, optionally with a lack of binding specificity for a ligand-binding polypeptide 320, and optionally within a fluidic medium, with the array. FIG. 3H depicts the coupling of a detectable affinity agent 360 with a binding entity 350 at one or more addresses of the plurality of addresses. Presence or absence of coupling of a detectable affinity agent 360 with a binding entity 350 may be detected by detecting presence or absence of a signal 349, optionally induced by an excitation source 344, from the detectable affinity agent 360 at each address of the plurality of addresses 315. Optionally, the method may further include repeating the procedure of FIGS. 3G-3H with additional pluralities of detectable affinity agents, optionally including detectable affinity agents with differing binding specificities. Optionally, the method may further include identifying a binding entity 350 at a particular address of the plurality of addresses 315 based upon the detected binding profiles of one or more detectable affinity agents. Optionally, the method may further comprise excluding identification of a binding entity 350 at a subset of addresses at which the presence of signal 345 was detected during the step depicted in FIG. 3B.

In some cases, a method, as set forth herein, may comprise identifying and/or characterizing an abnormal or unexpected polypeptide binding interaction. For example, an abnormal polypeptide binding interaction may occur between an array-coupled ligand-binding polypeptide and a binding ligand due to an atypical post-translational modification of the ligand-binding polypeptide or the binding ligand. In some cases, a method may comprise one or more of the steps of: a) detecting the presence of an abnormal polypeptide binding interaction at an address of a single-analyte array; and b) characterizing at least one binding entity forming the abnormal polypeptide binding interaction at the address of the single-analyte array. In some cases, characterizing at least one binding entity forming an abnormal polypeptide binding interaction may comprise one or more steps of: a) contacting the at least one binding entity with a plurality of affinity agents; b) detecting presence or absence of binding of an affinity agent of the plurality of affinity agents to the at least one binding entity; and c) characterizing the at least one binding entity based upon the detected presence or absence of binding of the affinity agent of the plurality of affinity agents.

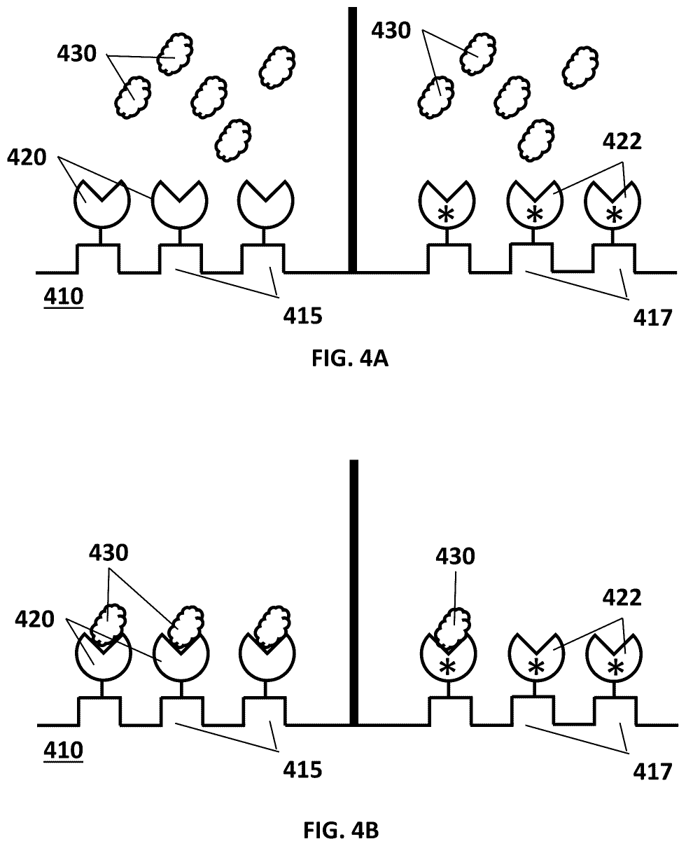

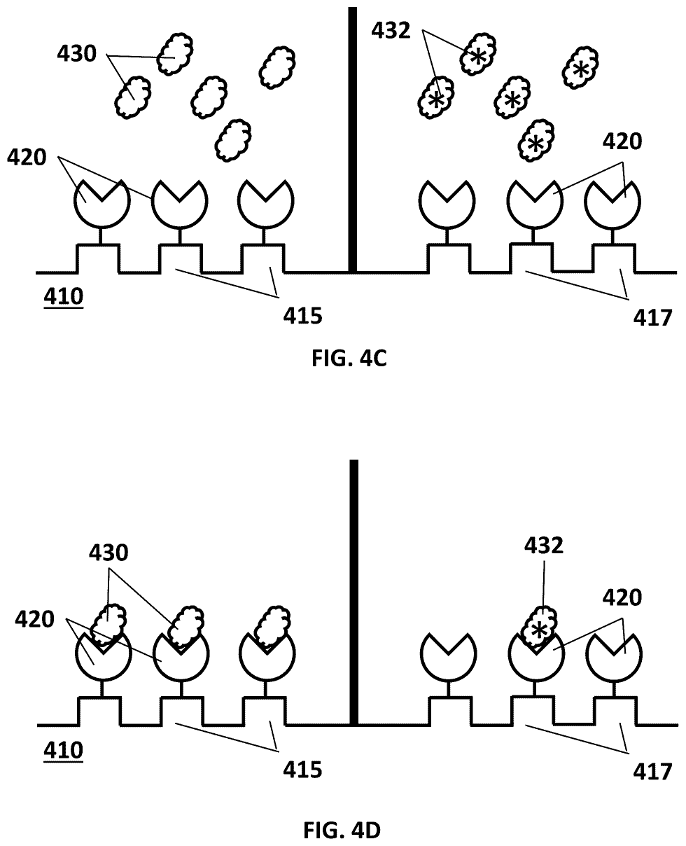

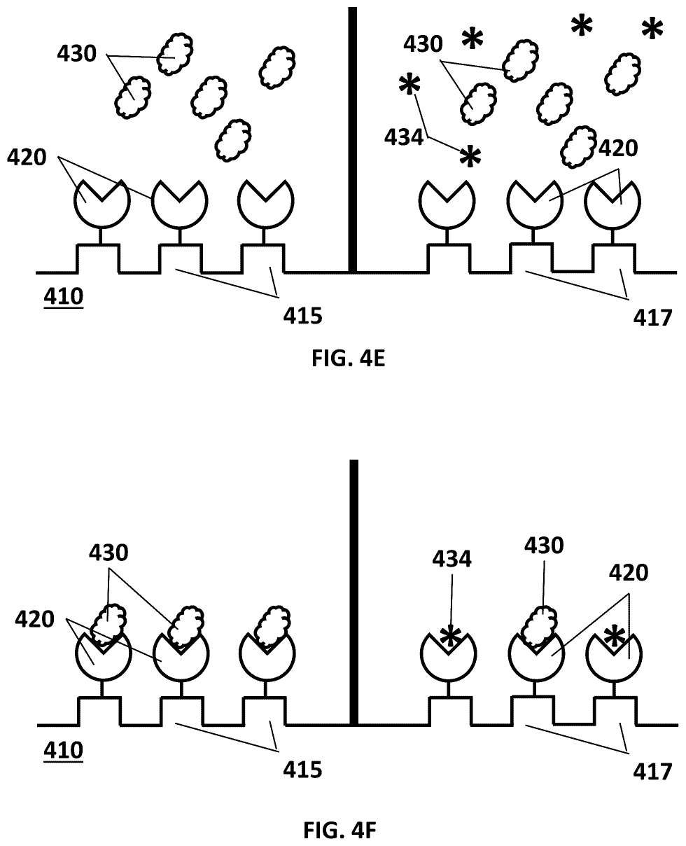

FIGS. 4A-4F illustrate additional embodiments of identifying polypeptide binding interactions utilizing arrays comprising pluralities of ligand-binding polypeptides. FIGS. 4A-4B depict a method of comparing binding between a first plurality of ligand-binding polypeptides and a second plurality of ligand-binding polypeptides (e.g., mutants of the first ligand-binding polypeptide, modified versions of the ligand-binding polypeptide, etc.). FIG. 4A shows a first configuration of an array comprising a solid support 410 that comprises a first plurality of addresses 415 and a second plurality of addresses, in which each address of the first and second plurality of addresses 415 and 417 is resolvable at single-analyte resolution. Each address of the first plurality of addresses 415 comprises a single first ligand-binding polypeptide 420 of a plurality of first ligand-binding polypeptides. Each address of the second plurality of addresses 417 comprises a single second ligand-binding polypeptide 422 of a plurality of second ligand-binding polypeptides. Optionally, the first plurality of addresses 415 may be fluidically isolated from the second plurality of addresses 417. A plurality of binding entities (e.g., polypeptides derived from a subject, binding ligands, candidate binding ligands, etc.) may be contacted with the first plurality of addresses 415 and the second plurality of addresses 417, optionally within a fluidic medium. FIG. 4B shows a second configuration of the array. One or more binding entities 430 of the plurality of binding entities may form a polypeptide binding interaction with one or more first ligand-binding polypeptides 420 of the first plurality of ligand-binding polypeptides. Likewise, one or more binding entities 430 of the plurality of binding entities may form a polypeptide binding interaction with one or more second ligand-binding polypeptides 422 of the second plurality of ligand-binding polypeptides. The first configuration and the second configuration may be distinguishable by a method set forth herein, such as the method shown in FIGS. 3A-3H.

FIGS. 4C-4D depict a method of comparing differences in polypeptide binding interactions for a plurality of ligand-binding polypeptides with a first plurality of binding entities (e.g., polypeptide binding entities, non-polypeptide binding entities, etc.) and a second plurality of binding entities (e.g., mutants of the polypeptide binding entities, modified versions of the polypeptide binding entities, chemically-substituted variants of non-polypeptide binding entities, etc.). FIG. 4C shows a first configuration of an array comprising a solid support 410 that comprises a first plurality of addresses 415 and a second plurality of addresses, in which each address of the first and second plurality of addresses 415 and 417 is resolvable at single-analyte resolution. Each address of the first and second plurality of addresses 415 and 417 comprises a ligand-binding polypeptide 420 of a plurality of ligand-binding polypeptides. Optionally, the first plurality of addresses 415 may be fluidically isolated from the second plurality of addresses 417. A first plurality of binding entities (e.g., polypeptides derived from a subject, binding ligands, candidate binding ligands, etc.) may be contacted with the first plurality of addresses 415, and a second plurality of binding entities may be contacted with the second plurality of addresses 417, optionally within a fluidic medium, in which the first plurality of binding entities differs from the second plurality of binding entities. FIG. 4D shows a second configuration of the array. One or more binding entities 430 of the first plurality of binding entities may form a polypeptide binding interaction with one or more ligand-binding polypeptides 420 at the first plurality of addresses. Likewise, one or more binding entities 432 of the second plurality of binding entities may form a polypeptide binding interaction with one or more ligand-binding polypeptides 420 at the second plurality of addresses 417. The first configuration and the second configuration may be distinguishable by a method set forth herein, such as the method shown in FIGS. 3A-3H.

FIGS. 4E-4F depict a method of comparing differences in polypeptide binding interactions for a plurality of ligand-binding polypeptides with a homogeneous plurality of binding entities (e.g., polypeptide binding entities, non-polypeptide binding entities, etc.) and a heterogeneous plurality of binding entities (e.g., a mixture of polypeptide binding entities and non-polypeptide binding entities). FIG. 4E shows a first configuration of an array comprising a solid support 410 that comprises a first plurality of addresses 415 and a second plurality of addresses, in which each address of the first and second plurality of addresses 415 and 417 is resolvable at single-analyte resolution. Each address of the first and second plurality of addresses 415 and 417 comprises a ligand-binding polypeptide 420 of a plurality of ligand-binding polypeptides. Optionally, the first plurality of addresses 415 may be fluidically isolated from the second plurality of addresses 417. A homogeneous plurality of binding entities comprising a first binding entity 430 (e.g., polypeptides derived from a subject, binding ligands, candidate binding ligands, etc.) may be contacted with the first plurality of addresses 415, and a heterogeneous plurality of binding entities comprising a first binding entity 430 and a second binding entity 434 may be contacted with the second plurality of addresses 417, optionally within a fluidic medium. FIG. 4F shows a second configuration of the array. One or more binding entities 430 of the homogeneous plurality of binding entities may form a polypeptide binding interaction with one or more ligand-binding polypeptides 420 at the first plurality of addresses 415. Likewise, one or more first binding entities 430 and one or more second binding entities 434 of the heterogeneous plurality of binding entities may form a polypeptide binding interaction with one or more ligand-binding polypeptides 420 at the second plurality of addresses 417. The first configuration and the second configuration may be distinguishable by a method set forth herein, such as the method shown in FIGS. 3A-3H.

A method, as set forth herein, may comprise forming and/or providing an array comprising a plurality of polypeptides. In some cases, the method may comprise providing an array comprising a plurality of polypeptides, in which the identity of at least one, some or all of the polypeptides on the array is unknown. In some cases, the method may comprise providing an array comprising a plurality of polypeptides, in which the identity of at least one, some or all of the polypeptides on the array is known. In some cases, the method may further comprise identifying a polypeptide of the plurality of polypeptides at one or more addresses. In some cases, the method may comprise providing an array comprising a plurality of polypeptides, in which the plurality of polypeptides comprises a ligand-binding polypeptide. In some cases, the method may comprise providing an array comprising a plurality of polypeptides, in which the plurality of polypeptides does not comprise a ligand-binding polypeptide. In some cases, the method may comprise providing an array comprising a plurality of polypeptides, in which the plurality of polypeptides comprises a plurality of candidate binding ligands for the ligand-binding polypeptide. In some cases, the method may comprise providing an array comprising a plurality of polypeptides, in which the plurality of polypeptides comprises a known and/or characterized binding ligand of the ligand-binding polypeptide. In some cases, the method may comprise providing an array comprising a plurality of polypeptides, in which the plurality of polypeptides comprises a variant of a known and/or characterized binding ligand of the ligand-binding polypeptide. In some cases, a variant of a known and/or characterized polypeptide binding ligand may comprise a native and/or engineered mutation. In some cases, a variant of a known and/or characterized polypeptide binding ligand may comprise a chemical modification of the binding ligand (e.g., methylation, ubiquitination, phosphorylation, etc.). In some cases, a variant of a known and/or characterized non-polypeptide binding ligand may comprise a chemical modification of the binding ligand (e.g., a methylation, a carboxylation, a phosphorylation, etc.). In some cases, a variant of a known and/or characterized non-polypeptide binding ligand may be uncharacterized with regard to one or more binding characteristics relative to a ligand-binding polypeptide. In some cases, the method may comprise providing an array comprising a plurality of polypeptides, in which the plurality of polypeptides comprises a known and/or characterized binding ligand and a variant of the known and/or characterized binding ligand of the ligand-binding polypeptide. In some cases, the method may comprise providing an array comprising a plurality of polypeptides, in which the plurality of polypeptides comprises a polypeptide derived and/or extracted from a subject (e.g., a human, a domesticated animal, a non-domesticated animal, a plant, a fungus, a bacteria, a protozoan, a virus, an archaea). In some cases, the method may comprise providing an array comprising a plurality of polypeptides, in which the plurality of polypeptides comprises a polypeptide from a sample derived or extracted from a subject (e.g., a sample of blood, cerebrospinal fluid, synovial fluid, urine, tears, mucus, a tissue sample, etc.).

A method, composition or system, as set forth herein, may utilize a detectable probe that is contacted with an array of binding targets. The detectable probes may be selected for a particular method based upon one or more characteristics, the characteristics including but not limited to: 1) solubility in a fluidic medium; 2) stability in a fluidic medium; 3) binding affinity for a target (e.g., binding dissocation constant, association rate constant, dissociation rate constant, etc.); 4) binding avidity for a target; 5) intensity or magnitude of a detectable signal; and 6) stability and/or duration of a detectable signal. In some configurations, a detectable probe may comprise a detectable label coupled directly to a binding entity (e.g., an affinity reagent, a ligand-binding polypeptide, a binding ligand, etc.). A detectable label may be coupled to a binding entity by, for example direct conjugation of the detectable label with the chemical entity (e.g., reaction of an amine-reactive fluorophore with a polypeptide) or attachment of the detectable label to the binding entity by a linker (e.g., a bifunctional linker, a nucleic acid linker, etc.).

In some configurations, a detectable probe may comprise: a) a retaining component; b) one or more binding entities (e.g., an affinity reagent, a ligand-binding polypeptide, a binding ligand, etc.) coupled to the retaining component; and c) one or more detectable labels coupled to the retaining component. In a particular configuration, a detectable probe may comprise: a) a retaining component; b) two or more binding entities (e.g., an affinity reagent, a ligand-binding polypeptide, a binding ligand, etc.) coupled to the retaining component; and c) one or more detectable labels coupled to the retaining component. Such a configuration may be particularly advantageous due to a potential increased avidity effect between the two or more binding entities and a binding target (e.g., a binding ligand, a ligand-binding polypeptide). A retaining component may be selected for a detectable probe based upon one or more of: 1) providing a plurality of coupling sites for the one or more binding entities and the one or more detectable labels; and 2) providing tunable spacing or orientation of the one or more binding entities and the one or more detectable labels. In some configurations, a retaining component may comprise a structured nucleic acid particle (SNAP) or a nanoparticle. The SNAP may comprise a nucleic acid origami or a nucleic acid nanoball. The nanoparticle may comprise a fluorescently-labeled nanoparticle, a polymeric nanoparticle, a dendrimer, a branched polymer, or a quantum dot.

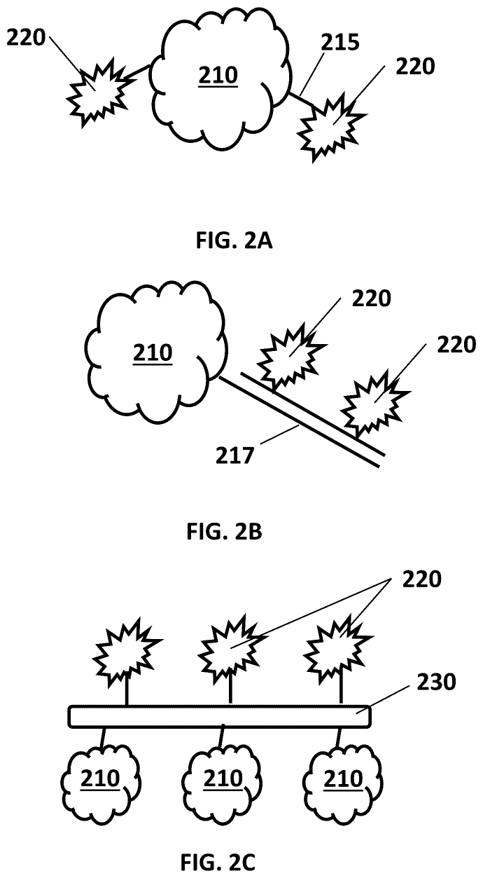

FIGS. 2A-2C depict various configurations of detectable probes that comprise a binding entity. FIG. 2A shows a binding entity (e.g., a ligand-binding polypeptide, a polypeptide binding ligand, etc.) 210 that is coupled to one or more detectable labels 220 (e.g., fluorophores), optionally via a linker 215 (e.g., a hydrocarbon chain). FIG. 2B shows a binding entity (e.g., a ligand-binding polypeptide, a polypeptide binding ligand, etc.) 210 that is coupled to one or more detectable labels 220 by a nucleic acid linker 217 comprising a pair of complementary oligonucleotides. FIG. 2C shows a detectable probe comprising a retaining component 230 (e.g., a structured nucleic acid particle, a nanoparticle) that is coupled to one or more binding entities (e.g., a ligand-binding polypeptide, a polypeptide binding ligand, etc.) 210 and one or more detectable labels 220. The skilled person will readily recognize that the disclosed detectable probe configurations, and variations thereof, may be adapted to produce detectable probes that comprise any useful binding entity, including polypeptide and non-polypeptide binding ligands.

Methods, as set forth herein, utilizing single-analyte arrays of ligand-binding polypeptides may involve a step of detecting the presence or absence of a polypeptide binding interaction at each address at single molecule resolution. A polypeptide binding interaction at an address on an array may be determined in several fashions, including for example: 1) detecting absence of a binding site of a ligand-binding polypeptide; 2) detecting presence of a conformational change in the ligand-binding polypeptide associated with the binding of a binding ligand, for example as indicated by presence of a ligand-binding polypeptide epitope that is buried in an unbound state; and/or 3) detecting presence of a chemical structure (e.g., one or more functional groups, or a polypeptide epitope) associated with a binding ligand but not associated with a ligand-binding polypeptide. In some cases, a polypeptide binding interaction may be determined by two or more measurements that independently provide evidence of the presence or absence of a polypeptide binding interaction. For example, a polypeptide binding interaction between a ligand-binding polypeptide and a polypeptide binding ligand may be evidenced by no detected binding of an affinity agent that binds a binding site of a ligand-binding polypeptide and detected binding of an affinity agent that has a binding specificity for an epitope not present in the ligand-binding polypeptide.

Polypeptide binding interactions between ligand-binding polypeptides and binding ligands may be detected by the formation of polypeptide complexes and the subsequent detection of the polypeptide complexes on a solid support at single-analyte resolution. A polypeptide complex may comprise a ligand-binding polypeptide coupled to one or more binding ligands. In some cases, a polypeptide complex may be formed before forming an array comprising the polypeptide complex at an address on the array. For example, a polypeptide complex comprising an albumin polypeptide coupled to an insulin peptide may be extracted from a blood sample, then the polypeptide complex may be deposited at an address on a solid support. In another example, a ligand-binding polypeptide extracted from a subject may be combined after extraction with a binding ligand for the ligand-binding polypeptide to form a polypeptide complex, then the polypeptide complex may be deposited at an address on a solid support. In other cases, a polypeptide complex may be formed on a single-analyte array. For example, an array comprising a plurality of ligand-binding polypeptides may be contacted with one or more binding ligands to form one or more polypeptide complexes at one or more addresses on the array. A method, as set forth herein, may comprise a step of stabilizing a polypeptide complex. In some cases, stabilizing a polypeptide complex may comprise cross-linking a ligand-binding polypeptide to a binding ligand of the ligand-binding polypeptide. Cross-linking may occur by any suitable method and/or reagent, such as the use of dimethyl suberimidate, N-hydroxysuccinimide ester, or formaldehyde. In other cases, stabilizing a polypeptide complex may comprise contacting the polypeptide complex with a stabilizing medium, such as a suitable salt or buffer solution.

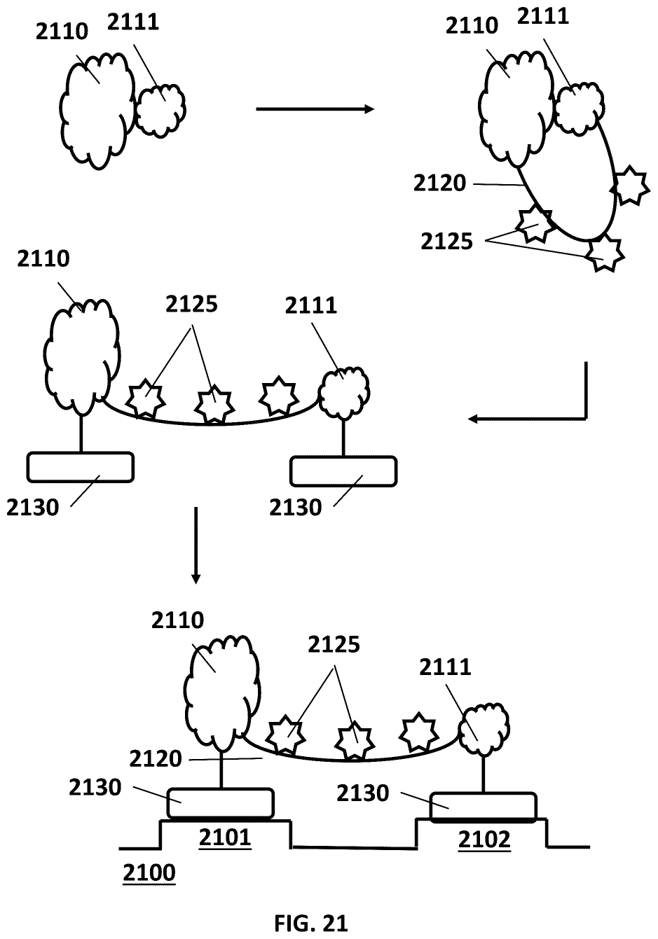

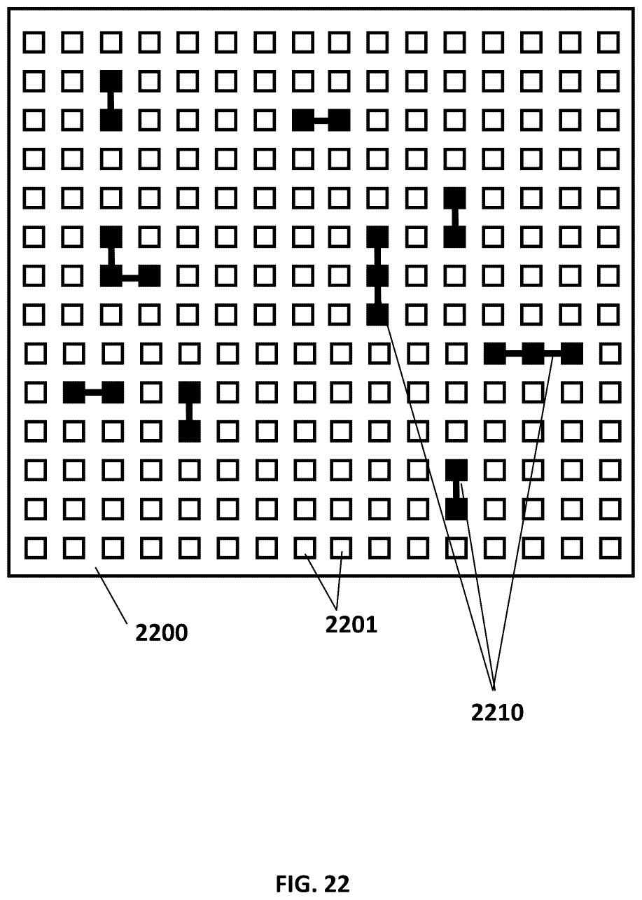

FIG. 21 depicts a method for detecting a polypeptide binding interaction. Initially, a polypeptide complex comprising polypeptides 2110 and 2111 is formed (e.g., in vivo, in vitro, etc.). Subsequently, the polypeptide complex is cross-linked by a cross-linking agent comprising a molecular chain 2120 and a plurality of detectable labels 2125 that are coupled to the molecular chain 2120. The molecular chain 2120 is of sufficient length to permit dissociation of the polypeptide complex into separate polypeptides. After cross-linking, polypeptides 2110 and 2111 are each attached to a different linking moiety 2130, in which each linking moiety 2130 is configured to couple a polypeptide to an analyte binding site of an array. The polypeptides are dissociated so that each polypeptide can be deposited at a separate analyte binding site on the array. Subsequently, the polypeptides are deposited on an array comprising a solid support 2100 by coupling the linking moieties 2130 to analyte binding sites 2101 or 2102 on solid support 2100. Polypeptide 2110 is localized at the array address corresponding to analyte binding site 2101, and polypeptide 2111 is localized at the array address corresponding to analyte binding site 2102. The molecular chain 2120 provides a detectable signal from detectable labels 2125 that identify analyte binding sites 2101 and 2102 as comprising polypeptides from a cross-linked polypeptide complex. FIG. 22 depicts a top-down view of an array 2200 comprising binding sites 2201 with detectable traces 2210 highlighting associated analyte binding sites comprising cross-linked polypeptides, per the method of FIG. 21.

Single-analyte, array-based methods for detecting polypeptide binding interactions may be useful for characterizing polypeptide complexes. Methods provided herein permit deposition of intact polypeptide complexes directly on an array or, alternatively deposition of binding entities on an array followed by formation of polypeptide complexes that include the previously deposited binding entities. Both approaches may provide useful biological information. Polypeptide complex formation on arrays may permit rapid, broad analysis of potential polypeptide binding interactions between ligand-binding polypeptides and candidate binding ligands. For example, an albumin or globulin can be screened against millions or billions of polypeptide or non-polypeptide variants to find variants with enhanced binding specificity. In contrast, pre-deposition complexing may provide which polypeptide binding interactions are likely to occur in a biological system. For example, albumin may bind to certain species in situ on an array-based system, but complexing between albumin and the certain species may not be involved in samples collected from a subject due to low binding affinity or competitive effects with other binding ligands. Moreover, single-analyte, array-based methods provide a platform for capturing polypeptide complexes, then identifying and/or quantifying the binding ligands, including low copy number binding ligands. Low copy number biomarkers (e.g., disease biomarkers) that bind to albumin, for example, may be undetectable in blood samples by traditional analysis (e.g., mass spectrometry) because the low copy number interactions are lost in the data noise of higher frequency binding interactions. The methods provided herein may provide a high-sensitivity, non-destructive and/or non-disruptive approach to identifying polypeptide binding interactions with low copy number species, as well as identifying potential competitive interactions or dissociative interactions that disrupt the stability of polypeptide complexes.

In an aspect, provided herein is a method comprising: a) providing an array comprising a plurality of polypeptide complexes, in which each polypeptide complex of the plurality of polypeptide complexes comprises a ligand-binding polypeptide coupled to a binding ligand, in which the array comprises a plurality of addresses, in which each address of the plurality of addresses is resolvable at single-analyte resolution; in which each polypeptide complex is coupled to an address of the plurality of addresses, and optionally in which the ligand-binding polypeptide comprises an albumin or a globulin; b) contacting the array with a plurality of detectable probes, in which a detectable probe of the plurality of detectable probes comprises a binding specificity for a polypeptide complex; and c) determining presence or absence of the polypeptide complex of the plurality of polypeptide complexes at each address of the plurality of addresses, in which determining presence or absence of the polypeptide complex of the plurality of polypeptide complexes comprises detecting signal from the detectable probe of the plurality of detectable probes. A detectable probe with a binding specificity for a polypeptide complex may comprise a detectable probe and/or affinity agent as set forth herein. For example, a detectable probe for detecting a polypeptide complex may comprise an affinity agent with a binding specificity for an epitope associated with a binding site of a ligand-binding polypeptide. In such cases, the absence of detectable probe binding may be evidence of a polypeptide complex being present. In another example, a detectable probe for detecting a polypeptide complex may comprise an affinity agent with a binding specificity for an epitope associated with a binding ligand but not associated with a ligand-binding polypeptide. In such cases, presence of detectable probe binding may be evidence of a polypeptide complex being present.

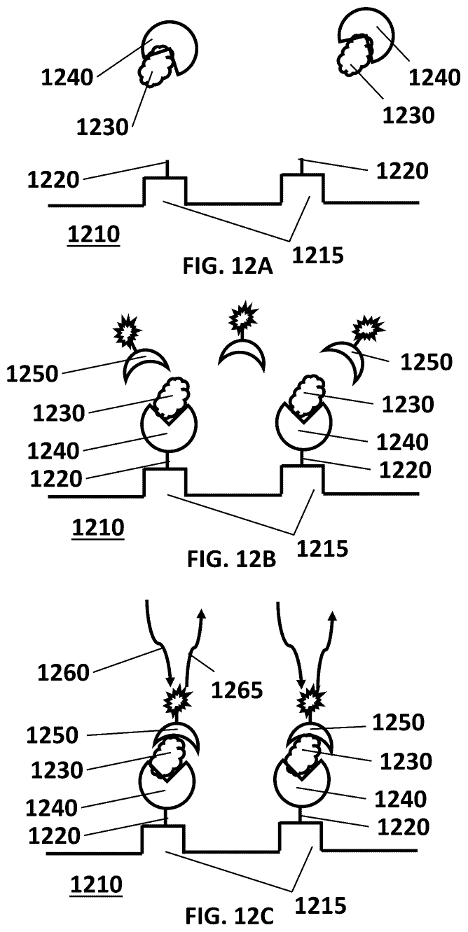

FIGS. 12A-12C illustrate a method of forming a single-analyte array of polypeptide complexes by directly depositing a plurality of polypeptide complexes on a solid support. FIG. 12A shows an array comprising a solid support 1210 containing a plurality of addresses 1215. Each address is coupled a polypeptide complex, optionally via a linker 1220 that is coupled at each address of the plurality of addresses 1215. A plurality of polypeptide complexes 1240 is contacted with the solid support 1210, optionally within a fluidic medium, in which each polypeptide complex comprising a ligand-binding polypeptide 1240 that forms a polypeptide binding interaction with a binding ligand 1230. As shown in FIG. 12B, each address of the plurality of addresses 1215 has coupled a single polypeptide complex, optionally via the linker 1220. A plurality of detectable affinity agents is contacted with the solid support 1210, optionally within a fluidic medium, in which a detectable affinity agent 1250 of the plurality of detectable affinity agents comprises a lack of binding specificity for the ligand-binding polypeptide 1240. As shown in FIG. 12C, one or more detectable affinity agents 1250 have coupled to one or more binding ligands 1230. Presence or absence of the detectable affinity agent 1250 at each address of the plurality of addresses 1215 may be measured by detecting the presence or absence of signal 1265 at each address. The signal 1265 is optionally induced by an excitation source 1260 (e.g., a light source). Optionally, the method may further comprise additional cycles of contacting the array with detectable affinity agents and detecting the presence or absence of binding at each address. Optionally, the method may further comprise identifying one or more binding ligands 1230 at one or more addresses of the plurality of addresses 1215 based upon the measured binding profiles of the one or more detectable affinity agents.

A method, as set forth herein, may comprise a step of disrupting a polypeptide complex before coupling a heterogeneous plurality of polypeptides to a solid support, in which the polypeptide complex comprises a ligand-binding polypeptide and a polypeptide binding ligand, and in which the heterogeneous plurality of polypeptides comprises the ligand-binding polypeptide and the polypeptide binding ligand. For example, it may be advantageous to disrupt a polypeptide complex to increase the likelihood of detecting and/or identifying the binding ligand in some methods set forth herein. In another example, free polypeptide complexes formed during a competitive binding method may be captured in a fluid phase that is removed from a first array, then the complexes can be disrupted before coupling each component of the free polypeptide complex to a second array for further analysis. In some cases, disrupting the polypeptide complex may comprise denaturing the polypeptide complex. Denaturing a polypeptide complex may occur via contacting a polypeptide complex with a fluidic medium comprising a denaturing agent (e.g., urea, guanidinium chloride, trichloroacetic acid, sodium dodecyl sulfate, dithiothreitol, etc.) or heating a polypeptide complex. In other cases, disrupting a polypeptide complex may comprise the steps of: a) contacting the polypeptide complex with a competitive binding ligand, in which the competitive binding ligand is a binding ligand for a ligand-binding polypeptide; and b) coupling the competitive binding ligand to the ligand-binding polypeptide, thereby releasing the polypeptide binding ligand. In some cases, a competitive binding ligand may comprise a non-polypeptide molecule.

A method of the present disclosure may detect and/or measure competitive binding to characterize the polypeptide binding interactions of ligand-binding polypeptides. Competitive binding methods may be characterized as comprising forming a polypeptide-binding interaction between a first binding entity (e.g., a ligand-binding polypeptide, a binding ligand) and a second binding entity of two or more species of second binding entities (e.g., two differing ligand-binding polypeptides, two differing binding ligands, etc.), in which a first binding entity has an opportunity to form a polypeptide binding interaction with each binding entity of the two or more species of second binding entity. Competitive binding methods may be varied according to type of competitor, type of binding competition, and assay sequence method. Table I sets forth differences between variations of competitive binding methods. Each competitive binding method may be distinguished by a competitor type (i.e., differing species of binding entity competing to participate in a polypeptide binding interaction), binding competition type (i.e., formation or disruption of a polypeptide binding interaction), and assay sequencing (i.e., the timing of binding competitors being provided to the assay to participate in polypeptide binding interactions).

TABLE I

| | | |
|---|---|---|
| Competitor | Ligand-binding polypeptide- | Binding ligand-utilizes two |
| Type | utilizes two or more variants | or more variants of binding |
| | of a ligand-binding | ligand or two different |
| | polypeptide or two different | species of binding ligand |
| | species of ligand-binding | |
| | polypeptides | |
| Binding | Constructive-assay may | Disruptive-assay may result |
| Competition | result in formation of | in dissociation of polypeptide |
| | polypeptide complexes from | complexes or change in the |
| | arrays of uncoupled binding | composition of polypeptide |
| | entities | complexes |
| Assay | Simultaneous-assay may | Sequential-polypeptide |
| Sequence | provide each of two or more | complexes may be formed |
| | binding entities at the same | with a first binding entity, |
| | time and allow each to | then a second binding entity |
| | compete for binding partners | may be subsequently |
| | | introduced to displace or |
| | | replace the first binding entity |

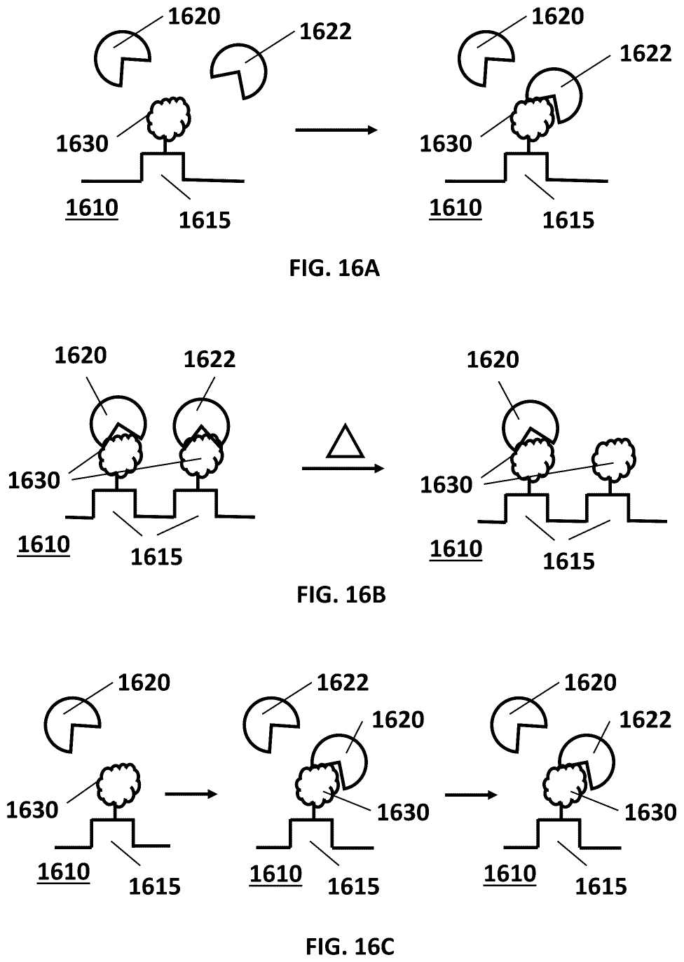

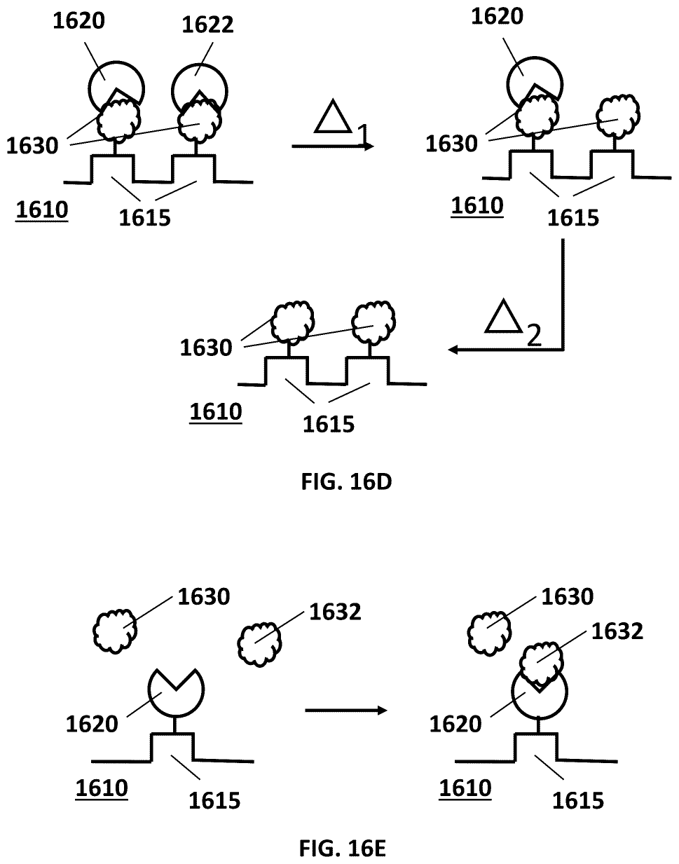

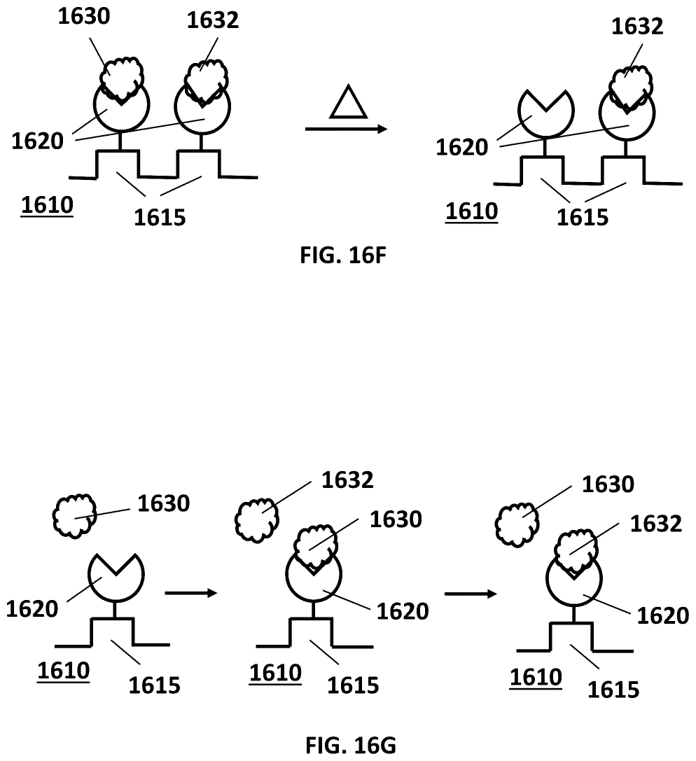

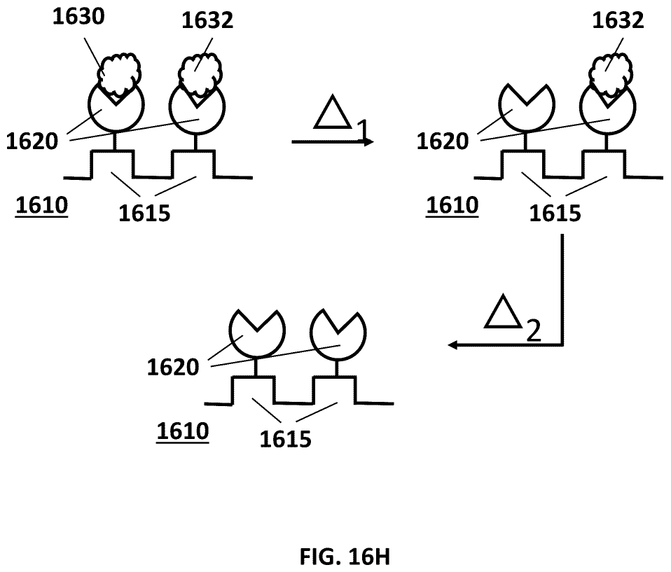

FIGS. 16A-16H illustrate variations of competitive binding methods, as set forth in Table I. FIG. 16A depicts a constructive competitive binding assay with ligand-binding polypeptide competitors and simultaneous competition. A single-analyte array comprising a solid support 1610 may comprise an address 1615 containing a coupled binding ligand 1630. The solid support 1610 may be simultaneously contacted with a first ligand-binding polypeptide 1620 and a second ligand-binding polypeptide 1622. After contacting the single-analyte array with the ligand-binding polypeptides, a polypeptide binding interaction may be detected, for example, an interaction between the binding ligand 1630 and the second ligand-binding polypeptide 1622. FIG. 16B depicts a disruptive competitive binding assay with ligand-binding polypeptide competitors and simultaneous competition. A single-analyte array comprising a solid support 1610 may comprise two or more addresses 1615 with each address containing a coupled binding ligand 1630. A first ligand-binding polypeptide 1620 may bind a binding ligand 1630 at an address 1615, and a second ligand-binding polypeptide 1622 may bind a binding ligand 1630 at a different address 1615. The solid support may be exposed to a differing environmental condition Δ (e.g., a temperature change, an altered fluid composition, etc.) that causes dissociation of the second ligand-binding polypeptide 1622 but not the first ligand-binding polypeptide 1620. FIG. 16C depicts a constructive competitive binding assay with ligand-binding polypeptide competitors and sequential competition. A single-analyte array comprising a solid support 1610 may comprise an address 1615 containing a coupled binding ligand 1630. A ligand-binding polypeptide may be contacted to the solid support 1610, facilitating formation of a first polypeptide binding interaction between the binding ligand 1630 and the first ligand-binding polypeptide 1620. Subsequently, a second ligand-binding polypeptide 1622 may be contacted with the solid support 1610, possibly causing disruption of the first polypeptide binding interaction, and formation of a second polypeptide binding interaction between the binding ligand 1630 and the second ligand-binding polypeptide 1622. FIG. 16D depicts a disruptive competitive binding assay with ligand-binding polypeptide competitors and sequential competition. A single-analyte array comprising a solid support 1610 may comprise two or more addresses 1615 with each address containing a coupled binding ligand 1630. The solid support may be exposed to a first differing environmental condition Δ₁ (e.g., a temperature change, an altered fluid composition, etc.) that causes dissociation of the second ligand-binding polypeptide 1622 but not the first ligand-binding polypeptide 1620. Subsequently, the solid support may be exposed to a second differing environmental condition Δ₂ that causes dissociation of the first ligand-binding polypeptide 1620.

FIG. 16E depicts a constructive competitive binding assay with binding ligand competitors and simultaneous competition. A single-analyte array comprising a solid support 1610 may comprise an address 1615 containing a coupled ligand-binding polypeptide 1620. The solid support 1610 may be simultaneously contacted with a first binding ligand 1630 and a second binding ligand 1632. After contacting the single-analyte array with the binding ligands, a polypeptide binding interaction may be detected, for example an interaction between the second binding ligand 1632 and the ligand-binding polypeptide 1620. FIG. 16F depicts a disruptive competitive binding assay with binding ligand competitors and simultaneous competition. A single-analyte array comprising a solid support 1610 may comprise two or more addresses 1615 with each address containing a coupled ligand-binding polypeptide 1620. A first binding ligand 1630 may bind a ligand-binding polypeptide 1620 at an address 1615, and a second binding ligand 1632 may bind a ligand-binding polypeptide 1620 at a different address 1615. The solid support may be exposed to a differing environmental condition Δ (e.g., a temperature change, an altered fluid composition, etc.) that causes dissociation of the first binding ligand 1630 but not the second ligand-binding polypeptide 1632. FIG. 16G depicts a constructive competitive binding assay with binding ligand competitors and sequential competition. A single-analyte array comprising a solid support 1610 may comprise an address 1615 containing a coupled ligand-binding polypeptide 1620. A first binding ligand 1630 may be contacted to the solid support 1610, facilitating formation of a first polypeptide binding interaction between the binding ligand 1630 and the ligand-binding polypeptide 1620. Subsequently, a second binding ligand 1632 may be contacted with the solid support 1610, possibly causing disruption of the first polypeptide binding interaction, and formation of a second polypeptide binding interaction between the second binding ligand 1632 and the ligand-binding polypeptide 1620. FIG. 16H depicts a disruptive competitive binding assay with ligand-binding polypeptide competitors and sequential competition. A single-analyte array comprising a solid support 1610 may comprise two or more addresses 1615 with each address containing a coupled ligand-binding polypeptide 1620. The solid support may be exposed to a first differing environmental condition Δ₁ (e.g., a temperature change, an altered fluid composition, etc.) that causes dissociation of the first binding ligand 1630 but not the second binding ligand 1632. Subsequently, the solid support may be exposed to a second differing environmental condition Δ₂ that causes dissociation of the second binding ligand 1632. The skilled person will readily recognize that the above-described competitive binding methods can be arranged in numerous fashions, including the presence of more than two binding competitors.

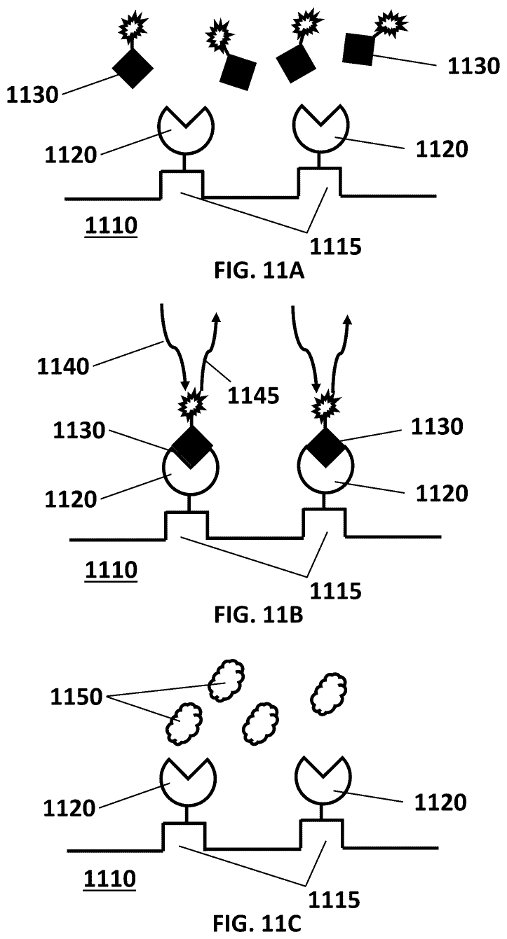

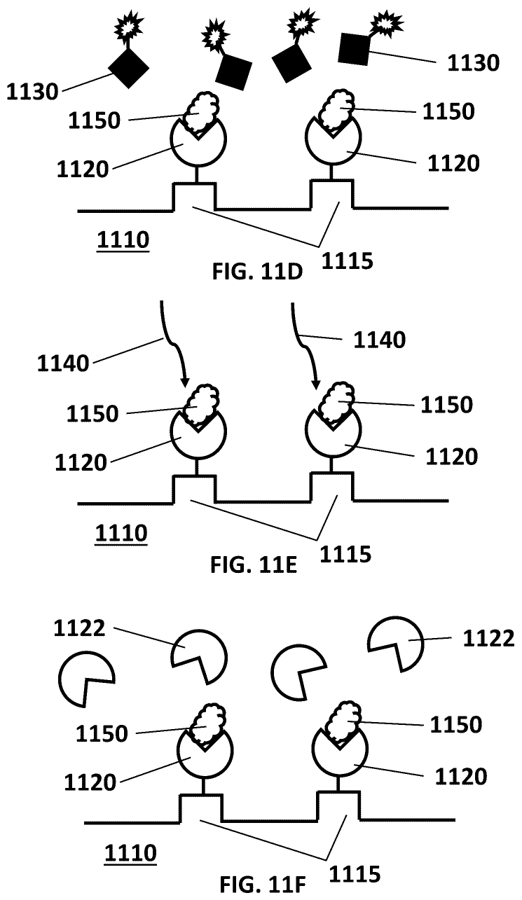

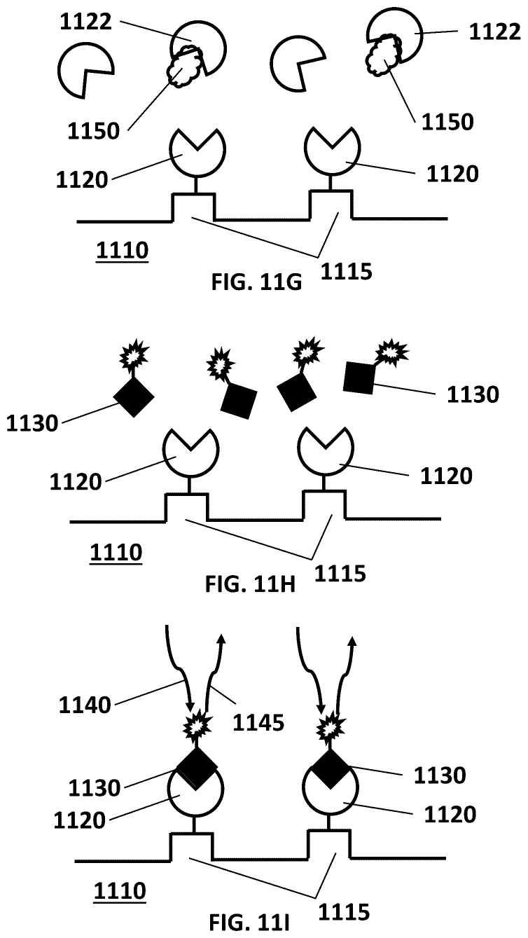

FIGS. 11A-11I illustrate a method of detecting the dissociation of a polypeptide complex by a competitive binding assay. FIG. 11A shows an array comprising a solid support 1110 comprising a plurality of addresses 1115. Each address of the plurality of addresses 1115 is coupled to a single ligand-binding polypeptide 1120. A plurality of detectable probes is contacted with the solid support 1110, optionally within a fluidic medium, in which a detectable probe 1130 of the plurality of detectable probes comprises a binding specificity for the ligand-binding polypeptide 1120. FIG. 11B shows one or more detectable probes 1130 coupled to ligand-binding polypeptides 1120 at one or more addresses of the plurality of addresses 1115. Presence or absence of a detectable probe 1130 is determined at each address of the plurality of addresses 1115 by detecting a signal 1145 produced by the detectable probe 1130. Optionally, the signal 1145 is induced by an excitation source 1140 (e.g., a light source). FIG. 11C shows a plurality of binding ligands being contacted with the solid support 1110, optionally within a fluidic medium. FIG. 11D shows the array comprising one or more addresses containing a polypeptide complex comprising a ligand-binding polypeptide 1120 forming a polypeptide binding interaction with a binding ligand 1150. A plurality of detectable probes is contacted with the solid support 1110, optionally within a fluidic medium, in which a detectable probe 1130 of the plurality of detectable probes comprises a binding specificity for the ligand-binding polypeptide 1120. Due to the presence of binding ligands 1150 at one or more addresses, binding of a detectable probe 1130 at the one or more addresses is inhibited. FIG. 11E shows the detection of presence or absence of a detectable probe 1130 at each address of the plurality of addresses 1115. An optional excitation field 1140 is provided, but no signal is detected from addresses comprising a polypeptide complex. As shown in FIG. 11F, a plurality of free ligand-binding polypeptide is contacted with the solid support 1110. As shown in FIG. 11G, one or more free polypeptide complexes are formed by dissociating a binding ligand 1150 from one or more array-based polypeptide complexes, in which each free polypeptide complex comprises a free ligand-binding polypeptide 1122 forming a polypeptide binding interaction with a binding ligand 1150. As shown in FIG. 11H, plurality of detectable probes is contacted with the solid support 1110, optionally within a fluidic medium, in which a detectable probe 1130 of the plurality of detectable probes comprises a binding specificity for the ligand-binding polypeptide 1120. FIG. 11I shows one or more detectable probes 1130 coupled to ligand-binding polypeptides 1120 at one or more addresses of the plurality of addresses 1115. Presence or absence of a detectable probe 1130 is determined at each address of the plurality of addresses 1115 by detecting a signal 1145 produced by the detectable probe 1130. Optionally, the signal 1145 is induced by an excitation source 1140 (e.g., a light source). Alternating patterns of presence or absence of signal 1145 from detectable probes may be utilized to determine the formation and dissociation of polypeptide complexes on the single-analyte array.

In another aspect, provided herein is a competitive binding method comprising: a) providing an array comprising a plurality of addresses, in which each address of the plurality of addresses comprises a coupled polypeptide complex, in which each address is resolvable from each other address, and in which each coupled polypeptide complex comprises a ligand-binding polypeptide and a binding ligand of the ligand-binding polypeptide; b) detecting presence or absence of a coupled polypeptide complex at each address of the plurality of addresses; c) contacting the array with a plurality of free ligand-binding polypeptides, thereby transferring at least one binding ligand of the plurality of binding ligands from a coupled polypeptide complex to a free ligand-binding polypeptide; and d) optionally detecting an absence of a binding ligand at one or more addresses of the plurality of addresses.

In another aspect, provided herein is a competitive binding method comprising: a) providing an array comprising a plurality of binding ligands, in which each binding ligand of the plurality of binding ligands is located at an address on the array, and in which each address of the array is resolvable from each other address on the array at single-analyte resolution; b) contacting the array with a mixture comprising a first plurality of ligand-binding polypeptides and a second plurality of ligand-binding polypeptides, in which a ligand-binding polypeptide of the first plurality of ligand-binding polypeptides comprises a first binding specificity, and in which a ligand-binding polypeptide of the second plurality of ligand-binding polypeptides comprises a second binding specificity; c) forming a first polypeptide complex at a first address and optionally forming a second polypeptide complex, in which the first polypeptide complex comprises a binding ligand coupled to the ligand-binding polypeptide of the first plurality of ligand-binding polypeptides, and in which the second polypeptide complex comprises a binding ligand coupled to the ligand-binding polypeptide of the second plurality of ligand-binding polypeptides; d) identifying the first address comprising the first polypeptide complex; and e) determining the presence or absence of the first polypeptide complex or the second polypeptide complex at each address other than the first address.

In another aspect, provided herein is a competitive binding method comprising: a) providing an array comprising a plurality of ligand-binding polypeptides, in which each ligand-binding polypeptide of the plurality of ligand-binding polypeptides is located at an address on the array, and in which each address of the array is resolvable from each other address on the array at single-analyte resolution; b) contacting the array with a mixture comprising a first plurality of binding ligands and a second plurality of binding ligands, in which a ligand-binding polypeptide of the plurality of ligand-binding polypeptides comprises a first binding affinity for a binding ligand of the first plurality of binding ligands, and in which the ligand-binding polypeptide of the plurality of ligand-binding polypeptides comprises a second binding affinity for a binding ligand of the second plurality of binding ligands; c) forming a first polypeptide complex at a first address and optionally forming a second polypeptide complex at a second address, in which the first polypeptide complex comprises a ligand-binding polypeptide coupled to the binding ligand of the first plurality of binding ligands, and in which the second polypeptide complex comprises a ligand-binding polypeptide coupled to the binding ligand of the second plurality of binding ligands; d) identifying the first address comprising the first polypeptide complex; and e) determining the presence or absence of the first polypeptide complex or the second polypeptide complex at each address other than the first address.

In another aspect, provided herein is a competitive binding method comprising: a) providing an array comprising a plurality of addresses, in which each address of the plurality of addresses comprises a coupled first polypeptide complex, in which each address is resolvable from each other address at single-analyte resolution, in which each first polypeptide complex comprises a ligand-binding polypeptide and a binding ligand of the ligand-binding polypeptide, and in which the ligand-binding polypeptide comprises a first binding affinity for the binding ligand; b) contacting the array with a plurality of free binding ligands, in which the free binding ligand comprises a different chemical structure than the binding ligand, and in which the ligand-binding polypeptide comprises a second binding affinity for the free binding ligand; c) exchanging a binding ligand with a free binding ligand, thereby forming one or more second polypeptide complexes, in which a second polypeptide complex of the one or more second polypeptide complexes comprises the ligand-binding polypeptide coupled to the free binding ligand; and d) detecting a presence or absence of the second polypeptide complex at each address on the array.

The skilled person will readily recognize that the methods of array formation, methods of forming polypeptide binding interactions, and methods of detecting and/or characterizing polypeptide binding interactions, as set forth herein, may be readily adapted for competitive binding assays by the addition of one or more competitive binding entities (e.g., competitive binding ligands, competitive ligand-binding polypeptides, etc.). In some cases of the competitive binding methods, as set forth herein, it may be advantageous to identify presence or absence of polypeptide complexes at each address and optionally identify the composition of each detected polypeptide complex before and/or after contacting an array with a competitive binding entity. In some cases, one or more steps of a competitive binding method, as set forth herein, may be repeated. For example, a step of contacting an array with a competitive binding entity may be repeated. In other cases, a competitive binding method may comprise a step of contacting an array with a fluidic medium comprising a previously-displaced binding entity and identifying any addresses in which a competitive binding entity is displaced in turn.

A competitive binding method may generate a plurality of free species (e.g., free species in a fluidic medium in contact with an array), such as free competitive binding entities, displaced binding entities, and free polypeptide complexes. A competitive binding method may further comprise the steps of: a) capturing a free species downstream from the solid support; and b) identifying the free species. In some cases, a competitive binding method may further comprise, after contacting an array with a plurality of free ligand-binding polypeptides, performing the steps of: a) capturing a free ligand-binding polypeptide of the plurality of ligand-binding polypeptides downstream from the solid support; and b) detecting presence or absence of a binding ligand coupled to the free ligand-binding polypeptide. In some cases, the competitive binding method may further comprise identifying a binding ligand coupled to the free ligand-binding polypeptide. In some cases, identifying the binding ligand coupled to the free ligand-binding polypeptide comprises a bulk identification assay. In some cases, identifying the binding ligand coupled to the free ligand-binding polypeptide comprises performing a mass spectrometry analysis of the free ligand-binding polypeptide and binding ligand. In some cases, identifying the binding ligand coupled to the free ligand-binding polypeptide comprises performing a single-molecule polypeptide assay, as set forth herein. For example, a free polypeptide complex may be coupled to a second array and subject to an analysis method, as set forth herein. In some cases, identifying a binding ligand coupled to a free ligand-binding polypeptide may comprise one or more of the steps of a) dissociating a polypeptide complex, in which the polypeptide complex comprises a binding ligand coupled to a free ligand-binding polypeptide; b) adding a detectable tag (e.g., a nucleic acid barcode, a peptide barcode) to a portion of the binding ligand; c) sequencing the portion of the binding ligand; and d) identifying the binding ligand of the binding ligand based upon sequencing the portion of the binding ligand. In some cases, sequencing the binding ligand may comprise a fluorosequencing assay or an affinity agent-based sequencing assay. In some cases, a detectable tag may be added to a portion of the binding ligand before the binding ligand is bound by the free ligand-binding polypeptide (e.g., adding a detectable tag while bound to the original polypeptide complex).

A method, as set forth herein, may utilize a second single-analyte array. In another aspect, provided herein is a method comprising one or more steps of: a) contacting a plurality of first binding entities with a first solid support comprising a plurality of addresses, in which each address comprises a single second binding entity of a plurality of second binding entities, and in which each address is resolvable at single-analyte resolution; b) binding a first binding entity of the plurality of first binding entities to a second binding entity at an address on the solid support; c) removing one or more unbound first binding entities from the first solid support; d) binding the one or more unbound first binding entities to a second solid support; and e) characterizing a first binding entity of the one or more unbound first binding entities bound to the solid support.

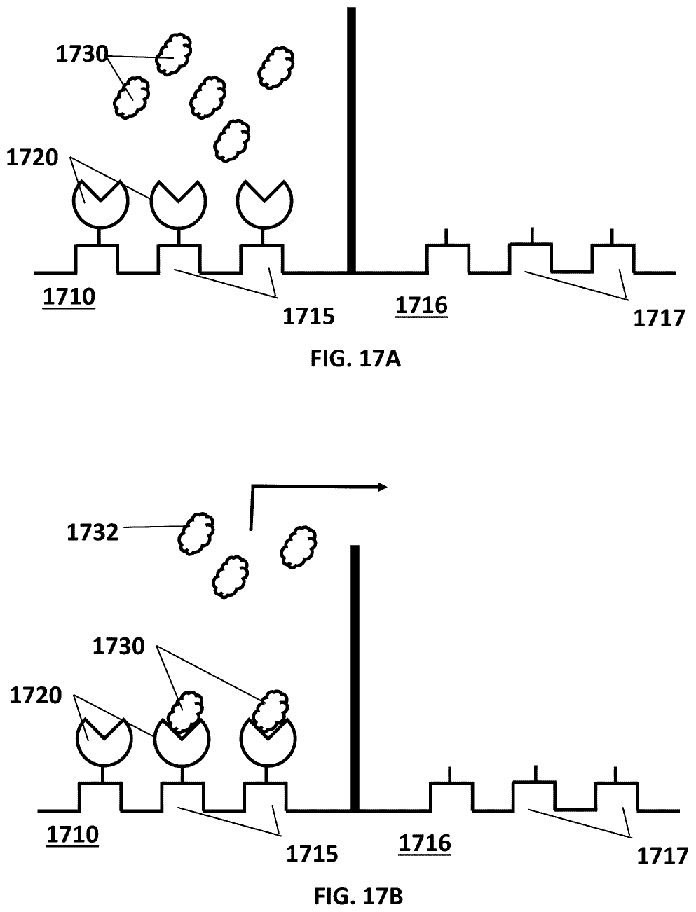

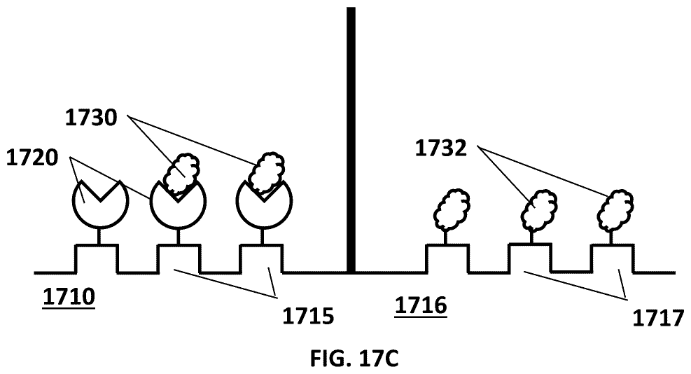

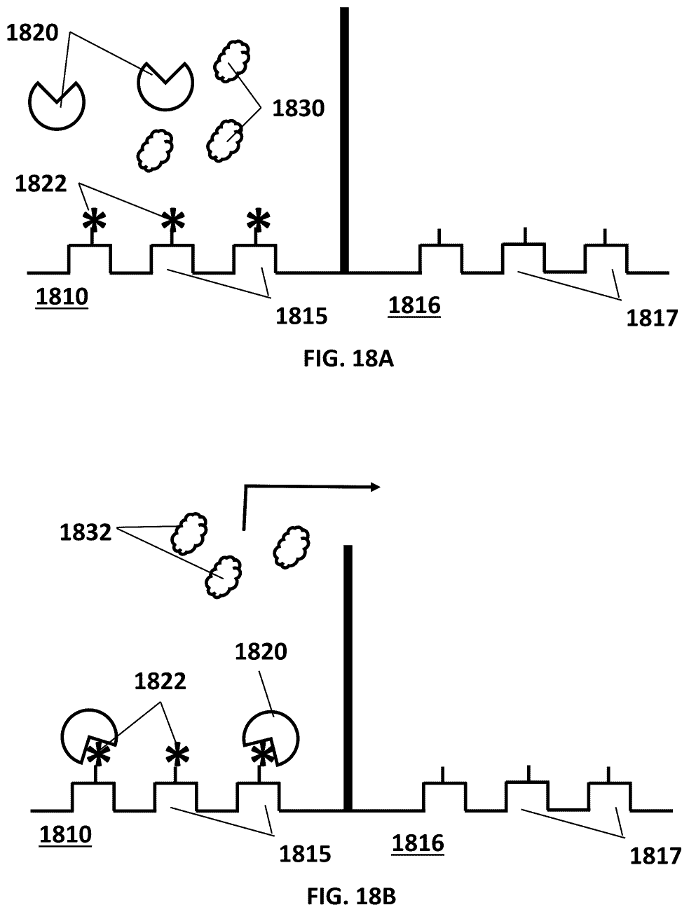

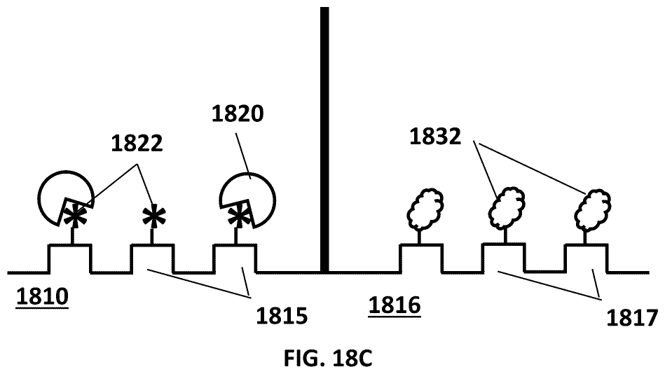

FIGS. 17A-17C and 18A-18C depict methods of utilizing two single-analyte arrays to characterize polypeptide binding interactions. FIGS. 17A-17C depict a method that utilizes a single-analyte array comprising ligand-binding polypeptides at each address on the array. FIG. 17A illustrates a system comprising a first solid support 1710 comprising a first plurality of addresses 1715, and a second solid support 1716 comprising a second plurality of addresses 1717. Each address of the first plurality of addresses 1715 contains a single, coupled ligand-binding polypeptide 1720. The first solid support 1710 is contacted with a plurality of polypeptides 1730, thereby facilitating formation of a polypeptide binding interaction between a polypeptide 1730 and a ligand-binding polypeptide 1720. As shown in FIG. 17B, after forming polypeptide binding interactions involving one or more polypeptides 1730, a remaining fraction of unbound polypeptides 1732 is removed (e.g., by a rinsing fluid) to the second solid support 1716. As shown in FIG. 17C, the remaining fraction of unbound polypeptides 1732 may be coupled to addresses 1717 on the second solid support 1716. Subsequently, the first array or the second array may undergo a characterization assay, as set forth herein, for example to identify one or more polypeptides that did not bind with a ligand-binding polypeptide.

FIGS. 18A-18C depict a method that utilizes an array of binding ligands for a ligand-binding polypeptide to remove ligand-binding polypeptides from a plurality of polypeptides. FIG. 18A illustrates a system comprising a first solid support 1810 comprising a first plurality of addresses 1815, and a second solid support 1816 comprising a second plurality of addresses 1817. Each address of the first plurality of addresses 1815 contains a single, coupled binding ligand 1822. The first solid support 1810 is contacted with a plurality of polypeptides containing ligand-binding polypeptides 1820 and non-ligand binding polypeptides 1830, thereby facilitating formation of a polypeptide binding interaction between a binding ligand 1822 and a ligand-binding polypeptide 1820. As shown in FIG. 18B, after forming polypeptide binding interactions involving one or more ligand-binding polypeptides 1820, a remaining fraction of unbound polypeptides 1832 is removed (e.g., by a rinsing fluid) to the second solid support 1816. As shown in FIG. 18C, the remaining fraction of unbound polypeptides 1832 may be coupled to addresses 1817 on the second solid support 1816. Subsequently, the first array or the second array may undergo a characterization assay, as set forth herein, for example to identify one or more polypeptides that did not bind with a ligand-binding polypeptide.

A method that utilize a second single-analyte array may be useful for multiple types of assays, including: 1) facilitating the removal of common polypeptide or non-polypeptide species from a sample, thereby increasing the relative amounts of low copy number species on the second array; 2) identifying and/or characterizing binding entities that did not participate in a known or expected polypeptide binding interaction (e.g., capturing on a second array an albumin that did not bind a known albumin binding ligand on a first array, and characterizing possible modifications to the albumin structure on the second array); and 3) reducing the complexity of identification and/or characterization on a second array by depleting particular species from a mixture of species via capture on a first array. The skilled person will recognize that, in some configurations, particular species may be depleted on a non-single analyte solid support (e.g., an affinity chromatography column. In other configurations, it may be advantageous to deplete a particular species via capture on a first single-analyte array and then characterize species captured on the first array and the second array.

A method, as set forth herein, may include a longitudinal or temporal series of measurements. A longitudinal series of measurements may comprise measurements of multiple differing samples, for example from a cohort of subjects, or several samples collected individually from a single subject. For example, a longitudinal series of measurements may comprise blood-extracted polypeptide samples, with each sample collected from a different human suspected of having a medical condition (e.g., diabetes). In another example, a longitudinal series of measurements may comprise multiple polypeptide samples derived from differing bodily fluids (e.g., blood, cerebrospinal fluid, synovial fluid, etc.) of a human suspected of having a medical condition (e.g., an inflammatory disorder). A temporal series of measurements may comprise a time sequence of measurements from a single subject or from multiple subjects. For example, a temporal series of measurements may comprise two or more polypeptide samples collected from a subject at differing times. In another example, a temporal series of measurements may comprise a single sample that is measured by a method, as set forth herein at two differing times. A longitudinal or temporal series of measurements may be performed on a plurality of single-analyte arrays, in which each single-analyte array contains a differing sample from each other array. A longitudinal or temporal series of measurements may be performed collectively on a single single-analyte array, for example by including an identifying label (e.g., a fluorescent label) or tag (e.g., a peptide or nucleic acid barcode) that uniquely identifies a sample from which a binding entity was derived.

A method of the present disclosure may be utilized to increase the detection efficiency of low copy number polypeptides (e.g., polypeptides comprising no more than 10000, 5000, 2500, 1000, 500, 250, 100, 50, 25, 10, or less in a sample) at single-analyte resolution for polypeptide systems with a broad range of polypeptide concentrations. An exemplary polypeptide system with a broad range of polypeptide concentrations is blood serum, which can contain over 50% to 60% serum albumin by weight relative to total protein weight, and can contain a further 5% to 10% globulins by weight relative to total protein weight. Accordingly, a single-analyte array comprising individual blood serum polypeptides may have up to 70% of array addresses occupied solely by serum albumin and globulins, thereby increasing the difficulty of detecting low copy number polypeptides, including possible clinically-relevant biomarkers, amongst the bulk of serum albumin and globulins. One proteomic approach to detection of low copy number polypeptides involves performing one or more separation steps to remove a fraction of the high-copy number polypeptides (e.g., albumins, globulins, etc.). Surprisingly, a method, as set forth herein, may be useful for increasing the detection efficiency of low-copy number polypeptides without necessitating separation or removal of high-copy number polypeptides.

In another aspect, provided herein is a method comprising: a) providing an array comprising a plurality of polypeptides, in which each polypeptide of the plurality of polypeptides is located at an address that is resolvable from each other address on the array, and in which each address of the array is detectable at single molecule resolution, in which the plurality of polypeptides comprises a ligand-binding polypeptide, in which the plurality of polypeptides comprises two or more polypeptide species; b) detecting presence or absence of binding of an affinity agent of a first plurality of affinity agents to a polypeptide of the plurality of polypeptides at each address of the plurality of addresses, in which the affinity agent comprises a binding specificity for the ligand-binding polypeptide; c) determining presence or absence of the ligand-binding polypeptide at a subset of addresses of the plurality of addresses beyond a threshold measure of confidence; and d) detecting presence or absence of binding of an affinity agent of a second plurality of affinity agents at each address of the plurality of addresses excluding the subset of addresses where presence of the ligand-binding polypeptide is determined beyond the threshold measure of confidence. Such a method may be particularly advantageous for reducing the complexity of identifying the presence and/or identity of low copy number polypeptides on an array by rapidly identifying high copy number polypeptides and removing addresses corresponding to the high copy number polypeptides from additional analysis.

In some cases, a method of detecting and/or identifying low copy number binding ligands may be bifurcated into first identifying addresses on an array containing high copy number ligand-binding polypeptides, then focusing subsequent analysis on detecting and/or identifying low copy number binding ligands at addresses other than those containing high copy number ligand-binding polypeptides. In some cases, an analysis method may utilize a second plurality of affinity agents, in which the second plurality of affinity agents may comprise an affinity agent with a lack of binding specificity for the ligand-binding polypeptide. In particular cases, a method may comprise contacting an array with one or more additional pluralities of affinity agents, in which each additional plurality of affinity agents comprises an affinity agent with a lack of binding specificity for the ligand-binding polypeptide. Such affinity agents may be chosen to increase the likelihood of binding to a target other than the ligand binding polypeptide. In some cases, a method may further comprise identifying a polypeptide at an address of the plurality of addresses excluding the subset of addresses where the presence of the ligand-binding polypeptide was determined.

A method, as set forth herein, may utilize one or more affinity agents to characterize and/or identify a binding entity. An affinity agent may be particularly useful if it possesses one or more of the properties of: 1) having a known or characterized binding specificity for one or more binding entities or epitopes thereof; 2) having a known or characterized lack of binding specificity for a binding entity (e.g., a ligand-binding polypeptide); 3) having a known or characterized binding specificity for a binding site of a ligand-binding polypeptide; and 4) having a known or characterized binding specificity for a modification or alteration of a binding entity (e.g., a post-translational modification, a chemical derivative of a molecule, etc.). A binding specificity for an affinity reagent may be characterized in terms of a binding affinity for a binding entity or an epitope thereof, for example by a measure such as dissociation constant (K_{D}), dissociation rate constant (k_{off}), or association rate constant (kₒₙ). In some cases, a known or characterized lack of binding specificity for a binding entity, such as a ligand-binding polypeptide, may comprise having a probability of the affinity agent binding to the binding entity beneath a threshold value at a given physical condition (e.g., fluidic composition, temperature, etc.). In other cases, a known or characterized lack of binding specificity for a binding entity, such as a ligand-binding polypeptide, may comprise having a characterized binding affinity that fails to meet a criterium for binding specificity (e.g., possessing a dissociation constant above a threshold value, possessing a dissociation rate constant above a threshold value, possessing an association rate constant below a threshold value, etc.).

An affinity agent, as set forth herein, may be identified by any suitable method. Suitable affinity agents may include aptamers, peptamers, affinity agents, and fragments thereof. Exemplary methods of identifying affinity agents may include SELEX, ELONA, phage display, or antibody screens. In some cases, an affinity agent screening method may include a negative screen. For example, a screening method to identify an affinity agent with a lack of binding to a ligand-binding polypeptide may include a negative screen against the ligand-binding polypeptide. Additional details of affinity agent selection methods are disclosed, for example, in US 20200318101A1 and WO 2020106889A1, each of which is incorporated herein by reference. In some cases, an affinity agent or a panel of differing affinity agents may be selected by an in silico or bioinformatic selection. For example, a bioinformatic method may be applied to identify two or more affinity agents whose presence or absence of binding to a binding ligand comprises a high confidence binding profile for the binding ligand. In another example, a bioinformatic method may be applied to identify an affinity reagent with a lack of binding specificity for a ligand-binding polypeptide (e.g., an affinity agent with a binding specificity for an epitope not present in a ligand-binding polypeptide).

An affinity agent, as set forth herein, may comprise a binding specificity for a natural or engineered modification of a polypeptide, such as a post-translational modification of a ligand-binding polypeptide or a binding ligand. An affinity agent may comprise a component that has a known or characterized specificity for a glycosylation pattern (e.g., a lectin). An affinity agent may have a known or characterized binding specificity for an epitope comprising a post-translational modification (e.g., a disulfide bridge, a ubiquitin, etc.). An affinity agent may have a known or characterized binding specificity for an epitope that is formed in the absence of a post-translational modification. For example, an affinity agent may specifically bind an epitope that is exposed by the disruption of a disulfide bridge. An affinity agent may have a known or characterized binding specificity for an isoform of a binding entity. For example, an affinity agent may bind with high specificity to a glycosylated albumin but does not bind with high specificity to a non-glycosylated albumin.

A method, as set forth herein, may utilize a plurality of polypeptides, for example a plurality of ligand-binding polypeptides, a plurality of polypeptide binding ligands, or a plurality of competitive polypeptide binding ligands. A plurality of polypeptides may be utilized to form arrays of binding targets, to form polypeptide complexes on an array of binding targets, to form detectable probes, and any other purpose set forth herein. In some cases, a plurality of polypeptides may comprise a heterogeneous plurality of polypeptides, in which the heterogeneous plurality of polypeptides comprises at least two polypeptide species. In some cases, a plurality of polypeptides may comprise a heterogeneous plurality of polypeptides, in which the heterogeneous plurality of polypeptides comprises at least one polypeptide species and at least one non-polypeptide species (e.g., a polypeptide and a pharmaceutical binding ligand). In some cases, a heterogeneous plurality of polypeptides may comprise at least about 1%, 5%, 10%, 15%, 20%, 25%, 30%, 35%, 40%, 45%, 50%, 55%, 60%, 65%, 70%, 75%, 80%, 85%, 90%, 95%, 99%, 99.9%, 99.99%, 99.999%, 99.9999%, 99.99999%, or more of a polypeptide species (e.g., a ligand-binding polypeptide, a binding ligand, etc.) on a weight or molar basis relative to total polypeptide content. Alternatively or additionally, a heterogeneous plurality of polypeptides may comprise no more than about 99.99999%, 99.9999%, 99.999%, 99.99%, 99.9%, 99%, 95%, 90%, 85%, 80%, 75%, 70%, 65%, 60%, 55%, 50%, 45%, 40%, 35%, 30%, 25%, 20%, 15%, 10%, 5%, 1% or less of a polypeptide species (e.g., a ligand-binding polypeptide, a binding ligand, etc.) on a weight or molar basis relative to total polypeptide content. In some cases, a plurality of polypeptides may comprise a homogeneous plurality of polypeptides, in which the homogeneous plurality of polypeptides comprises one polypeptide species above a threshold level of purity, such as for example, about 90%, 95%, 99%, 99.9%, 99.99%, 99.999%, 99.9999%, 99.99999%, 99.999999%, 99.9999999%, 99.99999999%, or more on a weight or molar basis relative to total polypeptide content.

Single-analyte, array-based methods of identification of polypeptide binding interactions amongst heterogeneous mixtures of binding targets is useful for analysis of complex systems. Albumin- and/or globulin-containing fluids, such as blood and cerebrospinal fluid, may contain numerous impurities or extraneous species that are typically removed via potentially time-consuming or expensive separation methods. Such pre-analysis processing can potentially cause loss of species of interest due to poor separation or cause disruption of polypeptide complexes due to alteration of the chemical environment that destabilizes polypeptide binding interactions. The methods, as set forth herein, may provide pathways for scavenging species of interest directly from a sample comprising ligand-binding polypeptides without the need for additional purification steps. Moreover, the methods provided herein may permit heterogeneous mixtures comprising polypeptide or non-polypeptide impurities to be deposited on an array because the single-analyte nature of the methods provided allow high-sensitivity detection of binding interactions with even a single copy of a species or polypeptide complex present on the array.

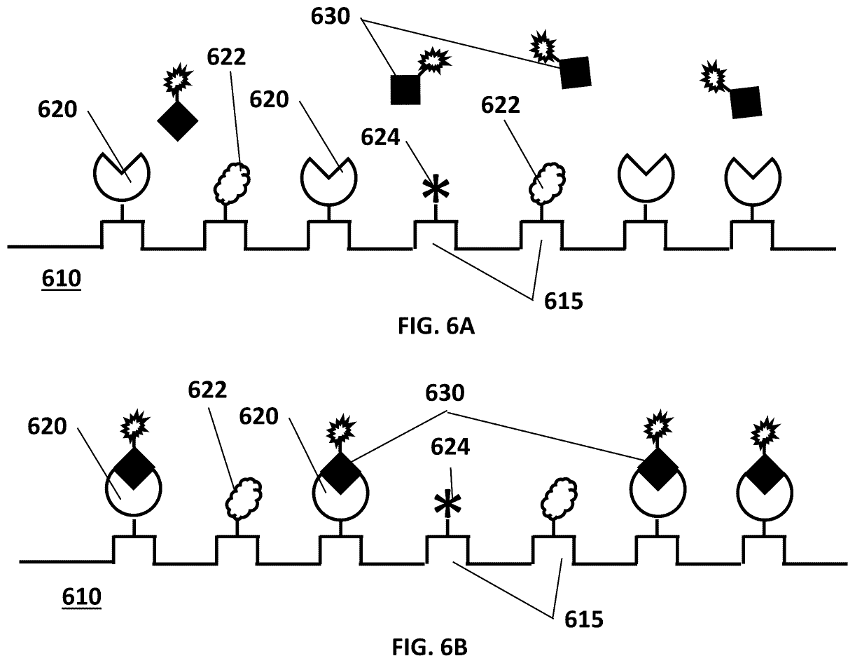

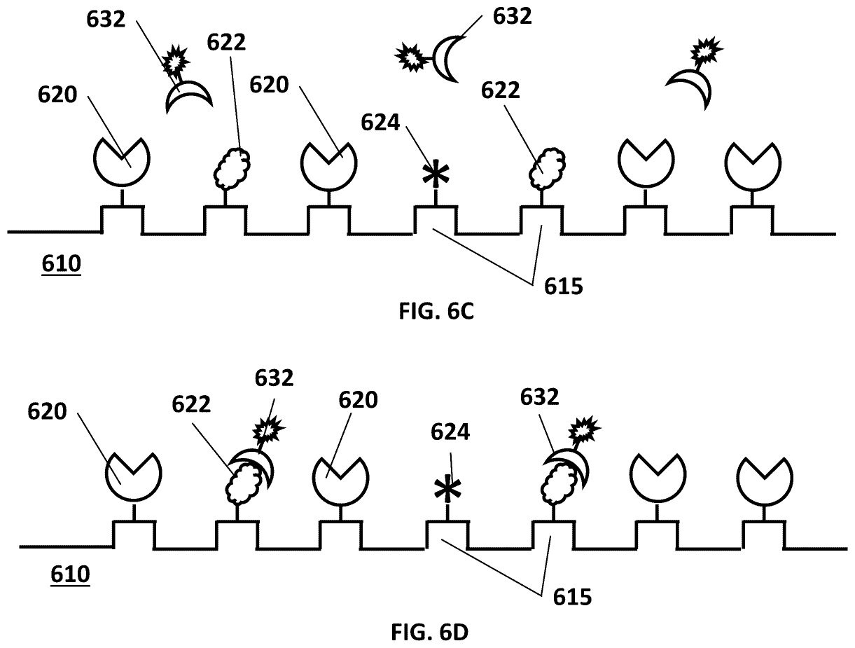

FIGS. 6A-6D depict a method of identifying a polypeptide binding interaction on an array comprising a heterogeneous plurality of binding targets (e.g., a plurality of polypeptides comprising two or more polypeptide species; a plurality of polypeptide species mixed with a plurality of non-polypeptide species, etc.). FIG. 6A depicts an array comprising a solid support 610 that contains a plurality of addresses 615. Each address of the plurality of addresses 615 comprises a single binding target of a heterogeneous plurality of binding targets. A first subset of addresses of the plurality of addresses 615 may comprise a ligand-binding polypeptide 620, a second subset of addresses of the plurality of addresses 615 may comprise a polypeptide binding ligand 622, and a third subset of addresses of the plurality of addresses 615 may comprise a non-polypeptide binding ligand 624. The array may be contacted with a plurality of detectable probes, optionally within a fluidic medium, in which each detectable probe 630 of the plurality of detectable probes comprises a binding specificity for a ligand-binding polypeptide 620. FIG. 6B depicts the array with one or more detectable probes 630 of the plurality of detectable probes forming polypeptide binding interactions with a ligand-binding polypeptide 620. Presence or absence of the polypeptide binding interaction may be determined by detecting a signal from a detectable probe 630 at a particular address of the first subset of addresses. FIG. 6C depicts contacting the solid support 610 with a plurality of detectable affinity agents, optionally within a fluidic medium, in which an affinity agent 632 of the plurality of affinity agents lacks a binding specificity for a ligand-binding polypeptide 620. FIG. 6D depicts the array with one or more detectable affinity agents 632 forming polypeptide binding interactions with a polypeptide binding ligand 622 of the plurality of polypeptide binding ligands. Optionally, the method may comprise repeating a step of binding a plurality of detectable probes or detectable affinity agents to the plurality of binding targets. Optionally, the method may comprise utilizing the detectable probe binding measurement to identify the first subset of addresses of the plurality of addresses 615 containing a ligand-binding polypeptide 620 of the plurality of ligand-binding polypeptides. Optionally, the method may comprise utilizing the detectable affinity agent binding measurement to identify the second subset of addresses of the plurality of addresses 615 containing a polypeptide binding target 622 of the plurality of polypeptide binding targets.

A method, as set forth herein, may comprise a comparative assay. A comparative assay may identify and/or quantify differences in a polypeptide binding interaction between a ligand-binding polypeptide provided by a subject and a ligand-binding polypeptide provided by a second subject or a control subject when the ligand-binding polypeptides are contacted with a binding ligand. A comparative assay may identify and/or quantify differences in a polypeptide binding interaction between a polypeptide provided by a subject and a polypeptide provided by a second subject (e.g. the second subject can be a control subject) when the binding ligands are contacted with a ligand-binding polypeptide. The skilled person will recognize that a method set forth herein may readily be adapted to a comparative assay. For example, a first plurality of polypeptides (e.g., ligand-binding polypeptides, binding ligands) from a first source (e.g., a human subject, a non-human subject, etc.) may be provided on a first solid support, and a second plurality of polypeptides (e.g., ligand-binding polypeptides, binding ligands) from a second source (e.g., a human subject, a non-human subject, a control sample, etc.) may be provided on a second solid support. Alternatively, a first plurality of polypeptides (e.g., ligand-binding polypeptides, binding ligands) from a first source (e.g., a human subject, a non-human subject, etc.) may be provided as a first plurality of detectable probes, and a second plurality of polypeptides (e.g., ligand-binding polypeptides, binding ligands) from a second source (e.g., a human subject, a non-human subject, a control sample, etc.) may be provided as a second plurality of detectable probes. In another example, a a first plurality of polypeptides (e.g., ligand-binding polypeptides, binding ligands) from a first source (e.g., a human subject, a non-human subject, etc.) may be contacted with a plurality of binding ligands on a solid support, and after removing any bound polypeptides of the first plurality of polypeptides, a second plurality of polypeptides (e.g., ligand-binding polypeptides, binding ligands) from a second source (e.g., a human subject, a non-human subject, a control sample, etc.) may be contacted with the plurality of binding entities on the solid support.

In another aspect, provided herein is a method comprising: a) providing a first plurality of ligand-binding polypeptides from a first source of polypeptides, and providing a second plurality of ligand-binding polypeptides from a second source of polypeptides; b) detecting presence or absence of binding of the first plurality of ligand-binding polypeptides and the second plurality of ligand-binding polypeptides to a binding ligand on a solid support at single-analyte resolution; and c) identifying a difference in a binding characteristic between the first plurality of ligand-binding polypeptides and the second plurality of ligand-binding polypeptides. A first source or a second source of a plurality of polypeptides may comprise a medical subject or a research subject. In some cases, a first source may comprise a first subject (e.g., a medical subject, a research subject) and a second source may comprise a second subject (e.g., a medical subject, a research subject). In some cases, a first source may comprise a subject (e.g., a medical subject, a research subject) and a second source may comprise a control sample.

A comparative assay may comprise a step of identifying a difference in a binding characteristic, in which the identifying comprises identifying a difference in binding specificity between the first plurality of polypeptides and the second plurality of polypeptides. A comparative assay may comprise a step of identifying a difference in binding characteristic, in which the identifying comprises identifying a difference in binding affinity between the first plurality of polypeptides and the second plurality of polypeptides. In some cases, identifying a difference in binding affinity comprises quantifying a difference in dissociation constant, association rate constant, or dissociation rate constant between a first plurality of polypeptides and a second plurality of polypeptides. A comparative assay may further comprise, based upon the identifying the difference in the binding characteristic, identifying a disease state or a health state of the medical subject. Identifying a disease state or a health state may comprise, for example, identifying a ligand-binding polypeptide with an increased or decreased binding affinity for a binding ligand, or identifying the presence or absence of a binding ligand that forms a polypeptide binding interaction with a ligand-binding polypeptide. For example, a comparative assay may identify the presence or absence of a diabetic disease state based upon the insulin binding behavior of serum albumin obtained from a medical subject, as compared to serum albumin from a healthy subject. In another example, a comparative assay may identify a pharmaceutical compound from a plurality of pharmaceutical compounds with a binding specificity for serum albumin. A comparative assay may further comprise, based upon identifying a disease state of the medical subject, identifying a method of treatment for the medical subject for the disease state.

A method, as set forth herein, may utilize a standard or control binding entity (e.g., a standard or control polypeptide, a standard or control non-polypeptide). In some cases, a standard or control binding entity may comprise a ligand-binding polypeptide. In particular cases, a standard or control binding entity may comprise a ligand-binding polypeptide that is expected to be included within an analyzed sample. For example, an array comprising a plurality of blood serum polypeptides may comprise an albumin standard or albumin control due to the expected presence of albumin in blood serum. A standard binding entity may comprise a polypeptide or non-polypeptide species that is combined with a plurality of binding entities for a purpose of monitoring of the plurality of binding entities (e.g., a purification efficiency standard, sample degradation standard, a storage standard, etc.). A control binding entity may comprise a polypeptide or non-polypeptide species that is added to a plurality of binding entities to provide a qualitative and/or quantitative comparison of a polypeptide binding interaction behavior between the plurality of binding entities and the control binding entity. A standard or control binding entity may be combined with a plurality of binding entities at any time before an array is analyzed. In some cases, a standard or control binding entity may be deposited on an array before a plurality of binding entities is deposited on an array. In some cases, a standard or control binding entity may be combined with a plurality of binding entities before the plurality of binding entities is deposited on an array.

In another aspect, provided herein is a method comprising: a) providing an array comprising a plurality of addresses, in which each address is resolvable from each other address at single-analyte resolution, in which each address of a first subset of the plurality of addresses is coupled to a binding entity of a plurality of binding entities, in which each address of a second subset of the plurality of addresses is coupled to a standard binding entity or a control binding entity of a plurality of standard or control binding entities; in which the plurality of binding entities is extracted from a subject, in which the plurality of binding entities comprises a first ligand-binding polypeptide, and in which the plurality of standard or control binding entities comprises a second ligand-binding polypeptide; b) contacting the array with a plurality of detectable probes, in which a detectable probe of the plurality of detectable probes comprises a binding ligand; and c) detecting presence or absence of a detectable probe of the plurality of detectable probes at each address of the plurality of addresses; and d) identifying presence or absence of a polypeptide binding interaction between the first ligand-binding polypeptide and the binding ligand or between the second ligand-binding polypeptide and the binding ligand based upon presence or absence of the detectable probe of the plurality of detectable probes at each address of the plurality of addresses.

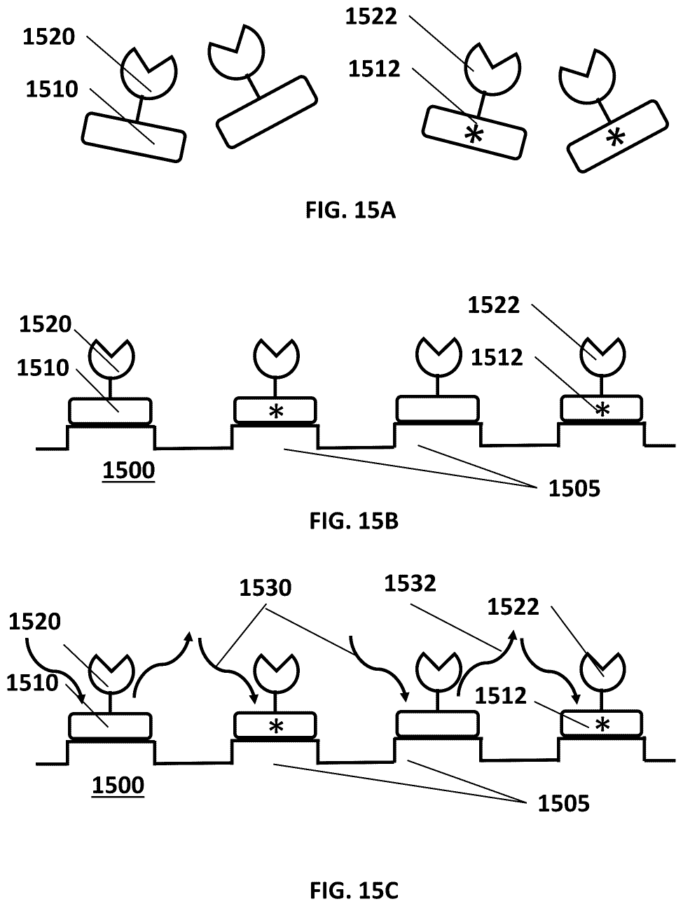

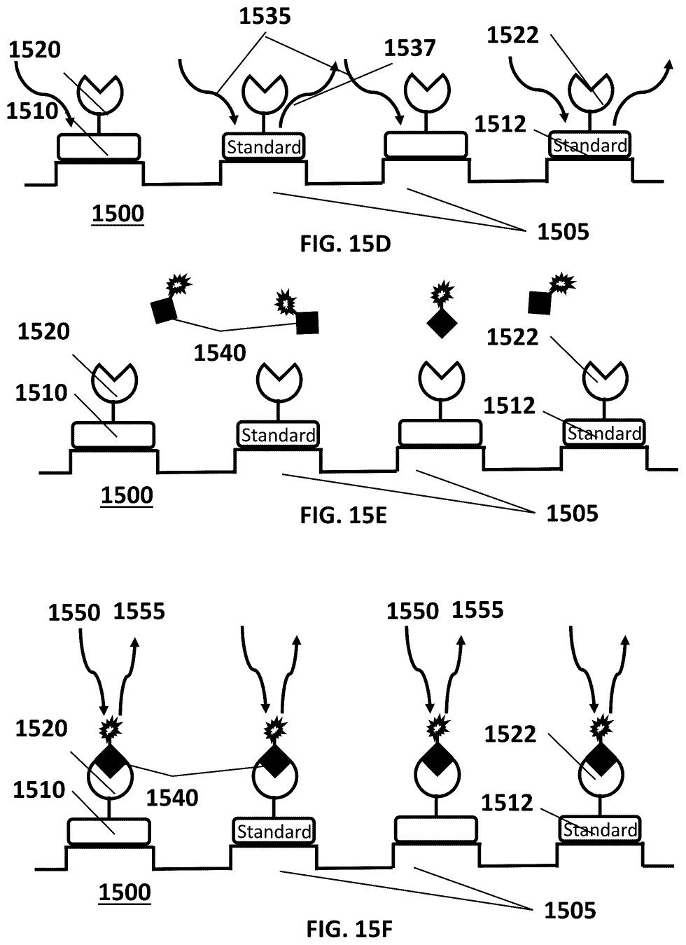

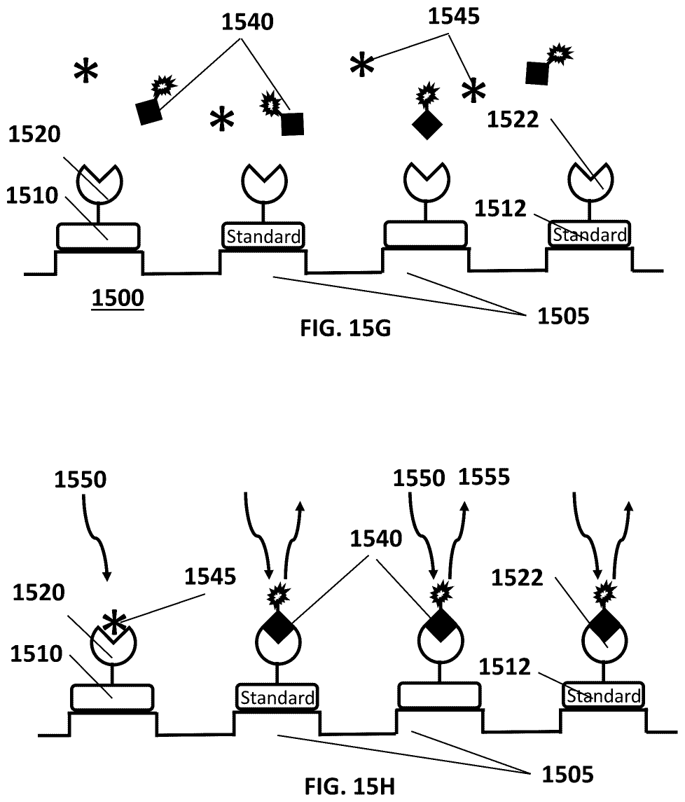

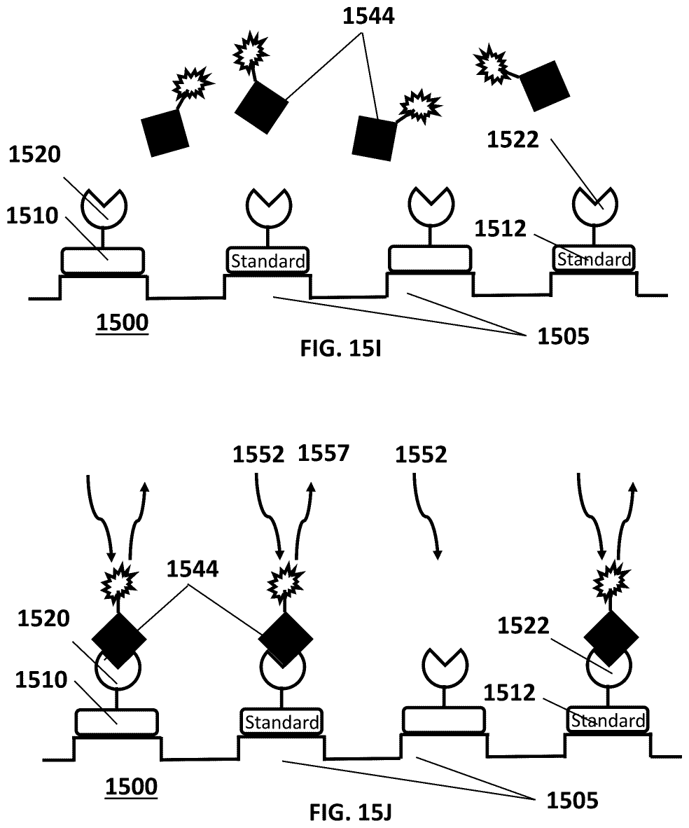

FIGS. 15A-15J depict a method of utilizing a standard or control polypeptide in a comparative assay. FIG. 15A depicts a first step in which a plurality of binding entities (e.g., ligand-binding polypeptides, binding ligands, etc.) are combined with a plurality of standard or binding entities. Optionally, a binding entity 1520 of the plurality of binding entities may be coupled to a first linker 1510, and/or a standard or control binding entity 1522 of the plurality of standard or control binding entities may be coupled to a second linker 1512, in which the first linker 1510 is distinguishable from the second linker 1512 at single-analyte resolution (e.g., containing different fluorophores, etc.). FIG. 15B shows the formation of an array comprising the plurality of binding entities and the plurality of standard or control binding entities. An array comprising a solid support 1500 contains a plurality of addresses 1505, in which each address is resolvable at single-analyte resolution. A first subset of addresses of the plurality of addresses 1505 couple to one or more linkers 1510 of the plurality of binding entities, and a second subset of addresses of the plurality of addresses 1505 couple to one or more linkers 1512 of the plurality of standard or control binding entities. FIGS. 15C-15D depict an optional set of steps comprising identifying the first subset of addresses comprising the plurality of binding entities and the second subset of addresses comprising the plurality of standard or control binding entities. FIG. 15C shows detection of the presence or absence of a first signal 1532 from linkers 1510 associated with binding entities 1520 of the plurality of binding entities at single-analyte resolution. An address at which a first signal 1532 is detected would be included in the first subset of addresses of the plurality of addresses 1505. Optionally, a first signal 1532 may be induced by a first excitation source 1530 (e.g., a light source). FIG. 15D shows detection of the presence or absence of a second signal 1537 from linkers 1512 associated with binding entities 1522 of the plurality of standard or control binding entities at single-analyte resolution. An address at which a second signal 1537 is detected would be included in the second subset of addresses of the plurality of addresses 1505. Optionally, a second signal 1537 may be induced by a second excitation source 1535 (e.g., a light source). FIGS. 15E-15J depict methods of comparing the binding behaviors of binding entities to the binding behaviors of standard or control binding entities. As shown in FIG. 15E, a plurality of first detectable probes may be contacted with the solid support 1500, optionally within a fluidic medium, in which a first detectable probe 1540 of the plurality of first detectable probes comprises a binding specificity for a binding entity 1520 of the plurality of binding entities and a binding specificity for a standard or control binding entity 1522 of the plurality of standard or control binding entities. FIG. 15F shows detecting of presence or absence of a first detectable probe 1540 of the plurality of first detectable probes at each address of the plurality of addresses 1505, in which the detecting comprises determining the presence or absence of a third signal 1555 from a detectable probe 1540 at each address at single-analyte resolution. Optionally, a third signal 1555 may be induced by a third excitation source 1550 (e.g., a light source). As shown in FIG. 15G, a heterogeneous mixture comprising a plurality of first detectable probes combined with a plurality of competitive binding ligands may be contacted with the solid support 1500, optionally within a fluidic medium, in which a first detectable probe 1540 of the plurality of first detectable probes comprises a binding specificity for a binding entity 1520 of the plurality of binding entities and a binding specificity for a standard or control binding entity 1522 of the plurality of standard or control binding entities. FIG. 15H shows detecting of the presence or absence of a first detectable probe 1540 of the plurality of first detectable probes at each address of the plurality of addresses 1505, in which the detecting comprises determining the presence or absence of a third signal 1555 from a first detectable probe 1540 at each address at single-analyte resolution. Optionally, a third signal 1555 may be induced by a third excitation source 1550 (e.g., a light source). Optionally, the competitive binding ligand may comprise a detectable label. Optionally, the method may further comprise a step of detecting the presence or absence of a signal from a competitive binding ligand 1542 at one or more addresses of the plurality of addresses. As shown in FIG. 15I, a plurality of second detectable probes may be contacted with the solid support 1500, optionally within a fluidic medium, in which a second detectable probe 1544 of the plurality of first detectable probes comprises, relative to a first detectable probe 1540, a differing binding specificity for a binding entity 1520 of the plurality of binding entities or a differing binding specificity for a standard or control binding entity 1522 of the plurality of standard or control binding entities. FIG. 15J shows detecting of the presence or absence of a second detectable probe 1544 of the plurality of second detectable probes at each address of the plurality of addresses 1505, in which the detecting comprises determining the presence or absence of a fourth signal 1555 from a second detectable probe 1544 at each address at single-analyte resolution. Optionally, a fourth signal 1557 may be induced by a fourth excitation source 1552 (e.g., a light source). Optionally, the method may further comprise identifying a difference in binding behavior between a binding entity 1520 and a standard or control binding entity 1522 based upon the detected binding profiles of the first plurality of detectable probes and/or the second plurality of detectable probes and/or the plurality of competitive binding ligands.

A single-analyte, array-based method may be utilized to identify an orientation of a binding target on an array. Orientation of a binding target at an address on an array may be important when determining the presence or absence of a polypeptide binding interaction at the address on the array. Binding target orientation may be important during an assay for various reasons, including: 1) particular orientations of a binding target may occlude a binding-related epitope of the binding target (e.g, a binding site of a ligand-binding polypeptide, a binding ligand epitope that is bound by the binding site of a ligand-binding polypeptide); and 2) variants of a binding target (e.g., mutants, atypical post-translational modifications, derivatives of a small molecule compound, etc.) may have different orientations for optimal binding with a complementary binding entity. As an example, for an array comprising serum albumins that are coupled to a solid support by linkers that conjugate to random lysine residues, each serum albumin on the array may be randomly oriented with respect to serum albumins at adjacent addresses on the array. Accordingly, certain serum albumins may be oriented such that a polypeptide binding site is occluded on the serum albumins at a subset of addresses on the array. Identification of a binding target orientation may improve analysis of polypeptide binding interaction data by allowing exclusion of some addresses that contain binding target orientations that inhibit or prevent formation of a polypeptide binding interaction. In some cases, a ligand-binding polypeptide may be configured to provide a consistent orientation of a structure and/or binding site when the ligand-binding polypeptide is coupled to an address of an array or coupled to a detectable probe. In some cases, orientation of a ligand-binding polypeptide may be controlled by engineering a functional site (e.g., incorporating a non-natural amino acid) at a specific location in the polypeptide structure, then coupling the ligand-binding polypeptide by the engineered functional site. In other cases, orientation of a ligand-binding polypeptide may be controlled by coupling the ligand-binding polypeptide to an array by a specific naturally-occurring residue of the polypeptide (e.g., an amino acid sidechain). In other cases, orientation of a ligand-binding polypeptide may be controlled by: a) coupling a binding ligand for a ligand-binding polypeptide to an address of a single-analyte array; b) binding the ligand-binding polypeptide to the binding ligand at the address on the single-analyte array by a binding site of the ligand-binding polypeptide; and c) coupling or cross-linking the ligand-binding polypeptide to the single-analyte array, thereby fixing an orientation of the ligand-binding polypeptide relative to the address on the single-analyte array.

In another aspect, provided herein is a method comprising: a) providing an array comprising a solid support that contains a plurality of addresses, in which each address of the plurality of addresses is resolvable at single-analyte resolution, and in which each address comprises a single binding target (e.g., a ligand-binding polypeptide, a binding ligand, etc.) of a plurality of binding targets; b) contacting the solid support with a plurality of detectable probes, in which a detectable probe of the plurality of detectable probes comprises a binding specificity for a binding epitope of the single binding target; c) detecting presence or absence of binding of the detectable probe of the plurality of detectable probes at each address of the plurality of addresses; d) repeating the contacting step and the detecting step for one or more additional pluralities of detectable probes, optionally in which a detectable probe of each plurality of detectable probes comprises a binding specificity for a different binding epitope of the single binding target; and e) based upon the two or more measurement outcomes for presence or absence of binding for two or more pluralities of detectable probes, identifying a subset of addresses comprising the single binding target with an occluded binding epitope. Optionally, the method may comprise identifying a first subset of addresses comprising a binding target with a first occluded epitope, and a second set of addresses comprising a binding target with a second occluded epitope. In some cases, a detectable probe of a plurality of detectable probes may comprise an affinity agent with a binding specificity for an epitope associated with a binding site. In other cases, a detectable probe of a plurality of detectable probes may comprise a binding ligand that is configured to bind to a binding site of a ligand-binding polypeptide. In some cases, a method may further comprise performing a method as set forth herein, in which the presence or absence of a polypeptide binding interaction may be determined at each address of the plurality of addresses, excluding a subset of addresses containing an occluded binding epitope.

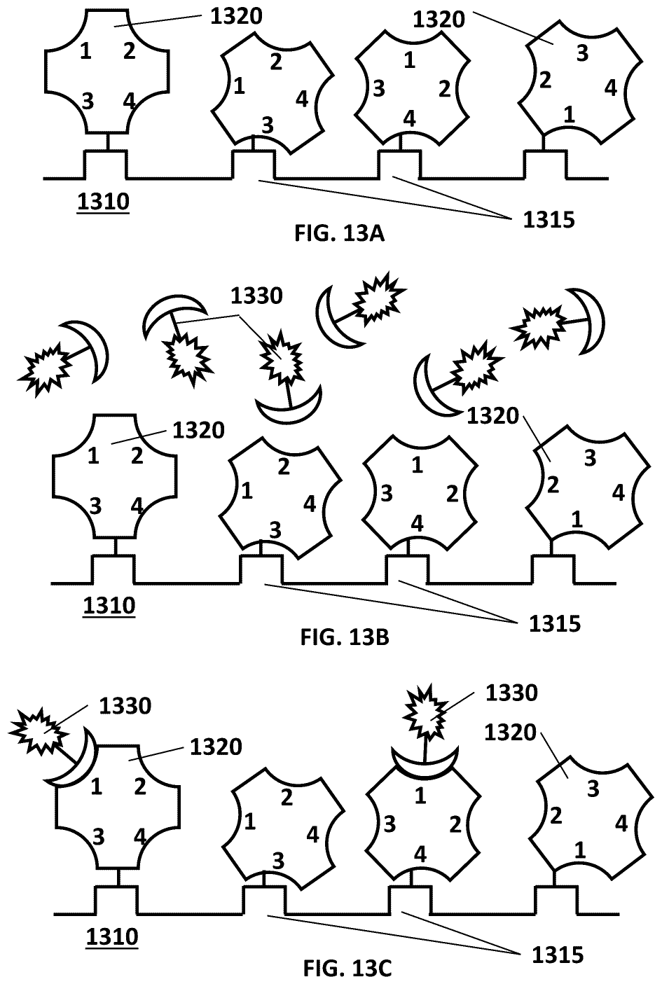

FIG. 13A-13C illustrate a method of identifying a binding specificity of a binding site on a ligand-binding polypeptide, including a ligand-binding polypeptide with multiple binding sites. FIG. 13A depicts a single-analyte array comprising a solid support 1310 containing a plurality of addresses 1315, in which each address of the plurality of addresses is resolvable at single-analyte resolution. Each address of the plurality of addresses 1315 is coupled to a single ligand-binding polypeptide 1320, in which the ligand-binding polypeptide contains 4 binding sites, labeled “1” through “4.” The orientation of a ligand-binding polypeptide 1320 is randomly varied on the array by linking the ligand-binding polypeptide 1320 by a random linking moiety (e.g., a random lysine residue, etc.) in the ligand-binding polypeptide 1320. FIG. 13B shows a plurality of detectable binding ligand probes is contacted with the solid support 1310, optionally within a fluidic medium, in which each detectable binding ligand 1330 probe comprises a known binding specificity for the ligand-binding polypeptide 1320. As shown in FIG. 13C, presence or absence of a detectable binding ligand probe 1330 may be detected at each address of the plurality of addresses to determine the presence or absence of a ligand-binding polypeptide 1320 and the binding ligand probe 1330. Optionally, the detectable binding ligand probe binding may be repeated for one or more detectable probes with differing binding site specificities. Optionally, the method may further comprise identifying an orientation of each ligand-binding polypeptide at each address of the plurality of addresses. An orientation may optionally be determined by depositing ligand-binding polypeptides with identical orientations on the array via distinguishable linkers that correspond to a single orientation, or may be determined by identifying presence or absence of epitopes associated with binding sites via binding of detectable affinity agents with a binding specificity for the associated epitope. Optionally, the method may further comprise identifying a binding site that is capable of forming a polypeptide binding interaction with the binding ligand probe 1330 based upon the presence or absence of probe binding. The skilled person will recognize that the provided method of identifying binding site specificity can also be implemented using detectable probes comprising a ligand-binding polypeptide. Multiple pluralities of detectable probes can be prepared, with each plurality of detectable probes comprising a particular orientation of ligand-binding polypeptides coupled to the probe (e.g., a probe comprising a retaining component that controls orientation of the ligand-binding polypeptide). The binding of each plurality of detectable probes may be tested against an array of binding ligands to determine which orientations inhibit binding.

Identification of polypeptide binding interactions may be utilized during a polypeptide assay, as set forth herein. In some cases, the presence of binding interactions between binding entities and array-bound polypeptides may be utilized to identify a fraction of array-bound polypeptides for further analysis. For example, during an analysis of an array-bound nucleus proteome, it may be advantageous to identify nucleic acid binding proteins by identifying binding interactions between polypeptides of the array-bound nucleus proteome and random or non-random pluralities of nucleic acids (e.g., DNAs, RNAs). In other cases, the absence of binding interactions between binding entities and array-bound polypeptides may be utilized to identify a fraction of array-bound polypeptides for further analysis. For example, it may be advantageous to identify polypeptides of a nucleus proteome that do not bind nucleic acids by first identifying binding interactions of polypeptides of an array-bound nucleus proteome with random or non-random nucleic acids (e.g., DNAs, RNAs), then excluding nucleic acid-binding polypeptides from further analysis.

In another aspect, a method, as set forth herein, may comprise one or more steps of: i) providing an array comprising a plurality of addresses, in which each address is resolvable from each other address at single-analyte resolution, in which each address of the plurality of addresses is coupled to a binding entity (e.g., a polypeptide) of a plurality of binding entities, ii) contacting the array with a plurality of binding ligands, iii) detecting a presence or an absence of a binding ligand of the plurality of binding ligands at each address of the plurality of addresses; and iv) based upon a presence or an absence of a binding ligand at an address of the plurality of addresses, identifying a binding entity of the plurality of binding entities. Optionally, identifying a binding entity of a plurality of binding entities at an address of a plurality of addresses may further comprise one or more steps of: i) contacting the binding entity with a plurality of affinity agents, ii) detecting a presence or an absence of an affinity agent of the plurality of affinity agents at the address of the plurality of addresses, and iii) based upon the presence or the absence of the binding ligand at the address of the plurality of addresses and based upon the presence or the absence of the affinity agent at the address of the plurality of addresses, identifying the binding entity of the plurality of binding entities.

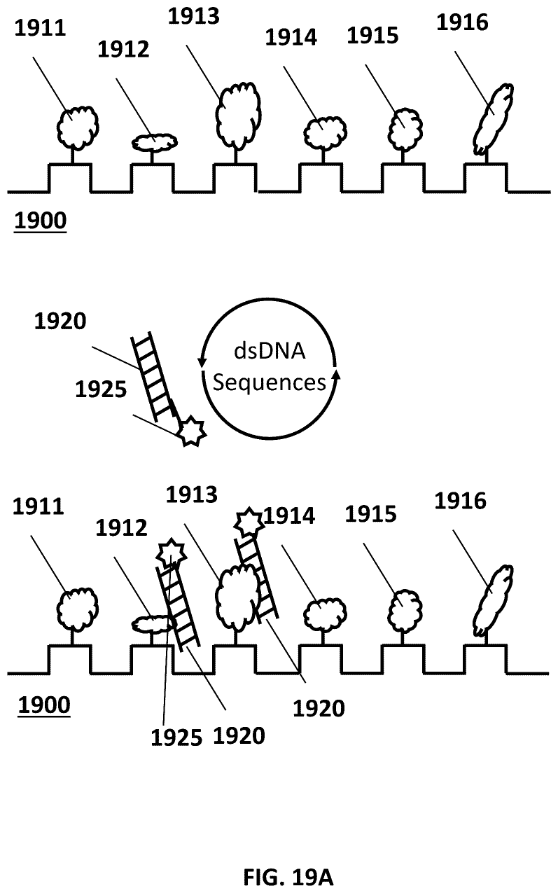

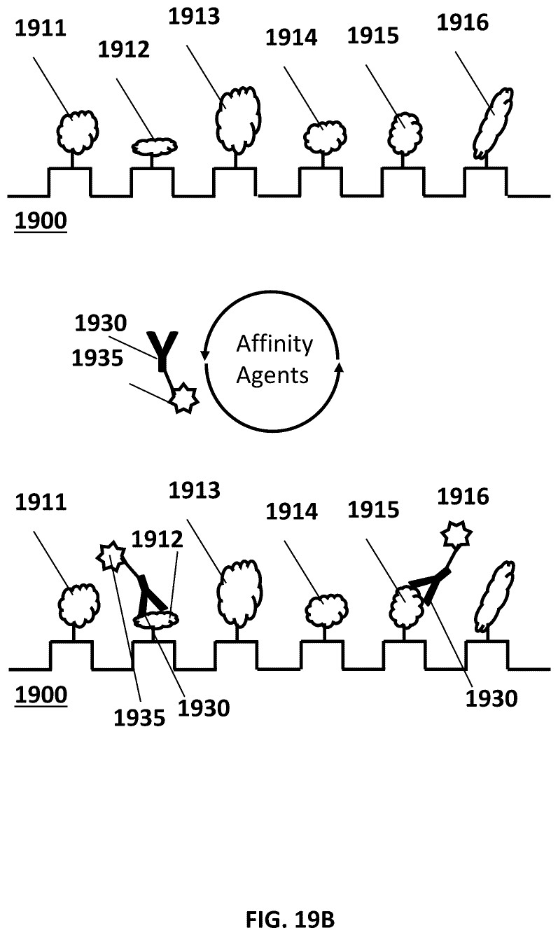

FIGS. 19A and 19B illustrate aspects of a method for utilizing a binding ligand to identify a polypeptide during a polypeptide assay. FIG. 19A depicts an array comprising a solid support 1900 with a plurality of analyte binding sites. Each analyte binding site comprises a coupled polypeptide (1911, 1912, 1913 1914, 1915, 1916, respectively), optionally in a native state folding conformation. The array comprising the coupled polypeptides is contacted with detectable molecules comprising double-stranded DNA sequences (dsDNA) 1920 and a detectable label 1925 (e.g., a fluorophore, a luminophore, a nucleic acid tag, etc.), thereby permitting dsDNA sequences to bind to any polypeptides that are configured to form a nucleic acid binding interaction. As shown in FIG. 19A, polypeptides 1912 and 1913 form binding interactions with dsDNA sequences 1920, thereby permitting detection of detectable signals from detectable labels 1925 at each array address corresponding to an analyte binding site at which a dsDNA sequence 1920 is bound. Accordingly, one or more addresses may be identified as containing a polypeptide that is configured to form a binding interaction with a dsDNA sequence 1920 based upon a detected signal from a bound detectable molecule. Optionally, the binding of dsDNA sequences 1920 may be repeated via cycles of contacting, binding, detecting, and removing dsDNA sequences 1920 from the array. Optionally, other detectable binding ligands (e.g., polypeptides, single-stranded DNA, RNA, saccharides, lipids, metabolites, small molecules, etc.) may be substituted for dsDNA sequences 1920.

FIG. 19B depicts an optional polypeptide identification process utilizing cycling of affinity agents to identify coupled polypeptides at array addresses. Before or after the process depicted in FIG. 19A, the array of polypeptides may be contacted with a plurality of detectable probes comprising an affinity agent 1930 and a detectable label 1935. Optionally, each affinity agent 1930 may be configured to bind to one or more epitopes, with each of the one or more epitopes occurring in two or more polypeptides (e.g., trimer or tetramer amino acid sequences). As shown in FIG. 19B, detectable probes bind to polypeptides 1912 and 1915, thereby permitting detection of detectable signals from detectable labels 1935 at each array address corresponding to an analyte binding site at which an affinity agent is bound. Optionally, a polypeptide identification assay may comprise cycling of a plurality of different detectable probes, each comprising a differing binding specificity. Based upon binding profiles of detectable probes and/or binding ligands, a polypeptide identity may be determined for each array address comprising an observable polypeptide.

The skilled person will readily recognize that innumerable variations of the methods set forth herein are possible. For example, methods of identifying polypeptide binding interactions may further comprise steps of forming arrays of binding targets or identifying one or more species that participate in a polypeptide binding interaction. Certain common steps or procedures of single-analyte, array-based methods are described hereafter.

A polypeptide of a plurality of polypeptides may comprise a native folding configuration state. A native folding configuration state may comprise polypeptide chain folding to produce a bioactive form of a polypeptide. A native folding configuration state may further comprise chemical modifications (e.g., disulfide bond formation, post-translational modifications, etc.) or chemical incorporations (e.g., inclusion of metal-containing moieties, inclusion of cofactors or coenzymes, etc.) that produce a bioactive form of a polypeptide. Alternatively, a polypeptide of a plurality of polypeptides may not comprise a native folding configuration state. A polypeptide of a plurality of polypeptides may comprise a partially-folded state or a denatured state. A polypeptide of a plurality of polypeptides may comprise a misfolded state (i.e., a folding state that produces a non-bioactive form of the polypeptide). In some cases, a polypeptide of a plurality of polypeptides may be provided in a native folding configuration state to identify a binding interaction of the polypeptide and provided in a partially- or fully-denatured state to determine an identity of the polypeptide. In other cases, a polypeptide of a plurality of polypeptides may be provided in a native folding configuration state to identify a binding interaction of the polypeptide and to determine an identity of the polypeptide.

A method, as set forth herein, may comprise one or more steps of: i) providing a polypeptide of a plurality of polypeptides at an address of a single-analyte array, as set forth herein, in which the polypeptide is provided in a native state; ii) identifying a polypeptide binding interaction between the polypeptide of the plurality of polypeptides and a binding ligand; iii) partially or fully denaturing the polypeptide of the plurality of polypeptides; and iv) identifying the polypeptide of the plurality of polypeptides by a polypeptide identification assay, as set forth herein. In some cases, partially or fully denaturing a polypeptide of a plurality of polypeptides may comprise contacting the polypeptide with a denaturing agent or chaotrope. Optionally, a method may further comprise a step of identifying one or more epitopes associated with a native state of a polypeptide. Optionally, a method may further comprise a step of identifying one or more epitopes associated with a partially- or fully-denatured state of a polypeptide. Optionally, a method may further comprise a step of identifying one or more epitopes associated with a misfolded state of a polypeptide. Optionally, a method may further comprise a step of folding a partially- or fully-denatured polypeptide to a native state. In some cases, a partially- or fully-denatured polypeptide may be folded to a native state in the presence of a folding facilitator (e.g., a chaperonin). In some cases, a method may comprise one or more steps of: i) optionally identifying a presence of an epitope associated with a partially- or fully-denatured state of a polypeptide, ii) optionally identifying an absence of an epitope associated with a native state of a polypeptide, iii) altering a folding state of the polypeptide (e.g., partially or fully folding the polypeptide, partially or fully denaturing the polypeptide), iv) optionally identifying a presence of an epitope associated with a partially- or fully-folded state (e.g., a native state) of the polypeptide, and iv) optionally detecting an absence of an epitope associated with a partially- or fully denatured state of the polypeptide.

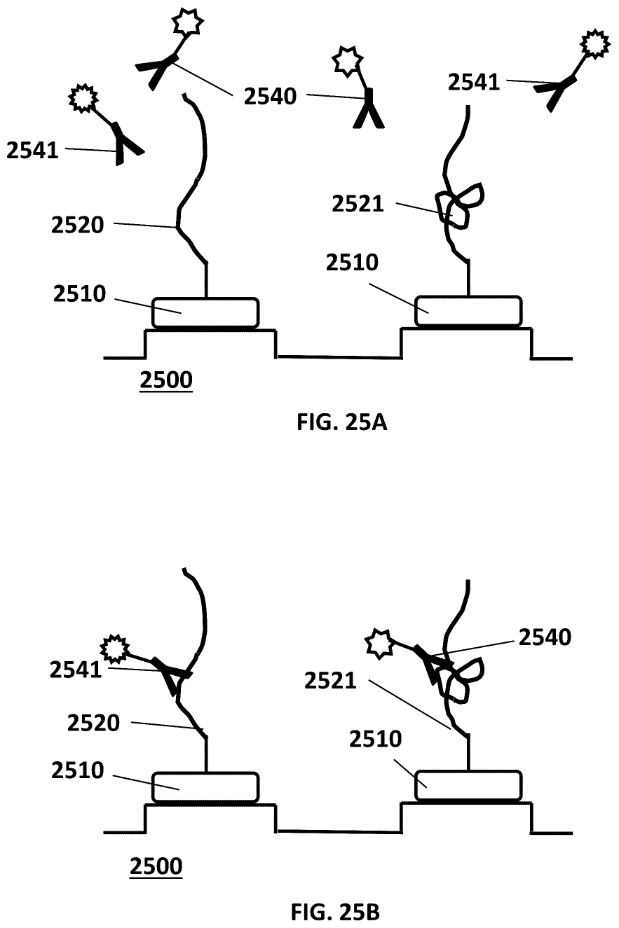

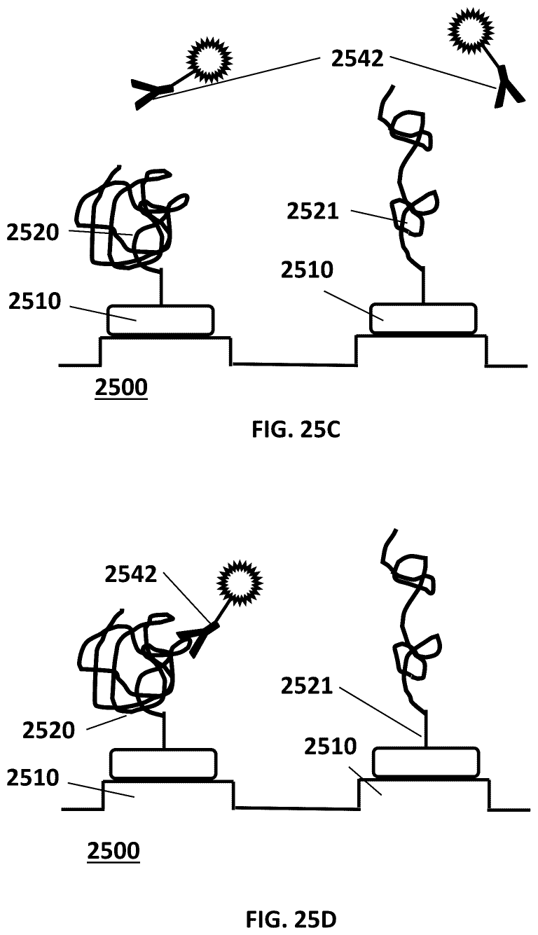

FIGS. 25A-25D depict a method of identifying folding states of polypeptides as part of a method, as set forth herein. FIG. 25A depicts an array comprising a solid support 2500 with a first coupled polypeptide 2520 in a fully-denatured state, and a second polypeptide 2521 in a partially-folded state. Both polypeptides are localized to different array addresses by linking moieties 2510. The array is contacted with a plurality of detectable probes, including a first detectable probe 2541 that is configured to bind a fully-denatured epitope, and a second detectable probe 2540 that is configured to bind a partially-folded epitope (e.g., an epitope comprising some secondary or tertiary structure). FIG. 25B depicts an array configuration in which detectable probe 2541 has bound to polypeptide 2520, and detectable probe 2540 has bound to polypeptide 2521. Accordingly, it may be determined that the left address comprises a fully-denatured polypeptide, while the right address comprises a partially-folded polypeptide. FIG. 25C depicts a subsequent assay step in which polypeptide have been folded or re-folded (e.g., via change in fluidic medium, via introduction of fold-mediating proteins, etc.). Such a step may occur to identify the affect of folding state on ligand-binding properties of a polypeptide. In FIG. 25C, polypeptide 2520 has adopted a native folding configuration state, while polypeptide 2521 has adopted a differing partially-folded configuration. The array is contacted with a detectable probe 2542 that is configured to bind an epitope of a native folding configuration of the polypeptide. FIG. 25D depicts an array configuration in which detectable probe 2542 has bound to polypeptide 2542 and has not bound to polypeptide 2521, thereby permitting identification of the native folding configuration state of polypeptide 2520.

A plurality of polypeptides, as utilized in a method, composition, or system set forth herein, may undergo one or more separations to remove a fraction from the sample. A plurality of polypeptides may undergo one or more separations to remove a non-polypeptide fraction from the sample (e.g., metal ions, non-metal ions, small molecule compounds, acidic compounds, basic compounds, peptides, nucleic acids, lipids, etc.). A plurality of polypeptides may undergo one or more separations to remove a fraction of polypeptides from the plurality of polypeptides. In some cases, a method may further comprise one or more steps of: a) extracting a plurality of polypeptides from a sample or subject; and b) separating a fraction of ligand-binding polypeptides from the plurality of polypeptides. In other cases, a method may further comprise one or more steps of: a) extracting a plurality of polypeptides from a sample or subject; and b) separating a fraction of candidate binding ligands from the plurality of polypeptides. In some cases, a method may further comprise discarding a fraction of ligand-binding polypeptides or a fraction of candidate binding ligands. A plurality of polypeptides may be purified, separated and/or fractionated by any suitable method, such as affinity chromatography, size-exclusion chromatography, high-pressure liquid chromatography, centrifugation, precipitation, etc.

A plurality of detectable probes, as set forth herein, may be prepared by coupling a binding entity to a detectable label. A plurality of detectable probes may be prepared by coupling a binding entity derived from a plurality of polypeptides or a fraction thereof to a detectable label. In some configurations, a method may further comprise preparing a detectable probe of a plurality of detectable probes by coupling a ligand-binding polypeptide of a plurality of ligand-binding polypeptides to a detectable label. For example, a separated fraction of ligand-binding polypeptides may be coupled to detectable labels to prepare a plurality of detectable probes. In other configurations, a method may further comprise preparing a detectable probe of a plurality of detectable probes by coupling a candidate binding ligand of a fraction of candidate binding ligands to a detectable label. For example, a separated fraction of candidate binding ligands may be coupled to detectable labels to prepare a plurality of detectable probes.

A detectable probe or an array, as set forth herein, may comprise a binding entity (e.g., an affinity reagent, a ligand-binding polypeptide, a binding ligand, etc.) derived from any conceivable source. A detectable probe or an array, as set forth herein, may comprise a binding entity derived from a native organism. For example, a detectable probe may comprise an albumin derived from a human blood sample. A detectable probe or an array, as set forth herein, may comprise a binding entity derived from an engineered organism. For example, a detectable probe may comprise an albumin produced by an engineered microorganism (e.g., E. coli, S. cerevisiae, etc.). A detectable probe or an array, as set forth herein, may comprise a native ligand-binding polypeptide. For example, a detectable probe of a plurality of detectable probes may comprise an albumin or globulin whose amino acid sequence occurs naturally within a population or cohort of subjects. A detectable probe or an array, as set forth herein, may comprise an engineered ligand-binding polypeptide. For example, a detectable probe of a plurality of detectable probes may comprise an albumin produced by an engineered microorganism. A detectable probe or an array, as set forth herein, may comprise a ligand-binding polypeptide comprising an engineered mutation relative to a native ligand-binding polypeptide. For example, a detectable probe of a plurality of detectable probes may comprise an albumin or globulin whose amino acid sequence does not occur naturally within a population or cohort of subjects. A detectable probe or an array, as set forth herein, may comprise a ligand-binding polypeptide comprising a post-translational modification and/or a post-synthesis modification. A post-translational modification may include myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminus amidation, arginylation, polyglutamylation, polyglyclyation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, proteolytic cleavage, isoaspartate formation, racemization, and protein splicing.

A binding entity of the present disclosure may be readily converted into a detectable probe by coupling a detectable label to the binding entity. Numerous methods, as set forth herein, may be readily adapted into a multiplex configuration by utilizing pluralities of binding entities comprising two or more detectable labels, in which a first type of binding entity is provided a first detectable label, and a second type of binding entity is provided a second detectable label. For example, a competitive binding assay may comprise simultaneously contacting an array comprising a plurality of ligand-binding polypeptides with a first plurality of binding ligands and a second plurality of binding ligands, in which each binding ligand of the first plurality of binding ligands comprises a first detectable label, and in which each binding ligand of the second plurality of binding ligands comprises a second detectable label. In another example, an array comprising a plurality of a single type of ligand-binding polypeptide (e.g., albumin, globulin, etc.) may be contacted with a plurality of polypeptide binding entities comprising a first proteomic sample and a second proteomic sample, in which each polypeptide of the first proteomic sample comprises a first detectable label, and in which each polypeptide of the second proteomic sample comprises a second detectable label.

A method, as set forth herein, may readily be extended into a binding kinetics or binding equilibrium assay. In some cases, a method may comprise one or more steps of: i) contacting a second plurality of binding entities to an array, as set forth herein, comprising a first plurality of binding entities, in which the second plurality of binding entities comprises a first measurable concentration, ii) contacting a second plurality of binding entities to an array, as set forth herein, comprising a first plurality of binding entities, in which the second plurality of binding entities comprises a second measurable concentration, iii) optionally contacting a second plurality of binding entities to an array, as set forth herein, comprising a first plurality of binding entities, in which the second plurality of binding entities comprises a third measurable concentration, iv) after contacting the second plurality of binding entities to the array, detecting a presence or absence of a binding interaction at each address of the array, and v) based upon the detected presence or absence of binding at each array address for each contacting of the second plurality of binding entities to the array, determining a kinetic or thermodynamic binding parameter (e.g., dissociation constant, association rate constant, dissociation rate constant, etc.) of a binding entity of the second plurality of binding entities for a binding entity of the first plurality of binding entities. Optionally, a binding kinetics or binding equilibrium assay may further comprise one or more steps of: i) altering a system property or parameter (e.g., contacting time, fluidic pH, fluidic ionic strength, fluidic composition, etc.), and ii) repeating one or more contactings of a second plurality of binding entities to the array with the altered system property or parameter. The skilled person will further recognize that a binding kinetics or binding equilibrium assay may be multiplexed by a multiplexing method set forth herein.

A method, as set forth herein, may comprise one or more steps of: a) contacting a plurality of detectable probes with an array comprising a plurality of addresses, in which each address of the plurality of addresses comprises a binding target (e.g., a ligand-binding polypeptide, a binding ligand); b) binding one or more detectable probes of the plurality of detectable probes to one or more binding targets at one or more addresses of the plurality of addresses on the array to form one or more polypeptide binding interactions at the one or more addresses; and c) detecting presence or absence of the one or more polypeptide binding interactions at the one or more addresses on the array. In some cases, detecting the presence or absence of the one or more polypeptide binding interactions at the one or more addresses comprises detecting signal or absence of signal from a detectable probe of the plurality of detectable probes at each address. The signal from a detectable probe may be observed by fluorescence measurement, luminescence measurement, luminescence lifetime measurement, or signal encoding (e.g., nucleic acid or peptide barcodes, etc.).

A method, as set forth herein, may comprise a step of providing an array comprising a plurality of binding targets (e.g., a plurality of polypeptides, a plurality a ligand-binding polypeptides, a plurality of binding ligands, a plurality of polypeptide complexes, etc.). In some cases, providing an array comprising a plurality of binding targets may comprise one or more steps of: a) contacting the plurality of binding targets with a plurality of linkers, in which each linker of the plurality of linkers is configured to couple to a binding target of the plurality of binding targets; b) contacting the plurality of linkers with a solid support comprising a plurality of addresses, in which each address of the plurality of addresses is configured to couple with a linker of the plurality of linkers; c) coupling a binding target of the plurality of binding targets to a single linker of the plurality of linkers; and d) coupling a single linker of the plurality of linkers to a address of the plurality of addresses. It will readily be recognized that an array formation method, such as the exemplary configuration set forth above or elsewhere herein, may occur in numerous variations. For example, each linker of a plurality of linkers (e.g., SNAPs, nanoparticles, etc.) may be coupled to an address of a solid support, then a binding target of a plurality of binding targets may be coupled to each linker of the plurality of linkers. In another example, one or more binding targets of a plurality of binding targets may be coupled with one or more linkers, then each linker of the one or more linkers may be coupled to an address on the solid support. In some cases, each address on a solid support may comprise a single linker and/or a single binding target.

An array, as utilized by a method, composition, or system set forth herein, may comprise a region comprising a heterogeneous mixture, in which the heterogeneous mixture comprises a plurality of binding targets, and in which a binding target of the plurality of binding targets may be coupled to an address on the array. A mixture of binding targets may be heterogeneous in the aspect that the mixture contains at least two distinct types of chemical species, in which each distinct species may become coupled to a different address of an array and may optionally be identifiable at single-analyte resolution (e.g., a mixture of polypeptides and lipids, a mixture of polypeptides and nucleic acids, a mixture of polypeptides and small molecule compounds, etc.). For example, an array may comprise a heterogeneous mixture of polypeptides and nucleic acids, in which each address of a plurality of addresses on the array comprises either a coupled polypeptide or a coupled nucleic acid. A mixture of binding targets may be heterogeneous in the aspect that the mixture contains at least two distinct types of polypeptide species that may become coupled to an address of an array and may optionally be identifiable at single-analyte resolution. For example, an array comprising a plurality of polypeptides from a blood sample may comprise a heterogeneous mixture of ligand-binding polypeptides, candidate binding ligands for a ligand-binding polypeptide, and non-binding polypeptides, in which each address of a plurality of addresses on the array comprises a coupled ligand-binding polypeptide, a coupled binding ligand for a ligand-binding polypeptide, or a non-binding polypeptide. Alternatively or additionally, an array, as utilized by a method set forth herein, may comprise a homogeneous mixture comprising a plurality of binding targets, in which a binding target of the plurality of binding targets may be coupled to an address on the array. A mixture of binding targets may be considered homogeneous if the purity of the plurality of binding targets exceeds a threshold purity level with regard to impurities. For example, a homogeneous mixture comprising a plurality of ligand-binding polypeptides may be considered homogeneous if the purity of the ligand-binding polypeptides relative to polypeptides other than ligand-binding polypeptides exceeds a threshold value. A threshold value for the purity of a homogeneous mixture of binding targets may be based upon a reference measure, such as a molar or mass basis. For example, a homogeneous mixture of binding targets may exceed a threshold purity of at least about 90%, 95%, 96%, 97%, 98%, 99%, 99.9%, 99.99%, 99.999%, 99.9999%, 99.99999%, 99.999999%, or greater on a mass basis relative to total binding target content. Alternatively or additionally, a homogeneous mixture of binding targets may have a purity of no more than about 99.999999%, 99.99999%, 99.9999%, 99.999%, 99.99%, 99.9%, 99%, 98%, 97%, 96%, 95%, 90%, or less on a mass basis relative to total binding target content.

In some cases, a plurality of binding targets may be derived from a sample comprising impurities (i.e., unwanted or extraneous species for an analysis that is to be performed). In particular cases, a plurality of binding targets may be extracted from a crude, unpurified, or incompletely purified sample. For example, a plurality of binding targets may be directly transferred from a crude cellular lysate to an array for an analysis by a method as set forth herein. It may be particularly advantageous to prepare an array of binding targets from a crude, unpurified, or incompletely purified sample in cases where purification may be difficult or expensive, or in cases where binding target collection is time-sensitive (e.g., time sequences of present polypeptides, rapidly degrading environments, etc.). Accordingly, arrays prepared from binding targets derived from crude, unpurified, or incompletely purified samples may comprise heterogeneous mixtures of binding targets depending upon the chemistry used to couple binding targets to addresses on a solid support. In another aspect, provided herein is a method comprising: a) providing an array comprising a plurality of binding targets (e.g., a plurality of polypeptides, a plurality of ligand-binding polypeptides, a plurality of binding ligands, a plurality of polypeptide complexes, etc.) and one or more impurities, in which each binding target of the plurality of binding targets is located at an address that is resolvable from each other address on the array, in which each of the one or more impurities is located at an address that is resolvable from each other address on the array, in which each address of the array is detectable at single-analyte resolution, in which the plurality of binding targets comprises a ligand-binding polypeptide, a binding ligand, or a candidate binding ligand, and optionally in which the ligand-binding polypeptide comprises an albumin or a globulin; b) determining presence or absence of an impurity of the one or more impurities at each address on the array beyond a threshold measure of confidence; and c) detecting presence or absence of binding of an affinity agent of a plurality of affinity agents to a binding target of the plurality of binding targets at each address of the plurality of addresses excluding a subset of addresses where presence of the impurity of the one or more impurities was determined beyond the threshold measure of confidence, in which the affinity agent comprises a binding specificity for the binding target.

A method, as set forth herein, may comprise identifying a binding target of a plurality of binding targets at an address on an array. A binding target of a plurality of binding targets may have its identity established before or after the presence or absence of a polypeptide binding interaction with a ligand-binding polypeptide is determined. In some cases, identifying a binding target of a plurality of binding targets at the address on an array may comprise one or more steps of: a) contacting the array with a first plurality of affinity agents, in which a binding property of an affinity agent of the first plurality of affinity agents is characterized with respect to a set of epitopes; b) detecting presence or absence of signal from the an affinity agent of the first plurality of affinity agents at an address on the array; c) contacting the array with one or more additional pluralities of affinity agents, in which a binding property of an affinity agent of each of the one or more additional pluralities of affinity agents is characterized with respect to one or more additional sets of epitopes; d) detecting presence or absence of signal from an affinity agent of each additional plurality of affinity agents at the address on the array; and e) identifying the binding target of the plurality of binding targets at the address based upon the observed presences or absences of signal from each plurality of affinity agents at the address. In some cases, identifying the binding target of the plurality of binding targets at the address based upon the observed presences or absences of signal from each plurality of affinity agents at the address may comprise the steps of: a) providing observed presences or absences of signal from each plurality of affinity agents at the address to a computer algorithm; and b) determining the identity of the binding target at the address by the computer algorithm based upon observed presences or absences of signal from each plurality of affinity agents at the address. Optionally, a method set forth herein may comprise identifying one or more impurities on an array. A method of identifying an impurity may comprise the steps of: a) contacting an array comprising an impurity coupled to an address on the array with a first plurality of affinity agents, in which an affinity agent of the plurality of affinity agents comprises a binding specificity for the impurity; b) detecting presence or absence of signal from the affinity agent of the first plurality of affinity agents at the address on the array; and c) identifying the impurity at the address on the array based upon the observed presence or absence of signal from the affinity agent of the plurality of affinity agents at the address on the array.

Methods, compositions, and systems, as set forth herein, utilizing single-analyte arrays or detectable probes of ligand-binding polypeptides may comprise ligand-binding polypeptides with one or more unique binding sites that are configured to form a polypeptide binding interaction with a binding ligand. A binding site may comprise a binding specificity for a single binding ligand. A binding site may comprise a binding specificity for two or more binding ligands. A binding site may comprise a binding specificity for two or more binding ligands with structural similarity (e.g., a small molecule and its derivatives, polypeptide isoforms, a polypeptide and mutants thereof, etc.). A binding site may comprise a binding specificity for two or more binding ligands with structural dissimilarity (e.g., a polypeptide and a lipid, a polypeptide and a nucleic acid, etc.).

A ligand-binding polypeptide may comprise two or more binding sites. A ligand-binding polypeptide may comprise about 1, 2, 3, 4, 5, 6, 7, 8, 9, 10, or more binding sites that are configured to form a polypeptide binding interaction with a binding ligand. A ligand-binding polypeptide may comprise at least about 1, 2, 3, 4, 5, 6, 7, 8, 9, 10, or more binding sites that are configured to form a polypeptide binding interaction with a binding ligand. Alternatively or additionally, a ligand-binding polypeptide may comprise no more than about 10, 9, 8, 7, 6, 5, 4, 3, 2, or fewer binding sites that are configured to form a polypeptide binding interaction with a binding ligand. A ligand-binding polypeptide may be configured to form a single polypeptide binding interaction at a time. A ligand-binding polypeptide may be configured to form two or more polypeptide binding interactions at a time. The number of polypeptide binding interactions that a ligand-binding polypeptide may form at a time may be controlled, in whole or in part, by a regulatory mechanism such as allosteric promotion or allosteric inhibition.

Numerous types of affinity agents may be advantageous for detecting a polypeptide binding interaction between a ligand-binding polypeptide and a binding ligand. Of particular interest are affinity agents that: 1) detect the presence or absence of an epitope of a ligand-binding polypeptide that is involved or present during a polypeptide binding interaction; and 2) detect the presence or absence of a binding ligand without binding to an epitope present in a ligand-binding polypeptide. Table II summarizes various types of affinity reagents by binding specificity and states how the presence or absence of a detectable signal from such an affinity agent would provide evidence for the presence or absence of a polypeptide binding interaction.

TABLE II

| | | |
|---|---|---|
| Binding Specificity | Signal Presence | Signal Absence |

Ligand-Binding Polypeptide-Specific

| | | |
|---|---|---|
| Binds an epitope associated with a | Binding site maybe unoccupied; | Binding site may be occupied; |
| ligand-binding polypeptide binding | polypeptide binding interaction | polypeptide binding interaction |
| site | unlikely | likely |
| Binds an epitope of a ligand-binding | Epitope maybe present; | Epitope may be absent; |
| polypeptide exposed by a binding- | polypeptide binding interaction | polypeptide binding interaction |
| induced conformational change | likely | unlikely |
| Binds an epitope of a ligand-binding | Epitope maybe present; | Epitope may be absent; |
| polypeptide screened by a binding- | polypeptide binding interaction | polypeptide binding interaction |
| induced conformational change | unlikely | likely |

Binding Ligand-Specific

| | | |
|---|---|---|
| Binds an epitope that is not present in | Epitope maybe present; binding | Epitope may be absent; binding |
| a ligand-binding polypeptide | ligand presence likely | ligand presence less likely |
| Binds a binding ligand at a structure | Structure may be present; binding | Structure may be absent, binding |
| that is not involved in forming a | ligand presence likely | ligand presence less likely |
| polypeptide binding interaction; does | | |
| not bind a ligand-binding polypeptide | | |

A method may comprise a step of detecting presence or absence of a polypeptide binding interaction at each address of an array, in which the detecting comprises the steps of: a) contacting the array with a first plurality of detectable affinity agents, in which each detectable affinity agent comprises a characterized binding affinity to a ligand-binding polypeptide of the plurality of ligand-binding polypeptides; and b) detecting presence or absence of a signal from a detectable affinity agent of the plurality of detectable affinity agents at each address. In some cases, an affinity agent of a first plurality of affinity agents may comprise a binding specificity for an epitope associated with a first binding site of a ligand-binding polypeptide. In other cases, an affinity agent of a first plurality of affinity agents may comprise a binding specificity for an epitope associated with a conformational change of a ligand-binding polypeptide due to a polypeptide binding interaction at a first binding site. An epitope associated with a conformational change of a ligand-binding polypeptide may include an epitope that is exposed by the conformational change (e.g., an epitope buried within the core of the protein when a polypeptide-binding interaction is not occurring). An epitope associated with a conformational change of a ligand-binding polypeptide may include an epitope that is screened or buried by the conformational change (e.g., a bindable epitope that becomes buried in the protein core when a polypeptide-binding interaction is occurring). In some cases, presence of a signal from an affinity agent with a binding specificity for an epitope associated with a first binding site of a ligand-binding polypeptide at an address on an array may indicate absence of a binding ligand of a plurality of binding ligands at a first binding site of a ligand-binding polypeptide. In some cases, absence of a signal from an affinity agent with a binding specificity for an epitope associated with a first binding site of a ligand-binding polypeptide at an address may indicate presence of a binding target of a plurality of binding targets at a first binding site of a ligand-binding polypeptide. In some cases, an affinity agent of a plurality of affinity agents may comprise a binding ligand for the ligand-binding polypeptide coupled to a detectable label.

A method may further comprise detecting presence or absence of a second polypeptide binding interaction at an address on an array. In some cases, presence or absence of a second polypeptide binding interaction may not depend upon presence or absence of a first polypeptide binding interaction. For example, a ligand-binding polypeptide may comprise two or more binding sites, in which a first binding site may form a polypeptide binding interaction independently of presence or absence of a second polypeptide binding interaction at a second binding site. In other cases, presence or absence of a second polypeptide binding interaction may depend upon presence or absence of a first polypeptide binding interaction. For example, a ligand-binding polypeptide may comprise a second binding site that becomes exposed or occluded by a conformational change caused by presence of a polypeptide binding interaction at a first binding site. A method further comprising the detecting presence or absence of a second polypeptide binding interaction at an address on an array may comprise the steps of: a) contacting an array with a second plurality of detectable affinity agents, in which each detectable affinity agent comprises a characterized binding specificity to an epitope associated with a second binding site of a ligand-binding polypeptide of a plurality of ligand-binding polypeptides; and b) detecting presence or absence of a signal from a detectable affinity agent of the second plurality of detectable affinity agents at each address. In some cases, presence of a signal at an address on the array may indicate absence of a binding ligand of the plurality of binding ligands at the second binding site of the ligand-binding polypeptide. In some cases, absence of a signal at an address on an array may indicate presence of a binding ligand of a plurality of binding ligands at the second binding site of the ligand-binding polypeptide.

A method may comprise a step of detecting presence or absence of a polypeptide binding interaction at each address of an array, in which the detecting comprises the steps of: a) contacting the array with a first plurality of detectable affinity agents, in which each detectable affinity agent comprises a characterized lack of binding to a ligand-binding polypeptide of the plurality of ligand-binding polypeptides; and b) detecting presence or absence of a first signal from a detectable affinity agent of the plurality of detectable affinity agents at each address. A useful affinity agent for such a method will have a property of having a binding specificity for a chemical structure or epitope that may be present in a binding ligand but is not present in a ligand-binding polypeptide. Accordingly, such affinity agents would predominantly bind and/or provide a detectable signal at an address primarily when a binding ligand is present, thereby indicating the formation of a polypeptide binding interaction between a ligand-binding polypeptide and the binding ligand. In some cases, presence of a first signal at an address on an array may indicate presence of a binding ligand of a plurality of binding ligands at a binding site of the ligand-binding polypeptide at the address. In some cases, absence of a first signal at an address on an array may indicate absence of a binding ligand of a plurality of binding ligands at a binding site of the ligand-binding polypeptide at the address.

A method, as set forth herein, may further comprise a step of identifying a binding ligand of a plurality of binding ligands at an address of the plurality of addresses. In some cases, a binding ligand may be identified by detecting presence or absence of binding of one or more pluralities of affinity agents at each address on an array, in which an affinity agent of each of the one or more pluralities of affinity agents comprises a binding specificity for a single binding ligand. For example, methods comprising the detecting of a lipid binding ligand bound to a ligand-binding polypeptide may utilize an affinity agent that binds to a chemical structure of the lipid binding ligand that is not involved in forming a polypeptide binding interaction with the ligand-binding polypeptide. In another example, methods comprising the detecting of a polypeptide binding ligand bound to a ligand-binding polypeptide may utilize an affinity agent that binds to a unique epitope of the polypeptide that is not involved in forming a polypeptide binding interaction with the ligand-binding polypeptide. In other cases, a binding ligand may be identified by detecting presence or absence of binding of one or more pluralities of affinity agents at each address on an array, in which an affinity agent of each of the one or more pluralities of affinity agents comprises a binding specificity for multiple binding ligands. Methods that utilize affinity agents with characterized binding specificities may determine an identity of a binding ligand by combining observed measurement outcomes of each plurality of affinity agents to infer the identity of the binding ligand, such as by a decoding method, as set forth herein. In some cases, identifying a binding ligand of a plurality of binding ligands at an address may comprise the steps of: a) providing one or more observed presences or absences of signal from each plurality of affinity agents at the address to a computer algorithm; and b) determining an identity of the binding ligand at the address by the computer algorithm based upon the one or more observed presences or absences of signal from each plurality of affinity agents at the address.

Identifying a binding ligand at an address on an array may further comprise the steps of: a) contacting the array with a second plurality of detectable affinity agents, in which each detectable affinity agent comprises a characterized lack of binding to a ligand-binding polypeptide of a plurality of ligand-binding polypeptides; b) detecting presence or absence of a second signal from a detectable affinity agent of the plurality of detectable affinity agents at each address; and c) based upon presence or absence of a first signal from a first affinity agent and presence or absence of the second signal from the second affinity agent, identifying a polypeptide of the plurality of polypeptides at an address of a plurality of addresses.

A detectable probe or an affinity agent may have a characterized binding affinity for a binding target. Binding affinity for a binding target may be determined quantitatively by a measure such as equilibrium dissociation constant (K_{D}), association rate constant (kₒₙ), or dissociation rate constant (k_{off}). A detectable probe or affinity agent may be selected to have a binding affinity that increases a likelihood of detection for a chosen detection method. For example, an affinity agent that is to be utilized in a fluorescence-based assay (e.g., fluorescence microscopy, luminescence lifetime, etc.) may be selected based upon a dissociation rate constant that indicates slow dissociation relative to a time-scale for the fluorescence-based detection. In another example, an affinity agent that is to be utilized in a signal encoding assay (e.g., nucleic acid barcode transfer) may be selected based upon an equilibrium dissociation constant that indicates minimal dissociation of complexes, thereby increasing likelihood of proper signal encoding. A detectable probe or affinity agent may have a characterized dissociation constant of no more than about 10 millimolar (mM), 1 mM, 100 micromolar (μM), 10 μM, 1 μM, 500 nanomolar (nM), 250 nM, 100 nM, 50 nM, 25 nM, 10 nM, 5 nM, 2.5 nM, 1 nM, 500 picomolar (pM), 250 pM, 100 pM, 50 pM, 25 pM, 10 pM, 5 pM, 2.5 pM, 1 pM, or less. Alternatively or additionally, a detectable probe or affinity agent may have a characterized dissociation constant of at least about 1 pM, 2.5 pM, 5 pM, 10 pM, 25 pM, 50 pM, 100 pM, 250 pM, 500 pM, 1 nM, 2.5 nM, 5 nM, 10 nM, 25 nM, 50 nM, 100 nM, 250 nM, 500 nM, 1 μM, 10 μM, 100 μM, 1 mM, 10 mM or more. A detectable probe or affinity agent may have a characterized dissociation rate constant of no more than about 1 per second (s⁻¹), 1×10⁻¹ s⁻¹, 2.5×10⁻¹ s⁻¹, 5×10⁻¹ s⁻¹, 1×10⁻² s⁻¹, 2.5×10⁻² s⁻¹, 5×10⁻² s⁻¹, 1×10⁻¹ s⁻³, 2.5×10⁻³ s⁻¹, 5×10⁻³ s⁻¹, 1×10⁻⁴ s⁻¹, 2.5×10⁻⁴ s⁻¹, 5×10⁻⁴ s⁻¹, 1×10⁻⁵ s⁻¹, 2.5×10⁻⁵ s⁻¹, 5×10⁻⁵ s⁻¹, 1×10⁻¹ s⁻⁶, or less. Alternatively or additionally, a detectable probe or affinity agent may have a characterized dissociation rate constant of at least about 1×10⁻⁶ s⁻¹, 5×10⁻⁵ s⁻¹, 2.5×10⁵ s⁻¹, 1×10⁵ s⁻¹, 5×10⁻⁴ s⁻¹, 2.5×10⁴ s⁻¹, 1×10⁻⁴ s⁻¹, 5×10⁻³ s⁻¹, 2.5×10⁻³ s⁻¹, 1×10⁻³ s⁻¹, 5×10⁻² s⁻¹, 2.5×10⁻² s⁻¹, 1×10⁻² s⁻¹, 5×10⁻¹ s⁻¹, 2.5×10⁻¹ s⁻¹, 1×10⁻¹ s⁻¹, 1 s⁻¹ or more. A detectable probe or affinity agent may have a characterized association rate constant of no more than about 1×10⁷ per second (s¹), 5×10⁶ s⁻¹, 2.5×10⁶ s⁻¹, 1×10⁶ s⁻¹, 5×10⁵ s⁻¹, 2.5×10⁵ s⁻¹, 1×10⁵ s⁻³, 5×10⁴ s⁻¹, 2.5×10⁴ s⁻¹, 1×10⁴ s⁻¹, 5×10³ s⁻¹, 2.5×10³ s⁻¹, 1×10³ s⁻¹, or less. Alternatively or additionally, a detectable probe or affinity agent may have a characterized association rate constant of at least about 1×10³ s⁻¹, 2.5×10³ s⁻¹, 5×10³ s⁻¹, 1×10⁴ s⁻¹, 2.5×10⁴ s⁻¹, 5×10⁴ s⁻¹, 1×10⁵ s⁻¹, 2.5×10⁵ s⁻¹, 5×10⁵ s⁻¹, 1×10⁶ s⁻¹, 2.5×10⁶ s⁻¹, 5×10⁶ s⁻¹, 1×10⁷ s⁻¹, or more.

A method, as set forth herein, may comprise identifying a binding entity (e.g., a polypeptide) at an address on an array. In some cases, identifying a binding entity at an address on an array may comprise detecting binding of a detectable probe (e.g., an affinity agent) at the address, in which the detectable probe comprises a binding specificity for the binding entity. In other cases, identifying a binding entity at an address on an array may comprise detecting presence or absence of binding of two or more detectable probes (e.g., two or more affinity agents) at the address, in which each of the detectable probes comprises a characterized binding specificity. In some cases, a method may further comprise providing data to an analysis algorithm regarding presence or absence of observed binding of an affinity agent of a first plurality of affinity agents at an address, and providing data to an analysis algorithm regarding presence or absence of observed binding of an affinity agent of a second plurality of affinity agents at the address. In some cases, an analysis algorithm may comprise one or more of an image analysis algorithm, a signal processing algorithm, and a polypeptide identification algorithm. In some cases, an algorithm may be configured to obtain data regarding presence or absence of observed binding of an affinity agent of a first plurality of affinity agents at an address, and obtain data regarding presence or absence of observed binding of an affinity agent of a second plurality of affinity agents at the address, and identify a binding entity (e.g., a polypeptide, a non-polypeptide) at the address based upon the obtained data.

A method, as set forth herein, may comprise providing an array comprising a plurality of binding targets (e.g., ligand-binding polypeptides, binding ligands, candidate binding ligands, etc.), in which the method comprises one or more of the steps of: a) providing the plurality of binding targets; and b) coupling the plurality of binding targets to a solid support, in which the solid support comprises a plurality of addresses, in which each address of the plurality of addresses is resolvable from each other address, and in which each address of the plurality of addresses comprises a single binding target of the plurality of binding targets. In some cases, coupling a plurality of binding targets to a solid support may comprise one or more steps of: a) coupling each binding target of the plurality of binding targets to a linker; and b) coupling each linker to a single address on the solid support, in which each address comprises a single linker. In some cases, a linker may comprise a nucleic acid. In some cases, coupling each linker to a single address on a solid support may comprise covalently attaching the linker to the solid support. In other cases, coupling each linker to a single address on a solid support may comprise non-covalently attaching the linker to the solid support.

A method, as set forth herein, may further comprise identifying and/or quantifying a binding characteristic of a polypeptide binding interaction. Identifying and/or quantifying a binding characteristic may comprise identifying and/or quantifying a binding specificity. In some cases, identifying and/or quantifying a binding specificity may comprise providing a list of binding ligands that form a polypeptide binding interaction with a ligand-binding polypeptide or polypeptide complexes. In other cases, identifying and/or quantifying a binding specificity may comprise providing a list of ligand-binding polypeptides or polypeptide complexes with a binding specificity for a binding ligand. Identifying and/or quantifying a binding characteristic may comprise quantifying a binding affinity of a ligand-binding polypeptide (e.g., a dissociation constant, an association rate constant, a dissociation rate constant, etc.). In some cases, quantifying a binding affinity of a ligand-binding polypeptide may comprise the steps of: a) detecting at a first time presence or absence at single-analyte resolution of a detectable probe at each address of a plurality of addresses on a solid support, in which each address comprises a binding target (e.g., a ligand-binding polypeptide, a binding ligand, a polypeptide complex), and in which the detectable probe comprises a complementary binding entity to the binding target; b) detecting at a second time presence or absence at single-analyte resolution of the detectable probe at each address of the plurality of addresses on the solid support; and c) based upon the presence or absence of the detectable probe at each address at the first time and the second time, calculating a binding affinity characteristic (e.g., K_{D}, kₒₙ, k_{off}, etc.) of the detectable probe-binding target pair. In other cases, quantifying a binding affinity of a ligand-binding polypeptide or a polypeptide complex may comprise the steps of: a) detecting in a first condition presence or absence at single-analyte resolution of a detectable probe at each address of a plurality of addresses on a solid support, in which each address comprises a binding target (e.g., a ligand-binding polypeptide, a binding ligand, a polypeptide complex), and in which the detectable probe comprises a complementary binding entity to the binding target; b) detecting in a second condition presence or absence at single-analyte resolution of the detectable probe at each address of the plurality of addresses on the solid support; and c) based upon the presence or absence of the detectable probe at each address at the first time and the second time, calculating a binding affinity characteristic of the detectable probe-binding target pair. In some cases, the first condition and/or the second condition may comprise a fluidic condition (e.g., chemical composition, ionic strength, pH, concentration, etc.) or a temperature.

A method, as set forth herein, may comprise a single-analyte array. An array may comprise one or more surfaces comprising one or more addresses that are resolvable from each other address on the array at single-analyte resolution. An array may comprise a plurality of addresses, in which each address of the plurality of addresses is resolvable from each other address on the array at single-analyte resolution. In some cases, an array may comprise a single surface comprising a plurality of addresses, in which each address of the plurality of addresses is resolvable from each other address on the array at single-analyte resolution. An array may comprise a plurality of binding entities or binding targets, in which each binding entity or binding target of the plurality of binding entities or binding targets is coupled to an address of a plurality of addresses on the array. An array may comprise a plurality of binding entities or a plurality of binding targets, in which each binding entity or binding target of the plurality of binding entities or binding targets is coupled to an address of a plurality of addresses on the array, and in which an address of the plurality of addresses comprises no more than one binding entity or binding target of the plurality of binding entities or binding targets. An array may comprise a plurality of binding entities or a plurality of binding targets, in which each binding entity or binding target of the plurality of binding entities or binding targets is coupled to an address of a plurality of addresses on the array, and in which each address of the plurality of addresses comprises no more than one binding entity or binding target of the plurality of binding entities or binding targets. An array may comprise a plurality of binding entities or a plurality of binding targets, in which each binding entity or binding target of the plurality of binding entities or binding targets is coupled to an address of a plurality of addresses on the array, and in which an address of the plurality of addresses comprises two or more binding entities or binding targets of the plurality of binding entities or binding targets. An array may comprise one or more interstitial regions. An array may comprise a plurality of interstitial regions. In some cases, an interstitial region may comprise a moiety that is configured to minimize non-specific binding of molecules to the interstitial region.

A method, as set forth herein, may utilize a single-analyte array of binding entities (e.g, ligand-binding polypeptide, binding ligands, candidate binding ligands, etc.) for the purpose of identifying polypeptide binding interactions at single-analyte resolution. An array may be formed by a deposition of a plurality of binding entities on a solid support. In some cases, a method of forming an array may include the step of providing a plurality of binding entities, in which each binding entity of the plurality of binding entities is coupled to a linker. In another aspect, provided herein is a method of forming a binding entity particle comprising: a) providing a composition comprising an uncoupled binding entity (e.g., a ligand-binding polypeptide, a binding ligand, a candidate binding ligand) and an uncoupled linker; and b) coupling the linker to the binding entity, thereby forming a binding entity particle. In some cases, the method may further comprise coupling the linker to a solid support. In particular cases, a linker may be coupled to a solid support before the linker is coupled to a binding entity. In other particular cases, a linker may not be coupled to a solid support before the linker is coupled to a binding entity. A similar method may be utilized to form a detectable probe comprising one or more binding entities. A method of forming a detectable probe may comprise: a) providing a composition comprising one or more uncoupled binding entities (e.g., a ligand-binding polypeptide, a binding ligand, a candidate binding ligand) and an uncoupled retaining component; and b) coupling the retaining component to the one or more binding entities, thereby forming a binding entity particle.

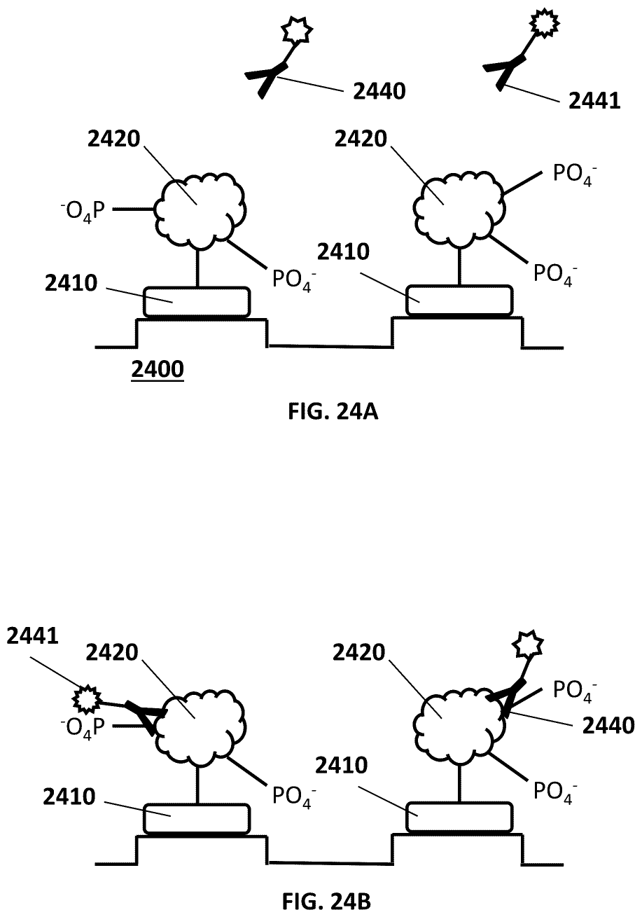

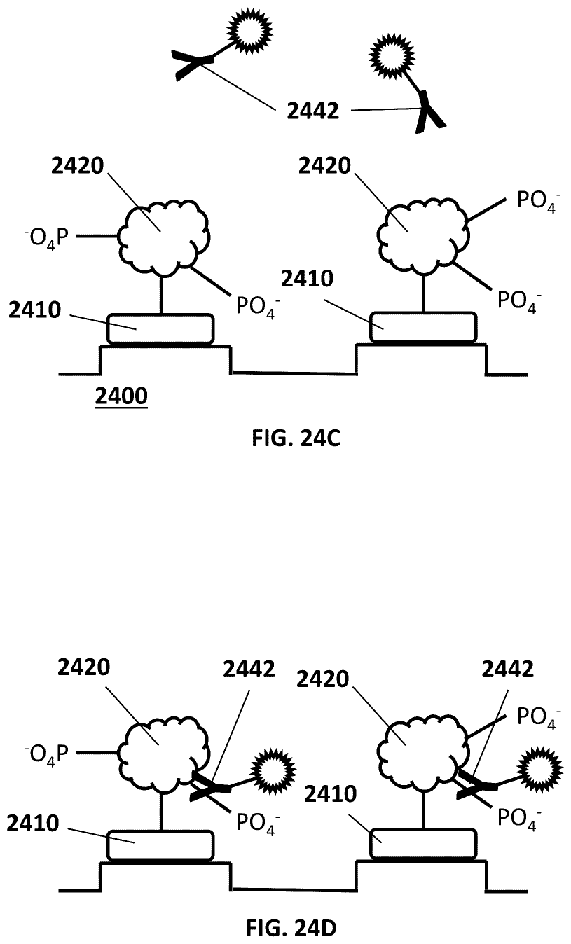

A method, as set forth herein, may comprise identifying a polypeptide by developing a binding profile of affinity agents for the polypeptide. In some cases, such a method may further comprise identifying a proteoform or isoform of the polypeptide based upon an affinity agent binding profile. For example, a method may comprise identifying two or more polypeptide isoforms in a sample, then determining if a binding ligand (e.g., a pharmaceutical compound) is bound by neither, one, or both isoforms. FIGS. 24A-24D depict a method of utilizing affinity agents to identify a polypeptide isoform. FIG. 24A depicts a solid support 2400 comprising polypeptides 2420 with differing phosphorylation patterns. Each polypeptide 2420 is coupled to the solid support 2400 by a linking moiety 2410. The array is contacted with detectable probes 2440 and 2441, with each detectable probe having a binding specificity for a differing phosphorylated epitope. FIG. 24B depicts an array configuration in which detectable probe 2440 has bound to the right isoform and detectable probe 2441 has bound to the left isoform. FIG. 24C depicts contacting the array with a plurality of third detectable probes 2442. FIG. 24D depicts an array configuration in which detectable probes 2442 have bound to both isoforms. Accordingly, it may be determined that both isoforms comprise one common phosphorylated epitope and two differing phosphorylated epitopes before or after a binding ligand interaction assay.

A method, as set forth herein, may utilize a binding entity that is separated or purified from a crude or partially-separated sample. A sample may be separated or purified to obtain one or more binding entities (e.g., a ligand-binding polypeptide, a binding ligand) from the sample. For example, a blood sample may be separated to obtain an albumin and/or globulin-rich fraction. A sample may be separated or purified to remove one or more impurities from one or more binding entities. For example, a sample may be desalted to remove an ionic species (e.g., Ca²⁺, Mg²⁺, Na⁺, K⁺, Fe²⁺, etc.) from one or more binding entities. A sample may be separated or purified to remove a particular species from one or more binding entities. For example, a blood sample may be purified to remove albumins and/or globulins from other serum proteins. A sample may be separated or purified by any suitable method, including but not limited to, centrifugation, precipitation, liquid-liquid separation, solid-phase separation, chromatography, desalting, and combinations thereof. Exemplary methods of preparing binding entities are described in “Clinical Diagnosis and Management by Laboratory Methods,” 16^{th} Ed., Campbell, et al. (1979), “Investigation of an Albumin-enriched Fraction of Human Serum and its Albuminome,” Gundry, et al., Proteomics Clin. Appl., (2007), or “Proteomic and Network Analysis of Human Serum Albuminome by Integrated Use of Quick Cross-linking and Two-step Precipitation,” Liu, et al., Nature Scientific Reports, (2017), each of which is incorporated by reference in its entirety. In some cases, a blood sample may be treated with an anti-coagulant (e.g., citrate, heparin, or EDTA) to prevent clotting or coagulation of species within the blood sample.

A fraction containing a plurality of binding entities, as set forth herein, may comprise one or more impurities. An impurity may refer to any unwanted entity included in a fraction containing a plurality of binding entities. An impurity may comprise a polypeptide impurity or a non-polypeptide impurity. An impurity may comprise a biomolecule, such as a saccharide, a polysaccharide, an amino acid, a protein, a peptide, a nucleotide, a nucleic acid, a vitamin, a cofactor, an ion, a pharmaceutical compound, a toxic compound, a venom, a nanoparticle, a microplastic, a derivative thereof, a degradation product thereof, or a combination thereof. A single-analyte array or a plurality of detectable probes may be prepared from a plurality of binding entities (e.g., ligand-binding polypeptides, binding ligands) that contains one or more impurities. A single-analyte array or a detectable probe of a plurality of detectable probes may contain an impurity. A method, as set forth herein, may utilize a fraction comprising a binding entity and one or more impurities. For example, a single-analyte array may be prepared by directly applying a fluid sample (e.g., blood, cerebrospinal fluid, synovial fluid, etc.) to a solid support and depositing a binding entity (e.g., albumin) and an impurity on separate addresses of the array. A method, as set forth herein, may be configured to function in the presence of an impurity. For example, one or more polypeptide binding interactions between an albumin detectable probe and a plurality of polypeptide candidate binding ligands may be observed on a single-analyte array comprising the plurality of polypeptide candidate binding ligands and one or more impurities (e.g., nucleic acids, lipids, etc.). A fraction containing a plurality of polypeptides or a single-analyte array derived therefrom may comprise impurities coupled to at least about 0.0000000001%, 0.000000001%, 0.00000001%, 0.0000001%, 0.000001%, 0.00001%, 0.0001%, 0.0005%, 0.001%, 0.005%, 0.01%, 0.05%, 0.1%, 0.5%, 1%, or more than about 1% of addresses on the single-analyte array. Alternatively or additionally, a fraction containing a plurality of polypeptides or a single-analyte array derived therefrom may comprise impurities coupled to no more than about 1%, 0.5%, 0.1%, 0.05%, 0.01%, 0.005%, 0.001%, 0.0005%, 0.0001%, 0.00001%, 0.000001%, 0.0000001%, 0.00000001%, 0.000000001%, 0.0000000001%, or less than about 0.0000000001% of addresses on the single-analyte array.

A method, as set forth herein, may utilize a control binding entity (e.g., a control ligand-binding polypeptide, a control binding ligand, etc.). The control binding entity may be derived or extracted from any appropriate source. A control binding entity may be derived or extracted from a differing subject than the subject from which a binding entity is derived or extracted. For example, a plurality of polypeptides may be extracted from a human subject, and a control polypeptide may be extracted from a second human subject. In another example, a plurality of polypeptides may be extracted from a human subject, and a control polypeptide may be extracted from a non-human subject. A control binding entity may be derived or extracted from the same subject from which a binding entity is derived or extracted. A control binding entity may be derived or extracted from a subject with a known or characterized disease or health state. For example, a control albumin may be extracted from a known non-diabetic subject to characterize an albumin sample from a subject with suspected diabetes. A control binding entity may be derived or extracted from a transgenic organism. A control binding entity may be derived or extracted from a healthy tissue or a healthy fluid of a subject from which a binding entity is derived. For example, a binding entity may be extracted from a sample of cerebrospinal fluid collected from a subject, and a control binding entity may be extracted from a blood sample collected from the same subject.

A method, as set forth herein, may comprise deriving or extracting a binding entity from a subject with a known, unknown, or suspected disease state or health state. A method, as set forth herein, may comprise identifying a disease state or a health state in a subject from which a sample comprising a binding entity is obtained. A disease state or a health state may be identified based upon presence or absence of a binding entity in a sample derived or extracted from a subject. A disease state or a health state may be identified based upon presence or absence of a polypeptide binding interaction between a first binding entity derived or extracted from a subject and a second binding entity on a single-analyte array. A disease state may include any disorder of molecular, cellular, or tissue structure or function within a subject that produces biomarkers, symptoms, or abnormal function within the subject. Exemplary disease states may include a cancer state, a cardiovascular disease state, an autoimmune disease state, an inflammatory state, a neuropsychiatric state, a bacterial infection state, a viral infection state, a parasitic infection state, a toxic response state, an envenomation state, or a combination thereof. Specific exemplary disease states may include, but are not limited to, diabetes, brain cancer, oral cancer, esophageal cancer, lung cancer, stomach cancer, liver cancer, gall bladder cancer, pancreatic cancer, colon cancer, leukemia, heart disease, lung disease, stroke, hypertension, hypotension, cirrhosis, kidney disease, arthritis, lupus, psoriasis, multiple sclerosis, celiac disease, Crohn's disease, major depressive disorder, schizophrenia, obsessive-compulsive disorder, viral pneumonia, bacterial pneumonia, viral meningitis, bacterial meningitis, viral gastroenteritis, bacterial gastroenteritis, or a combination thereof. A health state may include any identification or characterization of normal or expected structure or function in a subject. A health state may be determined by presence or absence of a biomarker associated with the health state. For example, an identified presence of a polypeptide biomarker bound to an albumin may be indicative of normal blood pressure. A health state may be determined by presence or absence of a polypeptide binding interaction associated with the health state. For example, a subject may be identified as non-diabetic based upon an absence of a polypeptide binding interaction that is characteristic of a post-translationally modified albumin polypeptide.

A method, as set forth herein, may utilize one or more fluidic media. A method, as set forth herein, may utilize one or more fluidic mediums to contact a binding entity (e.g., a ligand-binding polypeptide, a binding ligand) or an affinity agent with a solid support. A method, as set forth herein, may utilize one or more fluidic media to remove a binding entity (e.g., a ligand-binding polypeptide, a binding ligand) or an affinity agent with a solid support. For example, a fluidic medium may be utilized to rinse unbound affinity agents or detectable probes from a solid support. In another example, a fluidic medium may be utilized to disrupt a binding association, such as a polypeptide binding interaction or the binding of an affinity agent to a binding entity. A method, as set forth herein, may comprise one or more steps of contacting an array with a fluidic medium, in which the fluidic medium has a particular composition (e.g., pH, ionic strength, temperature, polarity, hydrophobicity, hydrophilicity, chemical composition, etc.). A method, as set forth herein, may comprise one or more steps of contacting an array with a fluidic medium, in which the fluidic medium comprises an ionic species (e.g., a monovalent ion, a polyvalent ion, an anion, a cation, a monatomic ion, a polyatomic ion, a metal ion, a non-metal ion, an organic ion, an inorganic ion, etc.). A method, as set forth herein, may comprise one or more steps of contacting an array with a fluidic medium, in which the fluidic medium comprises an ionic species that is configured to alter a polypeptide binding interaction (e.g., strengthen an interaction, facilitate an interaction, disrupt an interaction, etc.). For example, a solid support comprising a ligand-binding polypeptide may be contacted with a fluidic medium comprising an ionic species that mediates the formation of a polypeptide binding interaction between the ligand-binding polypeptide and a binding ligand of the ligand-binding polypeptide. In another example, a solid support comprising a polypeptide complex may be contacted with a fluidic medium comprising an ionic species that disrupts the polypeptide complex, thereby causing dissociation of one or more species from the polypeptide complex.

### Compositions and Systems for Assaying Polypeptide Binding Systems

Provided herein are compositions that may be advantageous for the analysis of polypeptide binding interactions of ligand-binding polypeptides by methods as set forth herein. The compositions may be particularly useful for preparing or analyzing single-analyte arrays of binding entities, such as ligand-binding polypeptides, binding ligands, or polypeptide complexes, in which each binding entity on the single-analyte array is resolvable at single-analyte resolution. Further, disclosed herein are systems that may incorporate one or more of the disclosed compositions, as set forth herein. The disclosed systems may be configured to implement a method as set forth herein, including identifying and/or quantifying a polypeptide binding interaction of a ligand-binding polypeptide at single-analyte resolution.

In an aspect, provided herein is a composition comprising: a) a solid support comprising a plurality of addresses, in which each address of the plurality of addresses is resolvable from each other address, and further comprising a plurality of interstitial regions that separate each address of the plurality of addresses from one or more adjacent addresses of the plurality of addresses; and b) a plurality of binding entities, in which each binding entity of the plurality of binding entities is coupled to an address of the plurality of addresses, and in which the interstitial regions of the plurality of interstitial regions are devoid of binding entities. A plurality of binding entities may comprise a plurality of ligand-binding polypeptides. A plurality of ligand-binding polypeptides may comprise a homogeneous plurality of ligand-binding polypeptides, in which each ligand-binding polypeptide of the plurality of ligand-binding polypeptides is of a same ligand-binding polypeptide species. A plurality of ligand-binding polypeptides may comprise a heterogeneous plurality of ligand-binding polypeptides, in which the plurality of ligand-binding polypeptides comprises two or more species of ligand-binding polypeptides. In some cases, a plurality of ligand-binding polypeptides may comprise an albumin or a globulin. A plurality of binding entities may comprise a plurality of binding ligands. A plurality of binding ligands may comprise a homogeneous plurality of binding ligands, in which each binding ligand of the plurality of binding ligands is of a same binding ligand species. A plurality of binding ligands may comprise a heterogeneous plurality of binding ligands, in which the plurality of binding ligands comprises two or more species of binding ligands.

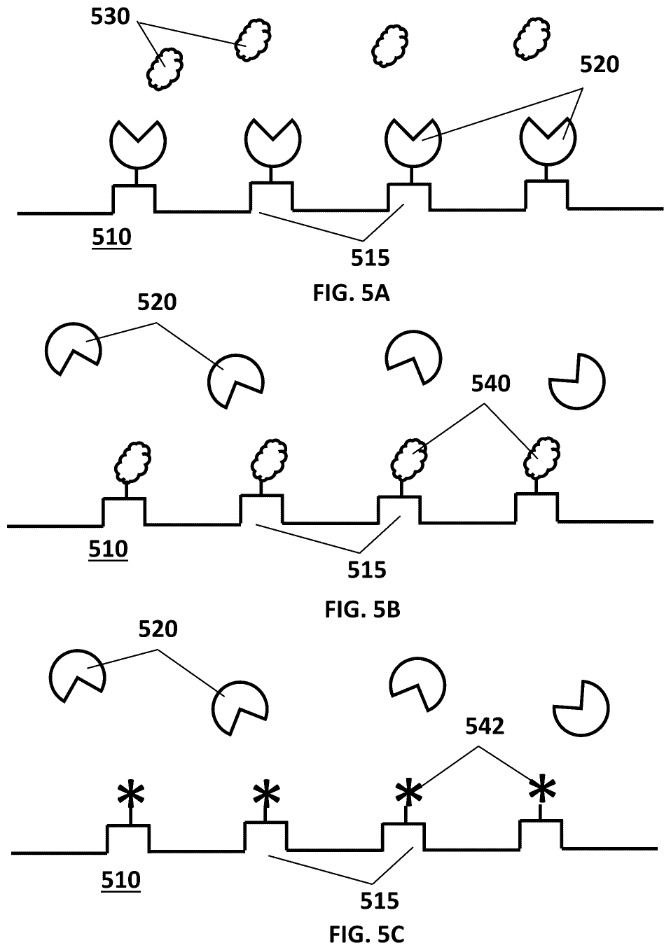

FIGS. 5A-5C illustrate multiple embodiments of arrays that may be useful for methods set forth herein. FIG. 5A depicts an array comprising a solid support 510 containing a plurality of addresses 515. Each address of the plurality of addresses 515 may comprise a single ligand-binding polypeptide 520 of a plurality of ligand-binding polypeptides. Optionally, the array or the solid support 510 may be contacted with a one or more binding entities 530 (e.g., a plurality of polypeptides extracted from a subject, binding ligands, candidate binding ligands, etc.). Optionally, the one or more binding entities 530 may be within a fluidic medium contacted with the array or solid support 510. FIG. 5B depicts an array comprising a solid support 510 containing a plurality of addresses 515. Each address of the plurality of addresses 515 may comprise a single polypeptide binding ligand 540 of a plurality of binding ligands. Optionally, the array or the solid support 510 may be contacted with a one or more ligand-binding polypeptides 520. Optionally, the one or more ligand-binding polypeptides 520 may be within a fluidic medium contacted with the array or solid support 510. FIG. 5C depicts an array comprising a solid support 510 containing a plurality of addresses 515. Each address of the plurality of addresses 515 may comprise a single non-polypeptide binding ligand 540 of a plurality of binding ligands. Optionally, the array or the solid support 510 may be contacted with a one or more ligand-binding polypeptides 520. Optionally, the one or more ligand-binding polypeptides 520 may be within a fluidic medium contacted with the array or solid support 510. The array embodiments depicted in FIGS. 5A-5C each comprise arrays comprising homogeneous pluralities of binding targets, however the arrays can also comprise heterogeneous pluralities of binding targets (e.g., pluralities comprising more than one species of binding target).

In some configurations, a composition may comprise: a) a solid support comprising a plurality of addresses, in which each address of the plurality of addresses is resolvable from each other address, and further comprising a plurality of interstitial regions that separate each address of the plurality of addresses from one or more adjacent addresses of the plurality of addresses; and b) a plurality of ligand-binding polypeptides, in which each ligand-binding polypeptide of the plurality of ligand-binding polypeptides is coupled to an address of the plurality of addresses, and in which the interstitial regions of the plurality of interstitial regions are devoid of ligand-binding polypeptides. In other cases, a composition may comprise: a) a solid support comprising a plurality of addresses, in which each address of the plurality of addresses is resolvable from each other address, and further comprising a plurality of interstitial regions that separate each address of the plurality of addresses from one or more adjacent addresses of the plurality of addresses; and b) a plurality of binding ligands, in which each binding ligand of the plurality of binding ligands is coupled to an address of the plurality of addresses, and in which the interstitial regions of the plurality of interstitial regions are devoid of binding ligands.

A composition, as set forth herein, may comprise a binding entity coupled to an address of an array. A composition may further comprise a linker that is configured to couple a binding entity (e.g., a ligand-binding polypeptide, a binding ligand) to a solid support, or an address thereof. In some cases, a linker may comprise a nucleic acid linker that is configured to couple a binding entity to the solid support. A nucleic acid linker may comprise an oligonucleotide. A nucleic acid linker may comprise a structured nucleic acid particle (SNAP). A SNAP may comprise a nucleic acid origami or a nucleic acid nanoball. In some cases, a nucleic acid linker may be covalently coupled to a solid support. In other cases, a nucleic acid linker may be non-covalently coupled to a solid support. In other cases, a linker may comprise a non-nucleic acid linker. A non-nucleic acid linker may comprise a moiety that is configured to prevent non-specific binding of molecules to an array (e.g., polyethylene glycol, polyethylene oxide, dextran, etc.). A non-nucleic acid linker may comprise a moiety that is configured to form a covalent interaction (e.g., a silane, etc.). In some cases, a linker may comprise a nucleic acid moiety and a non-nucleic acid moiety. For example, a binding entity may be coupled to a nucleic acid moiety (e.g., a SNAP), in which the nucleic acid moiety is coupled to a non-nucleic acid moiety (e.g., a surface-bound silane).

A composition, as set forth herein, may further comprise a plurality of binding entities in contact with an array or a solid support thereof. In some cases, a composition may further comprise a plurality of ligand-binding polypeptides in contact with an array or a solid support thereof, in which the array comprises a plurality of binding ligands. In other cases, a composition may further comprise a plurality of binding ligands in contact with an array or a solid support thereof, in which the array comprises a plurality of ligand-binding polypeptides. In some cases, a composition may further comprise a plurality of competitive binding ligands, in which the array comprises a plurality of binding entities. In some cases, a plurality of binding entities in contact with an array or solid support thereof may comprise one or more free binding entities. In some cases, a plurality of binding entities in contact with an array or solid support thereof may comprise a plurality of free binding entities. In other cases, a plurality of binding entities in contact with an array or solid support thereof may comprise one or more binding entities that are bound to one or more binding entities on the array or solid support thereof. In other cases, a plurality of binding entities in contact with an array or solid support thereof may comprise a plurality of binding entities that are bound to one or more binding entities on the array or solid support thereof. In some cases, a composition may comprise a fluidic medium in contact with an array or a solid support thereof, in which the fluidic medium comprises a plurality of binding entities.

A plurality of binding entities in contact with an array or a solid support thereof may comprise a plurality of polypeptides, in which a polypeptide of the plurality of polypeptides is configured to form a polypeptide binding interaction with a ligand-binding polypeptide of the plurality of ligand-binding polypeptides. A plurality of polypeptides may comprise a single species of polypeptides. A plurality of polypeptides may comprise two or more species of polypeptides. In some cases, a plurality of polypeptides may be derived or extracted from a subject (e.g., a medical patient, a research subject). In some cases, a plurality of polypeptides may be derived from a human subject.

In another aspect, a composition may comprise: a) a solid support comprising a plurality of addresses, in which each address of the plurality of addresses is resolvable from each other address, and further comprising a plurality of interstitial regions that separate each address of the plurality of addresses from one or more adjacent addresses of the plurality of addresses; and b) a plurality of polypeptide complexes, in which each polypeptide complex of the plurality of polypeptide-complexes comprises a ligand-binding polypeptide and a binding ligand of the ligand-binding polypeptide, in which each polypeptide complex of the plurality of polypeptide complexes is coupled to an address of the plurality of addresses, and in which each interstitial region of the plurality of interstitial regions does not comprise a polypeptide complex or a ligand-binding polypeptide. In some cases, the ligand-binding polypeptide may comprise an albumin or a globulin.

A composition comprising an array or a solid support thereof comprising a plurality of coupled binding entities (e.g., a plurality of ligand-binding polypeptides, a plurality of binding ligands, a plurality of polypeptide complexes) may further comprise a fluidic medium. A fluidic medium may comprise a binding entity that is configured to form a polypeptide complex with a coupled binding entity of a plurality of coupled binding entities on an array or solid support thereof. A fluidic medium may comprise a binding entity (e.g., a ligand-binding polypeptide, a binding ligand) that is configured to disrupt a polypeptide complex. For example, a fluidic medium may comprise a competitive binding ligand that causes a dissociation of a ligand-binding polypeptide from a coupled polypeptide complex. A fluidic medium may comprise a chemical species other than a binding entity that is configured to disrupt a polypeptide complex. For example, a fluidic medium may comprise a denaturant or a chaotrope that causes dissociation of a binding ligand from a coupled polypeptide complex.

A composition, as set forth herein, may further comprise a cross-linking molecule. A cross-linking molecule may comprise any molecule that is configured to covalently or non-covalently link a first entity to a second entity (e.g., a ligand-binding polypeptide to a binding ligand). A cross-linking molecule may include a chemical cross-linker or a photoinducible cross-linker. A cross-linking molecule may include bifunctional, trifunctional, and polyfunctional linkers, including homofunctional and heterefunctional linkers. A cross-linking molecule may comprise one or more Click-type functional groups that are configured to form a covalent bond with an entity by a Click-type reaction. A cross-linking molecule include a moiety that is linked by a non-covalent interaction (e.g., hybridized oligonucleotides, streptavidin-biotin, SpyTag-SpyCatcher, SdyTag-SdyCatcher, SnoopTag-Snoopcatcher, etc.). A composition, as set forth herein, may comprise a fluidic medium in contact with an array or a solid support thereof, in which the fluidic medium may comprise a cross-linking molecule. A composition, as set forth herein, may comprise a polypeptide complex, in which a ligand-binding polypeptide and a binding ligand of the polypeptide complex are coupled by a cross-linking molecule.

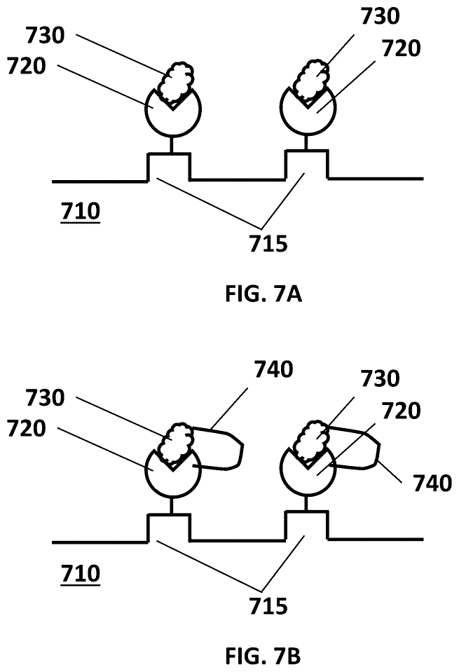

FIGS. 7A-7B illustrate examples of arrays comprising polypeptide complexes with and without cross-links. FIG. 7A shows an array comprising a solid support 710 that contains a plurality of addresses that are resolvable at single-analyte resolution. Each address of the plurality of addresses 715 is linked to a single polypeptide complex, in which each polypeptide complex comprises a ligand-binding polypeptide 720 and a binding ligand 730 of the ligand-binding polypeptide 720. In some configurations, a polypeptide complex may be formed on an array by contacting a binding ligand 730 with a ligand-binding polypeptide 720 that is coupled to an address of the plurality of addresses 715. In other configurations, a free polypeptide complex may be deposited on an array by contacting the free polypeptide complex, optionally within a fluidic medium, with the array, then coupling the free polypeptide complex to an address of the plurality of addresses 715. FIG. 7B shows an array comprising a solid support 710 that contains a plurality of addresses that are resolvable at single-analyte resolution. Each address of the plurality of addresses 715 is linked to a single polypeptide complex, in which each polypeptide complex comprises a ligand-binding polypeptide 720, a binding ligand 730 of the ligand-binding polypeptide 720, and a linker 740 that coupled the ligand-binding polypeptide 720 to the binding ligand 730. In some cases, a polypeptide complex may be cross-linked by a linker 740 after being deposited on the array In other cases, a polypeptide complex may be cross-linked by a linker 740 before being deposited on the array.

In another aspect, provided herein is a composition comprising: a) a solid support comprising a plurality of addresses, in which each address of the plurality of addresses is resolvable from each other address, and further comprising a plurality of interstitial regions that separate each address of the plurality of addresses from one or more adjacent addresses of the plurality of addresses; and b) a plurality of polypeptides, in which a single polypeptide of the plurality of polypeptides is coupled to each address of the plurality of addresses, in which each interstitial region of the plurality of interstitial regions does not comprise a polypeptide of the plurality of polypeptides, and in which the plurality of polypeptides comprises at least 50% albumin and globulin, by weight relative to total protein weight. In some configurations, a plurality of polypeptides may comprise at least about 50%, 55%, 60%, 65%, 70%, 75%, 80%, 85%, 90%, 95%, 99%, 99.9%, 99.99%, 99.999%, or more by weight relative to total protein weight. Alternatively or additionally, a plurality of polypeptides may comprise no more than about 99.999%, 99.99%, 99.9%, 99%, 95%, 90%, 85%, 80%, 75%, 70%, 65%, 60%, 55%, 50%, or less by weight relative to total protein weight. In some cases, a plurality of polypeptides may be derived from a single subject (e.g., a single human subject). In some configurations, a plurality of polypeptides may comprise a purified plurality of polypeptides (e.g., a polypeptide sample having had one or more polypeptide removed by a separation process, etc.). In other configurations, a plurality of polypeptides may not comprise a purified plurality of polypeptides. Surprisingly, a method set forth herein may not necessitate purification of polypeptides due to the ability to identify one or more impurities on an array at single-analyte resolution and exclude them from further analysis.

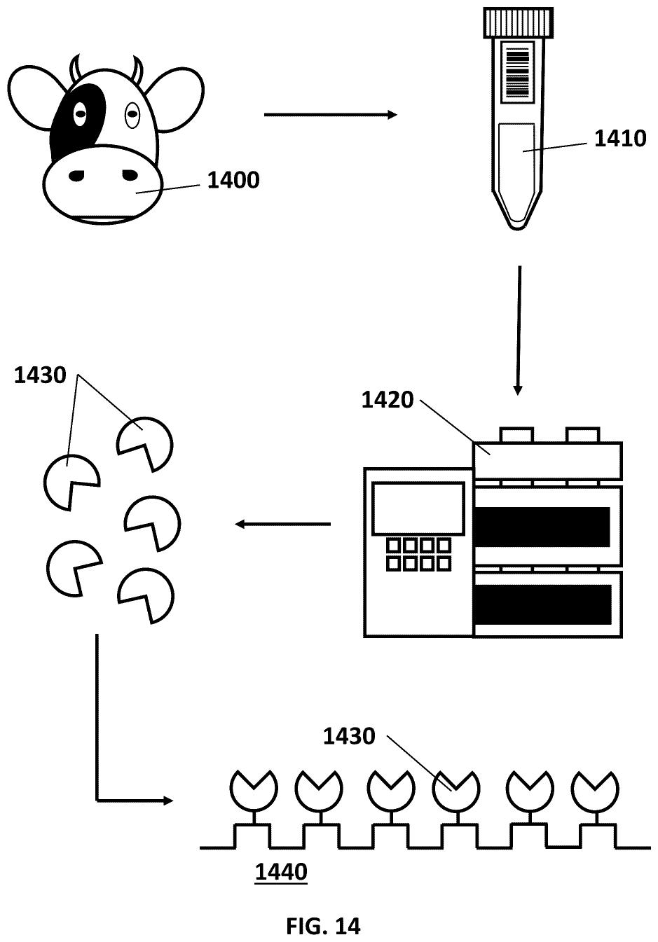

FIG. 14 depicts the formation of an array comprising a plurality of polypeptides extracted from a subject 1400. A sample 1410 (e.g., blood, cerebrospinal fluid, etc.) may be collected or extracted from the subject 1400. The sample may optionally undergo one or more purification processes 1420 to provide a plurality of polypeptides 1430. The plurality of polypeptide 1430 may be provided deposited on an array 1440 by a method as set forth herein.

In another aspect, provided herein is a composition comprising: a) a retaining component comprising one or more detectable labels; and b) two or more polypeptides coupled to the retaining component, in which the two or more polypeptides are independently selected from an albumin and a globulin. In some configuration, the two or more polypeptides may be derived and/or extracted from a single subject (e.g., a single human subject). Such a composition may be useful as a detectable probe for measuring polypeptide binding interactions between ligand-binding polypeptides derived and/or extracted from a subject and an array of binding ligands.

In another aspect, provided herein is a composition comprising: a) a plurality of polypeptides, in which a polypeptide of the plurality of polypeptides comprises a ligand-binding polypeptide selected from an albumin and a globulin; and b) a linker, in which the linker is configured to be coupled to the polypeptide of the plurality of polypeptides, and in which the linker is configured to be coupled to a solid support. In some configurations, a composition may further comprise a binding ligand for a ligand-binding polypeptide. In some configurations, a binding ligand may be a free binding ligand. In other configurations, a binding ligand may be bound to a polypeptide of a plurality of polypeptides. In another aspect, provided herein is a composition comprising: a) a plurality of polypeptides, in which a polypeptide of the plurality of polypeptides comprises a binding ligand of a ligand-binding polypeptide, in which the ligand-binding polypeptide is optionally selected from an albumin and a globulin; and b) a linker, in which the linker is configured to be coupled to the polypeptide of the plurality of polypeptides, and in which the linker is configured to be coupled to a solid support. In some configurations, a composition may further comprise a ligand-binding polypeptide that is configured to form a polypeptide-binding interaction with a binding ligand. In some configurations, a ligand-binding polypeptide may be a free ligand-binding polypeptide. In other configurations, a ligand-binding polypeptide may be bound to a polypeptide of a plurality of polypeptides.

A linker, as set forth herein, may comprise a coupling site that is configured to bind to a binding entity (e.g., a ligand-binding polypeptide, a binding ligand, a polypeptide complex). In some configurations, a coupling site may be configured to covalently bind to a binding entity. In other configurations, a coupling site may be configured to non-covalently bind to a binding entity. A linker may comprise a plurality of coupling sites, in which each coupling site may be configured to covalently bind to a different binding entity. A linker may comprise a plurality of coupling sites, in which each coupling site may be configured to non-covalently bind to a different binding entity. In some configurations, the linker may be coupled to a binding entity. In other configurations, the linker may not be coupled to a binding entity.

A linker, as set forth herein, may comprise a surface that is configured to be coupled to a solid support. In some configurations, a linker may comprise a surface that is configured to be coupled to a solid support by a non-covalent interaction selected from one or more of the group consisting of an electrostatic interaction, hydrogen bonding interaction, van der Waal's interaction, a magnetic interaction, a ligand-binding interaction, and a nucleic acid hybridization interaction. In other configurations, a linker may comprise a surface that is configured to be coupled to a solid support by a covalent interaction. In some configurations, a linker may comprise one or more passivating groups, in which the one or more passivating groups are configured to prevent non-specific binding of a molecule to the linker.

A composition, as set forth herein, may comprise a plurality of polypeptides. A plurality of polypeptides may be derived and/or extracted from a subject (e.g., a human subject, a domesticated animal subject, a non-domesticated animal subject, etc.). A plurality of polypeptides may be derived and/or extracted from an engineered organism. A plurality of polypeptides may comprise at least about 50%, 55%, 60%, 65%, 70%, 75%, 80%, 85%, 90%, 95%, 99%, 99.9%, 99.99%, 99.999%, or more of ligand-binding polypeptides by weight relative to total polypeptide weight. Alternatively or additionally, a plurality of polypeptides may comprise no more than about 99.999%, 99.99%, 99.9%, 99%, 95%, 90%, 85%, 80%, 75%, 70%, 65%, 60%, 55%, 50%, or less of ligand-binding polypeptides by weight relative to total polypeptide weight.

A composition, as set forth herein, may further comprise a solid support. In some configurations, a linker may be coupled to a solid support. In some configurations, a single linker may be coupled to an address on a solid support. In other configurations, a linker may not be coupled to a solid support. In some configurations, a linker coupled to a solid support may be coupled to a binding entity (e.g., a ligand-binding polypeptide, a binding ligand). In other configurations, a linker coupled to a solid support may not be coupled to a binding entity.

In another aspect, provided herein is a composition comprising: a) a solid support comprising a plurality of addresses, in which each address of the plurality of addresses is resolvable from each other address, and further comprising a plurality of interstitial regions that separate each address of the plurality of addresses from one or more adjacent addresses of the plurality of addresses; b) a plurality of ligand-binding polypeptides, in which each ligand-binding polypeptide of the plurality of ligand-binding polypeptides optionally comprises an albumin or a globulin, in which each ligand-binding polypeptide of the plurality of ligand-binding polypeptides is coupled to an address of the plurality of addresses, and in which the interstitial regions of the plurality of interstitial regions are devoid of ligand-binding polypeptides, and c) a plurality of standard polypeptides, in which each standard polypeptide is coupled to an address of the plurality of addresses, and in which the plurality of interstitial regions are devoid of standard polypeptides.

In another aspect, provided herein is a composition comprising: a) a solid support comprising a plurality of addresses, in which each address of the plurality of addresses is resolvable from each other address, and further comprising a plurality of interstitial regions that separate each address of the plurality of addresses from one or more adjacent addresses of the plurality of addresses; b) a plurality of ligand-binding polypeptides, in which each ligand-binding polypeptide of the plurality of ligand-binding polypeptides optionally comprises an albumin or a globulin, in which each ligand-binding polypeptide of the plurality of ligand-binding polypeptides is coupled to an address of the plurality of addresses, and in which the interstitial regions of the plurality of interstitial regions are devoid of ligand-binding polypeptides; and c) a fluidic medium in contact with the solid support, in which the fluidic medium comprises a plurality of binding ligands, in which each binding ligand of the plurality of binding ligands is configured to form a binding interaction with a ligand-binding polypeptide of the plurality of ligand-binding polypeptides.

In another aspect, provided herein is a composition comprising: a) a solid support comprising a plurality of addresses, in which each address of the plurality of addresses is resolvable from each other address, and further comprising a plurality of interstitial regions that separate each address of the plurality of addresses from one or more adjacent addresses of the plurality of addresses; b) a plurality of polypeptide complexes, in which each polypeptide complex comprises a ligand-binding polypeptide coupled to a binding ligand of the ligand-binding polypeptide, in which the ligand-binding polypeptide optionally comprises an albumin or a globulin, in which each polypeptide complex of the plurality of polypeptide complexes is coupled to an address of the plurality of addresses, and in which the interstitial regions of the plurality of interstitial regions are devoid of polypeptide complexes; and c) a fluidic medium in contact with the solid support, in which the fluidic medium comprises a polypeptide complex dissociating species, in which the polypeptide complex dissociating species is selected from the group comprising a salt, an acid, a base, a surfactant, a competitive binding ligand, and a free ligand-binding polypeptide.

In another aspect, provided herein is a composition comprising: a) a solid support comprising a plurality of addresses, in which each address of the plurality of addresses is resolvable from each other address, and further comprising a plurality of interstitial regions that separate each address of the plurality of addresses from one or more adjacent addresses of the plurality of addresses; b) a plurality of ligand-binding polypeptides, in which each ligand-binding polypeptide of the plurality of ligand-binding polypeptides optionally comprises an albumin or a globulin that is extracted from a subject, and in which each ligand-binding polypeptide of the plurality of ligand-binding polypeptides is coupled to an address of the plurality of addresses, and in which the interstitial regions of the plurality of interstitial regions are devoid of ligand-binding polypeptides, and c) a nucleic acid sequence derived from the subject, in which the nucleic acid sequence comprises a genomic sequence, and in which the nucleic acid sequence is coupled to an address of the plurality of addresses.

An array composition, as set forth herein, may be configured to provide quantitative information on the presence of ligand-binding polypeptides and/or binding ligands of ligand-binding polypeptides. An occupancy rate may be a useful characteristic for characterizing arrays or any data collected thereupon. In some cases, an occupancy rate may be useful for quantifying the number of array addresses comprising a particular binding entity. For example, an array comprising a plurality of polypeptides extracted from a human blood sample may have a characterized ligand-binding polypeptide occupancy rate of 0.55. This may be directly comparable to a known standard or average ligand-binding polypeptide occupancy rate of 0.6. In another example, an array comprising a plurality of polypeptides extracted from a sample may be analyzed to simultaneously determine a binding ligand occupancy rate, a ligand-binding polypeptide occupancy rate, and a polypeptide complex occupancy rate. In other cases, an occupancy rate may be useful for quantifying the number of array addresses that are capable of forming a polypeptide binding interaction. For example, an array comprising a plurality of ligand-binding polypeptides may have a characterized ligand-binding polypeptide occupancy rate of 0.99. When contacted with a first plurality of detectable probes comprising a first binding ligand, and a second plurality of detectable probes comprising a second binding ligand, a polypeptide complex occupancy rate for the first detectable probe may be 0.55, and a polypeptide complex occupancy rate for the second detectable probe may be 0.80, suggesting greater binding affinity for the second binding ligand.

An array may have a characterized occupancy rate (e.g., ligand-binding polypeptide occupancy rate, binding ligand occupancy rate, non-binding ligand occupancy rate, polypeptide complex occupancy rate, etc.) of at least about 0.000000001, 0.00000001, 0.0000001, 0.000001, 0.00001, 0.0001, 0.001, 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 0.99, 0.99, 0.999, 0.9999, 0.99999, 0.999999, or greater. Alternatively or additionally, an array may have a characterized occupancy rate of no more than about 0.999999, 0.99999, 0.9999, 0.999, 0.99, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.01, 0.001, 0.0001, 0.00001, 0.000001, 0.0000001, 0.00000001, 0.000000001, or less.

In another aspect, provided herein is a fluidic device, comprising: a) a solid substrate; b) a composition as set forth herein, in which the composition is disposed within the solid substrate; and c) a fluidic channel comprising a first port and a second port, in which the fluidic channel is configured to contact a fluidic medium with the composition, and in which the fluidic channel is configured to receive a portion of the fluidic medium through the first port and evacuate a portion of the fluidic medium through the second port. A substrate of a fluidic device may comprise a housing for the fluidic device. A fluidic device may comprise more than one array or solid support. For example, a fluidic device may comprise two fluidically isolated arrays for conducting assays simultaneously. For example, a fluidic device may comprise a first array for analyzing a first sample and a second array for analyzing a second sample or a control sample. In another example, a fluidic device may comprise two fluidically-coupled arrays for capturing binding entities released from a first array (e.g., a binding ligand dissociated from a polypeptide complex by a competitive binding ligand). A fluidic device may further comprise a second fluidic channel. In some configurations, a second fluidic channel may be in fluidic communication with a first fluidic channel. In other configurations, a second fluidic channel may not be in fluidic communication with a first fluidic channel. In some configurations, a second fluidic channel may comprise a second composition as set forth herein, in which the second composition may be disposed within a same solid substrate as a first composition. In other configurations, a second fluidic channel may comprise a second composition as set forth herein, in which the second composition is not disposed within a same solid substrate as a first composition. For example, a fluidic device may comprise two joined substrate, in which each substrate of the two joined substrates comprises an array that is configured to couple a plurality of binding entities. In some configurations, a second fluidic channel may comprise an unoccupied array that is configured to bind a plurality of polypeptides. For example, an unoccupied array may be configured downstream of a first array to capture binding entities released from a first array (e.g., a binding ligand dissociated from a polypeptide complex by a competitive binding ligand).

Optionally, at least a portion of a channel in a fluidic device can be configured to facilitate detection of analytes. For example, at least a portion of a channel can be transparent to radiation at a wavelength used for detection. For example, at least a portion of a channel can transmit excitation radiation or emission radiation used for optical detection techniques such as luminescence. In another example, at least a portion of a channel can include electronic detectors such as field effect transistors (FETs) or nanopores. A portion of a channel that is configured for detection can house an array of addresses. Alternatively, the portion can be downstream of an array such that analytes that interact with the array (or that are removed from the array) can be detected in a fluid that has been in contact with the array.

In another aspect, provided herein is a composition comprising: a) a solid support comprising a plurality of addresses, in which each address of the plurality of addresses is resolvable from each other address, and further comprising a plurality of interstitial regions that separate each address of the plurality of addresses from one or more adjacent addresses of the plurality of addresses; b) a plurality of binding entities, in which each address of a first fraction of the plurality of addresses comprises a single binding entity of the plurality of binding entities, and in which each binding entity of the plurality of binding entities is coupled to an address of the first fraction of the plurality of addresses; and c) one or more impurities, in which each address of a second fraction of the plurality of addresses comprises a single binding entity of the plurality of binding entities, and in which each binding entity of the plurality of binding entities is coupled to an address of the second fraction of the plurality of addresses. Such a composition may be useful for identifying polypeptide binding interactions from samples that have minimal, incomplete, or no removal of impurities from the sample. A first fraction of a plurality of addresses containing a binding entity may have a ratio to a second fraction of a plurality of addresses containing an impurity of at least about 1:1, 2:1, 5:1, 10:1, 25:1, 50:1, 100:1, 500:1, 1000:1, 10000:1, 100000:1, 1000000:1, 10000000:1, 100000000:1, 1000000000:1, 10000000000:1, or more than 10000000000:1. Alternatively or additionally, a first fraction of a plurality of addresses containing a binding entity may have a ratio to a second fraction of a plurality of addresses containing an impurity of no more than about 10000000000:1, 1000000000:1, 100000000:1, 10000000:1, 1000000:1, 100000:1, 10000:1, 1000:1, 500:1, 100:1, 50:1, 25:1, 10:1, 5:1, 2:1, 1:1, or less than 1:1. In some cases, a single-analyte array composition comprising an impurity may be useful for identifying a polypeptide binding interaction of a low copy number binding entity (e.g., a polypeptide binding ligand species or a ligand-binding polypeptide species that is present at no more than about 1000, 500, 250, 100, 50, 25, 20, 15, 10, 9, 8, 7, 6, 5, 4, 3, 2, or less than 2 addresses on a single-analyte array) in the presence of one or more impurities on a single-analyte array. A first fraction of a plurality of addresses containing a low copy number binding entity may have a ratio to a second fraction of a plurality of addresses containing an impurity of at least about 1:1, 2:1, 5:1, 10:1, 25:1, 50:1, 100:1, 500:1, 1000:1, 10000:1, 100000:1, 1000000:1, 10000000:1, 100000000:1, 1000000000:1, 10000000000:1, or more than 10000000000:1. Alternatively or additionally, a first fraction of a plurality of addresses containing a low copy number binding entity may have a ratio to a second fraction of a plurality of addresses containing an impurity of no more than about 10000000000:1, 1000000000:1, 100000000:1, 10000000:1, 1000000:1, 100000:1, 10000:1, 1000:1, 500:1, 100:1, 50:1, 25:1, 10:1, 5:1, 2:1, 1:1, or less than 1:1.

A composition or system of the present disclosure may be useful for identifying and/or quantifying a relative binding specificity and/or binding affinity of a ligand-binding polypeptide. In another aspect, provided herein is a system comprising: a) in a first configuration, a solid support comprising a plurality of addresses, in which each address of the plurality of addresses is resolvable from each other address, and further comprising a plurality of interstitial regions that separate each address of the plurality of addresses from one or more adjacent addresses of the plurality of addresses, in which each address of the plurality of addresses comprises a first polypeptide complex comprising a ligand-binding molecule coupled to a polypeptide, in which the ligand-binding polypeptide optionally comprises an albumin or a globulin, and in which the solid support is in contact with a fluidic medium comprising a competitive binding ligand, in which the competitive binding ligand is a binding ligand of the ligand-binding polypeptide; and b) in a second configuration, the solid support comprising the plurality of addresses, in which a subset of addresses of the plurality of addresses comprises a second polypeptide complex comprising the ligand-binding polypeptide and the competitive binding ligand, and in which the solid support is in contact with a fluidic medium comprising the polypeptide; in which the first configuration and the second configuration are distinguishable at single-analyte resolution. In some configurations, a polypeptide may comprise a first detectable label and a competitive binding ligand may comprise a second detectable label, in which the first detectable label is distinguishable from the second detectable label.

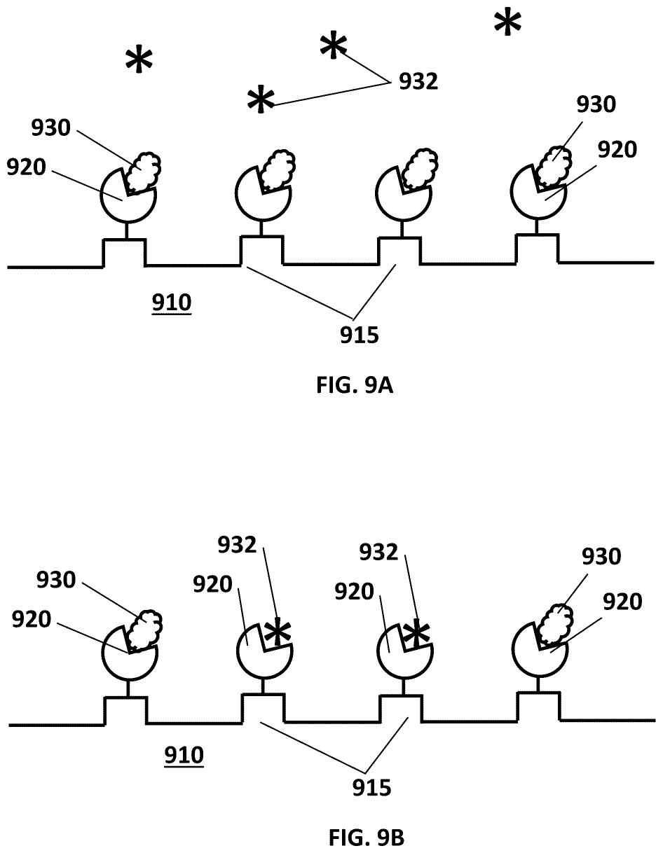

FIGS. 9A-9B illustrate the exchange of binding ligands in polypeptide complexes on a single-analyte array. FIG. 9A depicts a first configuration of an array comprising a solid support 910 containing a plurality of addresses 915. Each address of the plurality of addresses 915 comprises a polypeptide complex, in which the polypeptide complex comprises a ligand-binding polypeptide 920 that forms a polypeptide binding interaction with a binding ligand 930. A plurality of competitive binding ligands is contacted with the solid support 910, optionally within a fluidic medium. FIG. 9B depicts a second configuration of the array. One or more addresses of the plurality of addresses 915 comprise a polypeptide complex comprising the ligand-binding polypeptide 920 and the competitive binding ligand 932. The first configuration and the second configuration may be distinguishable at single-analyte resolution by a method set forth herein.

Alternatively, in another aspect, provided herein is a system comprising: a) in a first configuration, a solid support comprising a plurality of addresses, in which each address of the plurality of addresses is resolvable from each other address, and further comprising a plurality of interstitial regions that separate each address of the plurality of addresses from one or more adjacent addresses of the plurality of addresses, in which each address of the plurality of addresses comprises a first polypeptide complex comprising a ligand-binding molecule coupled to a polypeptide, in which the ligand-binding polypeptide optionally comprises an albumin or a globulin, and in which the solid support is in contact with a fluidic medium comprising a second ligand-binding polypeptide; and b) in a second configuration, the solid support comprising the plurality of addresses, in which a subset of addresses of the plurality of addresses comprises the ligand-binding polypeptide, and in which the solid support is in contact with a fluidic medium comprising a free second polypeptide complex, in which the free second polypeptide complex comprises the second ligand-binding polypeptide coupled to the polypeptide; in which the first configuration and the second configuration are distinguishable at single-analyte resolution. In some configurations, a polypeptide may comprise a first detectable label and a ligand-binding polypeptide may comprise a second detectable label, in which the first detectable label is distinguishable from the second detectable label.

A composition or system of the present disclosure may be useful for identifying and/or quantifying a formation of a polypeptide complex comprising two or more binding ligands. In another aspect, provided herein is a system comprising: in a first configuration, a solid support comprising a plurality of addresses, in which each address of the plurality of addresses is resolvable from each other address, and further comprising a plurality of interstitial regions that separate each address of the plurality of addresses from one or more adjacent addresses of the plurality of addresses, in which each address of the plurality of addresses comprises a first polypeptide complex comprising a ligand-binding molecule coupled to a first polypeptide, in which the ligand-binding polypeptide optionally comprises an albumin or a globulin, and in which the solid support is in contact with a fluidic medium comprising a second polypeptide, in which the second polypeptide is a binding ligand of the ligand-binding polypeptide; and b) in a second configuration, the solid support comprising the plurality of addresses, in which a subset of addresses of the plurality of addresses comprises a second polypeptide complex comprising the ligand-binding polypeptide, the first polypeptide, and the second polypeptide; in which the first configuration and the second configuration are distinguishable at single-analyte resolution. In some configurations, a first polypeptide may comprise a first detectable label and a second polypeptide may comprise a second detectable label, in which the first detectable label is distinguishable from the second detectable label.

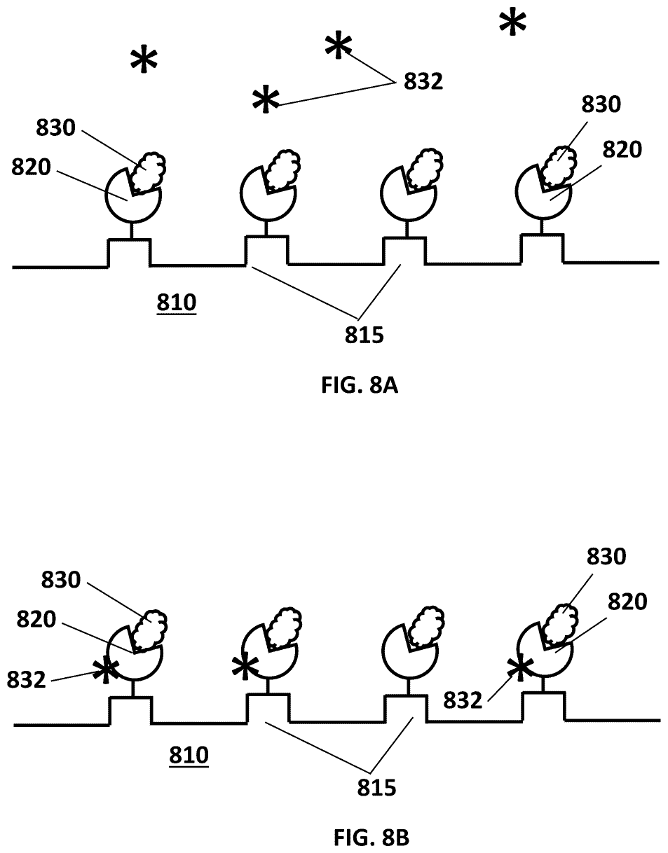

FIGS. 8A-8B illustrate the formation of polypeptide complexes comprising two or more binding ligands on a single-analyte array. FIG. 8A depicts a first configuration of an array comprising a solid support 810 containing a plurality of addresses 815. Each address of the plurality of addresses 815 comprises a polypeptide complex, in which the polypeptide complex comprises a ligand-binding polypeptide 820 that forms a polypeptide binding interaction with a first binding ligand 830. A plurality of second binding ligands is contacted with the solid support 810, optionally within a fluidic medium. FIG. 8B depicts a second configuration of the array. One or more addresses of the plurality of addresses 815 comprise a polypeptide complex comprising the ligand-binding polypeptide 820, the first binding ligand 830, and the second binding ligand 832. The first configuration and the second configuration may be distinguishable at single-analyte resolution by a method set forth herein.

Further provided herein are systems that are configured to implement a method, as set forth herein. A system may comprise an array comprising a plurality of binding entities, in which each binding entity is resolvable at single-analyte resolution. In some configurations, a system may comprise an array that is disposed within a fluidic device. A system may further comprise a single-analyte detection device that is configured to obtain measurements of a polypeptide binding interaction on an array at single-analyte resolution. In some configurations, a system may further comprise a fluid transfer system that provides and/or removes one or more fluids from an array. In some configurations, a system may further comprise a computer that is configured to received one or more measurements from a single-analyte detection device and identify and/or quantify one or more characteristics of a polypeptide binding interaction.

In another aspect, provided herein is a system comprising: a) a fluidic device as set forth herein; b) a fluid transfer device in fluidic communication with the fluidic device, in which the fluid transfer device is configured to deliver one or more fluidic media to the fluidic device, in which at least one fluidic medium of the one or more fluidic media comprises a plurality of detectable probes; c) a physical measurement device, in which the physical measurement device is configured to receive a physical measurement comprising a presence or absence of signal a detectable probe within the fluidic device; and d) a computer that is configured to receive the physical measurement from the physical measurement device, in which the computer is configured to identify one or more characteristics of a polypeptide binding interaction on a solid support within the fluidic device based upon the physical measurement.

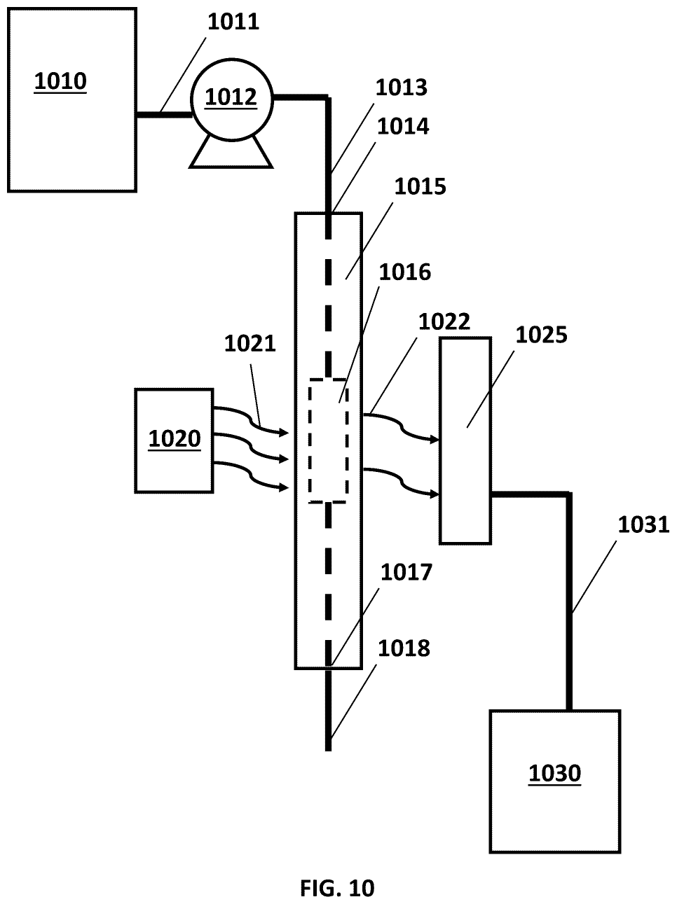

FIG. 10 depicts a schematic of a system that is configured to implement a method, as set forth herein, a single-analyte resolution. A fluidic device 1015 comprising a substrate material contains a fluidic channel 1016 within which a single-analyte array is disposed. The fluidic device 1015 is fluidically connected to a reservoir 1010 that provides one or more fluidic media to the fluidic device 1015. A fluid transfer device 1012 (e.g., a pump) transfers one or more fluidic media from the reservoir 1010 to the inlet port 1014 of the fluidic device 1015. Fluidic connections 1011 and 1013 (e.g., tubing, robotic injector, etc.) provide fluidic connectivity between the reservoir, fluid transfer device, and fluidic device. One or more fluidic media are withdrawn or discharged from the fluidic device 1015 by an exit fluidic connection 1018 that is fluidically connected to an outlet port 1017. The system further comprises a single-analyte sensor 1025 that is configured to detect one or more signals 1022 originating from addresses on the array disposed within the fluidic channel 1016. The one or more signals 1022 may be induced by an excitation source 1020 that provides an excitation field 1021 (e.g., light, heat, etc.) to the array disposed within the fluidic channel 1016. The single-analyte sensor is connected to a computer 1030 via a data transfer connection 1031 (e.g., wired or wireless connection). The computer 1030 is configured to determine one or more characteristics of a polypeptide binding interaction based upon the signals 1022 detected originating from the single-analyte array.

A system, as set forth herein, may comprise a fluidic device comprising an array of binding entities. In some configurations, an array may be disposed within a fluidic channel comprising a first port and a second port. A first port of a fluidic channel may be configured to deliver a fluidic medium to an array. A second port of a fluidic channel may be configured to withdraw a fluidic medium from an array. In some configurations, a fluidic device may be configured for reversible flow directions (e.g., fluid delivery and/or withdrawal through any port). In some configurations, a fluidic device may comprise two or more fluidically-isolated channels, in which each channel comprises an array comprising a plurality of binding entities. In some configurations, a fluidic device may comprise two or more fluidically-connected channels, in which each channel comprises an array comprising a plurality of binding entities. A fluidic device may further comprise one or more couplings that are configured to connect a fluidic device to a fluid transfer system.

A system, as set forth herein, may further comprise a fluid transfer system. A fluid transfer system may be configured to transfer one or more fluidic media to a fluidic device. A fluid transfer system may comprise one or more fluid displacement devices, such as positive-displacement pumps, vacuum pumps, compressors, blowers, etc. A fluid transfer system may comprise one or more fluid control devices, such as mass flow controllers, volumetric flow controllers, valves, and manifolds. A fluid transfer system may comprise one or more fluidic sensors that are configured to obtain measurements on fluid flow within the fluid transfer system, such as pressure sensors, flow sensors, bubble sensors, pH sensors, and temperature sensors. A fluid transfer system may comprise tubing or piping that is configured to provide fluidic connectivity between two or more components of the fluid transfer system. A fluid transfer system may comprise an automated or robotic fluid transfer device that is configured to transfer a fluidic medium from a first location to a second location, in which the first location and the second location are not fluidically connected by piping or tubing. A fluid transfer system may comprise a reservoir that is configured to comprise a fluidic medium. In some configurations, a fluid transfer system may comprise two or more reservoirs, in which each reservoir is configured to contain a fluidic medium. In some configurations, a reservoir may comprise a fluidic medium comprising a detectable probe or affinity agent, as set forth herein.

A system, as set forth herein, may further comprise a single-analyte detection device. A single-analyte detection device may be configured to obtain a physical measurement at single-analyte resolution from an array comprising a plurality of binding entities. In some configurations, a single-analyte detection device may comprise a sensor comprising a plurality of channels or pixels. A single-analyte detection device may comprise a sensor comprising a sufficient quantity of pixels or channels to resolve a plurality of addresses on an array at single-analyte resolution. A single-analyte detection device may further comprise an excitation device that is configured to provide an input of energy or mass to an array comprising a plurality of binding entities. In some cases, an input of energy may comprise an excitation field, such as light radiation, an electrical field, or a magnetic field. In some configurations, a mass input may be provided by a fluid transfer system (e.g., providing a substrate for a fluorescent enzyme, etc.). A single-analyte detection device may further comprise an excitation device that is configured to provide an input that produces a detectable signal from an array. A single-analyte detection device may comprise a measurement system that is configured to obtain a physical measurement of an array at single-analyte resolution (e.g., fluorescence microscopy, surface plasmon resonance, atomic force microscopy, transmission electron microscopy, etc.). In some configurations, a single-analyte detection device may comprise one or more optical components, such as light sources (e.g, lamps, lasers, etc.), mirrors, lenses, filters, etc. In some configurations, a single-analyte detection device may comprise an optical device that is configured to perform an optical signal measurement (e.g., fluorescence microscopy, confocal microscopy, luminescence lifetime measurement, etc.).

A system, as set forth herein, may comprise a computer. A computer may be configured to obtain one or more physical measurements from a single-analyte detection device. A computer may comprise one or more algorithms that are configured to determine a characteristic of a polypeptide binding interaction based upon one or more physical measurements obtained from a single-analyte device. In some configurations, an algorithm may comprise an image-processing algorithm, a signal processing algorithm, or a polypeptide identification algorithm. In some cases, a computer may comprise a data reduction algorithm, in which the data reduction algorithm is configured to reduce the number of calculations performed during an analysis of polypeptide binding interaction data. A data reduction algorithm may be configured to: a) obtain a first physical measurement comprising presence or absence of a first polypeptide binding interaction at each address of a plurality of addresses on an array; b) based upon presence or absence of the first polypeptide binding interaction at each address of the plurality of addresses on the array, identify presence or absence of a high-copy number polypeptide at a subset of addresses of the plurality of addresses on the array beyond a threshold measure of confidence; and c) obtain a second physical measurement comprising presence or absence of a second polypeptide binding interaction at each address of the plurality of addresses on the array; and d) identify a characteristic of a binding entity at each address of the plurality of addresses, excluding the subset of addresses. A computer may comprise one or more processors that are configured to implement an algorithm for a method, as set forth herein. A computer may comprise a plurality of processors, in which each processor is configured to implement one or more algorithms. In some configurations, a computer may comprise a processor that is located within an instrument that comprises a system, as set forth herein. In some configurations, a computer may comprise a cloud-based processor. A computer may comprise a data transfer device that is configured to provide one or more physical measurements from a single-analyte detection device to the computer. A data transfer device may comprise a hard-wired data transfer device, a wireless data transfer device, or a combination thereof.

A composition or system, as set forth herein, may comprise a fluidic medium. A fluidic medium may be configured to contact a binding entity (e.g., a ligand-binding polypeptide, a binding ligand) or an affinity agent with a solid support. A fluidic medium may be configured to remove a binding entity (e.g., a ligand-binding polypeptide, a binding ligand) or an affinity agent from a solid support. A fluidic medium may comprise a property that alters a polypeptide binding interaction (e.g., pH, ionic strength, composition, temperature, fluidic pressure, hydrophobicity, hydrophilicity, polarity, etc.). A fluidic medium may comprise a specific species that alters a polypeptide binding interaction (e.g., an ionic species, a surfactant species, a chaotropic species, etc.).

A fluid medium may comprise any of a variety of components, such as a solvent species, pH buffering species, a cationic species, an anionic species, a surfactant species, a denaturing species, or a combination thereof. A solvent species may include water, acetic acid, methanol, ethanol, n-propanol, isopropyl alcohol, n-butanol, formic acid, ammonia, propylene carbonate, nitromethane, dimethyl sulfoxide, acetonitrile, dimethylformamide, acetone, ethyl acetate, tetrahydrofuran, dichloromethane, chloroform, carbon tetrachloride, dimethyl ether, diethyl ether, 1-4, dioxane, toluene, benzene, cyclohexane, hexane, cyclopentane, pentane, or combinations thereof. A fluid medium may include a buffering species including, but not limited to, MES, Tris, Bis-tris, Bis-tris propane, ADA, ACES, PIPES, MOPSO, MOPS, BES, TES, HEPES, HEPBS, HEPPSO, DIPSO, MOBS, TAPSO, TAPS, TABS, POPSO, TEA, EPPS, Tricine, Gly-Gly, Bicine, AMPD, AMPSO, AMP, CHES, CAPSO, CAPS, and CABS. A fluid medium may include cationic species such as Na⁺, K⁺, Ag⁺, Cu⁺, NH₄⁺, Mg²⁺, Ca²⁺, Cu²⁺, Cd²⁺, Zn²⁺, Fe²⁺, Co²⁺, Ni²⁺, Cr²⁺, Mn²⁺, Ge²⁺, Sn²⁺, Al³⁺, Cr³⁺, Fe³⁺, Co³⁺, Ni³⁺, Ti³⁺, Mn³⁺, Si⁴⁺, V⁴⁺, Ti⁴⁺, Mn⁴⁺, Ge⁴⁺, Se⁴⁺, V⁵⁺, Mn⁵⁺, Mn⁶⁺, Se⁶⁺, and combinations thereof. A fluid medium may include anionic species such as F⁻, Cl⁻, Br⁻, ClO₃⁻, H₂PO₄⁻, HCO₃⁻, HSO₄⁻, OH⁻, I⁻, NO₃⁻, NO₂⁻, MnO₄⁻, SCN⁻, CO₃²⁻, CrO₄²⁻, Cr₂O₇²⁻, HPO₄²⁻, SO₄²⁻, SO₃²⁻, PO₄³⁻, and combinations thereof. A fluid medium may include a surfactant species, such as a cationic surfactant, an anionic surfactant, a zwitterionic surfactant, or an amphoteric surfactant. A fluid medium may include a surfactant species including, but not limited to, stearic acid, lauric acid, oleic acid, sodium dodecyl sulfate, sodium dodecyl benzene sulfonate, dodecylamine hydrochloride, hexadecyltrimethylammonium bromide, polyethylene oxide, nonylphenyl ethoxylates, Triton X, pentapropylene glycol monododecyl ether, octapropylene glycol monododecyl ether, pentaethylene glycol monododecyl ether, octaethylene glycol monododecyl ether, lauramide monoethylamine, lauramide diethylamine, octyl glucoside, decyl glucoside, lauryl glucoside, Tween 20, Tween 80, n-dodecyl-β-D-maltoside, nonoxynol 9, glycerol monolaurate, polyethoxylated tallow amine, poloxamer, digitonin, zonyl FSO, 2,5-dimethyl-3-hexyne-2,5-diol, Igepal CA630, Aerosol-OT, triethylamine hydrochloride, cetrimonium bromide, benzethonium chloride, octenidine dihydrochloride, cetylpyridinium chloride, adogen, dimethyldioctadecylammonium chloride, CHAPS, CHAPSO, cocamidopropyl betaine, amidosulfobetaine-16, lauryl-N,N-(dimethylammonio)butyrate, lauryl-N,N-(dimethyl)-glycinebetaine, hexadecyl phosphocholine, lauryldimethylamine N-oxide, lauryl-N,N-(dimethyl)-propanesulfonate, 3-(1-pyridinio)-1-propanesulfonate, 3-(4-tert-butyl-1-pyridinio)-1-propanesulfonate, N-laurylsarcosine, and combinations thereof.

A fluidic medium may be characterized by a pH. A fluidic medium may have a pH of at least about 0, 1, 2, 3, 4, 5, 6, 7, 8, 9, 10, 11, 12, 13, 14, or more than 14. Alternatively or additionally, a fluidic medium may have a pH of no more than about 14, 13, 12, 11, 10, 9, 8, 7, 6, 5, 4, 3, 2, 1, 0, or less than 0. A fluidic medium may be characterized by an ionic strength. An ionic strength may be determined based upon total ionic composition (e.g., a 1M salt solution), or with respect to a single ionic species (e.g., a solution containing 0.1M Mg²⁺). A fluidic medium may have an ionic strength (total, or specific to one or more species) of at least about 0.000001M, 0.00001M, 0.0001M, 0.001M, 0.01M, 0.1M, 0.2M, 0.3M, 0.4M, 0.5M, 0.6M, 0.7M, 0.8M, 0.9M, 1M, 1.5M, 2M, 2.5M, 3M, 3.5M, 4M, 4.5M, 5M, 6M, 7M, 8M, 9M, 10M, or more than 10M. Alternatively or additionally, a fluidic medium may have an ionic strength (total, or specific to one or more species) of no more than about 10M, 9M, 8M, 7M, 6M, 5M, 4.5M, 4M, 3.5M, 3M, 2.5M, 2M, 1.5M, 1M, 0.9M, 0.8M, 0.7M, 0.6M, 0.5M, 0.4M, 0.3M, 0.2M, 0.1M, 0.01M, 0.001M, 0.0001M, 0.00001M, 0.000001M, or less than 0.000001M. A fluidic medium may be characterized by a concentration of a non-ionic species (e.g., a surfactant, a chaotrope, etc.). An ionic strength may be determined based upon a total non-ionic composition (e.g., a 1M salt solution), or with respect to a single non-ionic species (e.g., a solution containing 0.1M Tween-20). A fluidic medium may have a non-ionic concentration (total, or specific to one or more species) of at least about 0.000001M, 0.00001M, 0.0001M, 0.001M, 0.01M, 0.1M, 0.2M, 0.3M, 0.4M, 0.5M, 0.6M, 0.7M, 0.8M, 0.9M, 1M, 1.5M, 2M, 2.5M, 3M, 3.5M, 4M, 4.5M, 5M, 6M, 7M, 8M, 9M, 10M, or more than 10M. Alternatively or additionally, a fluidic medium may have a non-ionic concentration (total, or specific to one or more species) of no more than about 10M, 9M, 8M, 7M, 6M, 5M, 4.5M, 4M, 3.5M, 3M, 2.5M, 2M, 1.5M, 1M, 0.9M, 0.8M, 0.7M, 0.6M, 0.5M, 0.4M, 0.3M, 0.2M, 0.1M, 0.01M, 0.001M, 0.0001M, 0.00001M, 0.000001M, or less than 0.000001M. A fluidic medium may be characterized by a temperature. A fluidic medium may have a temperature of at least about −80 degrees Centigrade (° C.), −70° C., −60° C., −50° C., −40° C., −30° C., −20° C., −10° C., −5° C., 0° C., 4° C., 10° C., 20° C., 30° C., 37° C., 40° C., 50° C., 60° C., 70° C., 80° C., 90° C., or at least about 95° C. Alternatively or additionally, a fluidic medium may have a temperature of no more than about 95° C., 90° C., 80° C., 70° C., 60° C., 50° C., 40° C., 37° C., 30° C., 20° C., 10° C., 4° C., 0° C., −5° C., −10° C., −20° C., −30° C., −40° C., −50° C., −60° C., −70° C., or about −80° C.

### Analyte Assay Compositions and Methods

The present disclosure provides compositions, apparatus and methods that can be useful for characterizing sample components, such as proteins, nucleic acids, cells or other species, by obtaining multiple separate and non-identical measurements of the sample components. In particular configurations, the individual measurements may not, by themselves, be sufficiently accurate or specific to make the characterization, but an aggregation of the multiple non-identical measurements can allow that characterization to be made with a high degree of accuracy, specificity and confidence. For example, the multiple separate measurements can include subjecting the sample to reagents that are promiscuous with regard to recognizing multiple components of the sample. Accordingly, a first measurement carried out using a first promiscuous reagent may perceive a first subset of sample components without distinguishing one component from another. A second measurement carried out using a second promiscuous reagent may perceive a second subset of sample components, again, without distinguishing one component from another. However, a comparison of the first and second measurements can distinguish: (i) a sample component that is uniquely present in the first subset but not the second; (ii) a sample component that is uniquely present in the second subset but not the first; (iii) a sample component that is uniquely present in both the first and second subsets; or (iv) a sample component that is uniquely absent in the first and second subsets. The number of promiscuous reagents used, the number of separate measurements acquired, and degree of reagent promiscuity (e.g. the diversity of components recognized by the reagent) can be adjusted to suit the component diversity expected for a particular sample.

For ease of explanation, the compositions, apparatus and methods of the present disclosure will be exemplified in the context of characterizing proteins using binding measurements. The examples set forth herein can be readily extended to characterizing other analytes (as an alternative or addition to proteins), or to the use of promiscuous reagents other than promiscuous binding agents. A composition, apparatus or method set forth herein can be used to characterize an analyte, or moiety thereof, with respect to any of a variety of characteristics or features including, for example, presence, absence, quantity (e.g. amount or concentration), chemical reactivity, molecular structure, structural integrity (e.g. full length or fragmented), maturation state (e.g. presence or absence of pre- or pro-sequence in a protein), location (e.g. in an analytical system, subcellular compartment, cell or natural environment), association with another analyte or moiety, binding affinity for another analyte or moiety, biological activity, chemical activity or the like. An analyte can be characterized with regard to a relatively generic characteristic such as the presence or absence of a common structural feature (e.g. amino acid sequence length, overall charge or overall pKa for a protein) or common moiety (e.g. a short primary sequence motif or post-translational modification for a protein). An analyte can be characterized with regard to a relatively specific characteristic such as a unique amino acid sequence (e.g. for the full length of the protein or a motif), an RNA or DNA sequence that encodes a protein (e.g. for the full length of the protein or a motif), or an enzymatic or other activity that identifies a protein. A characterization can be sufficiently specific to identify an analyte, for example, at a level that is considered adequate or unambiguous by those skilled in the art.

In particular configurations, a protein can be detected using one or more affinity agents having known or measurable binding affinity for the protein. For example, an affinity agent can bind a protein to form a complex and a signal produced by the complex can be detected. A protein that is detected by binding to a known affinity agent can be identified based on the known or predicted binding characteristics of the affinity agent. For example, an affinity agent that is known to selectively bind a candidate protein suspected of being in a sample, without substantially binding to other proteins in the sample, can be used to identify the candidate protein in the sample merely by observing the binding event. This one-to-one correlation of affinity agent to candidate protein can be used for identification of one or more proteins. However, as the protein complexity (i.e. the number and variety of different proteins) in a sample increases, or as the number of different candidate proteins to be identified increases, the time and resources to produce a commensurate variety of affinity agents having one-to-one specificity for the proteins approaches limits of practicality.

Methods set forth herein, can be advantageously employed to overcome these constraints. In particular configurations, the methods can be used to identify a number of different candidate proteins that exceeds the number of affinity agents used. For example, the number of candidate proteins identified can be at least 5×, 10×, 25×, 50×, 100× or more than the number of affinity agents used. This can be achieved, for example, by (1) using promiscuous affinity agents that bind to multiple different candidate proteins suspected of being present in a given sample, and (2) subjecting the protein sample to a set of promiscuous affinity agents that, taken as a whole, are expected to bind each protein in a different combination, such that each protein is expected to be encoded by a unique profile of binding and non-binding events. Promiscuity of an affinity agent is a characteristic that can be understood relative to a given population of proteins. Promiscuity can arise due to the affinity agent recognizing an epitope that is known to be present in a plurality of different candidate proteins, in which the candidate proteins are suspected of being present in the given population of proteins. For example, epitopes having relatively short amino acid lengths such as dimers, trimers, tetramers, pentamers or hexamers can be expected to occur in a substantial number of different proteins in the human proteome. Alternatively or additionally, a promiscuous affinity agent can recognize different epitopes (i.e. having a variety of different structures), the different epitopes being present in a plurality of different candidate proteins. For example, a promiscuous affinity agent that is designed or selected for its affinity toward a first trimer epitope may bind to a second epitope that has a different sequence of amino acids when compared to the first epitope.

Although performing a single binding reaction between a promiscuous affinity agent and a complex protein sample may yield ambiguous results regarding the identity of the different proteins to which it binds, the ambiguity can be resolved when the results are combined with other identifying information about those proteins. The identifying information can include characteristics of the protein such as length (i.e. number of amino acids), hydrophobicity, charge to mass ratio, isoelectric point, chromatographic fractionation behavior, enzymatic activity, presence or absence of post translational modifications or the like. The identifying information can include results of binding with other promiscuous affinity agents. For example, a plurality of different promiscuous affinity agents can be contacted with a complex population of proteins, in which the plurality is configured to produce a different binding profile for each candidate protein suspected of being present in the population. In this example, each of the affinity agents is distinguishable from the other affinity agents, for example, due to unique labeling (e.g. different affinity agents have different luminophore labels), unique spatial location (e.g. different affinity agents are located at different addresses in an array), and/or unique time of use (e.g. different affinity agents are delivered in series to a population of proteins). Accordingly, the plurality of promiscuous affinity agents produces a binding profile for each individual protein that can be decoded to identify a unique combination of epitopes present in the individual protein, and this can in turn be used to identify the individual protein as a particular candidate protein having the same or similar unique combination of epitopes. The binding profile can include observed binding events as well as observed non-binding events and this information can be compared to the presence and absence of epitopes, respectively, in a given candidate protein to make a positive identification.

In some configurations, distinct and reproducible binding profiles may be observed for some or even a substantial majority of proteins that are to be identified in a sample. However, in many cases one or more binding events produces inconclusive or even aberrant results and this, in turn, can yield ambiguous binding profiles. For example, observation of binding outcome for a single-molecule binding event can be particularly prone to ambiguities due to stochasticity in the behavior of single molecules when observed using certain detection hardware. The present disclosure provides methods that provide accurate protein identification despite ambiguities and imperfections that can arise in many contexts. In some configurations, methods for identifying, quantitating or otherwise characterizing one or more proteins in a sample utilize reference binding profiles for one or more candidate proteins that are suspected of being present in the sample. The reference binding profiles can include information regarding expected binding outcomes (e.g. binding or non-binding) for binding of one or more affinity agent with one or more candidate proteins. The information can include an a priori characteristic of a candidate protein, such as presence or absence of a particular epitope in the candidate protein or length of the candidate protein. Alternatively or additionally, the information can include empirically determined characteristics such as propensity or likelihood that the candidate protein will bind to a particular affinity agent despite lacking an a priori recognizable epitope for the affinity agent. Accordingly, a reference binding profile can include information regarding the propensity or likelihood of a given candidate protein generating a false positive or false negative binding result in the presence of a particular affinity agent, and such information can optionally be included for a plurality of affinity agents.

Methods set forth herein can be used to evaluate the degree of compatibility of one or more empirical binding profiles with one or more reference binding profiles to identify one or more candidate protein in a sample. For example, to identify a match, an empirical binding profile can be compared to reference binding profiles for many or all candidate proteins suspected of being in a given sample. In some configurations of the methods set forth herein, a match is determined based on the likelihood of the unknown protein being a particular candidate protein given the empirical binding pattern, or based on the probability of a particular candidate protein generating the empirical binding pattern. Optionally a score can be determined from the measurements that are acquired for the unknown protein with respect to many or all candidate proteins suspected of being in the sample. A digital or binary score that indicates one of two discrete states can be used. In particular configurations, the score can be non-digital or non-binary. For example, the score can be a value selected from a continuum of values such that an identity is made based on the score being above or below a threshold value. Moreover, a score can be a single value or a collection of values.

Methods, compositions and apparatus of the present disclosure can be advantageously deployed in a situation where proteins having identical primary structure generate different empirical binding profiles despite being subjected to the same set of affinity agents. For example, the methods, compositions and apparatus are well suited for single-molecule detection and other formats that are prone to stochastic variability. By evaluating the degree of compatibility of the empirical binding profiles with one or more reference binding profiles, proteins can be identified as being identical to the same candidate protein. Accordingly, the present disclosure provides compositions, apparatus and methods that overcome ambiguities and errors in observed binding outcomes to provide binding profiles that are useful for accurate identification of proteins. The methods can be advantageously deployed for complex samples including proteomes or subfractions thereof.

The present disclosure provides assays that are useful for detecting one or more analytes. Exemplary assays are set forth herein in the context of detecting proteins. Those skilled in the art will recognize that methods, compositions and apparatus set forth herein can be adapted for use with other analytes such as nucleic acids, polysaccharides, metabolites, vitamins, hormones, enzyme co-factors and others set forth herein or known in the art. Particular configurations of the methods, apparatus and compositions set forth herein can be made and used, for example, as set forth in U.S. Pat. No. 10,473,654 or US Pat. App. Pub. Nos. 2020/0318101 A1 or 2020/0286584 A1, each of which is incorporated herein by reference. Exemplary methods, systems and compositions are set forth in further detail below.

The present disclosure provides a method for identifying a candidate protein in a sample. The method can include steps of (a) contacting a plurality of different affinity agents with a plurality of proteins in a sample; (b) determining empirical binding profiles for individual proteins of the plurality of proteins, in which each of the empirical binding profiles comprise observed outcomes of binding or non-binding of the respective protein to the plurality of different affinity agents; (c) providing reference binding profiles for a plurality of candidate proteins; and (d) identifying a set of candidate proteins in the sample based on determining compatibility of the empirical binding profiles with the reference binding profiles. Optionally, a common candidate protein is identified from different empirical binding profiles for a plurality of candidate proteins in the set of candidate proteins.

In particular configurations, a method for identifying a candidate protein in a sample can include steps of (a) contacting a plurality of different affinity agents with a plurality of proteins in a sample, in which the plurality of proteins comprises a subset of proteins having identical primary structures; (b) determining empirical binding profiles for individual proteins of the plurality of proteins, in which each of the empirical binding profiles comprise observed outcomes of binding or non-binding of the respective protein to the plurality of different affinity agents, and in which different empirical binding profiles are generated for the proteins in the subset despite the proteins in the subset having identical primary structures; (c) providing reference binding profiles for a plurality of candidate proteins; and (d) identifying a set of candidate proteins in the sample based on determining compatibility of the empirical binding profiles with the reference binding profiles, in which the subset of proteins are identified to be the same candidate protein based on the degree of compatibility of a reference binding profile for the candidate protein with the different empirical binding profiles.

Optionally, a method for identifying a candidate protein in a sample can include steps of (a) contacting a plurality of different affinity agents with a plurality of proteins in a sample; (b) providing reference binding profiles for a set of candidate proteins, in which the reference binding profile for each said candidate protein comprises a plurality of the reference measurement outcomes for said candidate protein, in which each said reference measurement outcome comprises predicted outcome of binding or non-binding of said individual protein with the plurality of different affinity agents; (c) acquiring an empirical measurement outcome for an individual protein of the sample based on: (i) observation of binding or non-binding of the individual protein with an individual affinity agent of the plurality of different affinity agents, and (ii) determination of compatibility between the observed outcome and the reference measurement outcomes for the plurality of different affinity agents, whereby the empirical measurement outcome comprises an observed outcome that is compatible with a reference measurement outcome; (d) repeating step (c) for a plurality of the individual affinity agents, thereby generating an empirical binding profile for the individual protein, the empirical binding profile comprising a plurality of empirical measurement outcomes for the individual protein; and (e) identifying a candidate protein as being in the sample by determining an extent of compatibility between the plurality of empirical measurement outcomes for the individual protein and the reference binding profiles for the set of candidate proteins.

The present disclosure provides a method for locating proteins in an array of proteins. The method can include steps of (a) randomly attaching proteins to unique identifiers, thereby generating an array of different proteins, in which a unique identifier is attached to each said different protein; (b) contacting the array with a plurality of different affinity agents, whereby binding or non-binding of the affinity agents to the proteins produce signals associated with the unique identifiers; (c) determining empirical binding profiles from the signals associated with the unique identifiers, in which each of the empirical binding profiles comprises observed outcomes of binding or non-binding of the respective protein to the plurality of different affinity agents; (c) providing reference binding profiles for a plurality of candidate proteins; and (d) identifying a candidate protein attached to each of the unique identifiers based on determining compatibility of the empirical binding profiles with the reference binding profiles.

A composition, apparatus or method set forth herein can be used to identify proteins from a biological sample, such as a cell, organelle, tissue, or organism. As used herein, the terms “protein” or “polypeptide” refer to a molecule comprising two or more amino acids joined by a peptide bond. A protein may also be referred to as a polypeptide, oligopeptide or peptide. A protein can be a naturally-occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications.

A biological sample can be derived from a homogeneous culture or population of organisms or alternatively from a collection of several different organisms, for example, in a community or ecosystem. In particular configurations, the sample can be a proteome or subfraction of a proteome. A proteome or subfraction can have a complexity of at least 5, 10, 100, 1×10³, 1×10⁴, 2×10⁴, 3×10⁴ or more different native-length protein primary sequences. Alternatively or additionally, a proteome or subfraction can have a complexity that is at most 3×10⁴, 2×10⁴, 1×10⁴, 1×10³, 100, 10, 5 or less. A sample used herein need not be from a biological source and can instead be from a synthetic source, such as a library from a combinatorial synthesis or a library from an in vitro synthesis that exploits biological components. A synthetic sample can have a complexity similar to those set forth above for proteomes. A method set forth herein can detect, identify or characterize some or all proteins in a proteome or other sample including, for example, at least about 1%, 5%, 10%, 25%, 50%, 75%, 90% or 99% of the proteins in the sample.

Some configurations of the compositions, apparatus or methods set forth herein, can distinguish different proteoforms, such as proteins having the same primary structure (i.e. the same sequence of amino acids) but differing with respect to the number, type, or location of post-translational modifications. Methods of the present disclosure can be configured to identify a number, type, or location for one or more post-translational modifications in one or more proteins of a sample. Exemplary post-translational modifications include, but are not limited to, a phosphoryl, glycosyl (e.g. N-acetylglucosamine or polysialic acid), ubiquitin, acyl (e.g. myristoyl or palmitoyl), isoprenyl, prenyl, farnesyl, geranylgeranyl, lipoyl, acetyl, alkyl (e.g. methyl or ethyl), flavin, heme, phosphopantetheinyl, C-terminal amidation, hydroxyl, nucleotidyl, adenylyl, uridylyl, proprionyl, S-glutathionyl, sulfate, succinyl, carbamyl, carbonyl, SUMOyl, or nitrosyl moiety. A variety of post-translational modifications and methods for detection or characterization of proteoforms are set forth in U.S. Pat. App. Ser No. 63/193,486 or 63/139,739, each of which is incorporated herein by reference.

Any of a variety of affinity agents can be used in a composition, apparatus or method set forth herein. As used herein, the term “affinity agent” refers to a molecule or other substance that is capable of specifically or reproducibly binding to an analyte (e.g. protein). An affinity agent can be larger than, smaller than or the same size as the analyte. An affinity agent may form a reversible or irreversible bond with an analyte. An affinity agent may bind with an analyte in a covalent or non-covalent manner. Affinity agents may include reactive affinity agents, catalytic affinity agents (e.g., kinases, proteases, etc.) or non-reactive affinity agents (e.g., antibodies or fragments thereof). An affinity agent can be non-reactive and non-catalytic, thereby not permanently altering the chemical structure of an analyte to which it binds. Affinity agents that can be particularly useful for binding to proteins include, but are not limited to, antibodies or functional fragments thereof (e.g., Fab′ fragments, F(ab′)₂ fragments, single-chain variable fragments (scFv), di-scFv, tri-scFv, or microantibodies), affibodies, affilins, affimers, affitins, alphabodies, anticalins, avimers, DARPins, monobodies, nanoCLAMPs, or lectins or functional fragments thereof.

An affinity agent can be characterized, for example, prior to use in a method set forth herein, with respect to its binding properties. Exemplary binding properties that can be characterized include, but are not limited to, specificity, strength of binding; equilibrium binding constant (e.g. K_{A} or K_{D}); binding rate constant, such as association rate constant (kₒₙ) or dissociation rate constant (k_{off}); binding probability; or the like. Binding properties can be determined with regard to an epitope, a set of epitopes (e.g. a set of proteins having structural similarities), a protein, a set of proteins (e.g. a set of proteins having structural similarities), or a proteome.

As used herein, the term “epitope” refers to an affinity target within a protein, polypeptide or other analyte. Epitopes may comprise amino acid sequences that are sequentially adjacent in the primary structure of a protein or amino acids that are structurally adjacent in the secondary, tertiary or quaternary structure of a protein. An epitope can optionally be recognized by or bound to an antibody. However, an epitope need not necessarily be recognized by any antibody, for example, instead being recognized by an aptamer, mini-protein or other affinity agent. An epitope can optionally bind an antibody to elicit an immune response. However, an epitope need not necessarily participate in, nor be capable of, eliciting an immune response.

An affinity agent can include a label. Exemplary labels include, without limitation, a fluorophore, luminophore, chromophore, nanoparticle (e.g., gold, silver, carbon nanotubes), heavy atom, radioactive isotope, mass label, charge label, spin label, receptor, ligand, nucleic acid barcode, polypeptide barcode, polysaccharide barcode, or the like. A label can produce any of a variety of detectable signals including, for example, an optical signal such as absorbance of radiation, luminescence (e.g. fluorescence or phosphorescence) emission, luminescence lifetime, luminescence polarization, or the like; Rayleigh and/or Mie scattering; magnetic properties; electrical properties; charge; mass; radioactivity or the like. A label component may produce a signal with a characteristic frequency, intensity, polarity, duration, wavelength, sequence, or fingerprint. A label need not directly produce a signal. For example, a label can bind to a receptor or ligand having a moiety that produces a characteristic signal. Such labels can include, for example, nucleic acids that are encoded with a particular nucleotide sequence, avidin, biotin, non-peptide ligands of known receptors, or the like. An affinity agent may include a label that has an inducible signal. For example, an affinity agent may comprise a fluorophore that is induced by an exciting photon, an enzymatic group (e.g., horseradish peroxidase) that produces a fluorescent reaction product in the presence of a substrate, or a component of a proximity-based pair that may produce a signal when in close proximity to a complementary label (e.g., a Forster resonant energy transfer (FRET) fluorophore pair).

A method set forth herein can be carried out in a fluid phase or on a solid phase. For fluid phase configurations, a fluid containing one or more proteins can be mixed with another fluid containing one or more affinity agents. For solid phase configurations one or more proteins or affinity agents can be attached to a solid support. One or more components that will participate in a binding event can be contained in a fluid and the fluid can be delivered to a solid support, the solid support being attached to one or more other component that will participate in the binding event. As used herein, the term “solid support” refers to a substrate that is insoluble in aqueous liquid. Optionally, the substrate can be rigid. The substrate can be non-porous or porous. The substrate can optionally be capable of taking up a liquid (e.g. due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In particular configurations a flow cell contains the solid support such that fluids introduced to the flow cell can interact with a surface of the solid support to which one or more components of a binding event (or other reaction) is attached.

A method of the present disclosure can be carried out at single analyte resolution. As used herein, the term “single analyte” refers to an analyte (e.g. protein, nucleic acid, or affinity agent) that is individually manipulated or distinguished from other analytes. A single analyte can be a single molecule (e.g. single protein), a single complex of two or more molecules (e.g. a single protein attached to a structured nucleic acid particle or a single protein attached to an affinity agent), a single particle, or the like. A single analyte may be resolved from other analytes based on, for example, spatial or temporal separation from the other analytes. Accordingly, an analyte can be detected at “single-analyte resolution” which is the detection of, or ability to detect, the analyte on an individual basis, for example, as distinguished from its nearest neighbor in an array. Reference herein to a ‘single analyte’ in the context of a composition, apparatus or method does not necessarily exclude application of the composition, apparatus or method to multiple single analytes that are manipulated or distinguished individually, unless indicated contextually or explicitly to the contrary.

Alternatively to single-analyte resolution, a method can be carried out at ensemble-resolution or bulk-resolution. Bulk-resolution configurations acquire a composite signal from a plurality of different analytes or affinity agents in a vessel or on a surface. For example, a composite signal can be acquired from a population of different protein-affinity agent complexes in a well or cuvette, or on a solid support surface, such that individual complexes are not resolved from each other. Ensemble-resolution configurations acquire a composite signal from a first collection of proteins or affinity agents in a sample, such that the composite signal is distinguishable from signals generated by a second collection of proteins or affinity agents in the sample. For example, the ensembles can be located at different addresses in an array. Accordingly, the composite signal obtained from each address will be an average of signals from the ensemble, yet signals from different addresses can be distinguished from each other.

A composition, apparatus or method set forth herein can be configured to contact one or more proteins (e.g. an array of different proteins) with a plurality of different affinity agents. For example, a plurality of affinity agents (whether configured separately or as a pool) may comprise at least 2, 5, 10, 25, 50, 100, 250, 500 or more types of affinity agents, each type of affinity agent differing from the other types with respect to the epitope(s) recognized. Alternatively or additionally, a plurality of affinity agents may comprise at most 500, 250, 100, 50, 25, 10, 5, or 2 types of affinity agents, each type of affinity agent differing from the other types with respect to the epitope(s) recognized. Different types of affinity agents in a pool can be uniquely labeled such that the different types can be distinguished from each other. In some configurations, at least two, and up to all, of the different types of affinity agents in a pool may be indistinguishably labeled. Alternatively or additionally to the use of unique labels, different types of affinity agents can be delivered and detected serially when evaluating one or more proteins (e.g. in an array).

A method of the present disclosure can be performed in a multiplex format. In multiplexed configurations, different proteins can be attached to different unique identifiers (e.g. addresses in an array), and the proteins can be manipulated and detected in parallel. For example, a fluid containing one or more different affinity agents can be delivered to an array such that the proteins of the array are in simultaneous contact with the affinity agent(s). Moreover, a plurality of addresses can be observed in parallel allowing for rapid detection of binding events. A plurality of different proteins can have a complexity of at least 5, 10, 100, 1×10³, 1×10⁴, 2×10⁴, 3×10⁴ or more different native-length protein primary sequences. Alternatively or additionally, a proteome or proteome subfraction that is analyzed in a method set forth herein can have a complexity that is at most 3×10⁴, 2×10⁴, 1×10⁴, 1×10³, 100, 10, 5 or fewer different native-length protein primary sequences. The plurality of proteins can constitute a proteome or subfraction of a proteome. The total number of proteins of a sample that is detected, characterized or identified can differ from the number of different primary sequences in the sample, for example, due to the presence of multiple copies of at least some protein species. Moreover, the total number of proteins of a sample that is detected, characterized or identified can differ from the number of candidate proteins suspected of being in the sample, for example, due to the presence of multiple copies of at least some protein species, absence of some proteins in a source for the sample, or loss of some proteins prior to analysis.

A particularly useful multiplex format uses an array of proteins and/or affinity agents. As used herein, the term “array” refers to a population of analytes (e.g. proteins) that are attached to unique identifiers such that the analytes can be distinguished from each other. As used herein, the term “unique identifier” refers to a solid support (e.g. particle or bead), spatial address in an array, tag, label (e.g. luminophore), or barcode (e.g. nucleic acid barcode) that is attached to an analyte and that is distinct from other identifiers, throughout one or more steps of a process. The process can be an analytical process such as a method for detecting, identifying, characterizing or quantifying an analyte. Attachment to a unique identifier can be covalent or non-covalent (e.g. ionic bond, hydrogen bond, van der Waals forces etc.). A unique identifier can be exogenous to the analyte, for example, being synthetically attached to the analyte. Alternatively, a unique identifier can be endogenous to the analyte, for example, being attached or associated with the analyte in the native milieu of the analyte. An array can include different analytes that are each attached to different unique identifiers. An array can include different unique identifiers that are attached to the same or similar analytes. An array can include separate solid supports or separate addresses that each bear a different analyte, in which the different analytes can be identified according to the locations of the solid supports or addresses.

As used herein, the term “address,” when used in reference to an array, means a location in an array where a particular analyte (e.g. protein) is present. An address can contain a single analyte, or it can contain a population of several analytes of the same species (i.e. an ensemble of the analytes). Alternatively, an address can include a population of different analytes. Addresses are typically discrete. The discrete addresses can be contiguous, or they can be separated by interstitial spaces. An array useful herein can have, for example, addresses that are separated by an average distance of less than 100 microns, 10 microns, 1 micron, 100 nm, 10 nm or less. Alternatively or additionally, an array can have addresses that are separated by an average distance of at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses. Average spacing of addresses may be determined by any suitable method, such as optical microscopy, electron microscopy, atomic force microscopy, or surface profilometry.

A protein can be attached to a unique identifier using any of a variety of means. The attachment can be covalent or non-covalent. Exemplary covalent attachments include chemical linkers such as those achieved using click chemistry or other linkages known in the art or described in U.S. patent application Ser. No. 17/062,405, which is incorporated herein by reference. Non-covalent attachment can be mediated by receptor-ligand interactions (e.g. (strept)avidin-biotin, antibody-antigen, or complementary nucleic acid strands), for example, in which the receptor is attached to the unique identifier and the ligand is attached to the protein or vice versa. In particular configurations, a protein is attached to a solid support (e.g. an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers such as tags or addresses in an array are set forth in U.S. patent application Ser. Nos. 17/062,405 and 63/159,500, each of which is incorporated herein by reference.

A method of the present disclosure can include a step of assaying binding between a protein and affinity agent to determine a measurement outcome. As used herein, the term “measurement outcome” refers to information resulting from observation or examination of a process. For example, the measurement outcome for contacting an affinity agent with an analyte can be referred to as a “binding outcome.” A measurement outcome can be positive or negative. For example, observation of binding is a positive binding outcome and observation of non-binding is a negative binding outcome. A measurement outcome can be a null outcome in the event a positive or negative outcome does not result from a given measurement. An “empirical” measurement outcome includes information based on observation of a signal from an analytical technique. A “putative” measurement outcome includes information based on theoretical or a priori evaluation of an analytical technique or analytes.

Binding can be detected using any of a variety of techniques that are appropriate to the assay components used. For example, binding can be detected by acquiring a signal from a label attached to an affinity agent when bound to an observed protein, acquiring a signal from a label attached to protein when bound to an observed affinity agent, or signal(s) from labels attached to an affinity agent and protein. In some configurations a protein-affinity agent complex need not be directly detected, for example, in formats where a nucleic acid tag or other moiety is created or modified as a result of binding between the protein and affinity agent. Optical detection techniques such as luminescent intensity detection, luminescence lifetime detection, luminescence polarization detection, or surface plasmon resonance detection can be useful. Other detection techniques include, but are not limited to, electronic detection such as techniques that utilize a field-effect transistor (FET), ion-sensitive FET, or chemically-sensitive FET. Exemplary methods are set forth in U.S. Pat. No. 10,473,654 or U.S. Pat. App. Ser. No. 63/112,607 or 63/132,170, each of which is incorporated herein by reference.

A method of the present disclosure can include a step of determining an empirical binding profile for a protein. The empirical binding profile can include observed outcomes of binding or non-binding of the protein to a plurality of different affinity agents. In a multiplex format, an empirical binding profile can be determined for each of the proteins of a plurality of proteins, in which each of the empirical binding profiles comprise observed outcomes of binding or non-binding of the respective protein to a plurality of different affinity agents. As used herein, the term “binding profile” refers to a plurality of binding outcomes for a protein or other analyte. The binding outcomes can be obtained from independent binding observations, for example, independent binding outcomes can be acquired using different affinity agents, respectively. A binding profile can include empirical measurement outcomes, putative measurement outcomes or both. A binding profile can exclude empirical measurement outcomes or putative measurement outcomes.

A reference binding profile can include a plurality of putative binding outcomes for a candidate protein. Reference profiles can be provided for a plurality of different candidate proteins. The plurality of candidate proteins may comprise at least 10, 20, 30, 40, 50, 60, 70, 80, 90, 100, 200, 300, 400, 500, 600, 800, 1000, or more different candidate proteins. In some embodiments, one or more reference binding profiles can be stored in database. Particularly useful information that can be included in a database or in a reference binding profile includes, for example, binding characteristics for binding of one or more affinity agents to a protein. For example, the information can include a binding probability of each of a plurality of affinity agents to each of a plurality of candidate proteins. In some configurations, binding probabilities or other binding characteristics are derived empirically, for example, from binding experiments carried out between one or more known candidate proteins and known affinity agent(s). In some embodiments, binding probabilities or other binding characteristics are derived based on a priori information such as presence of a suspected epitope sequence in the structure (e.g. amino acid sequence) of a candidate protein. A reference binding profile for a candidate protein can include a probability or likelihood that an empirical measurement of the candidate protein would generate an observed measurement outcome. Additionally, or alternatively, a reference binding profile for a candidate protein can include a probability that an empirical measurement of the candidate protein would not generate an observed measurement outcome.

A reference binding profile can be used in a method or apparatus of the present disclosure. For example, one or more candidate protein can be identified in a sample by evaluating the degree of compatibility of an empirical binding profile for each candidate protein with one or more reference binding profiles. An empirical binding profile for an unknown protein can be compared to reference binding profiles for many or all candidate proteins suspected of being in a given sample, and the results of the comparison can be used to identify a candidate protein that is a match. In accordance with the present methods, the identity for a particular unknown protein can be determined based on the likelihood of every candidate protein being the unknown protein. The likelihood of a given candidate protein being the unknown protein can be determined based on the probability of each affinity agent binding to the given candidate protein.

In some configurations of the methods set forth herein, the empirical pattern for an unknown protein is assigned a score indicating the likelihood of the unknown protein being a particular candidate protein given the empirical binding pattern, and/or the score can indicate the probability of a particular candidate protein generating the empirical binding pattern. Optionally, a score can be determined for the unknown protein with respect to many or all candidate proteins suspected of being in the sample. The scores can be combined, the proportion of the total score contributed by the top matching score can be determined, and the proportion can be compared to a threshold value to determine whether an identification will be made.

A particularly useful score for evaluating degree of compatibility of a binding profile to a candidate protein is a proportion. For example, an observed binding profile can be compared to individual binding profiles expected for a set of candidate proteins, each comparison can be given a numerical score indicating goodness of fit, the scores can be summed, and the score for the best fit comparison can be divided by the sum to derive a proportion of the score contributed by the top match. A threshold can be applied to filter out incorrect identifications. By way of more specific example, each binding profile comparison can output a score between 0 and 1 (0 being lowest possible match and 1 being the highest possible match) indicating the likelihood of the detected protein being a particular candidate protein given the observed binding profile (or the score can indicate the probability of the particular candidate protein generating the observed binding profile), a score can be calculated from a comparison of the observed binding profile to each candidate protein suspected of being in an organism's proteome (e.g. a human proteome), and the threshold can be set at 0.9. As such, a given binding profile will only result in a candidate identification if exactly one protein matches well.

The scores that are used to identify a candidate protein can be determined using a machine learning process such as deep learning, statistical learning, supervised learning, unsupervised learning, clustering, expectation maximization, maximum likelihood estimation, Bayesian inference, linear regression, logistic regression, binary classification, multinomial classification, support vector machines (SVMs), neural networks, convolutional neural networks (CNNs), deep neural networks, cascading neural networks, k-Nearest Neighbor (k-NN) classification, random forests (RFs), classification and regression trees (CARTs) or pattern recognition algorithms. For example, the software may perform the one or more algorithms to analyze inputs such as (i) a priori binding characteristic of one or more affinity agents, (ii) empirically observed binding behavior of one or more affinity agents, (iii) putative binding outcomes or putative binding profiles for one or more candidate proteins, (iv) presence or absence of particular epitopes in candidate proteins, (v) characteristics of binding outcomes used to generate one or more binding profiles, (vi) information identifying a unique identifier (e.g. array address) for an empirically observed protein, and/or (vii) empirical binding outcomes or empirical binding profiles for one or more unknown proteins. Thus, the input to an algorithm of the present disclosure may include a database of information for one or more candidate proteins and a set of empirical binding outcomes for one or more unknown proteins. The output of the algorithm may include (i) a probability that a binding outcome or binding profile is observed given a hypothesized candidate protein identity, (ii) the most probable identity, selected from the set of candidate proteins, for an unknown protein, (iii) the probability of a candidate identification being correct given an observed empirical binding outcome or empirical binding profile, and/or (iv) a group of high-probability candidate protein identities and an associated probability that an unknown protein is one of the proteins in the group. Exemplary algorithms, and methods for characterizing proteins, are set forth, for example in US Pat App. Pub. No 2020/0286584 A1, which is incorporated herein by reference.

Accordingly, a method set forth herein can include a step of identifying one or more candidate proteins in a sample based on determination of the compatibility of an empirical binding profiles with one or more reference binding profiles for one or more candidate proteins. The method can be further configured to provide a confidence level that each of one or more candidate proteins is present in the sample. Decoding protein identity may be applied independently to each unknown protein in a sample, to generate a collection of candidate proteins identified in the sample. For example, the decoding approach may be applied independently to individual addresses of an array.

A composition or method as set forth herein may comprise one or more analytes that are coupled to a solid support or a surface thereof. As used herein, the term “solid support” refers to a substrate that is insoluble in aqueous liquid. Optionally, the substrate can be rigid. The substrate can be non-porous or porous. The substrate can optionally be capable of taking up a liquid (e.g. due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In some cases, a solid support may comprise silicon, fused silica, quartz, mica, or borosilicate glass.

A solid support or a surface thereof may be configured to display an analyte or a plurality of analytes. A solid support may contain one or more patterned, formed, or prepared surfaces that contain at least one address for displaying an analyte. In some cases, a solid support may contain one or more patterned, formed, or prepared surfaces that contain a plurality of addresses, with each address configured to display one or more analytes. Accordingly, an array as set forth herein may comprise a plurality of analytes coupled to a solid support or a surface thereof. In some configurations, a solid support or a surface thereof may be patterned or formed to produce an ordered or patterned array of addresses. The deposition of analytes on the ordered or patterned array of addresses may be controlled by interactions between the solid support and the analytes such as, for example, electrostatic interactions, magnetic interactions, hydrophobic interactions, hydrophilic interactions, covalent interactions, or non-covalent interactions. Accordingly, the coupling of an analyte at each address of the array of addresses may produce an ordered or patterned array of analytes whose average spacing between analytes is determined based upon the tolerance of the ordering or patterning of the solid support and the size of an analyte-binding region for each address. An ordered or patterned array of analytes may be characterized as having a regular geometry, such as a rectangular, triangular, polygonal, or annular grid. In other configurations, a solid support or a surface thereof may be unpatterned or unordered. The deposition of analytes on the unordered or unpatterned array of addresses may be controlled by interactions between the solid support and the analytes, or inter-analyte interactions such as, for example, steric repulsion, electrostatic repulsion, electrostatic attraction, magnetic repulsion, magnetic attraction, covalent interactions, or non-covalent interactions.

A solid support or a surface thereof may include a base substrate material and, optionally, one or more additional materials that are contacted or adhered with the substrate material. A solid support may comprise one or more additional materials that are deposited, coated, or inlayed onto the substrate material. Additional materials may be added to the substrate material to alter the properties of the substrate material. For example, materials may be added to alter the surface chemistry (e.g., hydrophobicity, hydrophilicity, non-specific binding, electrostatic properties), alter the optical properties (e.g., reflective properties, refractive properties), alter the electrical or magnetic properties (e.g., dielectric materials, conducting materials, electrically-insulating materials), or alter the heat transfer characteristics of the substrate material. Additional materials contacted or adhered with a substrate material may be ordered or patterned onto the substrate material to, for example, locate the additional material at addresses or locate the additional material at interstitial regions between addresses. Exemplary additional materials may include metals (e.g., gold, silver, copper, etc.), metal oxides (e.g., titanium oxide, silicon dioxide, alumina, iron oxides, etc.), metal nitrides (e.g., silicon nitride, aluminum nitride, boron nitride, gallium nitride, etc.), metal carbides (e.g., tungsten carbide, titanium carbide, iron carbide, etc.), metal sulfides (e.g., iron sulfide, silver sulfide, etc.), and organic moieties (e.g., polyethylene glycol (PEG), dextrans, chemically-reactive functional groups, etc.).

A method of the present disclosure can include the step of coupling one or more analytes to a solid support or a surface thereof prior to the measurement of a binding profile. The coupling of one or more analytes to a solid support surface may include covalent or non-covalent coupling of the one or more analytes to the solid support. Covalent coupling of an analyte to a solid support can include direct covalent coupling of an analyte to a solid support (e.g., formation of coordination bonds) or indirect covalent coupling between a reactive functional group of the analyte and a reactive functional group that is coupled to the solid support (e.g., a CLICK-type reaction). Non-covalent coupling can include the formation of any non-covalent interaction between an analyte and a solid support, including electrostatic or magnetic interactions, or non-covalent bonding interactions (e.g., ionic bonds, van der Waals interactions, hydrogen bonding, etc.). The skilled person will readily recognize that the particular analyte and the choice of solid support can affect the selection of a coupling chemistry for the compositions and methods set forth herein.

Accordingly, a coupling chemistry may be selected based upon the criterion that it provides a sufficiently stable coupling of an analyte to a solid support for a time scale that meets or exceeds the time scale of a method as set forth herein. For example, a polypeptide identification method can require a coupling of the analyte to the solid support for a sufficient amount of time to permit a series of empirical measurements of the analyte to occur. An analyte may be continuously coupled to a solid support for an observable length of time such as, for example, at least about 1 minute, 1 hour (hr), 3 hrs, 6 hrs, 12 hrs, 1 day, 1.5 days, 2 days, 3 days, 1 week (wk), 2 wks, 3 wks, 1 month, or more. The coupling of an analyte to a solid support can occur with a solution-phase chemistry that promotes the deposition of the analyte on the solid support. Coupling of an analyte to a solid support may occur under solution conditions that are optimized for any conceivable solution property, including solution composition, species concentrations, pH, ionic strength, solution temperature, etc. Solution composition can be varied by chemical species, such as buffer type, salts, acids, bases, and surfactants. In some configurations, species such as salts and surfactants may be selected to facilitate the formation of interactions between an analyte and a solid support. Covalent coupling methods for coupling an analyte to a solid support may include species such as catalyst, initiators, and promoters to facilitate particular reactive chemistries.

Coupling of an analyte to a solid support may be facilitated by a mediating group. A mediating group may modify the properties of the analyte to facilitate the coupling. Useful mediating groups have been set forth herein (e.g., SNAPs). In some configurations, a mediating group can be coupled to an analyte prior to coupling the analyte to a solid support. Accordingly, the mediating group may be chosen to increase the strength, control, or specificity of the coupling of the analyte to the solid support. In other configurations, a mediating group can be coupled to a solid support prior to coupling an analyte to the solid support. Accordingly, the mediating group may be chosen to provide a more favorable coupling chemistry than can be provided by the solid support alone.

The present disclosure provides compositions, apparatus and methods for detecting one or more proteins. A protein can be detected using one or more affinity agents having binding affinity for the protein. The affinity agent and the protein can bind each other to form a complex and, during or after formation, the complex can be detected. The complex can be detected directly, for example, due to a label that is present on the affinity agent or protein. In some configurations, the complex need not be directly detected, for example, in formats where the complex is formed and then the affinity agent, protein, or a label component that was present in the complex is detected.

Many protein detection methods, such as enzyme linked immunosorbent assay (ELISA), achieve high-confidence characterization of one or more protein in a sample by exploiting high specificity binding of antibodies, aptamers or other binding agents to the protein(s) and detecting the binding event while ignoring all other proteins in the sample. ELISA is generally carried out at low plex scale (e.g. from one to a hundred different proteins detected in parallel or in succession) but can be used at higher plexity. ELISA methods can be carried out by detecting immobilized binding agents and/or proteins in multiwell plates, on arrays, or on particles in microfluidic devices. Exemplary plate-based methods include, for example, the MULTI-ARRAY technology commercialized by MesoScale Diagnostics (Rockville, Maryland) or Simple Plex technology commercialized by Protein Simple (San Jose, CA). Exemplary, array-based methods include, but are not limited to those utilizing Simoa® Planar Array Technology or Simoa® Bead Technology, commercialized by Quanterix (Billerica, MA). Further exemplary array-based methods are set forth in U.S. Pat. Nos. 9,678,068; 9,395,359; 8,415,171; 8,236,574; or 8,222,047, each of which is incorporated herein by reference. Exemplary microfluidic detection methods include those commercialized by Luminex (Austin, Texas) under the trade name xMAP® technology or used on platforms identified as MAGPIX®, LUMINEX® 100/200 or FEXMAP 3D®.

Other detection methods that can also be used, for example at low plex scale, include procedures that employ SOMAmer reagents and SOMAscan assays commercialized by Soma Logic (Boulder, CO). In one configuration, a sample is contacted with aptamers that are capable of binding proteins with high specificity for the amino acid sequence of the proteins. The resulting aptamer-protein complexes can be separated from other sample components, for example, by attaching the complexes to beads (or other solid support) that are removed from other sample components. The aptamers can then be isolated and, because the aptamers are nucleic acids, the aptamers can be detected using any of a variety of methods known in the art for detecting nucleic acids, including for example, hybridization to nucleic acid arrays, PCR-based detection, or nucleic acid sequencing. Exemplary methods and compositions are set forth in U.S. Pat. Nos. 7,855,054; 7,964,356; 8,404,830; 8,945,830; 8,975,026; 8,975,388; 9,163,056; 9,938,314; 9,404,919; 9,926,566; 10,221,421; 10,239,908; 10,316,321 10,221,207 or 10,392,621, each of which is incorporated herein by reference.

In some detection assays, a protein can be cyclically modified and the modified products from individual cycles can be detected. In some configurations, a protein can be sequenced by a sequential process in which each cycle includes steps of labeling and removing the amino terminal amino acid of a protein and detecting the label. Accordingly, a method of detecting a protein can include steps of (i) exposing a terminal amino acid on the protein; (ii) detecting a change in signal from the protein; and (iii) identifying the type of amino acid that was removed based on the change detected in step (ii). The terminal amino acid can be exposed, for example, by removal of one or more amino acids from the amino terminus or carboxyl terminus of the protein. Steps (i) through (iii) can be repeated to produce a series of signal changes that is indicative of the sequence for the protein.

In a first configuration of the above method, one or more types of amino acids in the protein can be attached to a label that uniquely identifies the type of amino acid. In this configuration, the change in signal that identifies the amino acid can be loss of signal from the respective label. Exemplary compositions and techniques that can be used to remove amino acids from a protein and detect signal changes are those set forth in Swaminathan et al., Nature Biotech. 36:1076-1082 (2018); or U.S. Pat. No. 9,625,469 or 10,545,153, each of which is incorporated herein by reference. Methods and apparatus under development by Erisyon, Inc. (Austin, TX) may also be useful for detecting proteins.

In a second configuration of the above method, the terminal amino acid of the protein can be recognized by an affinity agent that is specific for the terminal amino acid or specific for a label moiety that is present on the terminal amino acid. The affinity agent can be detected on the array, for example, due to a label on the affinity agent. Optionally, the label is a nucleic acid barcode sequence that is added to a primer nucleic acid upon formation of a complex. The formation of the complex and identity of the terminal amino acid can be determined by decoding the barcode sequence. Exemplary affinity agents and detection methods are set forth in US Pat. App. Pub. No. 2019/0145982 A1; 2020/0348308 A1; or 2020/0348307 A1, each of which is incorporated herein by reference. Methods and apparatus under development by Encodia, Inc. (San Diego, CA) may also be useful for detecting proteins.

Cyclical removal of terminal amino acids from a protein can be carried out using an Edman-type sequencing reaction in which a phenyl isothiocyanate reacts with a N-terminal amino group under mildly alkaline conditions (e.g. about pH 8) to form a cyclical phenylthiocarbamoyl Edman complex derivative. The phenyl isothiocyanate may be substituted or unsubstituted with one or more functional groups, linker groups, or linker groups containing functional groups. An Edman-type sequencing reaction can include variations to reagents and conditions that yield a detectable removal of amino acids from a protein terminus, thereby facilitating determination of the amino acid sequence for a protein or portion thereof. For example, the phenyl group can be replaced with at least one aromatic, heteroaromatic or aliphatic group which may participate in an Edman-type sequencing reaction, non-limiting examples including: pyridine, pyrimidine, pyrazine, pyridazoline, fused aromatic groups such as naphthalene and quinoline), methyl or other alkyl groups or alkyl group derivatives (e.g., alkenyl, alkynyl, cyclo-alkyl). Under certain conditions, for example, acidic conditions of about pH 2, derivatized terminal amino acids may be cleaved, for example, as a thiazolinone derivative. The thiazolinone amino acid derivative under acidic conditions may form a more stable phenylthiohydantoin (PTH) or similar amino acid derivative which can be detected. This procedure can be repeated iteratively for residual protein to identify the subsequent N-terminal amino acid. Many variations of Edman-type degradation have been described and may be used including, for example, a one-step removal of an N-terminal amino acid using alkaline conditions (Chang, J. Y., FEBS LETTS., 1978, 91(1), 63-68). In some cases, Edman-type reactions may be thwarted by N-terminal modifications which may be selectively removed, for example, N-terminal acetylation or formylation (e.g., see Gheorghe M. T., Bergman T. (1995) in Methods in Protein Structure Analysis, Chapter 8: Deacetylation and internal cleavage of Proteins for N-terminal Sequence Analysis. Springer, Boston, MA. https://doi.org/10.1007/978-1-4899-1031-8_8).

Non-limiting examples of functional groups for substituted phenyl isothiocyanate may include ligands (e.g. biotin and biotin analogs) for known receptors, labels such as luminophores, or reactive groups such as click functionalities (e.g. compositions having an azide or acetylene moiety). The functional group may be a DNA, RNA, peptide or small molecule barcode or other tag which may be further processed and/or detected.

The removal of an amino terminal amino acid using Edman-type processes utilizes at least two main steps, the first step includes reacting an isothiocyanate or equivalent with protein N-terminal residues to form a relatively stable Edman complex, for example, a phenylthiocarbamoyl complex. The second step includes removing the derivatized N-terminal amino acid, for example, via heating. The protein, now having been shortened by one amino acid, may be detected, for example, by contacting the protein with a labeled affinity agent that is complementary to the amino terminus and examining the protein for binding to the agent, or by detecting loss of a label that was attached to the removed amino acid.

Edman-type processes can be carried out in a multiplex format to detect, characterize or identify a plurality of proteins. A method of detecting a protein can include steps of (i) exposing a terminal amino acid on a protein at an address of an array; (ii) binding an affinity agent to the terminal amino acid, where the affinity agent comprises a nucleic acid tag, and where a primer nucleic acid is present at the address; (iii) extending the primer nucleic acid, thereby producing an extended primer having a copy of the tag; and (iv) detecting the tag of the extended primer. The terminal amino acid can be exposed, for example, by removal of one or more amino acids from the amino terminus or carboxyl terminus of the protein. Steps (i) through (iv) can be repeated to produce a series of tags that is indicative of the sequence for the protein. The method can be applied to a plurality of proteins on the array and in parallel. Whatever the plexity, the extending of the primer can be carried out, for example, by polymerase-based extension of the primer, using the nucleic acid tag as a template. Alternatively, the extending of the primer can be carried out, for example, by ligase- or chemical-based ligation of the primer to a nucleic acid that is hybridized to the nucleic acid tag. The nucleic acid tag can be detected via hybridization to nucleic acid probes (e.g. in an array), amplification-based detections (e.g. PCR-based detection, or rolling circle amplification-based detection) or nucleic acid sequencing (e.g. cyclical reversible terminator methods, nanopore methods, or single molecule, real time detection methods). Exemplary methods that can be used for detecting proteins using nucleic acid tags are set forth in US Pat. App. Pub. No. 2019/0145982 A1; 2020/0348308 A1; or 2020/0348307 A1, each of which is incorporated herein by reference.

A protein can optionally be detected based on its enzymatic or biological activity. For example, a protein can be contacted with a reactant that is converted to a detectable product by an enzymatic activity of the protein. In other assay formats, a first protein having a known enzymatic function can be contacted with a second protein to determine if the second protein changes the enzymatic function of the first protein. As such, the first protein serves as a reporter system for detection of the second protein. Exemplary changes that can be observed include, but are not limited to, activation of the enzymatic function, inhibition of the enzymatic function, attenuation of the enzymatic function, degradation of the first protein or competition for a reactant or cofactor used by the first protein. Proteins can also be detected based on their binding interactions with other molecules such as proteins, nucleic acids, nucleotides, metabolites, hormones, vitamins, small molecules that participate in biological signal transduction pathways, biological receptors or the like. For example, a protein that participates in a signal transduction pathway can be identified as a particular candidate protein by detecting binding to a second protein that is known to be a binding partner for the candidate protein in the pathway.

The presence or absence of post-translational modifications (PTM) can be detected using a composition, apparatus or method set forth herein. A PTM can be detected using an affinity agent that recognizes the PTM or based on a chemical property of the PTM. Exemplary PTMs that can be detected, identified or characterized include, but are not limited to, myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglyclyation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, proteolytic cleavage, isoaspartate formation, racemization, and protein splicing.

PTMs may occur at particular amino acid residues of a protein. For example, the phosphate moiety of a particular proteoform can be present on a serine, threonine, tyrosine, histidine, cysteine, lysine, aspartate or glutamate residue of the protein. In other examples, an acetyl moiety can be present on the N-terminus or on a lysine; a serine or threonine residue can have an O-linked glycosyl moiety; an asparagine residue can have an N-linked glycosyl moiety; a proline, lysine, asparagine, aspartate or histidine amino acid can be hydroxylated; an arginine or lysine residue can be methylated; or the N-terminal methionine or at a lysine amino acid can be ubiquitinated.

In some configurations of the apparatus and methods set forth herein, one or more proteins can be detected on a solid support. For example, protein(s) can be attached to a support, the support can be contacted with detection agents (e.g. affinity agents) in solution, the agents can interact with the protein(s), thereby producing a detectable signal, and then the signal can be detected to determine the presence of the protein(s). In multiplexed versions of this approach, different proteins can be attached to different addresses in an array, and the probing and detection steps can occur in parallel. In another example, affinity agents can be attached to a solid support, the support can be contacted with proteins in solution, the proteins can interact with the affinity agents, thereby producing a detectable signal, and then the signal can be detected to determine the presence, quantity or characteristics of the proteins. This approach can also be multiplexed by attaching different affinity agents to different addresses of an array.

Proteins, affinity agents or other objects of interest can be attached to a solid support via covalent or non-covalent bonds. For example, a linker can be used to covalently attach a protein or other object of interest to an array. A particularly useful linker is a structured nucleic acid particle such as a nucleic acid nanoball (e.g. a concatemeric amplicon produced by rolling circle replication of a circular nucleic acid template) or a nucleic acid origami. For example, a plurality of proteins can be conjugated to a plurality of structured nucleic acid particles, such that each protein-conjugated particle forms an address in the array. Exemplary linkers for attaching proteins, or other objects of interest, to an array or other solid support are set forth in WO 2019/195633 A1 or U.S. Pat. App. Ser. No. 63/159,500, each of which is incorporated herein by reference.

A protein can be detected based on proximity of two or more affinity agents. For example, the two affinity agents can include two components each: a receptor component and a nucleic acid component. When the affinity agents bind in proximity to each other, for example, due to ligands for the respective receptors being on a single protein, or due to the ligands being present on two proteins that associate with each other, the nucleic acids can interact to cause a modification that is indicative of the two ligands being in proximity. Optionally, the modification can be extension of one of the nucleic acids using the other nucleic acid as a template. As another option, one of the nucleic acids can form a template that acts as splint to position other nucleic acids for ligation to an oligonucleotide. Exemplary methods are commercialized by Olink Proteomics AB (Uppsala Sweden) or set forth in U.S. Pat. Nos. 7,306,904; 7,351,528; 8,013,134; 8,268,554 or 9,777,315, each of which is incorporated herein by reference.

A method or apparatus of the present disclosure can optionally be configured for optical detection (e.g. luminescence detection). Analytes or other entities can be detected, and optionally distinguished from each other, based on measurable characteristics such as the wavelength of radiation that excites a luminophore, the wavelength of radiation emitted by a luminophore, the intensity of radiation emitted by a luminophore (e.g. at particular detection wavelength(s)), luminescence lifetime (e.g. the time that a luminophore remains in an excited state) or luminescence polarity. Other optical characteristics that can be detected, and optionally used to distinguish analytes, include, for example, absorbance of radiation, resonance Raman, radiation scattering, or the like. A luminophore can be an intrinsic moiety of a protein or other analyte to be detected, or the luminophore can be an exogenous moiety that has been synthetically added to a protein or other analyte.

A method or apparatus of the present disclosure can use a light sensing device that is appropriate for detecting a characteristic set forth herein or known in the art. Particularly useful components of a light sensing device can include, but are not limited to, optical sub-systems or components used in nucleic acid sequencing systems. Examples of useful sub systems and components thereof are set forth in US Pat. App. Pub. No. 2010/0111768 A1 or U.S. Pat. Nos. 7,329,860; 8,951,781 or 9,193,996, each of which is incorporated herein by reference. Other useful light sensing devices and components thereof are described in U.S. Pat. Nos. 5,888,737; 6,175,002; 5,695,934; 6,140,489; or 5,863,722; or US Pat. Pub. Nos. 2007/007991 A1, 2009/0247414 A1, or 2010/0111768; or WO2007/123744, each of which is incorporated herein by reference. Light sensing devices and components that can be used to detect luminophores based on luminescence lifetime are described, for example, in U.S. Pat. Nos. 9,678,012; 9,921,157; 10,605,730; 10,712,274; 10,775,305; or 10,895,534 each of which is incorporated herein by reference.

Luminescence lifetime can be detected using an integrated circuit having a photodetection region configured to receive incident photons and produce a plurality of charge carriers in response to the incident photons. The integrated circuit can include at least one charge carrier storage region and a charge carrier segregation structure configured to selectively direct charge carriers of the plurality of charge carriers directly into the charge carrier storage region based upon times at which the charge carriers are produced. See, for example, U.S. Pat. Nos. 9,606,058, 10,775,305, and 10,845,308, each of which is incorporated herein by reference. Optical sources that produce short optical pulses can be used for luminescence lifetime measurements. For example, a light source, such as a semiconductor laser or LED, can be driven with a bipolar waveform to generate optical pulses with FWHM durations as short as approximately 85 ps having suppressed tail emission. See, for example, in U.S. Pat. No. 10,605,730, which is incorporated herein by reference.

For configurations that use optical detection (e.g. luminescent detection), one or more analytes (e.g. proteins) may be immobilized on a surface, and this surface may be scanned with a microscope to detect any signal from the immobilized analytes. The microscope itself may comprise a digital camera or other luminescence detector configured to record, store, and analyze the data collected during the scan. A luminescence detector of the present disclosure can be configured for epiluminescent detection, total internal reflection (TIR) detection, waveguide assisted excitation, or the like.

A light sensing device may be based upon any suitable technology, and may be, for example, a charged coupled device (CCD) sensor that generates pixilated image data based upon photons impacting locations in the device. It will be understood that any of a variety of other light sensing devices may also be used including, but not limited to, a detector array configured for time delay integration (TDI) operation, a complementary metal oxide semiconductor (CMOS) detector, an avalanche photodiode (APD) detector, a Geiger-mode photon counter, a photomultiplier tube (PMT), charge injection device (CID) sensors, JOT image sensor (Quanta), or any other suitable detector. Light sensing devices can optionally be coupled with one or more excitation sources, for example, lasers, light emitting diodes (LEDs), arc lamps or other energy sources known in the art.

An optical detection system can be configured for single molecule detection. For example, waveguides or optical confinements can be used to deliver excitation radiation to locations of a solid support where analytes are located. Zero-mode waveguides can be particularly useful, examples of which are set forth in U.S. Pat. Nos. 7,181,122, 7,302,146, or 7,313,308, each of which is incorporated herein by reference. Analytes can be confined to surface features, for example, to facilitate single molecule resolution. For example, analytes can be distributed into wells having nanometer dimensions such as those set forth in U.S. Pat. No. 7,122,482 or 8,765,359, or US Pat. App. Pub. No 2013/0116153 A1, each of which is incorporated herein by reference. The wells can be configured for selective excitation, for example, as set forth in U.S. Pat. No. 8,798,414 or 9,347,829, each of which is incorporated herein by reference. Analytes can be distributed to nanometer-scale posts, such as high aspect ratio posts which can optionally be dielectric pillars that extend through a metallic layer to improve detection of an analyte attached to the pillar. See, for example, U.S. Pat. Nos. 8,148,264, 9,410,887 or 9,987,609, each of which is incorporated herein by reference. Further examples of nanostructures that can be used to detect analytes are those that change state in response to the concentration of analytes such that the analytes can be quantitated as set forth in WO 2020/176793 A1, which is incorporated herein by reference.

An apparatus or method set forth herein need not be configured for optical detection. For example, an electronic detector can be used for detection of protons or charged labels (see, for example, US Pat. App. Pub. Nos. 2009/0026082 A1; 2009/0127589 A1; 2010/0137143 A1; or 2010/0282617 A1, each of which is incorporated herein by reference in its entirety). A field effect transistor (FET) can be used to detect analytes or other entities, for example, based on the proximity of a field disrupting moiety to the FET. The field disrupting moiety can be due to an extrinsic label attached to an analyte or affinity agent, or the moiety can be intrinsic to the analyte or affinity agent being used. Surface plasmon resonance can be used to detect binding of analytes or affinity agents at or near a surface. Exemplary sensors and methods for attaching molecules to sensors are set forth in US Pat. App. Pub. Nos. 2017/0240962 A1; 2018/0051316 A1; 2018/0112265 A1; 2018/0155773 A1 or 2018/0305727 A1; or U.S. Pat. Nos. 9,164,053; 9,829,456; 10,036,064, each of which is incorporated herein by reference.

A composition, apparatus or method of the present disclosure can be used to characterize or identify at least about 0.0000001%, 0.000001%, 0.00001%, 0.0001%, 0.001%, 0.01%, 0.1%, 1%, 10%, 25%, 50%, 90%, 99%, 99.9%, 99.99%, 99.999%, 99.9999%, 99.99999%, 99.999999%, or more of all protein species in a proteome. Alternatively or additionally, a proteomic characterization method may characterize or no more than about 99.999999%, 99.99999%, 99.9999%, 99.999%, 99.99%, 99.9%, 99%, 90%, 50%, 25%, 10%, 1%, 0.1%, 0.01%, 0.001%, 0.0001%, 0.00001%, 0.000001%, 0.0000001%, or less of all protein species in a proteome.

A solid support or a surface thereof may comprise an average, minimum or maximum site pitch of at least about 10 nanometers (nm), 50 nm, 100 nm, 200 nm, 300 nm, 400 nm, 500 nm, 600 nm, 700 nm, 800 nm, 900 nm, 1 micron (μm), 1.1 μm, 1.2 μm, 1.3 μm, 1.4 μm, 1.5 μm, 1.6 μm, 1.7 μm, 1.8 μm, 1.9 μm, 2 μm, 2.1 μm, 2.2 μm, 2.3 μm, 2.4 μm, 2.5 μm, 2.6 μm, 2.7 μm, 2.8 μm, 2.9 μm, 3 μm, 3.1 μm, 3.2 μm, 3.3 μm, 3.4 μm, 3.5 μm, 3.6 μm, 3.7 μm, 3.8 μm, 3.9 μm, 4 μm, 4.5 μm, 5 μm, 10 μm, 20 μm, 30 μm, 40 μm, 50 μm, or more than 50 μm. Alternatively or additionally, a solid support or a surface thereof may comprise an average, minimum or maximum site pitch of no more than about 50 μm, 40 μm, 30 μm, 20 μm, 10 μm, 5 μm, 4.5 μm, 4.0 μm, 3.9 μm, 3.8 μm, 3.7 μm, 3.6 μm, 3.5 μm, 3.4 μm, 3.3 μm, 3.2 μm, 3.1 μm, 3.0 μm, 2.9 μm, 2.8 μm, 2.7 μm, 2.6 μm, 2.5 μm, 2.4 μm, 2.3 μm, 2.2 μm, 2.1 μm, 2 μm, 1.9 μm, 1.8 μm, 1.7 μm, 1.6 μm, 1.5 μm, 1.4 μm, 1.3 μm, 1.2 μm, 1.1 μm, 1 μm, 900 nm, 800 nm, 700 nm, 600 nm, 500 nm, 400 nm, 300 nm, 200 nm, 100 nm, 50 nm, 10 nm, or less than 10 nm. An average pitch may be chosen to achieve optical resolution of each site of a plurality of sites at single-analyte resolution. Accordingly, an average pitch may be determined based upon a spatial resolution of a method used to form a solid support (e.g., photolithography), a desired array density, and/or a necessary spatial separation between neighboring sites to obtain single-analyte resolution of moieties bound to each site.

A solid support or a surface thereof may comprise an average, minimum or maximum site size (e.g., width, length, diameter, etc.) of at least about 10 nanometers (nm), 50 nm, 100 nm, 200 nm, 300 nm, 400 nm, 500 nm, 600 nm, 700 nm, 800 nm, 900 nm, 1 micron (μm), 1.1 μm, 1.2 μm, 1.3 μm, 1.4 μm, 1.5 μm, 1.6 μm, 1.7 μm, 1.8 μm, 1.9 μm, 2 μm, 2.1 μm, 2.2 μm, 2.3 μm, 2.4 μm, 2.5 μm, 2.6 μm, 2.7 μm, 2.8 μm, 2.9 μm, 3 μm, 3.1 μm, 3.2 μm, 3.3 μm, 3.4 μm, 3.5 μm, 3.6 μm, 3.7 μm, 3.8 μm, 3.9 μm, 4 μm, 4.5 μm, 5 μm, 10 μm, 20 μm, 30 μm, 40 μm, 50 μm, or more than 50 μm. Alternatively or additionally, a solid support or a surface thereof may comprise an average, minimum or maximum site size of no more than 50 μm, 40 μm, 30 μm, 20 μm, 10 μm, 5 μm, 4.5 μm, 4.0 μm, 3.9 μm, 3.8 μm, 3.7 μm, 3.6 μm, 3.5 μm, 3.4 μm, 3.3 μm, 3.2 μm, 3.1 μm, 3.0 μm, 2.9 μm, 2.8 μm, 2.7 μm, 2.6 μm, 2.5 μm, 2.4 μm, 2.3 μm, 2.2 μm, 2.1 μm, 2 μm, 1.9 μm, 1.8 μm, 1.7 μm, 1.6 μm, 1.5 μm, 1.4 μm, 1.3 μm, 1.2 μm, 1.1 μm, 1 μm, 900 nm, 800 nm, 700 nm, 600 nm, 500 nm, 400 nm, 300 nm, 200 nm, 100 nm, 50 nm, 10 nm, or less than 10 nm. A site size may be determined based upon a spatial resolution of a method used to form a solid support (e.g., photolithography) and/or a size of an analyte or nucleic acid that is to be deposited on a site. An interstitial region separating a first site from a second site may have an average, minimum, or maximum dimension (e.g., length, width, diameter, etc.) of at least 1.5×, 2×, 3×, 4×, 5×, 10×, 20×, 50×, 100×, or more than 100× the average, minimum, or maximum site size.

In some configurations of the compositions, apparatus and methods set forth herein, one or more proteins can be present on a solid support, where the proteins can optionally be detected. For example, a protein can be attached to a solid support, the solid support can be contacted with a detection agent (e.g. affinity agent) in solution, the affinity agent can interact with the protein, thereby producing a detectable signal, and then the signal can be detected to determine the presence, absence, quantity, a characteristic or identity of the protein. In multiplexed versions of this approach, different proteins can be attached to different addresses in an array, and the detection steps can occur in parallel, such that proteins at each address are detected, quantified, characterized or identified. In another example, detection agents can be attached to a solid support, the support can be contacted with proteins in solution, the proteins can interact with the detection agents, thereby producing a detectable signal, and then the signal can be detected to determine the presence of the proteins. This approach can also be multiplexed by attaching different probes to different addresses of an array.

In multiplexed configurations, different proteins can be attached to different unique identifiers (e.g. addresses in an array), and the proteins can be manipulated and detected in parallel. For example, a fluid containing one or more different affinity agents can be delivered to an array such that the proteins of the array are in simultaneous contact with the affinity agent(s). Moreover, a plurality of addresses can be observed in parallel allowing for rapid detection of binding events. A plurality of different proteins can have a complexity of at least 5, 10, 100, 1×10³, 1×10⁴, 1×10⁵ or more different native-length protein primary sequences. Alternatively or additionally, a proteome, proteome subfraction or other protein sample that is analyzed in a method set forth herein can have a complexity that is at most 1×10⁵, 1×10⁴, 1×10³, 100, 10, 5 or fewer different native-length protein primary sequences. The total number of proteins of a sample that is detected, characterized or identified can differ from the number of different primary sequences in the sample, for example, due to the presence of multiple copies of at least some protein species. Moreover, the total number of proteins of a sample that is detected, characterized or identified can differ from the number of candidate proteins suspected of being in the sample, for example, due to the presence of multiple copies of at least some protein species, absence of some proteins in a source for the sample, or loss of some proteins prior to analysis.

A protein can be attached to a unique identifier using any of a variety of means. The attachment can be covalent or non-covalent. Exemplary covalent attachments include chemical linkers such as those achieved using click chemistry or other linkages known in the art or described in U.S. patent application Ser. No. 17/062,405, which is incorporated herein by reference. Non-covalent attachment can be mediated by receptor-ligand interactions (e.g. (strept)avidin-biotin, antibody-antigen, or complementary nucleic acid strands), for example, wherein the receptor is attached to the unique identifier and the ligand is attached to the protein or vice versa. In particular configurations, a protein is attached to a solid support (e.g. an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers such as tags or addresses in an array are set forth in U.S. patent application Ser. No. 17/062,405 and 63/159,500, each of which is incorporated herein by reference.

### EXAMPLES

### Example 1: Detectable Probes

A blood sample is collected from a human medical subject. A serum fraction containing serum proteins is purified from the blood sample via centrifugation of a whole blood sample obtained from the medical subject and collection of the resulting supernatant. The supernatant includes collected serum proteins as well as several impurities, including trace amounts of clotting factors, vitamins, cofactors, nucleic acids, triglycerides, cholesterols, electrolytes (e.g., Ca²⁺, Mg²⁺, Na⁺, K⁺, Fe²⁺, etc.), trace amounts of non-human antigens (e.g, viral antigens), and trace amounts of the medical subject's prescribed pharmaceuticals. The fraction containing the extracted serum proteins is modified to incorporate a transcyclooctene (TCO) moiety at some of the amine-containing amino acid sidechain of the present proteins. Each modified serum protein is conjugated to a structured nucleic acid particle (SNAP) containing a methyltetrazine (mTz) attachment moiety via a TCO-mTz Click-type reaction to form a plurality of serum protein conjugates. Certain impurities are also conjugated to SNAPs due to cross-reactivity with functionalization reagents and/or methyltetrazine. Less than 1% of conjugated SNAPs comprise non-polypeptide impurities. The mixture of SNAPs containing the plurality of serum protein conjugates is contacted with a patterned glass solid support with an amine-functionalized (positively-charged) surface, thereby depositing a SNAP containing a single serum protein conjugate or a single impurity conjugate at each patterned address on the solid support due to an electrostatic interaction between the functionalized surface of the address and the negatively-charged surface of the SNAP component.

The patterned array of serum protein conjugates is characterized by cyclical binding of affinity reagents. Each cycle comprises the steps of a) contacting the solid support with a plurality of fluorescently-labeled affinity agents in a fluidic medium, b) detecting presence or absence of fluorescent signal at each address on the solid support by confocal fluorescent microscopy, and c) removing any bound affinity agents from the array with a washing buffer. Each cycle utilizes a plurality of affinity agents that differs from each other plurality of affinity agents with respect to a binding property that is characterized with respect to a set of epitopes. After performing 200 unique cycles, measurement outcomes from the 200 cycles are provided to a computer decoding algorithm that is configured to identify a polypeptide that is present at each address on the patterned array. Identities of serum proteins are determined for −95% of addresses on the array to beyond a confidence level of 99.9%. Each address comprising an identified serum albumin is excluded from additional analysis.

Four pluralities of detectable probes are prepared. A detectable probe of the first plurality of detectable probes comprises four wild-type human serum albumin proteins conjugated to a fluorescently-labeled SNAP. A detectable probe of the second plurality of detectable probes comprises four glycosylated human serum albumin proteins conjugated to a fluorescently-labeled SNAP. A detectable probe of the third plurality of detectable probes comprises four wild-type human transcortin proteins conjugated to a fluorescently-labeled SNAP. A detectable probe of the fourth plurality of detectable probes comprises four wild-type vascular endothelial growth factor (VEGF) proteins conjugated to a fluorescently-labeled SNAP. Each protein is conjugated to a SNAP with four attachment sites by a TCO-mTz conjugation reaction.

Each plurality is separately contacted with the array to detect binding of the detectable probes. Each binding cycle consists of the steps of a) contacting the solid support with a plurality of detectable probes, b) detecting the presence or absence of fluorescent signal at each address on the solid support by confocal fluorescent microscopy, and c) removing any bound detectable probes from the array with a washing buffer. Each cycle uses one of the four pluralities of detectable probes until binding of all four pluralities has been tested. Measurement outcomes from the 4 cycles are provided to a computer decoding algorithm that is configured to identify the presence or absence of binding of each detectable probe at each address on the array, excluding addresses comprising identified serum albumins. The detectable probe binding data is combined with the protein identification data to identify which polypeptides formed polypeptide binding interactions with each detectable probe.

The two albumin-based detectable probes are observed to repeatably bind numerous polypeptide species with a high binding affinity (i.e., species where albumin binding is observed multiple times). Substantial differences do occur between the wild-type and glycosylated version, likely due to differences in binding behavior caused by the presence or absence of the glycosylated moieties. The transcortin and VEGF detectable probes are observed to bind fewer polypeptide species and with a low degree of binding affinity (i.e., detectable probe binding appears to occur randomly due to non-specific binding interactions).

### Example 2: Albumin Array

Two arrays of human serum albumins are prepared per the method described in Example 1, with purified human serum albumin substituted in place of the serum proteins. The first array contains wild-type human serum albumins. The second array contains a plurality containing millions of engineered mutants that are produced via transgenic E. coli strains. The provided samples of purified human serum albumins are characterized as 99% pure before array formation.

The albumin arrays are contacted with three pluralities of fluorescently-labeled probes comprising differing binding ligands of serum albumin (insulin, cholesterol, and retinol). Binding of each detectable probe is detected on the arrays via the method described in Example 1. Measurement outcomes are provided to a computer algorithm to determine which array addresses contain probe-binding albumins to a 99% confidence. A subset of albumin-containing addresses is determined and all other array addresses are excluded from further analysis.

Each array is subsequently contacted with fluidic media comprising pluralities of proteins extracted from king cobra venom. The venom proteins are incubated on each array, allowing polypeptide binding interactions to form between free venom proteins and coupled serum albumins. After forming polypeptide complexes on each array, the arrays are again contacted with the 3 detectable probes with binding specificity for human serum albumin. The measurement outcomes of each detectable probes on both arrays is provided to a computer algorithm. The algorithm identifies which addresses of the non-excluded addresses appear to have a bound venom protein based upon the absence of binding of the insulin probe but binding of at least one other non-polypeptide probe.

After identifying addresses with venom binding properties on the mutated arrays, the mutated array is subjected to a polypeptide identification assay, as described in Example 1. Affinity agent-based epitope binding data is used to identify a probable mutation present in each venom-binding variant of human serum albumin. The candidates are analyzed for common structural mutations that appear to enhance venom binding. Based upon the results, a series of candidate albumin-based anti-venom pharmaceuticals are identified for further testing.

### Example 3: Polypeptide Complex

A sample of human blood serum is combined with a photocleavable cross-linking agent. After cross-linking, serum proteins are purified. Only proteins larger than 66 kiloDaltons (kDa) are retained since only serum albumins and complexes of serum albumin are of interest to the analysis. Serum proteins are deposited on an array per the method of Example 1.

Serum proteins are analyzed by binding with two detectable probes. The first detectable probe specifically binds serum albumin at a non-binding site epitope. The second detectable probe specifically binds serum albumin at a polypeptide binding site. Measurement outcomes are analyzed to identify addresses where albumin is present (presence of binding of the first detectable probe) and the polypeptide binding site is occupied (absence of binding of the second detectable probe). Any addresses without this set of outcomes are excluded from further analysis.

Addresses containing identified polypeptide complexes are irradiated by a laser at the wavelength for cleaving the photocleavable linker. After cleaving each polypeptide complex, the array is contacted with a denaturing agent. The denaturing agent is rinsed from the array and dissociated proteins are isolated from the denaturing agent. The dissociated proteins are deposited on a second array per the method of Example 1.

Dissociated proteins are identified by the affinity agent method of Example 1. Identities of each observed polypeptide are provided as likely binding ligands of human serum albumin. Low copy number proteins are observed to be binding ligands of human serum albumin.

### Example 4: Competitive Binding

The method of Example 2 is followed, with the king cobra venom replaced with 4 different protein-containing mixtures, as shown in Table III:

| | Whole Serum | King Cobra | | |
|---|---|---|---|---|
| Mixture | Proteins | Venom | Acetaminophen | Ibuprofen |
| 1 | Yes | No | No | No |
| 2 | Yes | Yes | No | No |
| 3 | Yes | Yes | Yes | No |
| 4 | Yes | Yes | No | Yes |

The mixtures of whole serum proteins and venom allow free binding competition between all protein species for binding to serum albumins. Additionally, any inhibition effects of common analgesics (acetaminophen and ibuprofen) for pain management are tested.

After identifying and quantifying addresses with polypeptide complexes on each array, binding ligands are released from each complex by contacting each array with a denaturing agent. Dissociated binding ligands are isolated from withdrawn denaturing media and deposited on additional arrays. Dissociated binding ligands are identified per the method of Example 3. Binding data and binding ligand identities permit analysis of the effectiveness of various candidate anti-venoms under simulated real blood conditions.

### Example 5: Drug Efficacy and Dynamic Response

A medical patient receives a diagnosis of ulcerative colitis. A clinical provider for the medical patient recommends a method of treatment that includes an immune-modulating pharmaceutical compound. Candidate pharmaceuticals include small molecule immunomodulators (e.g., 6-mercaptopurine, azathioprine, methotrexate) and biologic immunomodulators (e.g., infliximab, adalimumab, golimumab, vedolizumab, ustekinumab). The medical provider recommends initially attempting a course of methotrexate, then switching to infliximab if the methotrexate is ineffective. The medical provider further orders a time series of blood protein measurements to monitor relevant cytokine levels as the pharmaceutical treatment proceeds.

An initial blood sample is collected from the medical patient before treatment commences, and subsequent blood samples are collected at days 3, 7, and 14 of methotrexate treatment. Each blood sample is centrifuged at 2500 revolutions per minute (rpm) for 15 minutes and the serum fraction is collected. Each serum fraction is combined with a heterobifunctional functionalization agent (NHS-PEG-azide) to functionalize the protein, then functionalized proteins are combined with a DNA origami tile containing a single dibenzocylooctylene (DBCO) protein binding site to form protein conjugates via a Click-type reaction between the azide functionality of the protein and the DBCO functionality of the DNA origami tile. Each fraction of protein conjugates is deposited on a single-analyte array within a fluidically-isolated lane of a microfluidic chip immediately after preparation such that lanes 1, 2, 3, and 4 correspond to serum protein fractions for days 0, 3, 7, and 14, respectively. Each single-analyte array is configured to accommodate 1 billion individual protein conjugate attachment sites.

After each protein conjugate fraction has been deposited on the chip, each array within a fluidic lane is contacted with a plurality of multivalent detectable probes that have a binding specificity for albumin. Each detectable probe contains 40 pendant fatty acids and 40 fluorophores that are each conjugated to a DNA origami tile. After contacting the detectable probes with an array for 1 minute, unbound probes are rinsed from the fluidic lane. After rinsing, the array is imaged via fluorescence microscopy to determine which array addresses have bound an albumin-specific probe. After imaging, the fluidic lane is contacted with a probe removal fluid, then rinsed to remove unbound probes. The detectable probe method is repeated four more times for each fluidic lane for a total of five rounds of probe binding.

Fluorescence imaging data of albumin-specific detactable probe binding for each lane is provided to a computer algorithm. The algorithm identifies array addresses that have bound a detectable probe via the detection of the presence of fluorescence at the array address. Presence or absence of probe binding at each array address for each of the five rounds is determined by the computer algorithm and results for each array are compiled. Any array address that is observed to bind the albumin probe at least twice amongst the five rounds is assigned an identity of albumin. Each array address with an identified albumin is excluded from further analysis.

After the five rounds of albumin-specific detectable probe binding, each array is contacted with a series of 100 protein identification affinity agents. Each affinity agent of the series of affinity agents has a characterized binding specificity for one or more trimer or quadramer amino acid protein epitopes that are not present in albumin. The selected set of 100 protein identification affinity agents are chosen based upon a likelihood of binding to epitopes that are present in cytokines that are correlated to ulcerative colitis (e.g., IL-5, IL-12, IL-13, IL-17, etc.). A plurality of each affinity agent of the series of affinity agents is contacted with an array in a fluidic lane. Each affinity agent is put through a cycle of incubation, rinsing, imaging, and removal as per the method of albumin-specific probe binding.

Fluorescence imaging data for the series of protein identification affinity agents for each lane is provided to a computer algorithm. The algorithm identifies array addresses that have bound an affinity agent via the detection of the presence of fluorescence at the array address. A binding profile (e.g., the presence or absence of affinity agent binding for each affinity agent) at each array address for each of the 100 rounds is determined by the computer algorithm and results for each array are compiled. The computer algorithm utilizes binding profiles for each array address that is not considered to contain an albumin to identify the protein present at the array address. Each address with an identified cytokine is counted by the computer algorithm for each single-analyte array.

After the 100 rounds of affinity agent binding, each array is finally contacted with a series of proteoform-specific affinity agents. Each proteoform-specific affinity agent has a characterized binding specificity for a particular proteoform of a cytokine of interest. Proteoform-specific affinity agent binding is measured as per other aforementioned affinity agents. Proteoform-specific affinity agent binding data is provided to a computer algorithm that determines the presence or absence of specific cytokine proteoforms at each array address that contains an identified cytokine.

After all binding data has been collected, cytokine data is compiled by the computer algorithm. The clinical provider is provided a data report containing total cytokine quantity, individual cytokine quantities for IL-5, IL-12, IL-13, and IL-17, and quantities of individual proteoforms for each measured cytokine on days 0, 3, 7, and 14. Based upon the data report, the clinical provider determines whether to alter the pharmaceutical treatment method of the medical patient.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

Notwithstanding the appended claims, the disclosure set forth herein is also defined by the following clauses:
1. A method of detecting a polypeptide binding interaction, the method comprising contacting a ligand-binding polypeptide and a binding ligand of the polypeptide in the presence of a solid support to form a polypeptide binding interaction, and detecting the polypeptide binding interaction on the solid support at single molecule resolution.
2. A method of detecting a polypeptide binding interaction, the method comprising:
   a. providing an array comprising a plurality of binding targets, wherein each binding target of the plurality of binding targets is located at an address on the array, and wherein each address of the array is resolvable from each other address on the array;
   b. contacting the array with a plurality of detectable probes, wherein each detectable probe comprises a ligand-binding polypeptide and a detectable label; and
   c. detecting presence or absence of the polypeptide binding interaction at each address at single molecule resolution, wherein the polypeptide binding interaction comprises binding of a detectable probe of the plurality of detectable probes to a binding target of the plurality of binding targets.
3. The method of clause 2, wherein the ligand-binding polypeptide comprises an albumin or a globulin.
4. The method of clause 2 or 3, wherein each detectable probe comprises:
   a. a retaining component;
   b. a plurality of ligand-binding polypeptides coupled to the retaining component; and
   c. one or more detectable labels coupled to the retaining component.
5. The method of clause 4, wherein the retaining component comprises a structured nucleic acid particle (SNAP) or a nanoparticle.
6. The method of clause 5, wherein the SNAP comprises a nucleic acid origami or a nucleic acid nanoball.
7. The method of clause 5, wherein the nanoparticle comprises a fluorescently-labeled nanoparticle, a polymeric nanoparticle, a dendrimer, a branched polymer, or a quantum dot.
8. The method of clause 2 or 3, wherein the detectable probe comprises the ligand-binding polypeptide conjugated to a detectable label.
9. The method of any one of clauses 2-8, wherein the plurality of binding targets comprises a plurality of polypeptides.
10. The method of clause 9, wherein the plurality of polypeptides comprises a polypeptide derived from a sample obtained from a human subject.
11. The method of clause 10, wherein the sample comprises a blood sample.
12. The method of clause 10, wherein the sample does not comprise a blood sample.
13. The method of any one of clauses 9-12, the method further comprising one or more of the steps of:
   a. extracting the plurality of polypeptides from the sample; and
   b. separating a fraction of ligand-binding polypeptides from the plurality of polypeptides.
14. The method of clause 13, further comprising discarding the fraction of ligand-binding polypeptides.
15. The method of any one of clauses 9-14, wherein the plurality of polypeptides comprises a standard polypeptide or a control polypeptide.
16. The method of any one of clauses 2-15, wherein the plurality of binding targets comprises a plurality of non-polypeptide binding targets.
17. The method of clause 16, wherein the plurality of non-polypeptide binding targets comprises a nucleic acid, a lipid, a plant-derived compound, or a pharmaceutical compound.
18. The method of any one of clauses 2-17, wherein a detectable probe of the plurality of detectable probes comprises a native ligand-binding polypeptide.
19. The method of any one of clauses 2-17, wherein a detectable probe of the plurality of detectable probes comprises an engineered ligand-binding polypeptide.
20. The method of any one of clauses 2-19, wherein the ligand-binding polypeptide comprises an engineered mutation relative to a native ligand-binding polypeptide.
21. The method of any one of clauses 2-19, wherein the ligand-binding polypeptide comprises a post-translational modification.
22. The method of clause 21, wherein the post-translational modification comprises a glycation, a cysteinylation, an S-nitrosylation, an S-transnitrosation, a dehydroalanine conversion, or an S-guanylation.
23. The method of any one of clauses 18-22, wherein the ligand-binding polypeptide is extracted from a subject.
24. The method of any one of clauses 2-23, wherein the detecting presence or absence of the polypeptide binding interaction at each address comprises detecting presence of signal or absence of signal from a detectable probe of the plurality of detectable probes at each address.
25. The method of clause 24, wherein the signal is observed by fluorescence measurement, luminescence measurement, luminescence lifetime measurement, or signal encoding.
26. The method of any one of clauses 2-25, wherein the providing the array comprising the plurality of binding targets comprises one or more steps of:
   a. contacting the plurality of binding targets with a plurality of linkers, wherein each linker of the plurality of linkers is configured to couple to a binding target of the plurality of binding targets;
   b. contacting the plurality of linkers with a solid support comprising a plurality of addresses, wherein each address of the plurality of addresses is configured to couple with a linker of the plurality of linkers;
   c. coupling a binding target of the plurality of binding targets to a single linker of the plurality of linkers; and
   d. coupling a single linker of the plurality of linkers to an address of the plurality of addresses.
27. The method of any one of clauses 1-26, further comprising identifying a binding target of the plurality of binding targets at an address on the array.
28. The method of clause 27, wherein identifying the binding target of the plurality of binding targets at the address on the array comprises one or more steps of:
   a. contacting the array with a first plurality of affinity agents, wherein a binding property of an affinity agent of the first plurality of affinity agents is characterized with respect to a set of epitopes;
   b. detecting presence or absence of signal from the affinity agent of the first plurality of affinity agents at the address on the array;
   c. contacting the array with one or more additional pluralities of affinity agents, wherein a binding property of an affinity agent of each of the one or more additional pluralities of affinity agents is characterized with respect to one or more additional sets of epitopes;
   d. detecting presence or absence of signal from an affinity agent of each additional plurality of affinity agents at the address on the array; and
   e. identifying the polypeptide of the plurality of polypeptides at the address based upon the observed presence or absence of signal from at least a set of the observed pluralities of affinity agents at the address.
29. The method of clause 28, wherein step e) comprises:
   a. providing the observed presence or absence of signal from each plurality of affinity agents at the address to a computer algorithm; and
   b. determining the identity of the binding target at the address by the computer algorithm based upon the observed presence or absence of signal from each plurality of affinity agents at the address.
30. The method of any one of clauses 2-29, wherein each address of the plurality of addresses comprising a single binding target of the plurality of binding targets.
31. A method of detecting a polypeptide binding interaction, the method comprising:
   a. providing an array comprising a plurality of addresses, wherein each address of the plurality of addresses comprises a ligand-binding polypeptide, and wherein each address on the array is resolvable from each other address on the array;
   b. contacting the array with a plurality of binding entities; and
   c. detecting presence or absence of the polypeptide binding interaction at each address at single analyte resolution, wherein the polypeptide binding interaction comprises binding of a binding entity of the plurality of binding entities with the ligand-binding polypeptide.
32. The method of clause 31, wherein the ligand-binding polypeptide comprises an albumin or a globulin.
33. The method of clause 31 or 32, wherein detecting the presence or the absence of the polypeptide binding interaction at each address comprises the steps of:
   a. contacting the array with a first plurality of detectable affinity agents, wherein a detectable affinity agent comprises a characterized lack of binding specificity to a ligand-binding polypeptide of the plurality of ligand-binding polypeptides; and
   b. detecting presence or absence of a first signal from a detectable affinity agent of the plurality of detectable affinity agents at each address.
34. The method of clause 33, wherein the characterized lack of binding of the detectable affinity agent to the ligand-binding polypeptide comprises a dissociation constant (K_{D}) of the detectable affinity agent from the ligand-binding polypeptide of at least 10 milliMolar (mM).
35. The method of clause 33, wherein the characterized lack of binding of the detectable affinity agent to the ligand-binding polypeptide comprises a dissociation rate constant (k_{off}) of the detectable affinity agent from the ligand-binding polypeptide of at least 1×10⁻² s⁻¹.
36. The method of any one of clauses 31-35, wherein presence of the first signal at an address indicates presence of a binding entity of the plurality of binding entities at a binding site of the ligand-binding polypeptide.
37. The method of any one of clauses 26-28, wherein absence of the first signal at an address indicates absence of a binding target of the plurality of binding targets at a binding site of the ligand-binding polypeptide.
38. The method of any one of clauses 31-37, further comprising the steps of:
   a. contacting the array with a second plurality of detectable affinity agents, wherein each detectable affinity agent comprises a characterized lack of binding to a ligand-binding polypeptide of the plurality of ligand-binding polypeptides;
   b. detecting presence or absence of a second signal from a detectable affinity agent of the second plurality of detectable affinity agents at each address; and
   c. based upon presence or absence of the first signal and presence or absence of the second signal, identifying a binding entity of the plurality of binding entities at an address of the plurality of addresses.
39. The method of clause 38, wherein detecting presence or absence of the polypeptide binding interaction at each address comprises the steps of:
   a. contacting the array with a first plurality of detectable affinity agents, wherein a detectable affinity agent of the first plurality of detectable affinity agents comprises a characterized binding affinity to a ligand-binding polypeptide of the plurality of ligand-binding polypeptides; and
   b. detecting presence or absence of a signal from a detectable affinity agent of the plurality of detectable affinity agents at each address.
40. The method of clause 39, wherein the characterized binding affinity of the detectable affinity agent to the ligand-binding polypeptide comprises a characterized dissociation constant of the detectable affinity agent from the ligand-binding polypeptide of no more than 1 micromolar (μM).
41. The method of clause 39, wherein the characterized binding affinity of the detectable affinity agent to the ligand-binding polypeptide comprises a characterized dissociation rate constant of the detectable affinity agent from the ligand-binding polypeptide of no more than 1×10⁻³ s⁻¹.
42. The method of any one of clauses 39-41, wherein the affinity agent of the first plurality of affinity agents comprises a binding specificity for an epitope associated with a first binding site of the ligand-binding polypeptide.
43. The method of clause 42, wherein presence of the signal at the address indicates absence of a binding entity of the plurality of binding entities at the first binding site of the ligand-binding polypeptide.
44. The method of clause 42, wherein absence of the signal at the address indicates presence of a binding entity of the plurality of binding entities at the first binding site of the ligand-binding polypeptide.
45. The method of any one of clauses 39-44, further comprising the steps of:
   a. contacting the array with a second plurality of detectable affinity agents, wherein a detectable affinity agent of the second plurality of detectable affinity agents comprises a characterized binding specificity to an epitope associated with a second binding site of the ligand-binding polypeptide of the plurality of ligand-binding polypeptides; and
   b. detecting presence or absence of a signal from a detectable affinity agent of the second plurality of detectable affinity agents at each address.
46. The method of clause 38, wherein presence of the signal at the address indicates absence of a binding entity of the plurality of binding entities at the second binding site of the ligand-binding polypeptide.
47. The method of clause 35, wherein absence of the signal at the address indicates presence of a binding entity of the plurality of binding entities at the second binding site of the ligand-binding polypeptide.
48. The method of any one of clauses 39-47, further comprising identifying a binding entity of the plurality of binding entities at an address of the plurality of addresses.
49. The method of clause 48, wherein identifying the binding entity of the plurality of binding entities at the address comprises:
   a. providing the observed presence or absence of signal from each plurality of affinity agents at the address to a computer algorithm; and
   b. determining the identity of the polypeptide at the address by the computer algorithm based upon the observed presence or absence of signal from each plurality of affinity agents at the address.
50. The method of any one of clauses 39-49, wherein an affinity agent of the first plurality of affinity agents comprises a binding ligand for the ligand-binding polypeptide, wherein the binding ligand is coupled to a detectable label.
51. The method of any one of clauses 31-50, wherein the plurality of binding entities comprises a plurality of polypeptides.
52. The method of clause 51, wherein the plurality of polypeptides comprises a polypeptide extracted from a sample obtained from a human subject.
53. The method of clause 52, wherein the sample comprises a blood sample.
54. The method of clause 52, wherein the sample does not comprise a blood sample.
55. The method of any one of clauses 31-54, wherein a ligand-binding polypeptide of the plurality of ligand-binding polypeptides comprises a native ligand-binding polypeptide.
56. The method of any one of clauses 31-54, wherein a ligand-binding polypeptide of the plurality of ligand-binding polypeptides comprises an engineered ligand-binding polypeptide.
57. The method of any one of clauses 31-56, wherein a ligand-binding polypeptide of the plurality of ligand-binding polypeptides comprises an engineered mutation relative to a native ligand-binding polypeptide.
58. The method of any one of clauses 31-57, wherein the ligand-binding polypeptide comprises a post-translational modification.
59. The method of clause 58, wherein the post-translational modification comprises a glycation, a cysteinylation, an S-nitrosylation, an S-transnitrosation, a dehydroalanine conversion, or an S-guanylation.
60. The method of any one of clauses 31-59, wherein the plurality of binding entities comprises a plurality of non-polypeptide binding entities.
61. The method of clause 60, wherein the plurality of non-polypeptide binding entities comprise a nucleic acid, a lipid, a plant-derived compound, or a pharmaceutical compound.
62. The method of any one of clauses 31-61, wherein the array has a polypeptide occupancy rate of at least 0.000001.
63. The method of clause 62, wherein the array has a polypeptide occupancy rate of at least 0.001.
64. The method of clause 63, wherein the array has a polypeptide occupancy rate of at least 0.1.
65. The method of any one of clauses 31-64, wherein each address of the plurality of addresses comprises a single ligand-binding polypeptide of the plurality of ligand-binding polypeptides.
66. A composition, comprising:
   a. a solid support comprising a plurality of addresses wherein each address of the plurality of addresses is resolvable from each other address, and further comprising a plurality of interstitial regions that separate each address of the plurality of addresses from one or more adjacent addresses of the plurality of addresses; and
   b. a plurality of ligand-binding polypeptides, wherein each ligand-binding polypeptide of the plurality of ligand-binding polypeptides comprises an albumin or a globulin, and wherein each ligand-binding polypeptide of the plurality of ligand-binding polypeptides is coupled to an address of the plurality of addresses, and wherein the interstitial regions of the plurality of interstitial regions are devoid of ligand-binding polypeptides.
67. The composition of clause 66, further comprising a nucleic acid linker that couples the ligand-binding polypeptide to the solid support.
68. The composition of clause 67, wherein the nucleic acid linker comprises an oligonucleotide.
69. The composition of clause 67, wherein the nucleic acid linker comprises a structured nucleic acid particle (SNAP).
70. The composition of clause 69, wherein the SNAP comprises a nucleic acid origami or a nucleic acid nanoball.
71. The composition of any one of clauses 66-70, wherein the nucleic acid linker is covalently coupled to the solid support.
72. The composition of any one of clauses 66-70, wherein the nucleic acid linker is non-covalently coupled to the solid support.
73. The composition of any one of clauses 66-72, further comprising a plurality of binding entities, wherein each binding entity of the plurality of binding entities forms a polypeptide binding interaction with a ligand-binding polypeptide of the plurality of ligand-binding polypeptides.
74. The composition of clause 73, wherein the plurality of binding entities comprises a plurality of polypeptide binding entities.
75. The composition of clause 73 or 74, wherein the plurality of binding entities comprises a plurality of non-polypeptide binding entities.
76. The composition of clause 73, wherein the plurality of binding entities comprises two or more species of binding entities.
77. The composition of clause 76, wherein the two or more species of binding entities comprise two or more species of polypeptides extracted from a human subject.
78. The composition of clause 73, wherein the plurality of binding entities comprises a single species of binding entity.
79. The composition of any one of clauses 73-78, further comprising a competitive binding ligand that forms a polypeptide binding interaction with a ligand-binding polypeptide of the plurality of ligand-binding polypeptides.
80. The composition of any one of clauses 66-79, wherein a ligand-binding polypeptide of the plurality of ligand-binding polypeptides comprises a non-engineered ligand-binding polypeptide.
81. The composition of any one of clauses 66-79, wherein a ligand-binding polypeptide of the plurality of ligand-binding polypeptides comprises an engineered ligand-binding polypeptide.
82. The composition of any one of clauses 66-81, wherein a ligand-binding polypeptide of the plurality of ligand-binding polypeptides comprises a mutation relative to a native ligand-binding polypeptide.
83. The composition of any one of clauses 66-82, wherein a ligand-binding polypeptide of the plurality of ligand-binding polypeptides comprises a post-translational modification.
84. The composition of clause 83, wherein the post-translational modification comprises a glycation, a cysteinylation, an S-nitrosylation, an S-transnitrosation, a dehydroalanine conversion, or an S-guanylation.
85. The composition of any one of clauses 66-84, wherein a ligand-binding polypeptide of the plurality of ligand-binding polypeptides is extracted from a sample obtained from a human subject.
86. The composition of clause 85, wherein the sample comprises a blood sample.
87. The composition of clause 85, wherein the sample does not comprise a blood sample.
88. The composition of any one of clauses 66-87, wherein each address of the plurality of addresses comprises a single ligand-binding polypeptide of the plurality of ligand-binding polypeptides.
89. A composition, comprising:
   a. a solid support comprising a plurality of addresses wherein each address of the plurality of addresses is resolvable from each other address, and further comprising a plurality of interstitial regions that separate each address of the plurality of addresses from one or more adjacent addresses of the plurality of addresses; and
   b. a plurality of polypeptide complexes, wherein each polypeptide complex of the plurality of polypeptide-complexes comprises a ligand-binding polypeptide and a binding ligand of the ligand-binding polypeptide, wherein each polypeptide complex of the plurality of polypeptide complexes is coupled to an address of the plurality of addresses, and wherein each interstitial region of the plurality of interstitial regions does not comprise a polypeptide complex or a ligand-binding polypeptide.
90. The composition of clause 89, further comprising a fluid in contact with the array, wherein the fluid comprises a plurality of free ligand-binding polypeptides.
91. The composition of clause 89 or 90, wherein the fluid further comprises a competitive binding ligand, wherein the competitive binding ligand comprises a binding ligand for the ligand-binding polypeptide.
92. The composition of any one of clauses 89-91, wherein a ligand-binding polypeptide of the plurality of ligand-binding polypeptides comprises a native ligand-binding polypeptide.
93. The composition of any one of clauses 89-91, wherein a ligand-binding polypeptide of the plurality of ligand-binding polypeptides comprises an engineered ligand-binding polypeptide.
94. The composition of any one of clauses 89-93, wherein a ligand-binding polypeptide of the plurality of ligand-binding polypeptides comprises an engineered mutation relative to a native ligand-binding polypeptide.
95. The composition of any one of clauses 89-94, wherein a ligand-binding polypeptide of the plurality of ligand-binding polypeptides comprises a post-translational modification.
96. The composition of clause 95, wherein the post-translational modification comprises a glycation, a cysteinylation, an S-nitrosylation, an S-transnitrosation, a dehydroalanine conversion, or an S-guanylation.
97. The composition of any one of clauses 89-96, wherein a ligand-binding polypeptide of the plurality of ligand-binding polypeptides is extracted from a sample obtained from a human subject.
98. The composition of clause 97, wherein the sample comprises a blood sample.
99. The composition of clause 97, wherein the sample does not comprise a blood sample.
100. The composition of any one of clauses 89-99, further comprising a cross-linking molecule.
101. The composition of clause 100, wherein the cross-linking molecule is configured to couple the binding ligand to the ligand-binding polypeptide.
102. The composition of clause 101, wherein the cross-linking molecule is in fluidic communication with the solid support.
103. The composition of clause 100, wherein the cross-linking molecule comprises a cross-link between the binding ligand and the ligand-binding molecule.
104. The composition of any one of clauses 89-103, wherein each address of the plurality of addresses comprises a single polypeptide complex of the plurality of polypeptide complexes.
105. A system, comprising:
   a. in a first configuration, a solid support comprising a plurality of addresses wherein each address of the plurality of addresses is resolvable from each other address, and further comprising a plurality of interstitial regions that separate each address of the plurality of addresses from one or more adjacent addresses of the plurality of addresses, wherein each address of the plurality of addresses comprises a first polypeptide complex comprising a ligand-binding molecule coupled to a binding entity, wherein the ligand-binding polypeptide comprises an albumin or a globulin, and wherein the solid support is in contact with a fluid comprising a competitive binding ligand, wherein the competitive binding ligand is a binding ligand of the ligand-binding polypeptide; and
   b. in a second configuration, the solid support comprising the plurality of addresses, wherein a subset of addresses of the plurality of addresses comprises a second polypeptide complex comprising the ligand-binding polypeptide and the competitive binding ligand, and wherein the solid support is in contact with a fluid comprising the binding entity;
      wherein the first configuration and the second configuration are distinguishable at single-analyte resolution.
106. The composition of clause 105, wherein the polypeptide comprises a first detectable label and the competitive binding ligand comprises a second detectable label, wherein the first detectable label is distinguishable from the second detectable label.
107. The composition of clause 105 or 106, wherein the binding entity comprises a polypeptide binding ligand.
108. The composition of clause 105 or 106, wherein the binding entity comprises a non-polypeptide binding ligand.
109. A system, comprising:
   a. in a first configuration, a solid support comprising a plurality of addresses wherein each address of the plurality of addresses is resolvable from each other address, and further comprising a plurality of interstitial regions that separate each address of the plurality of addresses from one or more adjacent addresses of the plurality of addresses, wherein each address of the plurality of addresses comprises a first polypeptide complex comprising a ligand-binding molecule coupled to a first binding entity, wherein the ligand-binding polypeptide comprises an albumin or a globulin, and wherein the solid support is in contact with a fluid comprising a second binding entity, wherein the second binding entity is a binding ligand of the ligand-binding polypeptide; and
   b. in a second configuration, the solid support comprising the plurality of addresses, wherein a subset of addresses of the plurality of addresses comprises a second polypeptide complex, wherein the second polypeptide complex comprises the ligand-binding polypeptide, the first binding entity, and the second binding entity;
      wherein the first configuration and the second configuration are distinguishable at single-analyte resolution.
110. The composition of clause 109, wherein the first binding entity comprises a first detectable label and the second binding entity comprises a second detectable label, wherein the first detectable label is distinguishable from the second detectable label.
111. The composition of clause 109 or 110, wherein the first binding entity comprises a polypeptide binding ligand.
112. The composition of clause 109 or 110, wherein the first binding entity comprises a non-polypeptide binding ligand.
113. The composition of any one of clauses 109-112, wherein the second binding entity comprises a polypeptide binding ligand.
114. The composition of any one of clauses 109-112, wherein the second binding entity comprises a non-polypeptide binding ligand.
115. A method of characterizing a ligand-binding interaction, comprising:
   a. providing an array comprising a plurality of addresses, wherein each address of the plurality of addresses comprises a coupled polypeptide complex, wherein each address is resolvable from each other address, and wherein each polypeptide complex comprises a ligand-binding polypeptide and a binding ligand of the ligand-binding polypeptide, wherein the ligand-binding polypeptide comprises an albumin or a globulin;
   b. detecting presence or absence of the coupled polypeptide complex at each address of the plurality of addresses; and
   c. contacting the array with a plurality of free ligand-binding polypeptides, thereby transferring at least one binding ligand of the plurality of binding ligands from a coupled polypeptide complex to a free ligand-binding polypeptide of the plurality of free ligand-binding polypeptides.
116. The method of clause 115, wherein the binding ligand comprises a polypeptide species.
117. The method of clause 116, wherein the polypeptide species comprises a hormone species.
118. The method of clause 115, wherein the binding ligand comprises a non-polypeptide species.
119. The method of clause 118, wherein the non-polypeptide species comprises a monatomic ion, a polyatomic ion, a lipid, a flavonoid, a retinoid, or a pharmaceutical compound.
120. The method of any one of clauses 115-119, wherein a free ligand-binding polypeptide of the plurality of free ligand-binding polypeptides comprises an altered ligand-binding polypeptide relative to the ligand-binding polypeptide of the plurality of polypeptides.
121. The method of clause 120, wherein the altered ligand-binding polypeptide comprises an engineered amino acid mutation relative to the ligand-binding polypeptide of the plurality of polypeptides.
122. The method of clause 120 or 121, wherein the altered ligand-binding polypeptide comprises a post-translational modification relative to the ligand-binding polypeptide of the plurality of polypeptides.
123. The method of any one of clauses 115-122, comprising performing step b) before step c).
124. The method of clause 123, comprising repeating step b) after step c).
125. The method of any one of clauses 115-122, comprising performing step b) after step c).
126. The method of any one of clauses 115-125, wherein detecting the presence or absence of the coupled polypeptide complex comprises:
   a. contacting the array with a plurality of detectable affinity agents, wherein each detectable affinity agent comprises a characterized binding affinity to a ligand-binding polypeptide of the plurality of ligand-binding polypeptides; and
   b. detecting presence or absence of a signal from a detectable affinity agent of the plurality of detectable affinity agents at each address.
127. The method of clause 126, wherein the characterized binding affinity of the detectable affinity agent to the ligand-binding polypeptide comprises a dissociation constant (K_{D}) of the detectable affinity agent from the ligand-binding polypeptide of no more than 1 micromolar (μM).
128. The method of clause 126, wherein the characterized binding affinity of the detectable affinity agent to the ligand-binding polypeptide comprises a dissociation rate constant (k_{off}) of the detectable affinity agent from the ligand-binding polypeptide of no more than 1×10⁻³ per second (s⁻¹).
129. The method of any one of clauses 126-128, wherein the detectable affinity agent of the plurality of detectable affinity agents comprises a binding specificity for an epitope associated with a binding site of the ligand-binding polypeptide.
130. The method of clause 129, wherein presence of the signal at the address indicates absence of a binding ligand of the plurality of binding ligands at the binding site of the ligand-binding polypeptide.
131. The method of clause 129, wherein absence of the signal at the address indicates presence of a binding ligand of the plurality of binding ligands at the binding site of the ligand-binding polypeptide.
132. The method of any one of clauses 115-131, further comprising, before contacting the array with a plurality of free ligand-binding polypeptides, identifying the binding ligand bound to the ligand-binding polypeptide at an address of the plurality of addresses.
133. The method of any one of clauses 115-132, further comprising, after contacting the array with a plurality of free ligand-binding polypeptides, detecting presence of the binding ligand bound to the free ligand-binding polypeptide.
134. The method of clause 133, further comprising capturing the free ligand-binding polypeptide of the plurality of ligand-binding polypeptides from a fluid phase removed from contact with the solid support.
135. The method of clause 133, further comprising identifying the binding ligand bound to the free ligand-binding polypeptide.
136. The method of clause 134, wherein identifying the binding ligand bound to the free ligand-binding polypeptide comprises performing a mass spectrometry analysis of the free ligand-binding polypeptide or the binding ligand.
137. The method of clause 134, wherein identifying the binding ligand bound to the free ligand-binding polypeptide comprises performing a single-analyte polypeptide assay.
138. A method, comprising:
   a. providing an array comprising a plurality of binding targets, wherein each binding target of the plurality of binding targets is located at an address that is resolvable from each other address on the array, and wherein each address of the array is detectable at single-analyte resolution, wherein the plurality of binding targets comprises a ligand-binding polypeptide, wherein the plurality of binding targets comprises two or more species, and wherein the ligand-binding polypeptide comprises an albumin or a globulin;
   b. detecting presence or absence of binding of an affinity agent of a first plurality of affinity agents to a polypeptide of the plurality of polypeptides at each address of the plurality of addresses, wherein the affinity agent comprises a binding specificity for the ligand-binding polypeptide;
   c. determining presence or absence of the ligand-binding polypeptide at each address of the plurality of addresses beyond a threshold measure of confidence; and
   d. detecting presence or absence of binding of an affinity agent of a second plurality of affinity agents at each address of the plurality of addresses excluding a subset of addresses where presence of the ligand-binding polypeptide is determined beyond the threshold measure of confidence.
139. The method of clause 137, wherein the plurality of binding targets comprises a plurality of polypeptides.
140. The method of clause 138, wherein the plurality of binding targets further comprises a plurality of non-polypeptides.
141. The method of clause 138, wherein the plurality of polypeptides is derived from a sample obtained from a human subject.
142. The composition of clause 140, wherein the sample comprises a blood sample.
143. The composition of clause 140, wherein the sample does not comprise a blood sample.
144. The method of any one of clauses 137-142, wherein the providing the array comprising the plurality of binding targets comprises one or more of the steps of:
   a. providing the plurality of binding targets; and
   b. coupling the plurality of binding targets to a solid support, wherein the solid support comprises a plurality of addresses, wherein each address of the plurality of addresses is resolvable from each other address, and wherein each address of the plurality of addresses comprises a single binding target of the plurality of binding targets.
145. The method of clause 143, wherein the coupling the plurality of binding targets to the solid support comprises one or more steps of:
   a. coupling each binding target of the plurality of binding targets to a linker; and
   b. coupling each linker to a single address of the plurality of addresses on the solid support, wherein each address comprises a single linker.
146. The method of clause 143, wherein the linker comprises a nucleic acid.
147. The method of clause 144 or 145, wherein the coupling each linker to the single address on the solid support comprises covalently attaching the linker to the solid support.
148. The method of clause 144 or 145, wherein the coupling each linker to the single address on the solid support comprises non-covalently attaching the linker to the solid support.
149. The method of any one of clauses 143-147, further comprising disrupting a polypeptide complex before coupling the plurality of binding targets to the solid support, wherein the polypeptide complex comprises a ligand-binding polypeptide and a binding ligand.
150. The method of clause 148, wherein disrupting the polypeptide complex comprises denaturing the polypeptide complex.
151. The method of clause 148, wherein the disrupting the polypeptide complex comprises the steps of:
   a. contacting the polypeptide complex with a competitive binding ligand, wherein the competitive binding ligand is a binding ligand for the ligand-binding polypeptide; and
   b. binding the competitive binding ligand to the ligand-binding polypeptide, thereby releasing the binding ligand.
152. The method of clause 150, wherein the competitive binding ligand comprises a non-polypeptide molecule.
153. The method of any one of clauses 137-151, wherein the plurality of binding targets comprises at least 10% ligand-binding polypeptides by weight.
154. The method of clause 152, wherein the plurality of binding targets comprises at least 25% ligand-binding polypeptides by weight.
155. The method of clause 130, wherein the plurality of binding targets comprises at least 50% ligand-binding polypeptides by weight.
156. The method of any one of clauses 137-154, wherein the second plurality of affinity agents comprises an affinity agent that does not bind the ligand-binding polypeptide.
157. The method of any one of clauses 137-155, further comprising identifying a binding target at an address of the plurality of addresses excluding the subset of addresses where the presence of the ligand-binding polypeptide was determined.
158. The method of clause 156, wherein the identifying the binding target at the address of the plurality of addresses further comprises providing data to an analysis algorithm regarding presence or absence of observed binding of the affinity agent of the first plurality of affinity agents at each address, and providing data to an analysis algorithm regarding presence or absence of observed binding of the affinity agent of the second plurality of affinity agents at each address.
159. The method of clause 157, wherein the analysis algorithm comprises one or more of an image analysis algorithm, a signal processing algorithm, and a polypeptide identification algorithm.
160. A composition, comprising:
   a. a solid support comprising a plurality of addresses wherein each address of the plurality of addresses is resolvable from each other address, and further comprising a plurality of interstitial regions that separate each address of the plurality of addresses from one or more adjacent addresses of the plurality of addresses; and
   b. a plurality of polypeptides, wherein a single polypeptide of the plurality of polypeptides is coupled to each address of the plurality of addresses, wherein each interstitial region of the plurality of interstitial regions does not comprise a polypeptide of the plurality of polypeptides, wherein the plurality of polypeptides is derived from a single human subject, and wherein the plurality of polypeptides comprises at least 75% albumin and globulin, by weight.
161. A detectable probe composition comprising:
   a. a retaining component comprising one or more detectable labels; and
   b. two or more polypeptides coupled to the retaining component, wherein the two or more polypeptides are independently selected from an albumin and a globulin, and wherein the two or more polypeptides are derived from a single human subject.
162. A composition, comprising:
   a. a plurality of polypeptides, wherein a polypeptide of the plurality of polypeptides comprises a ligand-binding polypeptide selected from an albumin and a globulin; and
   b. a linker, wherein the linker is configured to couple to the polypeptide of the plurality of polypeptides, and wherein the linker is configured to be coupled to a solid support.
163. The composition of clause 161, wherein the plurality of polypeptides comprises at least about 50% ligand-binding polypeptides by weight relative to total polypeptide content.
164. The composition of clause 162, wherein the plurality of polypeptides comprises at least about 90% ligand-binding polypeptides by weight relative to total polypeptide content.
165. The composition of any one of clauses 161-163, wherein the linker comprises a nanoparticle.
166. The composition of clause 164, wherein the nanoparticle comprises a polymeric nanoparticle, a nucleic acid nanoparticle, an organic nanoparticle, a metallic nanoparticle, or a semiconductor nanoparticle.
167. The composition of clause 165, wherein the nucleic acid nanoparticle comprises a structured nucleic acid nanoparticle selected from a nucleic acid origami and a nucleic acid nanoball.
168. The composition of any one of clauses 161-166, wherein the linker comprises a coupling site that is configured to bind to the ligand-binding polypeptide.
169. The composition of any one of clauses 161-167, wherein the coupling site is configured to covalently bind to a polypeptide of the plurality of polypeptides.
170. The composition of any one of clauses 161-167, wherein the coupling site is configured to non-covalently bind to a polypeptide of the plurality of polypeptides.
171. The composition of any one of clauses 167-169, wherein the linker comprises a plurality of coupling sites, wherein each coupling site is configured to covalently bind to a different polypeptide of the plurality of polypeptides.
172. The composition of any one of clauses 161-170, wherein the linker is coupled to the a polypeptide of the plurality of polypeptides.
173. The composition of any one of clauses 161-171, wherein the linker comprises one or more detectable labels that are configured to provide a detectable signal.
174. The composition of any one of clauses 161-172, wherein the linker comprises a surface that is configured to couple to a solid support.
175. The composition of clause 173, wherein the surface is configured to couple to the solid support by a non-covalent interaction selected from the group consisting of an electrostatic interaction, a magnetic interaction, a ligand-binding interaction, and a nucleic acid hybridization interaction.
176. The composition of clause 173, wherein the surface is configured to couple to the solid support by a covalent interaction.
177. The composition of any one of clauses 161-175, wherein the linker comprises one or more passivating groups.
178. The composition of any one of clauses 161-176, wherein the plurality of polypeptides further comprises a polypeptide binding ligand for the ligand-binding polypeptide.
179. The composition of clause 177, wherein the polypeptide binding ligand is bound to the ligand-binding polypeptide.
180. The composition of clause 177 or 178, further comprising a cross-linking moiety.
181. The composition of clause 179, wherein the cross-linking moiety forms a cross-link between the polypeptide binding ligand and the ligand-binding polypeptide.
182. The composition of any one of clauses 173-180, further comprising a fluid that is configured to be contacted to the solid support.
183. The composition of any one of clauses 173-181, further comprising the solid support.
184. The composition of clause 182, wherein the linker is coupled to the solid support.
185. The composition of clause 183, wherein the ligand-binding polypeptide is not coupled to the linker.
186. The composition of clause 183, wherein the ligand-binding polypeptide is coupled to the linker.
187. A method of forming a ligand-binding polypeptide particle, comprising:
   a. providing a composition of any one of clauses 161-185; and
   b. coupling linker to the ligand-binding polypeptide, thereby forming a ligand-binding polypeptide particle.
188. The method of clause 186, further comprising coupling the linker to a solid support.
189. The method of clause 187, further comprising coupling one or more additional binding entities to the ligand-binding polypeptide.
190. A composition, comprising:
   a. a solid support comprising a plurality of addresses wherein each address of the plurality of addresses is resolvable from each other address, and further comprising a plurality of interstitial regions that separate each address of the plurality of addresses from one or more adjacent addresses of the plurality of addresses;
   b. a plurality of ligand-binding polypeptides, wherein each ligand-binding polypeptide of the plurality of ligand-binding polypeptides is coupled to a first subset of addresses of the plurality of addresses, and wherein the interstitial regions of the plurality of interstitial regions are devoid of ligand-binding polypeptides; and
   c. a plurality of standard polypeptides, wherein each standard polypeptide is coupled to a second subset of addresses of the plurality of addresses, and wherein the plurality of interstitial regions are devoid of standard polypeptides.
191. A composition, comprising:
   a. a solid support comprising a plurality of addresses wherein each address of the plurality of addresses is resolvable from each other address, and further comprising a plurality of interstitial regions that separate each address of the plurality of addresses from one or more adjacent addresses of the plurality of addresses;
   b. a plurality of ligand-binding polypeptides, wherein each ligand-binding polypeptide of the plurality of ligand-binding polypeptides is coupled to an address of the plurality of addresses, and wherein the interstitial regions of the plurality of interstitial regions are devoid of ligand-binding polypeptides; and
   c. a fluid in contact with the solid support, wherein the fluid comprises a plurality of binding ligands, wherein a binding ligand of the plurality of binding ligands is configured to form a binding interaction with a ligand-binding polypeptide of the plurality of ligand-binding polypeptides.
192. A composition, comprising:
   a. a solid support comprising a plurality of addresses wherein each address of the plurality of addresses is resolvable from each other address, and further comprising a plurality of interstitial regions that separate each address of the plurality of addresses from one or more adjacent addresses of the plurality of addresses;
   b. a plurality of polypeptide complexes, wherein each polypeptide complex comprises a ligand-binding polypeptide coupled to a binding ligand of the ligand-binding polypeptide, wherein each polypeptide complex of the plurality of polypeptide complexes is coupled to an address of the plurality of addresses, and wherein the interstitial regions of the plurality of interstitial regions are devoid of polypeptide complexes; and
   c. a fluid in contact with the solid support, wherein the fluid comprises a polypeptide complex dissociating species, wherein the polypeptide complex dissociating species is selected from the group comprising a salt, an acid, a base, a surfactant, a competitive binding ligand, and a free ligand-binding polypeptide.
193. A composition, comprising:
   a. a solid support comprising a plurality of addresses wherein each address of the plurality of addresses is resolvable from each other address, and further comprising a plurality of interstitial regions that separate each address of the plurality of addresses from one or more adjacent addresses of the plurality of addresses;
   b. a plurality of ligand-binding polypeptides, wherein each ligand-binding polypeptide of the plurality of ligand-binding polypeptides comprises an albumin or a globulin that is extracted from a human subject, and wherein each ligand-binding polypeptide of the plurality of ligand-binding polypeptides is coupled to an address of the plurality of addresses, and wherein the interstitial regions of the plurality of interstitial regions are devoid of ligand-binding polypeptides; and
   c. a nucleic acid sequence derived from the human subject, wherein the nucleic acid sequence comprises a genomic sequence, and wherein the nucleic acid sequence is coupled to an address of the plurality of addresses.
194. A fluidic device, comprising:
   a. a solid substrate;
   b. a composition of any one of clauses 66-104, wherein the composition is disposed within the solid substrate; and
   c. a fluidic channel comprising a first port and a second port, wherein the fluidic channel is configured to contact a fluid with the composition, and wherein the fluidic channel is configured to receive a portion of the fluid through the first port and evacuate a portion of the fluid through the second port.
195. The fluidic device of clause 193, further comprising a second fluidic channel.
196. The fluidic device of clause 194, wherein the second fluidic channel is in fluidic communication with the fluidic channel.
197. The fluidic device of clause 194, wherein the second fluidic channel is not in fluidic communication with the fluidic channel.
198. The fluidic device of any one of clauses 193-196, wherein the second fluidic channel comprises a second composition any one of clauses 66-104, wherein the second composition is disposed within the solid substrate.
199. The fluidic device of any one of clauses 194-197, wherein the second fluidic channel comprises an unoccupied array that is configured to bind a plurality of polypeptides.
200. A system, comprising:
   a. a fluidic device of any one of clauses 193-198;
   b. a fluid transfer device in fluidic communication with the fluidic device, wherein the fluid transfer device is configured to deliver one or more fluids to the fluidic device, wherein at least one fluid of the one or more fluids comprises a plurality of affinity agents;
   c. a physical measurement device, wherein the physical measurement device is configured to receive a physical measurement comprising a presence or absence of signal from the fluidic device; and
   d. a computer that is configured to receive the physical measurement from the physical measurement device, wherein the computer is configured to identify one or more characteristics of a polypeptide coupled to a solid support within the fluidic device based upon the physical measurement.
201. A method, comprising:
   a. providing an array comprising a plurality of polypeptides and one or more impurities, wherein each polypeptide of the plurality of polypeptides is located at an address that is resolvable from each other address on the array, wherein each of the one or more impurities is located at an address that is resolvable from each other address on the array, wherein each address of the array is detectable at single-analyte resolution, wherein the plurality of polypeptides comprises a ligand-binding polypeptide;
   b. determining presence or absence of an impurity of the one or more impurities at a subset of addresses on the array beyond a threshold measure of confidence; and
   c. detecting presence or absence of binding of an affinity agent of a plurality of affinity agents to a polypeptide of the plurality of polypeptides at each address of the plurality of addresses excluding the subset of addresses where presence of the one or more impurities was determined beyond the threshold measure of confidence, wherein the affinity agent comprises a binding specificity for the ligand-binding polypeptide.
202. A method, comprising:
   a. providing an array comprising a plurality of polypeptide complexes, wherein each polypeptide complex of the plurality of polypeptide complexes comprises a ligand-binding polypeptide coupled to a binding ligand, wherein the array comprises a plurality of addresses, wherein each address of the plurality of addresses is resolvable at single-analyte resolution; wherein each polypeptide complex is coupled to an address of the plurality of addresses, and wherein the ligand-binding polypeptide comprises an albumin or a globulin;
   b. contacting the array with a plurality of detectable probes, wherein a detectable probe of the plurality of detectable probes comprises a binding specificity for a polypeptide complex; and
   c. determining presence or absence of the polypeptide complex of the plurality of polypeptide complexes at each address of the plurality of addresses, wherein determining presence or absence of the polypeptide complex of the plurality of polypeptide complexes comprises detecting signal from the detectable probe of the plurality of detectable probes.
203. A method, comprising:
   a. providing an array comprising a plurality of binding ligands, wherein each binding ligand of the plurality of binding ligands is located at an address on the array, and wherein each address of the array is resolvable from each other address on the array;
   b. contacting the array with a mixture comprising a first plurality of ligand-binding polypeptides and a second plurality of ligand-binding polypeptides, wherein a ligand-binding polypeptide of the first plurality of ligand-binding polypeptides comprises a first binding specificity, and wherein a ligand-binding polypeptide of the second plurality of ligand-binding polypeptides comprises a second binding specificity;
   c. forming a first polypeptide complex at a first address and optionally forming a second polypeptide complex, wherein the first polypeptide complex comprises a binding ligand coupled to the ligand-binding polypeptide of the first plurality of ligand-binding polypeptides, and wherein the second polypeptide complex comprises a binding ligand coupled to the ligand-binding polypeptide of the second plurality of ligand-binding polypeptides;
   d. identifying the first address comprising the first polypeptide complex; and
   e. determining the presence or absence of the first polypeptide complex or the second polypeptide complex at one or more addresses other than the first address.
204. A method of characterizing a ligand-binding interaction, comprising:
   a. providing an array comprising a plurality of addresses, wherein each address of the plurality of addresses comprises a coupled first polypeptide complex, wherein each address is resolvable from each other address, wherein each first polypeptide complex comprises a ligand-binding polypeptide and a binding ligand of the ligand-binding polypeptide, and wherein the ligand-binding polypeptide comprises a first binding affinity for the binding ligand;
   b. contacting the array with a plurality of free binding ligands, wherein the free binding ligand comprises a different chemical structure than the binding ligand, and wherein the ligand-binding polypeptide comprises a second binding affinity for the free binding ligand;
   c. exchanging a binding ligand with a free binding ligand, thereby forming one or more second polypeptide complexes, wherein a second polypeptide complex of the one or more second polypeptide complexes comprises the ligand-binding polypeptide coupled to the free binding ligand; and
   d. detecting presence or absence of the second polypeptide complex at one or more addresses on the array.
205. A method, comprising:
   a. providing a first plurality of ligand-binding polypeptides from a first source of polypeptides, and providing a second plurality of ligand-binding polypeptides from a second source of polypeptides;
   b. detecting presence or absence of binding of the first plurality of ligand-binding polypeptides and the second plurality of ligand-binding polypeptides to a binding ligand on a solid support at single-analyte resolution; and
   c. identifying a difference in a binding characteristic between the first plurality of ligand-binding polypeptides and the second plurality of ligand-binding polypeptides.
206. The method of clause 204, wherein the first source or the second source comprises a medical subject.
207. The method of clause 205, wherein the first source comprises a first medical subject and the second source comprises a second medical subject.
208. The method of clause 206, wherein the first source comprises a medical subject and the second source comprises a control sample.
209. The method of any one of clauses 204-207, wherein identifying a difference in binding characteristic comprises identifying a difference in binding specificity between the first plurality of polypeptides and the second plurality of polypeptides.
210. The method of any one of clauses 204-208, wherein identifying a difference in binding characteristic comprises identifying a difference in binding affinity between the first plurality of polypeptides and the second plurality of polypeptides.
211. The method of clause 209, wherein identifying a difference in binding affinity comprises quantifying a difference in dissociation constant, association rate constant, or dissociation rate constant between the first plurality of polypeptides and the second plurality of polypeptides.
212. The method of any one of clauses 208-210, wherein the method further comprises, based upon identifying the difference in the binding characteristic, identifying a disease state or a health state of the medical subject.
213. The method of clause 211, further comprising, based upon identifying a disease state of the medical subject, identifying a method of treatment for the medical subject for the disease state.
214. A method, comprising:
   a. providing an array comprising a plurality of addresses, wherein each address is resolvable from each other address at single-analyte resolution, wherein each address of a first subset of the plurality of addresses is coupled to a polypeptide of a plurality of polypeptides, wherein each address of a second subset of the plurality of addresses is coupled to a standard polypeptide of a plurality of standard polypeptides; wherein the plurality of polypeptides is extracted from a subject, wherein the plurality of polypeptides comprises a first ligand-binding polypeptide, and wherein the plurality of standard polypeptides comprises a second ligand-binding polypeptide;
   b. contacting the array with a plurality of detectable probes, wherein a detectable probe of the plurality of detectable probes comprises a binding ligand;
   c. detecting presence or absence of a detectable probe of the plurality of detectable probes at each address of the plurality of addresses; and
   d. identifying presence or absence of a polypeptide binding interaction between the first ligand-binding polypeptide and the binding ligand or between the second ligand-binding polypeptide and the binding ligand based upon presence or absence of the detectable probe of the plurality of detectable probes at each address of the plurality of addresses.

What is claimed is:

1. A method of characterizing an interaction between analytes, comprising:
providing an array of a first plurality of different analytes, wherein individual molecules of the first plurality of different analytes are disposed at an individually addressable locations on the array, and wherein each of the individual molecules of the first plurality of different analytes has a known identity;
contacting the individual molecules of the first plurality of different analytes with at least a second analyte; and
characterizing any interaction between the at least one second analyte and each of the individual molecules of the first plurality of different analytes.

2. The method of claim 1, wherein the contacting step comprises separately contacting the individual molecules of the first plurality of different analytes with at least a second analyte in the presence and absence of at least a third analyte, and wherein the characterizing step comprises characterizing an impact of the third analyte on the interaction between the second analyte and each of the individual molecules of the first plurality of different analytes.

3. The method of claim 2, wherein the contacting step additionally comprises separately contacting the individual molecules of the first plurality of different analytes with at least a second analyte in the presence and absence of at least a fourth analyte, and wherein the characterizing step comprises characterizing an impact of the fourth analyte on the interaction between the second analyte and each of the individual molecules of the first plurality of different analytes.

4. The method of claim 2, wherein the third analyte comprises a competitor with the second analyte for interaction with at least one of the individual molecules of the first plurality of different analytes.

5. The method of claim 4, wherein the fourth analyte comprises a competitor with the second analyte for interaction with at least one of the individual molecules of the first plurality of different analytes.

6. The method of claim 3, wherein at least one of the second and third analytes comprises a detectable label, and the characterizing step comprises detecting a presence or absence of the detectable label at the individually addressable locations on the array.

7. The method of claim 3, wherein at least one of the second and third analytes comprises a candidate binding ligand for the first plurality of individual analytes.

8. The method of claim 7, wherein the candidate binding ligand comprises a pharmaceutical compound.

9. The method of claim 7, wherein the candidate binding ligand comprises a polypeptide.

10. The method of claim 1, wherein the first plurality of different analytes comprises a plurality of different polypeptides.

11. The method of claim 10, wherein the plurality of different polypeptides comprises at least 1×10³ different polypeptides.

12. The method of claim 10, wherein the plurality of different polypeptides comprises at least 1×10⁴ different polypeptides.

13. The method of claim 10, wherein the plurality of different polypeptides comprises at least 1×10⁵ different polypeptides.

14. The method of claim 10, wherein each of the plurality of different polypeptides is coupled to an individually addressable location on the array through a chemical linker.

15. The method of claim 10, wherein each of the plurality of different polypeptides is coupled to an individually addressable location on the array through a non-covalent linker.

16. The method of claim 10, wherein each of the plurality of different polypeptides is coupled to an individually addressable location on the array through a nucleic acid molecule.

17. The method of claim 16, wherein each of the plurality of different polypeptides is coupled to an individually addressable location on the array through a structured nucleic acid particle.

18. The method of claim 1, wherein the individually addressable locations on the array are individually optically resolvable.

19. The method of claim 1, wherein the array comprises a plurality of wells disposed in a substrate, and each well is individually addressable.

20. A method of characterizing an interaction between analytes, comprising:
providing an array that comprises a plurality of first analytes disposed upon a surface of the array, wherein individual analyte molecules are located in individually addressable locations on the surface of the array;
identifying a plurality of first analytes at the individually addressable locations on the array surface;
contacting the surface of the array with one or more second analytes;
detecting the presence or absence of interaction between the first plurality of analytes and the one or more second analytes; and
characterizing an interaction between each of the individual analyte molecules of the plurality of first analytes and the one or more second analytes.
